(19) United States
(12) Patent Application Publication
(10) Pub. No.: US 2018/0148756 A1
(43) Pub. Date: May 31, 2018

(54) METHODS, COMPOSITIONS, AND KITS FOR NUCLEIC ACID ANALYSIS

(71) Applicant: TOMA Biosciences, Inc., Foster City, CA (US)

(72) Inventors: Austin SO, Pleasanton, CA (US); Michael Y. LUCERO, South San Francisco, CA (US)

(21) Appl. No.: 15/829,445

(22) Filed: Dec. 1, 2017

Related U.S. Application Data
(63) Continuation of application No. 14/769,047, filed on Aug. 19, 2015, now abandoned.
(60) Provisional application No. 61/767,718, filed on Feb. 21, 2013.
(60) Provisional application No. 61/769,683, filed on Feb. 26, 2013.
(60) Provisional application No. 61/777,702, filed on Mar. 12, 2013.
(60) Provisional application No. 61/780,578, filed on Mar. 13, 2013.
(60) Provisional application No. 61/824,894, filed on May 17, 2013.
(60) Provisional application No. 61/870,634, filed on Aug. 27, 2013.

(51) Int. Cl. C12P 19/34; C12Q 1/6806; C12Q 1/6886

(52) U.S. Cl. CPC C12P 19/34; C12Q 1/6806; C12Q 2600/158; C12Q 2600/106; C12Q 2535/122; C12Q 2600/156; C12Q 2521/501; C12Q 2525/204; C12Q 1/6886

(57) ABSTRACT

Aspects of the invention relate to methods and kits for assessing cancer. Some aspects of the invention relate to methods and kits for preparing a sample library for sequencing. Some aspects of the invention relate to methods and kits for allele detection. Some aspects of the invention relate to high efficiency ligation methods and kits. Some aspects of the invention relate to sensitive detection of amplicons.

### CROSS-REFERENCE

This application is a continuation application of U.S. application Ser. No. 14/769,047, filed Aug. 19, 2015, which is a National Stage of International Application Serial No. PCT/US2014/017832, filed Feb. 21, 2014, which claims the benefit of U.S. Provisional Application No. 61/767,718, filed Feb. 21, 2013, U.S. Provisional Application No. 61/769,683, filed Feb. 26, 2013, U.S. Provisional Application No. 61/777,702, filed Mar. 12, 2013, U.S. Provisional Application No. 61/780,578, filed Mar. 13, 2013, U.S. Provisional Application No. 61/824,894, filed May 17, 2013, and U.S. Provisional Application No. 61/870,634, filed Aug. 27, 2013, which applications are incorporated herein by reference.

### SEQUENCE LISTING

The instant application contains a Sequence Listing which has been submitted electronically in ASCII format and is hereby incorporated by reference in its entirety. Said ASCII copy, created on Feb. 19, 2014, is named 44288-703.601_SL.txt and is 1,580,104 bytes in size.

### BACKGROUND OF THE INVENTION

Cancer poses serious challenges for modern medicine. In 2007, it has been estimated that cancer caused about 13% of all human deaths worldwide (7.9 million). Cancer encompasses a broad group of various diseases, generally involving unregulated cell growth. In cancer, cells can divide and grow uncontrollably, can form malignant tumors, and can invade nearby parts of the body. Cancer can also spread to more distant parts of the body, for example, via the lymphatic system or bloodstream. There are over 200 different known cancers that afflict humans. Many cancers are associated with mutations, for example, mutations in cancer-related genes. The mutational status of a cancer can vary widely from one individual subject to another, and even from one tumor cell to another tumor cell in the same subject. Knowledge of these mutations can aid in the selection of cancer therapy, and can also aid in informing disease prognosis and/or disease status. A tumor biopsy can be sequenced to provide information on mutational status of cancer-related genes; however, procedures for tumor biopsies can be surgically invasive and costly to a patient. Furthermore, reliance on a tumor biopsy is of limited utility for monitoring cancer status of a subject if the subject has tumor cells that are difficult to biopsy (e.g., if a tumor is small).

The discovery that cell-free DNA floating in blood plasma and serum can harbor tumor-associated mutations opened up the possibility that analysis of cell-free DNA could aid in cancer diagnosis. However, analysis of cell-free tumor DNA, as currently practiced utilizes untargeted sequencing or complicated PCR amplification (e.g., on magnetic beads), resulting in high costs due to expensive reagents and systems.

The detection and/or measurement of mutations is widely practiced in the life sciences. For example, mutations such as single nucleotide polymorphisms (SNPs) are associated with a number of diseases, including, e.g., cancer, neurodegenerative diseases, infectious diseases, autoimmune diseases, anemia, and cystic fibrosis. Current methods for detecting mutations generally involve the amplification of target polynucleotides. For example, target-specific primers can selectively amplify regions suspected of harboring a mutation, and the resulting amplicons can be sequenced to interrogate the mutation. By way of other example, assays may utilize intercalating dyes that fluoresce in the presence of double stranded DNA (dsDNA), or may utilize Taqman probes designed to hybridize specifically to a particular polynucleotide sequence. These assays generally suffer from poor specificity and/or sensitivity, particularly for mutations affecting small nucleotide sequences (e.g., SNPs or small insertions/deletions).

A typical method of detection involves the use of detectable probes that are designed to bind to and enable detection of reaction products. Often, the detectable probe includes a detectable moiety and can further include a quencher moiety that inhibits the detectable moiety from emitting a detectable signal.

Such probe assays can utilize real-time PCR or endpoint digital PCR. Real-time PCR assays generally refer to assays in which detection occurs at each cycle of PCR. In endpoint digital PCR, template molecules are generally distributed across a large number of partitions, each containing the components required for amplification and detection. Following completion of PCR, the partitions can be interrogated individually for the presence or absence of a detectable signal which typically is generated during successful amplification. Digital PCR can permit simple counting statistics to be applied to obtain a very precise and accurate quantitation of a template nucleic acid of interest in a sample. Any means of generating a signal specific to a target polynucleotide of interest can be amenable to endpoint digital PCR.

Typically, the assays described above employ the use of probes that are designed to hybridize to a target polynucleotide during the PCR reaction. PCR reactions generally involve a thermal cycling process, in which a reaction mixture is subjected to repeated thermal cycles of controlled temperature shifts corresponding to distinct phases of the PCR reaction. The phases of a typical thermal cycle include (i) a denaturation phase, which typically involves heating the reaction mixture to a high temperature (e.g., 90-100° C.) in order to melt double-stranded nucleic acid into single stranded nucleic acid, (ii) an annealing phase, which typically involves lowering the reaction temperature to about 3→5 degrees Celsius below the Tm of the reaction primers (e.g., to about 55-65° C. in order to allow annealing of primers to a single-stranded template, and (iii) an extension phase, which typically involves bringing the reaction temperature to an optimal temperature for extension of a primer by a polymerase. An optimal extension phase temperature for, e.g., Taq polymerase, is about 72° C. During the extension phase, the polymerase typically synthesizes a new nucleic acid strand complementary to the template strand. PCR thermal cycling can be preceded by a “hot-start” phase, in which a reaction temperature is typically brought up to >90° C. in order to heat-activate a polymerase. Following thermal cycling, the reaction mixture can undergo a final extension phase to ensure that any remaining single-stranded nucleic acid is fully extended. Following thermal cycling (e.g., following a final extension phase), the temperature of the reaction mixture can be cooled to room temperature or lower to terminate the PCR reaction and stabilize the PCR products.

The annealing of a probe during a PCR reaction can impact primer annealing and extension, as full extension requires sufficient endonucleolytic activity of the polymerase in order to hydrolyze the annealed probe. Therefore, probe hybridization during a PCR reaction can negatively impact amplification efficiency and potentially affect the sensitivity and accuracy of the resulting data. Such issues can reduce the sensitivity of assays designed for the detection and quantitation of rare mutations, e.g., rare copy number variation events, rare SNPs, etc.

Furthermore, the assays described above typically employ the use of probes with a higher melting temperature (Tm) compared to the Tm of the PCR primers. Such high Tm probes are generally employed to favor hybridization of the probe to the target polynucleotide during PCR, to ensure that each PCR cycle will result in generation of a detectable signal. However, these Tm constraints make it difficult to design allele-specific probes with high discriminative ability, as the energy penalty of a probe/template mismatch is relatively small compared to the overall binding energy of the probe to the template. The above limitations can result in inaccurate mutation calling and hamper the detection and/or quantitation of mutant alleles.

A wide range of molecular biology applications involve the ligation of nucleic acids. Ligation is a ubiquitous molecular biology tool which is useful in the labeling of nucleic acids, molecular cloning applications, array hybridization, nucleic acid barcoding, and preparation of nucleic acid libraries (e.g., for sequencing). These applications are utilized for a wide variety of purposes, ranging from biotechnology to diagnostics, forensics, epidemiology, and research.

Ligation is generally catalyzed by a ligase enzyme. An exemplary ligation reaction is a nucleoside triphosphate-dependent ligation catalyzed by an nucleoside triphosphate-dependent ligase. A typical nucleoside triphosphate-dependent ligation generally occurs in a single reaction mixture comprising the nucleoside triphosphate-dependent ligase (E), a “donor” nucleic acid molecule, an “acceptor” nucleic acid molecule, ATP, and other reaction components. The ligation reaction can result in the formation of a covalent bond between the acceptor and the donor. The mechanism of nucleoside triphosphate-dependent ligation generally proceeds by three steps in the reaction mixture. A first step typically involves reversible transfer of a nucleoside monophosphate (NMP) to an active site of a ligase in the presence of the nucleoside triphosphate (NTP). For example, an adenosine monophosphate (AMP) can be transferred from ATP to an active site of the ligase (E), thereby releasing pyrophosphate (PPi) and activating the ligase. Alternatively, a guanosine monophosphate (GMP) can be transferred from GTP to an active site of the ligase (E).)

E+NTP E⇔NMP+PPi (1)

(wherein N=A,C,G,T)

The active ligase, in the presence of a 5′ or 3′ phosphorylated “donor” nucleic acid molecule, can then bind to the donor and reversibly transfer the NMP to the phosphate group of the donor, thereby adenylating the donor.

E−NMP+p-Donor+⇔E:NMP·P-Donor′ (2)

In the presence of an “acceptor” nucleic acid molecule comprising a 3′ or 5′ OH group, the ligase can then catalyze the formation of a phosphodiester bond between a phosphate of the NMP P of the donor and the OH group of the acceptor, thereby releasing NMP.

Acceptor-OH+E:NMP·P-Donor⇒Acceptor-Pi-Donor′+E+NMP (3)

While highly ubiquitous in several molecular biology applications, ligation is generally a highly inefficient process, resulting in yields <10%. Such inefficient processes result in significant loss of starting material, and can often necessitate additional steps (e.g., PCR expansion) to increase yield, thereby introducing the potential for errors associated with the additional steps and generally reducing quality, accuracy, and precision of results.

By way of example only, sequencing of polynucleotides is widely utilized in the life sciences for a wide variety of applications, such as biotechnology, diagnostics, forensics, and epidemiology. Sequencing can involve whole genome sequencing or targeted sequencing, in which genomic regions of interest are selectively captured from a sample prior to sequencing. Several target capture methodologies have been developed and integrated with high throughput sequencing systems, e.g., next-generation sequencing methods. Generally, targeted sequencing methods involve two separate steps, a target capture step and a library preparation step. Whole genome sequencing also generally involves a library preparation step. Preparation of a nucleic acid library for sequencing on a next-generation sequencing platform often involves ligation of two distinct adaptor oligonucleotides onto nucleic acid molecules, a multi-step process that has been predicted to result in about 1% of the nucleic acid molecules in a sample being correctly adapted. This major loss of nucleic acids, due to inefficient adaptor ligation, can result in zero representation of several genomic regions of interest in the resulting library. Furthermore, inefficient adaptor ligation can necessitate pre-amplification of library members to obtain enough material for sequencing to a desired read depth. This pre-amplification step has been shown to be a major source of bias in the final library, as inherent differences in PCR efficiency can result in over-representation of some genomic regions and underrepresentation of other genomic regions (see, e.g., Aird et al. Genome Biology 2011, 12:R18, hereby incorporated by reference in its entirety). Furthermore, pre-amplification of the nucleic acid library can introduce sequencing errors due to an intrinsic error rate of nucleic acid polymerases. The introduction of pre-amplification bias and errors resulting from pre-amplification can have deleterious consequences on diagnostic sequencing applications, in which the accurate detection of rare mutations from a limited starting sample is often desired.

### SUMMARY OF THE INVENTION

Aspects of the invention relate to methods and kits for assessing cancer. Some aspects of the invention relate to methods and kits for preparing a sample library for sequencing. Some aspects of the invention relate to methods and kits for allele detection. Some aspects of the invention relate to high efficiency ligation methods and kits. Some aspects of the invention relate to sensitive detection of amplicons.

In some instances, the invention provides a method of assessing cancer, comprising: (a) determining the presence, absence, and/or amount of each of a subset of genes in a sample derived from a sample from a subject, wherein the subset is determined by (i) performing targeted sequencing on a set of genes on a solid tissue sample from the subject wherein the solid tissue sample is known or suspected of comprising cancerous tissue; (ii) determining a profile of genetic abnormalities for the set of genes based on the sequencing; and (iii) selecting a subset of 2, 3, or 4, but no more than 4 genes of the set of genes based on the profile for the set, wherein the subset is specific to the individual; and (b) from the results of step (a) determining the status of the cancer in the subject.

The method can comprise (a) determining the presence, absence, and/or amount of each of a subset of genes in a sample derived from a fluid sample in a subject, wherein the subset is determined by (i) performing targeted sequencing on a set of genes from an unfixed solid tissue sample from the subject wherein the solid tissue sample is known or suspected of comprising cancerous tissue; (ii) determining a profile of genetic abnormalities for the set of genes based on the sequencing; and (iii) selecting a subset of the set of genes based on the profile for the set, wherein the subset is specific to the individual; and (b) from the results of step (a) determining the status of the cancer in the subject.

In a related embodiment, the method comprises (a) determining the presence, absence, and/or amount of each of a subset of genes in a sample derived from a fluid sample in a subject, wherein the subset is determined by (i) performing targeted sequencing on a set of genes from a bodily fluid sample from the subject wherein the bodily fluid sample is known or suspected of comprising tumor-derived nucleic acid; (ii) determining a profile of genetic abnormalities for the set of genes based on the sequencing; and (iii) selecting a subset of the set of genes based on the profile for the set, wherein the subset is specific to the individual; and (b) from the results of step (a) determining the status of the cancer in the subject.

In practicing any of the methods described herein, the set of genes comprises at least 10, 20, 30, 40, 50, 60, 70, 80, 90, 100, 150, 200, 300, 400, 500, 600, 700, 800, 900, or 1000 genes. The set of genes can be selected from the group consisting of: ABCA1, BRAF, CHD5, EP300, FLT1, ITPA, MYC, PIK3R1, SKP2, TP53, ABCA7, BRCA1, CHEK1, EPHA3, FLT3, JAK1, MYCL1, PIK3R2, SLC19A1, TP73, ABCB1, BRCA2, CHEK2, EPHA5, FLT4, JAK2, MYCN, PKHD1, SLC1A6, TPM3, ABCC2, BRIP1, CLTC, EPHA6, FN1, JAK3, MYH2, PLCB1, SLC22A2, TPMT, ABCC3, BUB1B, COL1A1, EPHA7, FOS, JUN, MYH9, PLCG1, SLCO1B3, TPO, ABCC4, Clorf144, COPS5, EPHA8, FOXO1, KBTBD11, NAV3, PLCG2, SMAD2, TPR, ABCG2, CABLES1, CREB1, EPHB1, FOXO3, KDM6A, NBN, PML, SMAD3, TRIO, ABL1, CACNA2D1, CREBBP, EPHB4, FOXP4, KDR, NCOA2, PMS2, SMAD4, TRRAP, ABL2, CAMKV, CRKL, EPHB6, GAB1, KIT, NEK11, PPARG, SMARCA4, TSC1, ACVR1B, CARD11, CRLF2, EPO, GATA1, KLF6, NF1, PPARGC1A, SMARCB1, TSC2, ACVR2A, CARM1, CSF1R, ERBB2, GLI1, KLHDC4, NF2, PPP1R3A, SMO, TTK, ADCY9, CAV1, CSMD3, ERBB3, GLI3, KRAS, NKX2-1, PPP2R1A, SOCS1, TYK2, AGAP2, CBFA2T3, CSNK1G2, ERBB4, GNA11, LMO2, NOS2, PPP2R1B, SOD2, TYMS, AKT1, CBL, CTNNA1, ERCC1, GNAQ, LRP1B, NOS3, PRKAA2, SOS1, UGT1A1, AKT2, CCND1, CTNNA2, ERCC2, GNAS, LRP2, NOTCH1, PRKCA, SOX10, UMPS, AKT3, CCND2, CTNNB1, ERCC3, GPR124, LRP6, NOTCH2, PRKCZ, SOX2, USP9×, ALK, CCND3, CYFIP1, ERCC4, GPR133, LTK, NOTCH3, PRKDC, SP1, VEGF, ANAPC5, CCNE1, CYLD, ERCC5, GRB2, MAN1B1, NPM1, PTCH1, SPRY2, VEGFA, APC, CD40LG, CYP19A1, ERCC6, GSK3B, MAP2K1, NQO1, PTCH2, SRC, VHL,APC2, CD44, CYP1B1, ERG, GSTP1, MAP2K2, NR3C1, PTEN, ST6GAL2, WRN, AR, CD79A, CYP2C19, ERN2, GUCY1A2, MAP2K4, NRAS, PTGS2, STAT1, WT1, ARAF, CD79B, CYP2C8, ESR1, HDAC1, MAP2K7, NRP2, PTPN11, STAT3, XPA, ARFRP1, CDC42, CYP2D6, ESR2, HDAC2, MAP3K1, NTRK1, PTPRB, STK11, XPC, ARID1A, CDC42BPB, CYP3A4, ETV4, HGF, MAPK1, NTRK2, PTPRD, SUFU, ZFY, ATM, CDC73, CYP3A5, EWSR1, HIF1A, MAPK3, NTRK3, RAD50, SULT1A1, ZNF521,ATP5A1, CDH1, DACH2, EXT1, HM13, MAPK8, OMA1, RAD51, SUZ12, ATR, CDH10, DCC, EZH2, HMGA1, MARK3, OR10R2, RAF1, TAF1, AURKA, CDH2, DCLK3, FANCA, HNF1A, MCL1, PAK3, RARA, TBX22, AURKB, CDH20, DDB2, FANCD2, HOXA3, MDM2, PARP1, RB1, TCF12, BAI3, CDH5, DDR2, FANCE, HOXA9, MDM4, PAX5, REM1, TCF3, BAP1, CDK2, DGKB, FANCF, HRAS, MECOM, PCDH15, RET, TCF4, BARD1, CDK4, DGKZ, FAS, HSP90AA1, MEN1, PCDH18, RICTOR, TEK, BAX, CDK6, DIRAS3, FBXW7, IDH1, MET, PCNA, RIPK1, TEP1, BCL11A, CDK7, DLG3, FCGR3A, IDH2, MITF, PDGFA, ROR1, TERT, BCL2, CDK8, DLL1, FES, IFNG, MLH1, PDGFB, ROR2, TET2, BCL2A1, CDKN1A, DNMT1, FGFR1, IGF1R, MLL, PDGFRA, ROS1, TGFBR2, BCL2L1, CDKN1B, DNMT3A, FGFR2, IGF2R, MLL3, PDGFRB, RPS6KA2, THBS1, BCL2L2, CDKN2A, DNMT3B, FGFR3, IKBKE, MPL, PDZRN3, RPTOR, TNFAIP3, BCL3, CDKN2B, DOT1L, FGFR4, IKZFl, MRE11A, PHLPP2, RSPO2, TNKS, BCL6, CDKN2C, DPYD, FH, IL2RG, MSH2, PIK3C3, RSPO3, TNKS2, BCR, CDKN2D, E2F1, FHOD3, INHBA, MSH6, PIK3CA, RUNX1, TNNI3K, BIRC5, CDX2, EED, FIGF, INSR, MTHFR, PIK3CB, SDHB, TNR, BIRC6, CEBPA, EGF, FLG2, IRS1, MTOR, PIK3CD, SF3B1, TOP1, BLM, CERK, EGFR, FLNC, IRS2, MUTYH, PIK3CG, SHC1, and TOP2A.

The sample can be selected from the group consisting of: blood, serum, plasma, urine, sweat, tears, saliva, sputum, components thereof or any combination thereof. Steps (a) and (b) can be performed at a plurality of time points to monitor the status of the cancer over time. One time point can be prior to a first administration of a cancer therapy and a subsequent time point can be subsequent to a first administration.

The method can further comprise generating a report communicating the profile of genetic abnormalities for the set of genes and communicating the report to a caregiver. The report can comprise a list of one or more therapeutic candidates based on the profile. The report can be generated within two weeks from collection of the solid tissue sample. In some instances, the report is generated within 1 week from collection of the solid tissue sample. In some embodiments, the report comprises copy number alterations of the set of genes. In some embodiments, the report comprises a description of a therapeutic agent targeting an abnormality. The method can further comprise generating a report communicating the profile of the subset of genes at each of the plurality of time points.

In some embodiments of any of the methods herein, the determining comprises the step of diluting nucleic acid molecules from the sample into discrete reaction volumes, wherein the discrete reaction volumes contain on average less than 10, 5, 4, 3, 2, or 1 nucleic acid molecule from the sample. In some embodiments the discrete reaction volumes contain 0-10 molecules of the nucleic acid from the sample. The discrete reaction volumes can be droplets in an emulsion. The discrete reaction volumes can further comprise primers for allelic discrimination of the genetic abnormalities in the subset of genes

Determining the status can comprise quantifying the number of nucleic acids harboring the genetic abnormalities in the subset of genes. The step of targeted sequencing can comprise preparing a DNA library from the solid tissue sample in less than 8, 7, 6, 5, or 4 hours. In some embodiments, preparing does not require exponential PCR amplification prior to sequencing of the library. In some embodiments the preparing comprises a linear amplification step. In some embodiments the preparing does not require amplification.

In some embodiments, the step of targeted sequencing comprises (a) contacting a single-stranded DNA fragment from the solid tissue sample with a target-specific oligonucleotide comprising (i) a region specific for a region of a cancer-related gene and (ii) an adaptor sequence specific for coupling to a sequencing platform; (b) performing a hybridization reaction to join the target specific oligonucleotides to a single-stranded DNA fragment containing a region of complementarity to the target-specific oligonucleotide; (c) performing an extension reaction to create an extension product comprising the region and comprising the adaptor; and (d) sequencing the extension product. Contacting can occur with the target-specific oligonucleotide attached to a sequencing platform. Contacting can occur with the target-specific oligonucleotide free in a solution.

In some aspects, the present invention provides methods and kits for the sensitive detection of a mutation in a target polynucleotide. The invention provides an oligonucleotide primer, comprising a probe-binding region and a template binding region. In some embodiments, the template binding region is at least 50% complementary to a template nucleic acid suspected of harboring a mutation. In some embodiments, a portion of the template binding region at least partially overlays a locus of the suspected mutation. In some embodiments, the oligonucleotide primer upon hybridization to the template nucleic acid is extendable by a polymerase if the mutation is present but is not extendable by the polymerase if the mutation is not present. In some embodiments, the template binding region comprises a 3′ terminal region that overlays the mutation locus. In some embodiments, the 3′ terminal region that overlays the mutation locus comprises 1, 2, 3, 4, 5, or more than 5 bases of the 3′-end of the template binding region. In some embodiments, the mutation is a single nucleotide polymorphism (SNP).

In particular embodiments, the 3′ terminal region comprises a base that overlays the SNP locus. In some embodiments, the base is complementary to a mutant allele of the SNP locus. In some embodiments, the base is complementary to a wild-type allele of the SNP locus. In some embodiments, the probe-binding region does not hybridize to any genomic sequence from the subject. In some embodiments, the polymerase is a DNA polymerase lacking 3′ to 5′ exonuclease activity.

The invention also provides a kit comprising: (a) an oligonucleotide primer, wherein the oligonucleotide primer comprises (i) a probe-binding region and a template binding region that is at least 70% complementary to a template nucleic acid suspected of harboring a mutation, wherein a portion of the template binding region at least partially overlays locus of the suspected mutation, wherein the oligonucleotide primer upon hybridization to the template nucleic acid is extendable by a polymerase if the mutation is present but is not extendable by the polymerase if the mutation is not present; and (b) instructions for use. In some embodiments, the mutation is a single nucleotide polymorphism (SNP). In some embodiments, the template binding region comprises a 3′ terminal base that overlays the SNP locus. In some embodiments, the 3′ terminal base is complementary to a mutant allele of the SNP locus. In some embodiments, the 3′ terminal base is complementary to a wild-type allele of the SNP locus. In some embodiments, the probe-binding region does not hybridize to any genomic sequence from the subject. In some embodiments, the kit further comprises a reporter probe that is at least 70% complementary to the probe binding region. In some embodiments, the reporter probe comprises a detectable moiety and a quencher moiety, wherein the quencher moiety suppresses detection of the detectable moiety when the reporter probe is intact. In some embodiments, the kit further comprises a reverse primer that is at least 70% complementary to a reverse complement sequence downstream of the locus. In some embodiments, the kit further comprises a polymerase.

In some embodiments, the polymerase is a thermostable polymerase having a 5′ to 3′ exonuclease activity and not having a 3′ to 5′ exonuclease activity. In some embodiments, the kit further comprises (i) one or more alternative oligonucleotide primers, wherein the one or more alternative oligonucleotide primers each comprises a distinct probe binding region and a template binding region that is at least 70% complementary to the template nucleic acid, wherein a portion of the template binding region at least partially overlays the locus, wherein the alternative oligonucleotide primer upon hybridization to the template nucleic acid is extendable by a polymerase if an alternative allele is present but is not extendable by the polymerase if the alternative allele is not present. In some embodiments, the kit further comprises one or more alternative reporter probes, wherein each of the alternative reporter probes is at least 70% complementary to one of the distinct probe binding regions but not to any other probe binding region of the kit. In some embodiments, each of the alternative reporter probes comprises an alternative detectable moiety and a quencher moiety, wherein each of the detectable moieties of the kit is detectably distinct from any other detectable moiety of the kit. In some embodiments, a hybridization product consisting of the oligonucleotide primer and reporter probe has a Tm that is at least 10 degrees higher than a Tm of a hybridization product consisting of the oligonucleotide primer and the template nucleic acid.

In another aspect, the invention provides a method of detecting a mutation in a target polynucleotide region, comprising: (a) selectively hybridizing an oligonucleotide primer to the target polynucleotide region, wherein the oligonucleotide primer comprises (i) a probe-binding region, and (ii) a template binding region that is at least 70% complementary to a template nucleic acid suspected of harboring a mutation, wherein a portion of the template binding region at least partially overlays a locus of the suspected mutation, and wherein the oligonucleotide primer upon hybridization to the template nucleic acid is extendable by a polymerase if the mutation is present but is not extendable by the polymerase if the mutation is not present; (b) extending the hybridized oligonucleotide primer to form an extension product; and (c) detecting the extension product, whereby the detecting indicates the presence of the mutation. In some embodiments, extending comprises extending with a DNA polymerase that does not comprise 3′ to 5′ exonuclease activity.

In some embodiments, detecting comprises selectively hybridizing a reporter probe to the probe binding region. In some embodiments, the reporter probe comprises a detectable moiety and a quencher moiety, wherein the quencher moiety suppresses detection of the detectable moiety when the reporter probe is intact. In some embodiments, detecting further comprises separating the detectable moiety from the quencher moiety of the hybridized reporter probe. In some embodiments, the method further comprises amplifying the extension product with a reverse primer that is capable of hybridizing to a region of the extension product downstream of the locus. In some embodiments, amplifying comprises amplifying with a DNA polymerase that comprises 5′ to 3′ exonuclease activity. In some embodiments, the method further comprises selectively hybridizing one or more alternative oligonucleotide primers to the target polynucleotide region, wherein the one or more alternative oligonucleotide primers each comprises a distinct probe binding region and a template binding region that is at least 70% complementary to the template nucleic acid, wherein a portion of the template binding region at least partially overlays the locus, wherein the alternative oligonucleotide primer upon hybridization to the template nucleic acid is extendable by a polymerase if an alternative allele is present but is not extendable by the polymerase if the alternative allele is not present. In some embodiments, detecting further comprises selectively hybridizing one or more alternative reporter probes to the one or more alternative oligonucleotide primers, wherein each of the alternative reporter probes is at least 70% complementary to one of the distinct probe binding regions but not to any other of the probe binding regions. In some embodiments, each of the alternative reporter probes comprises an alternative detectable moiety and a quencher moiety, wherein each of the alternative detectable moieties is detectably distinct from any other of the detectable moieties. In some embodiments, the mutation is a single nucleotide polymorphism (SNP). In some embodiments, the template binding region comprises a 3′ terminal region comprising a base that overlays the SNP locus. In some embodiments, wherein the base is complementary to a mutant allele of the SNP locus.

In some embodiments, the base is complementary to a wild-type allele of the SNP locus. In some embodiments, the probe-binding region does not hybridize to the target polynucleotide region. In some embodiments, a hybridization product of the oligonucleotide primer and reporter probe has a Tm that is at least 10 degrees higher than a Tm of a hybridization product between the oligonucleotide primer and target polynucleotide. In some embodiments, a concentration of the reporter probe is at least 10× a concentration of the forward primer. In some embodiments, the nucleic acid sample is subdivided into a plurality of discrete reaction volumes prior to steps b-c. In some embodiments, the method further comprises detection of the detectable moiety in each of the reaction volumes. In some embodiments, the method further comprises counting a number of the reaction volumes wherein the detectable moiety is detected. In some embodiments, the nucleic acid sample is subdivided such that the plurality of discrete reaction volumes contain an average of <1, 1, or more than 1 template nucleic acid molecule. In some embodiments, the method further comprises providing a conclusion and transmitting the conclusion over a network.

The invention also provides a composition comprising (a) an oligonucleotide primer hybridized to a template nucleic acid, wherein the template nucleic acid comprises a wild-type allele at a locus, wherein the 3′ terminal region of the oligonucleotide primer overlays the locus and is not complementary to the wild-type allele; and (b) an intact reporter probe comprising a detectable and quencher moiety, wherein the intact reporter probe is hybridized to the oligonucleotide primer.

The invention also provides a method, comprising: (a) hybridizing a target-selective oligonucleotide (TSO) to a single-stranded DNA (ssDNA) fragment in an ssDNA library to create a hybridization product; and (b) extending the hybridization product to create a double stranded extension product, wherein the TSO comprises (i) a sequence that is complementary to a single target region and (ii) a first single-stranded adaptor sequence located at a first end of the TSO but not to both ends of the TSO, and wherein the ssDNA fragment comprises a second single-stranded adaptor sequence but does not comprise the first single-stranded adaptor sequence. In some embodiments, the ssDNA fragment is ligated to a second single-stranded adaptor sequence by a ligation method comprising over 10%, 50%, 70%, or 90% ligation efficiency. In some embodiments, the ssDNA fragment is ligated to a second single-stranded adaptor sequence by a single-stranded ligation method. In some embodiments, the second single-stranded adaptor sequence is located at a first end of the ssDNA fragment but not at both ends of the ssDNA fragment. In some embodiments, the amplifying comprises linear amplification. In some embodiments, the second single-stranded adaptor sequence is located at a first end of the ssDNA fragment but not at both ends of the ssDNA fragment. In some embodiments, the first end of the ssDNA fragment is a 5′ end. In some embodiments, the first adaptor sequence comprises a barcode sequence. In some embodiments, the first or second adaptor sequence comprises a barcode sequence. In some embodiments, the first end of the TSO is a 5′ end. In some embodiments, the first or second adaptor sequence comprises a sequence that is at least 70% identical to a support-bound oligonucleotide conjugated to a solid support. In some embodiments, the solid support is coupled to a sequencing platform. In some embodiments, the first or second adaptor sequence comprises a binding site for a sequencing primer. In some embodiments, the method further comprises annealing the extension products to the support-bound oligonucleotides. In some embodiments, the method further comprises amplifying the annealed extension products. In some embodiments, the method further comprises sequencing the annealed extension products. In some embodiments, the ssDNA library comprises genomic DNA fragments. In some embodiments, the ssDNA library comprises cDNA fragments. In some embodiments, the method further comprises removing unhybridized TSOs and unhybridized ssDNA library members. In some embodiments, steps (a) and (b) are performed when the ssDNA library members and the TSOs are free-floating in a solution.

In some embodiments, the single target region flanks a genomic region. In some embodiments, the genomic region comprises a portion of an exon region from a cancer-related gene. In some embodiments, the cancer-related gene is selected from the group consisting of ABCA1, BRAF, CHD5, EP300, FLT1, ITPA, MYC, PIK3R1, SKP2, TP53, ABCA7, BRCA1, CHEK1, EPHA3, FLT3, JAK1, MYCL1, PIK3R2, SLC19A1, TP73, ABCB1, BRCA2, CHEK2, EPHA5, FLT4, JAK2, MYCN, PKHD1, SLC1A6, TPM3, ABCC2, BRIP1, CLTC, EPHA6, FN1, JAK3, MYH2, PLCB1, SLC22A2, TPMT, ABCC3, BUB1B, COL1A1, EPHA7, FOS, JUN, MYH9, PLCG1, SLCO1B3, TPO, ABCC4, Clorf144, COPS5, EPHA8, FOXO1, KBTBD11, NAV3, PLCG2, SMAD2, TPR, ABCG2, CABLES1, CREB1, EPHB1, FOXO3, KDM6A, NBN, PML, SMAD3, TRIO, ABL1, CACNA2D1, CREBBP, EPHB4, FOXP4, KDR, NCOA2, PMS2, SMAD4, TRRAP, ABL2, CAMKV, CRKL, EPHB6, GAB1, KIT, NEK11, PPARG, SMARCA4, TSC1, ACVR1B, CARD11, CRLF2, EPO, GATA1, KLF6, NF1, PPARGC1A, SMARCB1, TSC2, ACVR2A, CARM1, CSF1R, ERBB2, GLI1, KLHDC4, NF2, PPP1R3A, SMO, TTK, ADCY9, CAV1, CSMD3, ERBB3, GLI3, KRAS, NKX2-1, PPP2R1A, SOCS1, TYK2, AGAP2, CBFA2T3, CSNK1G2, ERBB4, GNA11, LMO2, NOS2, PPP2R1B, SOD2, TYMS, AKT1, CBL, CTNNA1, ERCC1, GNAQ, LRP1B, NOS3, PRKAA2, SOS1, UGT1A1, AKT2, CCND1, CTNNA2, ERCC2, GNAS, LRP2, NOTCH1, PRKCA, SOX10, LIMPS, AKT3, CCND2, CTNNB1, ERCC3, GPR124, LRP6, NOTCH2, PRKCZ, SOX2, USP9×, ALK, CCND3, CYFIP1, ERCC4, GPR133, LTK, NOTCH3, PRKDC, SP1, VEGF, ANAPC5, CCNE1, CYLD, ERCC5, GRB2, MAN1B1, NPM1, PTCH1, SPRY2, VEGFA, APC, CD40LG, CYP19A1, ERCC6, GSK3B, MAP2K1, NQO1, PTCH2, SRC, VHL,APC2, CD44, CYP1B1, ERG, GSTP1, MAP2K2, NR3C1, PTEN, ST6GAL2, WRN, AR, CD79A, CYP2C19, ERN2, GUCY1A2, MAP2K4, NRAS, PTGS2, STAT1, WT1, ARAF, CD79B, CYP2C8, ESR1, HDAC1, MAP2K7, NRP2, PTPN11, STAT3, XPA, ARFRP1, CDC42, CYP2D6, ESR2, HDAC2, MAP3K1, NTRK1, PTPRB, STK11, XPC, ARID1A, CDC42BPB, CYP3A4, ETV4, HGF, MAPK1, NTRK2, PTPRD, SUFU, ZFY, ATM, CDC73, CYP3A5, EWSR1, HIF1A, MAPK3, NTRK3, RAD50, SULT1A1, ZNF521,ATP5A1, CDH1, DACH2, EXT1, HM13, MAPK8, OMA1, RAD51, SUZ12, ATR, CDH10, DCC, EZH2, HMGA1, MARK3, OR1OR2, RAF1, TAF1, AURKA, CDH2, DCLK3, FANCA, HNF1A, MCL1, PAK3, RARA, TBX22, AURKB, CDH20, DDB2, FANCD2, HOXA3, MDM2, PARP1, RB1, TCF12, BAI3, CDH5, DDR2, FANCE, HOXA9, MDM4, PAX5, REM1, TCF3, BAP1, CDK2, DGKB, FANCF, HRAS, MECOM, PCDH15, RET, TCF4, BARD1, CDK4, DGKZ, FAS, HSP90AA1, MEN1, PCDH18, RICTOR, TEK, BAX, CDK6, DIRAS3, FBXW7, IDH1, MET, PCNA, RIPK1, TEP1, BCL11A, CDK7, DLG3, FCGR3A, IDH2, MITF, PDGFA, ROR1, TERT, BCL2, CDK8, DLL1, FES, IFNG, MLH1, PDGFB, ROR2, TET2, BCL2A1, CDKN1A, DNMT1, FGFR1, IGF1R, MLL, PDGFRA, ROS1, TGFBR2, BCL2L1, CDKN1B, DNMT3A, FGFR2, IGF2R, MLL3, PDGFRB, RPS6KA2, THBS1, BCL2L2, CDKN2A, DNMT3B, FGFR3, IKBKE, MPL, PDZRN3, RPTOR, TNFAIP3, BCL3, CDKN2B, DOT1L, FGFR4, IKZF1, MRE11A, PHLPP2, RSPO2, TNKS, BCL6, CDKN2C, DPYD, FH, IL2RG, MSH2, PIK3C3, RSPO3, TNKS2, BCR, CDKN2D, E2F1, FHOD3, INHBA, MSH6, PIK3CA, RUNX1, TNNI3K, BIRC5, CDX2, EED, FIGF, INSR, MTHFR, PIK3CB, SDHB, TNR, BIRC6, CEBPA, EGF, FLG2, IRS1, MTOR, PIK3CD, SF3B1, TOP1, BLM, CERK, EGFR, FLNC, IRS2, MUTYH, PIK3CG, SHC1, and TOP2A.

In some embodiments, the ligation method with over 10%, 50%, 70%, or 90% efficiency is a single-stranded ligation method. In some embodiments, the ligation method comprises uses of an RNA ligase. In some embodiments, the RNA ligase is CircLigase or CircLigase II.

The invention also provides a method of preparing a single-stranded DNA library, comprising: (a) denaturing a double stranded DNA fragment into single stranded DNA (ssDNA) fragments; (b) removing 5′ phosphates from the ssDNA fragments; (c) ligating single-stranded primer docking oligonucleotides (pdo's) to 3′ ends of the ssDNA fragments, (d) hybridizing primers to the pdo's, wherein the primers comprise a sequence complementary to the adaptor oligonucleotide sequence and comprise a first adaptor sequence that is at least 70% identical to a support-bound oligonucleotide coupled to a sequencing platform; (e) extending the hybridized primers to create duplexes, wherein each duplex comprises an ss fragment and an extended primer strand; (f) denaturing the double-stranded extension product, wherein the denaturing results in release of the extended primer strands from the immobilized capturing reagent and retention of the ssDNA fragments on the immobilized capturing reagent; and (g) collecting the extended primer strands. In some embodiments, the method comprises repeating steps d-f in a linear amplification reaction, wherein the extended primer strands comprise the ss DNA library. In some embodiments, step (c) results in ligation of at least 50% of the ssDNA fragments to the pdo's. In some embodiments, the ligating is performed using an ATP-dependent ligase. In some embodiments, the ATP-dependent ligase is an RNA ligase. In some embodiments, the RNA ligase is CircLigase or CircLigase II. In some embodiments, the pdo's are adenylated. In some embodiments, the extending is performed using a proofreading DNA polymerase.

The invention also provides a method of preparing a single-stranded DNA library, comprising: denaturing a double stranded DNA fragment into single stranded DNA (ssDNA) fragments; ligating a first single-stranded adaptor sequence to a first end of the ssDNA fragments; and ligating a second single-stranded adaptor sequence to a second end of the ssDNA fragments.

The invention also provides a kit, comprising: a primer docking oligonucleotide (pdo); a primer, wherein the primer comprises a sequence that is at least 70% complementary to the pdo sequence and further comprises a first adaptor sequence that is at least 70% identical to a first support-bound oligonucleotide coupled to a sequencing platform; and instructions for use. In some embodiments, the kit further comprises an ATP-dependent ligase. In some embodiments, the ATP-dependent ligase is an RNA ligase. In some embodiments, the RNA ligase is CircLigase or CircLigase II. In some embodiments, the kit further comprises a proofreading DNA polymerase. In some embodiments, the kit further comprises the immobilized capturing reagent. In some embodiments, the first adaptor sequence comprises a sequence that is at least 70% complementary to a first sequencing primer. In some embodiments, the first adaptor sequence comprises a barcode sequence. In some embodiments, the kit further comprises a target-selective oligonucleotide (TSO). In some embodiments, the TSO further comprises a second adaptor sequence located at a first end of the TSO but not a second end of the TSO. In some embodiments, the first end of the TSO is a 5′ end. In some embodiments, the second adaptor sequence comprises a sequence that is at least 70% identical to a second support-bound oligonucleotide coupled to a sequencing platform. In some embodiments, the second adaptor sequence comprises a binding site for a sequencing primer.

The invention also provides a kit, comprising: a first adaptor oligonucleotide, wherein the first adaptor comprises a sequence that is at least 70% complementary to a first support-bound oligonucleotide coupled to a sequencing platform; a second adaptor oligonucleotide, wherein the second adaptor comprises a sequence that is distinct from the first adaptor oligonucleotide; an RNA ligase; and instructions for use. In some embodiments, the second adaptor comprises a sequence that is at least 70% complementary to a sequencing primer. In some embodiments, the second adaptor comprises a sequence that is at least 70% complementary to a second support-bound oligonucleotide coupled to a sequencing platform. In some embodiments, the first adaptor comprises a sequence that is at least 70% complementary to a sequencing primer. In some embodiments, one of the first or second adaptor comprises a barcode sequence. In some embodiments, the first adaptor comprises a 3′terminal blocking group that prevents the formation of a covalent bond between the 3′ terminal base and another nucleotide. In some embodiments, the 3′ terminal blocking group is dideoxy-dNTP, alkyl, amino-alkyl, fluorophore digeoxygenin, or biotin. In some embodiments, the first adaptor comprises a 5′polyadenylation sequence. In some embodiments, the RNA ligase is truncated or mutated ligase 2 from T4 or Mth. In some embodiments, the kit further comprises a second RNA ligase. In some embodiments, the second RNA ligase is CircLigase or CircLigase II.

The invention provides methods and kits for conducting a high-efficiency ligation reactions. Such methods and kits can be used for a wide range of applications.

The invention provides a method of conducting a high-efficiency ligation reaction, comprising ligating a plurality of acceptor nucleic acid molecules to a first end of at least 10%, 20%, 30%, 40%, 50%, 60%, 70%, 80%, or 90% of a plurality of donor nucleic acid molecules. In some embodiments, the plurality of donor nucleic acid molecules is present in a reaction mixture at a concentration of >10 nM.

In another aspect, the invention provides a method of conducting a high-efficiency ligation reaction, comprising ligating a plurality of acceptor nucleic acid molecules to a first end of over 10%, 20%, 30%, 40%, 50%, 60%, 70%, 80%, or 90% of a plurality of donor nucleic acid molecules, wherein one of the donor or acceptor nucleic acid molecules is >120 nt long.

In another aspect, the invention provides a method of conducting a high-efficiency ligation reaction, comprising ligating a plurality of donor nucleic acid molecules to a first end of at least 10%, 20%, 30%, 40%, 50%, 60%, 70%, 80%, or 90% of a plurality of acceptor nucleic acid molecules. In some embodiments, the plurality of donor nucleic acid molecules is present in a reaction mixture at a concentration of >10 nM.

In another aspect, the invention provides a method of conducting a high-efficiency ligation reaction, comprising ligating a plurality of donor nucleic acid molecules to a first end of at least 10%, 20%, 30%, 40%, 50%, 60%, 70%, 80%, or 90% of a plurality of acceptor nucleic acid molecules, wherein one of the donor or acceptor nucleic acid molecules is >120 nt long.

In some embodiments of the high efficiency ligation methods, the acceptor nucleic acid molecules are the donor nucleic acid molecules. In some embodiments, the method comprises (a) transferring a nucleoside monophosphate (NMP) to an amount of a donor nucleic acid molecules in a reaction mixture for a time sufficient to effect an accumulation of NMP-carrying donor nucleic acid molecules; and (b) effecting formation of a covalent bond between an NMP-carrying donor nucleic acid molecules and an acceptor nucleic acid molecule, wherein steps (a) and (b) are carried out sequentially in the reaction mixture. In some embodiments, the transferring results in transfer of an NMP to at least 10%, 20%, 30%, 40%, 50%, 60%, 70%, 80%, or 90% of the donor nucleic acid molecules. In some embodiments, a 3′ terminal region of at least one member of the donor nucleic acid molecules is an unmodified 3′ terminal region. In some embodiments, the reaction mixture comprises (a) an amount of an nucleoside triphosphate (NTP)-dependent ligase that is at least equimolar to the amount of donor nucleic acid molecules; and (b) NTP that is present in an amount that is at least 10-fold higher than a Michaelis constant (Km) of the NTP-dependent ligase. In some embodiments, the NTP-dependent ligase is an RNA ligase. In some embodiments the NTP-dependent ligase is an ATP-dependent RNA ligase. In some embodiments, the RNA ligase is a thermophilic RNA ligase. In some embodiments, the RNA ligase is T4 RNA ligase. In some embodiments, the ATP-dependent RNA ligase is MthRn1, CircLigase, or CircLigase II. In some embodiments the NTP-dependent ligase is a GTP-dependent ligase, e.g., is RTcB. In some embodiments, a 3′ terminal region of a donor nucleic acid molecule is modified with a 3′ terminal blocking group. In some embodiments, wherein effecting formation of a covalent bond comprises adding to the reaction mixture: the acceptor nucleic acid molecule; and Mn²⁺. In some embodiments, the Mn²⁺ is present in an amount that is at least 2.5 mM. In some embodiments, the method further comprises reducing concentration of the NTP in the reaction mixture. In some embodiments, reducing concentration comprises reducing concentration of the NTP by at least 1.5 fold, 2-fold, 3-fold, 4-fold, 5-fold, 6-fold, 7-fold, 8-fold, 9-fold, or 10-fold. In some embodiments, reducing concentration comprises adding to the reaction mixture an amount of liquid sufficient to dilute the NTP at least 1.5 fold, 2-fold, 3-fold, 4-fold, 5-fold, 6-fold, 7-fold, 8-fold, 9-fold, or 10-fold. In some embodiments, the donor nucleic acid molecules comprises nucleic acid molecules isolated from a biological source and wherein the acceptor nucleic acid molecules comprises an adaptor sequence. In some embodiments, the acceptor nucleic acid molecules comprises nucleic acid isolated from a biological subject and wherein the donor nucleic acid molecules comprises an adaptor sequence. In some embodiments, the acceptor nucleic acid molecules comprises nucleic acid isolated from a biological subject and wherein the donor nucleic acid molecules comprises a barcode sequence. In some embodiments, the donor nucleic acid molecules comprises nucleic acid isolated from a biological subject and wherein the acceptor nucleic acid molecules comprises a barcode sequence. In some embodiments, the acceptor nucleic acid molecules or donor nucleic acid molecules comprise a detectable tag. In some embodiments, the NMP is AMP. In some embodiments, the NMP is GMP. In some embodiments, the NTP is ATP. In some embodiments, the NTP is GTP.

In another aspect, the invention provides a method of preparing a nucleic acid library, comprising ligating an oligonucleotide sequence to a first end of at least 10%, 20%, 30%, 40%, 50%, 60%, 70%, 80%, or 90% of a plurality of template nucleic acid molecules to create the nucleic acid library, wherein one of the template nucleic acid molecules is >120 nt long. In some embodiments, the oligonucleotide sequence is an adaptor sequence. In some embodiments, the method further comprises sequencing the nucleic acid library. In some embodiments, the oligonucleotide sequence comprises a detectable label. In some embodiments, the method comprises analyzing the nucleic acid library by array hybridization.

In one aspect, the invention provides a method of preparing a nucleic acid library, comprising (a) ligating an adaptor sequence to a first end of at least 10%, 20%, 30%, 40%, 50%, 60%, 70%, 80%, or 90% of a plurality of template nucleic acid molecules to create the nucleic acid library; and (b) sequencing the nucleic acid library. In some embodiments, sequencing is performed without pre-amplification of the nucleic acid library. In some embodiments, the plurality of template nucleic acid molecules comprises genomic DNA (gDNA). In some embodiments, the gDNA is isolated from a solid tissue sample. In some embodiments, the gDNA is isolated from plasma, serum, sputum, saliva, urine, or sweat. In some embodiments, the plurality of template nucleic acid molecules comprises single-stranded nucleic acid fragments. In some embodiments, the method comprises ligating an adaptor sequence to a first end of at least 50%, 60%, 70%, 80%, 90%, 95% of the plurality of template nucleic acid molecules.

In some embodiments, the ligating comprises the steps of: (a) transferring a NMP to an amount of a first population of nucleic acids (reactant 1) in a first reaction mixture for a time sufficient to effect an accumulation of NMP-carrying reactant 1; and (b) effecting formation of a covalent bond between the NMP-carrying reactant 1 and a second population of nucleic acids (reactant 2), wherein the reactant 1 is either (i) the plurality of template nucleic acids or (ii) the sequencing adaptor, wherein the reactant 2 is the other of (i) the plurality of template nucleic acids or (ii) the sequencing adaptor, and wherein the adenylated reactant 1 is not purified prior to the effecting formation of a covalent bond. In some embodiments, the transferring results in transfer of NMP to at least 10%, 20%, 30%, 40%, 50%, 60%, 70%, 80%, or 90% of reactant 1. In some embodiments, a 3′ terminal region of at least one member of the reactant 1 is an unmodified 3′ terminal region. In some embodiments, the first reaction mixture comprises (a) an amount of an NTP-dependent ligase that is at least equimolar to the amount of reactant 1; and (b) NTP that is present in an amount that is at least 10-fold higher than a Michaelis constant (Km) of the NTP-dependent ligase. The NTP-dependent ligase can be any of the foregoing NTP-dependent ligases. In some embodiments, the NTP-dependent ligase is an RNA ligase. In some embodiments, the RNA ligase is a thermophilic RNA ligase. In some embodiments the NTP dependent ligase is an ATP dependent RNA ligase. In some embodiments the ATP dependent RNA ligase is MthRn1, T4 RNA ligase, CircLigase, or CircLigase II. In some embodiments, the NTP-dependent ligase is a GTP dependent ligase. The GTP-dependent ligase can be RtcB. In some embodiments, a 3′ terminal region of at least one member of reactant 1 is modified with a 3′ terminal blocking group. In some embodiments, effecting formation of a covalent bond comprises adding to the first reaction mixture: a cation; the reactant 2; and a liquid in an amount sufficient to dilute the NTP at least 10-fold. In some embodiments, the cation is Mn²⁺. In some embodiments, the method further comprises ligating a second adaptor sequence to a second end of at least 10%, 20%, 30%, 40%, 50%, 60%, 70%, 80%, or 90% of the plurality of template nucleic acid molecules. In some embodiments, the method further comprises (a) hybridizing a target-selective oligonucleotide (tso) to a member of the DNA library, wherein the target-selective oligonucleotide comprises (i) a sequence specific for a region of gDNA and (ii) a second adaptor sequence; and (b) extending the hybridized tso to create a double-stranded library member comprising the first and second adaptor. In some embodiments, the tso comprises a sequence having at least 70% identity or complementarity to a region of a cancer-related gene. In some embodiments, the sequencing comprises massively parallel sequencing. In some embodiments, the ligating is performed using a reaction protocol that can be performed in less than 3 hours.

In another aspect, the invention provides kits for performing a high efficiency ligation. In some embodiments, the kit comprises an NTP-dependent ligase; a cation; NTP; and instructions for carrying out any of the methods described herein.

The invention also provides a method of tracking tumor-specific mutations using tumor genomic DNA (gDNA) isolated from a subject's tumor and normal gDNA isolated from non-tumor tissue from the subject; comprising: (a) sequencing a DNA library prepared from the tumor gDNA without pre-amplification to produce a first dataset; (b) sequencing a DNA library prepared from the normal gDNA without pre-amplification to produce a second dataset; (c) analyzing the first and second dataset to identify one or more tumor-specific mutations in the subject; and (d) detecting the presence or absence of the tumor-specific mutations in cell-free DNA isolated from a liquid sample from the subject. In some embodiments, the liquid sample is selected from the group consisting of plasma, serum, sputum, saliva, urine, and sweat. In some embodiments, the DNA library of step (a) or (b) is prepared using any of the methods described herein. In some embodiments, the sequencing comprises sequencing at least 200 cancer-related In some embodiments, the cancer-related genes are selected from the group consisting of ABCA1, BRAF, CHD5, EP300, FLT1, ITPA, MYC, PIK3R1, SKP2, TP53, ABCA7, BRCA1, CHEK1, EPHA3, FLT3, JAK1, MYCL1, PIK3R2, SLC19A1, TP73, ABCB1, BRCA2, CHEK2, EPHA5, FLT4, JAK2, MYCN, PKHD1, SLC1A6, TPM3, ABCC2, BRIP1, CLTC, EPHA6, FN1, JAK3, MYH2, PLCB1, SLC22A2, TPMT, ABCC3, BUB1B, COL1A1, EPHA7, FOS, JUN, MYH9, PLCG1, SLCO1B3, TPO, ABCC4, Clorf144, COPS5, EPHA8, FOXO1, KBTBD11, NAV3, PLCG2, SMAD2, TPR, ABCG2, CABLES1, CREB1, EPHB1, FOXO3, KDM6A, NBN, PML, SMAD3, TRIO, ABL1, CACNA2D1, CREBBP, EPHB4, FOXP4, KDR, NCOA2, PMS2, SMAD4, TRRAP, ABL2, CAMKV, CRKL, EPHB6, GAB1, KIT, NEK11, PPARG, SMARCA4, TSC1, ACVR1B, CARD11, CRLF2, EPO, GATA1, KLF6, NF1, PPARGC1A, SMARCB1, TSC2, ACVR2A, CARM1, CSF1R, ERBB2, GLI1, KLHDC4, NF2, PPP1R3A, SMO, TTK, ADCY9, CAV1, CSMD3, ERBB3, GLI3, KRAS, NKX2-1, PPP2R1A, SOCS1, TYK2, AGAP2, CBFA2T3, CSNK1G2, ERBB4, GNA11, LMO2, NOS2, PPP2R1B, SOD2, TYMS, AKT1, CBL, CTNNA1, ERCC1, GNAQ, LRP1B, NOS3, PRKAA2, SOS1, UGT1A1, AKT2, CCND1, CTNNA2, ERCC2, GNAS, LRP2, NOTCH1, PRKCA, SOX10, UMPS, AKT3, CCND2, CTNNB1, ERCC3, GPR124, LRP6, NOTCH2, PRKCZ, SOX2, USP9×, ALK, CCND3, CYFIP1, ERCC4, GPR133, LTK, NOTCH3, PRKDC, SP1, VEGF, ANAPC5, CCNE1, CYLD, ERCC5, GRB2, MAN1B1, NPM1, PTCH1, SPRY2, VEGFA, APC, CD40LG, CYP19A1, ERCC6, GSK3B, MAP2K1, NQO1, PTCH2, SRC, VHL,APC2, CD44, CYP1B1, ERG, GSTP1, MAP2K2, NR3C1, PTEN, ST6GAL2, WRN, AR, CD79A, CYP2C19, ERN2, GUCY1A2, MAP2K4, NRAS, PTGS2, STAT1, WT1, ARAF, CD79B, CYP2C8, ESR1, HDAC1, MAP2K7, NRP2, PTPN11, STAT3, XPA, ARFRP1, CDC42, CYP2D6, ESR2, HDAC2, MAP3K1, NTRK1, PTPRB, STK11, XPC, ARID1A, CDC42BPB, CYP3A4, ETV4, HGF, MAPK1, NTRK2, PTPRD, SUFU, ZFY, ATM, CDC73, CYP3A5, EWSR1, HIF1A, MAPK3, NTRK3, RAD50, SULT1A1, ZNF521,ATP5A1, CDH1, DACH2, EXT1, HM13, MAPK8, OMA1, RAD51, SUZ12, ATR, CDH10, DCC, EZH2, HMGA1, MARK3, OR1OR2, RAF1, TAF1, AURKA, CDH2, DCLK3, FANCA, HNF1A, MCL1, PAK3, RARA, TBX22, AURKB, CDH20, DDB2, FANCD2, HOXA3, MDM2, PARP1, RB1, TCF12, BAI3, CDH5, DDR2, FANCE, HOXA9, MDM4, PAX5, REM1, TCF3, BAP1, CDK2, DGKB, FANCF, HRAS, MECOM, PCDH15, RET, TCF4, BARD1, CDK4, DGKZ, FAS, HSP90AA1, MEN1, PCDH18, RICTOR, TEK, BAX, CDK6, DIRAS3, FBXW7, IDH1, MET, PCNA, RIPK1, TEP1, BCL11A, CDK7, DLG3, FCGR3A, IDH2, MITF, PDGFA, ROR1, TERT, BCL2, CDK8, DLL1, FES, IFNG, MLH1, PDGFB, ROR2, TET2, BCL2A1, CDKN1A, DNMT1, FGFR1, IGF1R, MLL, PDGFRA, ROS1, TGFBR2, BCL2L1, CDKN1B, DNMT3A, FGFR2, IGF2R, MLL3, PDGFRB, RPS6KA2, THBS1, BCL2L2, CDKN2A, DNMT3B, FGFR3, IKBKE, MPL, PDZRN3, RPTOR, TNFAIP3, BCL3, CDKN2B, DOT1L, FGFR4, IKZF1, MRE11A, PHLPP2, RSPO2, TNKS, BCL6, CDKN2C, DPYD, FH, IL2RG, MSH2, PIK3C3, RSPO3, TNKS2, BCR, CDKN2D, E2F1, FHOD3, INHBA, MSH6, PIK3CA, RUNX1, TNNI3K, BIRC5, CDX2, EED, FIGF, INSR, MTHFR, PIK3CB, SDHB, TNR, BIRC6, CEBPA, EGF, FLG2, IRS1, MTOR, PIK3CD, SF3B1, TOP1, BLM, CERK, EGFR, FLNC, IRS2, MUTYH, PIK3CG, SHC1, and TOP2A.

In some embodiments, the method further comprises generating a report communicating a profile of the tumor-specific mutations. In some embodiments, detecting the presence or absence of the tumor-specific mutations in cell-free DNA isolated from a liquid sample from the subject is performed at a plurality of time points. In some embodiments, one time point is prior to a first administration of a cancer therapy and a second time point is subsequent to the first administration. In some embodiments, the method further comprises generating a report communicating the profile of tumor-specific mutations at the plurality of time points. In some embodiments, the report comprises a list of one or more therapeutic candidates targeting a gene that harbors one of the tumor-specific mutations. In some embodiments, the report is generated 1 week from isolating the gDNA. In some embodiments, the mutations comprise copy number variation. In some embodiments, the detecting comprises sequencing the cell-free DNA. In some embodiments, the method comprises sequencing at least 10 cancer-related genes present in the cell-free DNA, wherein one of the at least 10 cancer-related genes is identified as harboring a tumor-specific mutation. In some embodiments, the method comprises sequencing at least 100 cancer-related genes present in the cell-free DNA, wherein one of the at least 100 cancer-related genes is identified as harboring a tumor-specific mutation. In some embodiments, sequencing comprises sequencing by any of the methods described herein.

In some aspects, the invention provides an oligonucleotide probe with a low melting temperature (Tm), e.g., a low Tm probe, comprising: a detectable moiety; a quencher moiety; and a melting temperature (Tm) below 50° C. In some embodiments, the low Tm probe has a length of 8-30 nucleotides. In some embodiments, the detectable moiety is quenched at a temperature of 55° C. or higher In some embodiments, the low Tm probe does not hybridize to a complementary template nucleic acid at an ambient temperature above 55° C. In some embodiments, the quencher moiety quenches the detectable moiety if the probe is not hybridized to a template strand. In some embodiments, the Tm of the low Tm probe is between 30-45° C. In some embodiments, the fluorophore moiety and quencher moiety low Tm probe are spaced at least seven nucleotides apart. In some embodiments, the low Tm probe comprises a nucleotide with a Tm enhancing base. In some embodiments the nucleotide with a Tm enhancing base is a Superbase, locked nucleotide, or bridge nucleotide. In some embodiments, the detectable moiety of the low Tm probe comprises a fluorophore.

In some embodiments, the low Tm probe has a length of at least 15 nucleotides. In some embodiments, the low Tm probe has a GC content of at least 40%. In some embodiments, the low Tm probe has a GC content that is less than 80%. In some embodiments, the low Tm probe has a GC content that is less than 50%. In some embodiments, the low Tm probe has a GC content that is less than 40%.

In some embodiments, the low Tm probe has a length of less than 15 nucleotides. In some embodiments, the low Tm probe has a GC content of less than 40%. In some embodiments, the low Tm probe has a GC content that is at least 40%. In some embodiments, the low Tm probe has a GC content that is between 40-80%. In some embodiments, the low Tm probe has a GC content of less than 40%, and further comprising a superbase, a locked or bridged nucleotide.

In some embodiments, the low Tm probe comprises a sequence having at least 70% complementarity or identity to a nucleotide sequence of at least 10 contiguous nucleotides contained in a gene selected from the group consisting of ABCA1, BRAF, CHD5, EP300, FLT1, ITPA, MYC, PIK3R1, SKP2, TP53, ABCA7, BRCA1, CHEK1, EPHA3, FLT3, JAK1, MYCL1, PIK3R2, SLC19A1, TP73, ABCB1, BRCA2, CHEK2, EPHA5, FLT4, JAK2, MYCN, PKHD1, SLC1A6, TPM3, ABCC2, BRIP1, CLTC, EPHA6, FN1, JAK3, MYH2, PLCB1, SLC22A2, TPMT, ABCC3, BUB1B, COL1A1, EPHA7, FOS, JUN, MYH9, PLCG1, SLCO1B3, TPO, ABCC4, Clorf144, COPS5, EPHA8, FOXO1, KBTBD11, NAV3, PLCG2, SMAD2, TPR, ABCG2, CABLES1, CREB1, EPHB1, FOXO3, KDM6A, NBN, PML, SMAD3, TRIO, ABL1, CACNA2D1, CREBBP, EPHB4, FOXP4, KDR, NCOA2, PMS2, SMAD4, TRRAP, ABL2, CAMKV, CRKL, EPHB6, GAB1, KIT, NEK11, PPARG, SMARCA4, TSC1, ACVR1B, CARD11, CRLF2, EPO, GATA1, KLF6, NF1, PPARGC1A, SMARCB1, TSC2, ACVR2A, CARM1, CSF1R, ERBB2, GLI1, KLHDC4, NF2, PPP1R3A, SMO, TTK, ADCY9, CAV1, CSMD3, ERBB3, GLI3, KRAS, NKX2-1, PPP2R1A, SOCS1, TYK2, AGAP2, CBFA2T3, CSNK1G2, ERBB4, GNA11, LMO2, NOS2, PPP2R1B, SOD2, TYMS, AKT1, CBL, CTNNA1, ERCC1, GNAQ, LRP1B, NOS3, PRKAA2, SOS1, UGT1A1, AKT2, CCND1, CTNNA2, ERCC2, GNAS, LRP2, NOTCH1, PRKCA, SOX10, UMPS, AKT3, CCND2, CTNNB1, ERCC3, GPR124, LRP6, NOTCH2, PRKCZ, SOX2, USP9×, ALK, CCND3, CYFIP1, ERCC4, GPR133, LTK, NOTCH3, PRKDC, SP1, VEGF, ANAPC5, CCNE1, CYLD, ERCC5, GRB2, MAN1B1, NPM1, PTCH1, SPRY2, VEGFA, APC, CD40LG, CYP19A1, ERCC6, GSK3B, MAP2K1, NQO1, PTCH2, SRC, VHL,APC2, CD44, CYP1B1, ERG, GSTP1, MAP2K2, NR3C1, PTEN, ST6GAL2, WRN, AR, CD79A, CYP2C19, ERN2, GUCY1A2, MAP2K4, NRAS, PTGS2, STAT1, WT1, ARAF, CD79B, CYP2C8, ESR1, HDAC1, MAP2K7, NRP2, PTPN11, STAT3, XPA, ARFRP1, CDC42, CYP2D6, ESR2, HDAC2, MAP3K1, NTRK1, PTPRB, STK11, XPC, ARID1A, CDC42BPB, CYP3A4, ETV4, HGF, MAPK1, NTRK2, PTPRD, SUFU, ZFY, ATM, CDC73, CYP3A5, EWSR1, HIF1A, MAPK3, NTRK3, RAD50, SULT1A1, ZNF521,ATP5A1, CDH1, DACH2, EXT1, HM13, MAPK8, OMA1, RAD51, SUZ12, ATR, CDH10, DCC, EZH2, HMGA1, MARK3, OR1OR2, RAF1, TAF1, AURKA, CDH2, DCLK3, FANCA, HNF1A, MCL1, PAK3, RARA, TBX22, AURKB, CDH20, DDB2, FANCD2, HOXA3, MDM2, PARP1, RB1, TCF12, BAI3, CDH5, DDR2, FANCE, HOXA9, MDM4, PAX5, REM1, TCF3, BAP1, CDK2, DGKB, FANCF, HRAS, MECOM, PCDH15, RET, TCF4, BARD1, CDK4, DGKZ, FAS, HSP90AA1, MEN1, PCDH18, RICTOR, TEK, BAX, CDK6, DIRAS3, FBXW7, IDH1, MET, PCNA, RIPK1, TEP1, BCL11A, CDK7, DLG3, FCGR3A, IDH2, MITF, PDGFA, ROR1, TERT, BCL2, CDK8, DLL1, FES, IFNG, MLH1, PDGFB, ROR2, TET2, BCL2A1, CDKN1A, DNMT1, FGFR1, IGF1R, MLL, PDGFRA, ROS1, TGFBR2, BCL2L1, CDKN1B, DNMT3A, FGFR2, IGF2R, MLL3, PDGFRB, RPS6KA2, THBS1, BCL2L2, CDKN2A, DNMT3B, FGFR3, IKBKE, MPL, PDZRN3, RPTOR, TNFAIP3, BCL3, CDKN2B, DOT1L, FGFR4, IKZF1, MRE11A, PHLPP2, RSPO2, TNKS, BCL6, CDKN2C, DPYD, FH, IL2RG, MSH2, PIK3C3, RSPO3, TNKS2, BCR, CDKN2D, E2F1, FHOD3, INHBA, MSH6, PIK3CA, RUNX1, TNNI3K, BIRC5, CDX2, EED, FIGF, INSR, MTHFR, HADH, RPP30, ZFP3, PIK3CB, SDHB, TNR, BIRC6, CEBPA, EGF, FLG2, IRS1, MTOR, PIK3CD, SF3B1, TOP1, BLM, CERK, EGFR, FLNC, IRS2, MUTYH, PIK3CG, SHC1, and TOP2A.

In some aspects, the invention also provides a reaction mixture comprising at least one primer/probe set, wherein the primer/probe set comprises: a forward primer designed to hybridize to a genomic region at a first location; and a low Tm probe as described herein. In some embodiments, the reaction mixture further comprises a reverse primer designed to hybridize to the genomic region at a second location. In some embodiments, the low Tm probe has a Tm that is at least 15° C. lower than the Tm of the forward primer. In some embodiments, the low Tm probe has a Tm that is at least 15° C. lower than an average of the Tm of the first primer and the Tm of the second primer. In some embodiments, the low Tm probe is designed to hybridize to the genomic region at a third location located between the first and second location. In some embodiments the reverse primer is present in an amount that is at least 2 to 10-fold less than an amount of the forward primer. In some embodiments the reverse primer is present in an amount that is no more than 2-fold different than an amount of the forward primer.

In some embodiments, the reaction mixture further comprises a nucleic acid sample isolated from a biological sample. In some embodiments, the biological sample is a sample isolated from a subject. In some embodiments, the subject is a human subject. In some embodiments, the human subject is diagnosed, suspected of having, or suspected of being at increased risk for a disease. In some embodiments, the disease is cancer. In some embodiments, the template nucleic acid comprises a genomic region. In some embodiments, the template nucleic acid comprises DNA, RNA, or cDNA. In some embodiments, the reaction mixture further comprises a polymerase. In some embodiments, the polymerase is a DNA polymerase. In some embodiments, the reaction mixture comprises (a) a first template nucleic acid; (b) an amount of a forward primer; (c) an amount of a reverse primer, wherein the amount of reverse primer is at least 2 to 10-fold less than the amount of the forward primer; and (d) a low Tm probe.

In some embodiments, the reaction mixture comprises a plurality of primer/probe sets. In some embodiments, wherein each primer/probe set of the plurality is specific for a different region of genomic DNA. In some embodiments, the genomic region is associated with a disease-related mutation. In some embodiments, the mutation comprises a copy number variation. In some embodiments, the mutation comprises a single nucleotide polymorphism (SNP), insertion, deletion, or inversion. In some embodiments, wherein one of the forward or reverse primers overlays the SNP, insertion, deletion, or inversion. In some embodiments, the low Tm probe overlays the SNP, insertion, deletion, or inversion. In some embodiments, the disease is a cancer.

In some embodiments, the primer/probe set comprises a plurality of low Tm probes, wherein each low Tm probe is an allele-specific probe designed to bind with greater avidity to a sequence comprising one specific allele of the genomic region as compared to a sequence comprising any other allele of the genomic region, wherein each allele-specific probe is specific for a different allele.

In some embodiments, each of the allele-specific probes each comprise a spectrally distinct fluorophore.

In some embodiments, the difference in binding energy of an allele specific probe to the one specific allele as compared to a binding energy of the allele specific probe to any other allele is more than 1% of the overall binding energy of the low Tm probe to the genomic region. In some embodiments, the low Tm probe is a beacon probe. In some embodiments, the low Tm probe is a Pleiades probe.

In a related aspect, the invention provides a method, the method comprising partitioning a reaction mixture comprising a low Tm probe as described herein into a plurality of reaction volumes; and performing, in at least one of the reaction volumes, a PCR amplification reaction comprising multiple rounds of thermal cycling, wherein the low Tm probe does not affect efficiency of the PCR amplification reaction.

In some embodiments, the low Tm probe does not hybridize to a template nucleic acid or PCR reaction product during an annealing phase or extension phase of the PCR amplification reaction. In some embodiments, the method further comprises cooling at least one of the reaction volumes to below 50° C., wherein the cooling enables hybridization of the low Tm probe to a template nucleic acid or PCR reaction product. In some embodiments the template nucleic acid or PCR reaction product comprises a sequence having at least 70% complementarity to the low Tm probe.

In some embodiments, the method comprises cooling at least one of the reaction volumes to below 37° C., wherein the cooling enables hybridization of at least 5%, 10%, 20%, 30%, 40%, 50%, 60%, 70% of an amount of low Tm probes to nucleic acids comprising a sequence having at least 70% complementarity to the low Tm probe.In some embodiments, the partitioning results in each reaction volume containing on average <1, 1, or more than 1 molecule of template nucleic acid. In some embodiments, the partitioning results in each reaction volume containing on average 1 or more molecules of template nucleic acid.

In some embodiments, the method comprises performing an exponential PCR amplification reaction and a linear PCR amplification reaction in at least one of the reaction volumes.

In some embodiments, the exponential PCR amplification and the linear PCR amplification reaction occurs sequentially without adding or removing components from the reaction volumes.

In some embodiments, the PCR amplification reaction results in at least 1%, 5%, 10%, 20%, 30%, 40%, or 50% of the amplification products being single-stranded amplification products.

In some embodiments, the reaction volumes are droplets. In some embodiments, the hybridization results in emission of fluorescence from the low Tm probe. In some embodiments, the method further comprises detecting the presence or absence of the fluorescence in at least one of the reaction volumes. In some embodiments, the method comprises measuring intensity of the fluorescence in the reaction volumes. In some embodiments, the method further comprises determining a number and/or fraction of fluorescence-positive reaction volumes. In some embodiments, the method comprises determining the presence, absence, or amount of one or more mutations in the sample based on the number and/or fraction of fluorescence-positive reaction volumes. In some embodiments, the one or more mutations comprises a SNP, deletion, insertion, or inversion. In some embodiments, the one or more mutations comprises a copy number variation of a gene. In some embodiments, the one or more mutations comprises a disease-related mutation. In some embodiments, the disease is cancer. In some embodiments, the one or more mutations comprises a mutation of one or more genes selected from the group consisting of ABCA1, BRAF, CHD5, EP300, FLT1, ITPA, MYC, PIK3R1, SKP2, TP53, ABCA7, BRCA1, CHEK1, EPHA3, FLT3, JAK1, MYCL1, PIK3R2, SLC19A1, TP73, ABCB1, BRCA2, CHEK2, EPHA5, FLT4, JAK2, MYCN, PKHD1, SLC1A6, TPM3, ABCC2, BRIP1, CLTC, EPHA6, FN1, JAK3, MYH2, PLCB1, SLC22A2, TPMT, ABCC3, BUB1B, COL1A1, EPHA7, FOS, JUN, MYH9, PLCG1, SLCO1B3, TPO, ABCC4, Clorf144, COPS5, EPHA8, FOXO1, KBTBD11, NAV3, PLCG2, SMAD2, TPR, ABCG2, CABLES1, CREB1, EPHB1, FOXO3, KDM6A, NBN, PML, SMAD3, TRIO, ABL1, CACNA2D1, CREBBP, EPHB4, FOXP4, KDR, NCOA2, PMS2, SMAD4, TRRAP, ABL2, CAMKV, CRKL, EPHB6, GAB1, KIT, NEK11, PPARG, SMARCA4, TSC1, ACVR1B, CARD11, CRLF2, EPO, GATA1, KLF6, NF1, PPARGC1A, SMARCB1, TSC2, ACVR2A, CARM1, CSF1R, ERBB2, GLI1, KLHDC4, NF2, PPP1R3A, SMO, TTK, ADCY9, CAV1, CSMD3, ERBB3, GLI3, KRAS, NKX2-1, PPP2R1A, SOCS1, TYK2, AGAP2, CBFA2T3, CSNK1G2, ERBB4, GNA11, LMO2, NOS2, PPP2R1B, SOD2, TYMS, AKT1, CBL, CTNNA1, ERCC1, GNAQ, LRP1B, NOS3, PRKAA2, SOS1, UGT1A1, AKT2, CCND1, CTNNA2, ERCC2, GNAS, LRP2, NOTCH1, PRKCA, SOX10, UMPS, AKT3, CCND2, CTNNB1, ERCC3, GPR124, LRP6, NOTCH2, PRKCZ, SOX2, USP9×, ALK, CCND3, CYFIP1, ERCC4, GPR133, LTK, NOTCH3, PRKDC, SP1, VEGF, ANAPC5, CCNE1, CYLD, ERCC5, GRB2, MAN1B1, NPM1, PTCH1, SPRY2, VEGFA, APC, CD40LG, CYP19A1, ERCC6, GSK3B, MAP2K1, NQO1, PTCH2, SRC, VHL,APC2, CD44, CYP1B1, ERG, GSTP1, MAP2K2, NR3C1, PTEN, ST6GAL2, WRN, AR, CD79A, CYP2C19, ERN2, GUCY1A2, MAP2K4, NRAS, PTGS2, STAT1, WT1, ARAF, CD79B, CYP2C8, ESR1, HDAC1, MAP2K7, NRP2, PTPN11, STAT3, XPA, ARFRP1, CDC42, CYP2D6, ESR2, HDAC2, MAP3K1, NTRK1, PTPRB, STK11, XPC, ARID1A, CDC42BPB, CYP3A4, ETV4, HGF, MAPK1, NTRK2, PTPRD, SUFU, ZFY, ATM, CDC73, CYP3A5, EWSR1, HIF1A, MAPK3, NTRK3, RAD50, SULT1A1, ZNF521,ATP5A1, CDH1, DACH2, EXT1, HM13, MAPK8, OMA1, RAD51, SUZ12, ATR, CDH10, DCC, EZH2, HMGA1, MARK3, OR10R2, RAF1, TAF1, AURKA, CDH2, DCLK3, FANCA, HNF1A, MCL1, PAK3, RARA, TBX22, AURKB, CDH20, DDB2, FANCD2, HOXA3, MDM2, PARP1, RB1, TCF12, BAI3, CDH5, DDR2, FANCE, HOXA9, MDM4, PAX5, REM1, TCF3, BAP1, CDK2, DGKB, FANCF, HRAS, MECOM, PCDH15, RET, TCF4, BARD1, CDK4, DGKZ, FAS, HSP90AA1, MEN1, PCDH18, RICTOR, TEK, BAX, CDK6, DIRAS3, FBXW7, IDH1, MET, PCNA, RIPK1, TEP1, BCL11A, CDK7, DLG3, FCGR3A, IDH2, MITF, PDGFA, ROR1, TERT, BCL2, CDK8, DLL1, FES, IFNG, MLH1, PDGFB, ROR2, TET2, BCL2A1, CDKN1A, DNMT1, FGFR1, IGF1R, MLL, PDGFRA, ROS1, TGFBR2, BCL2L1, CDKN1B, DNMT3A, FGFR2, IGF2R, MLL3, PDGFRB, RPS6KA2, THBS1, BCL2L2, CDKN2A, DNMT3B, FGFR3, IKBKE, MPL, PDZRN3, RPTOR, TNFAIP3, BCL3, CDKN2B, DOT1L, FGFR4, IKZF1, MRE11A, PHLPP2, RSPO2, TNKS, BCL6, CDKN2C, DPYD, FH, IL2RG, MSH2, PIK3C3, RSPO3, TNKS2, BCR, CDKN2D, E2F1, FHOD3, INHBA, MSH6, PIK3CA, RUNX1, TNNI3K, BIRC5, CDX2, EED, FIGF, INSR, MTHFR, HADH, RPP30, ZFP3, PIK3CB, SDHB, TNR, BIRC6, CEBPA, EGF, FLG2, IRS1, MTOR, PIK3CD, SF3B1, TOP1, BLM, CERK, EGFR, FLNC, IRS2, MUTYH, PIK3CG, SHC1, and TOP2A.

In some embodiments, the one or more mutations comprises a mutation of one or more genes selected from the group consisting of DDR2, EGFR, AURKA, VEGFA, FGFR1, CDK4, EFBB2, CDK6, JAK2, MET, BRAF, ERBB3, and SRC.

In some embodiments, the method comprises generating a report communicating a profile of the presence, absence, and/or level of the mutation in the sample. In some embodiments, the report further comprises a description of a therapeutic agent targeting the mutation.

In a related aspect, the invention provides a computer system, comprising: a memory unit configured to receive data from a sample, wherein the data is generated by any of the foregoing methods employing a low Tm probe; computer executable instructions for analysis of the data; and computer executable instructions to determine the presence, absence, or amount of a mutation in the sample based on the analysis. In some embodiments, the computer system further comprises computer executable instructions to generate a report of the presence, absence, or amount of a mutation in the sample. In some embodiments, the computer system further comprises computer executable instructions to generate a report of therapeutic options based on the presence, absence, or amount of a mutation in the sample. In some embodiments, the computer system further comprises a user interface configured to communicate or display the report to a user.

In yet another related aspect, the invention provides a kit, comprising: at least one primer/probe set, wherein the primer/probe set comprises (i) a forward primer designed to hybridize to a genomic region at a first location, (ii) a reverse primer designed to hybridize to the genomic region at a second location, and (iii) a low Tm probe described herein, wherein the low Tm probe is designed to hybridize to the genomic region at a third location.

The invention also provides a method of treating cancer in a subject in need thereof, comprising: (a) obtaining a biological sample from the subject; (b) from a nucleic acid sample isolated from the biological sample, determining a presence or absence of a copy number variation (CNV) in at least five genes selected from the group consisting of MET, FGFR1, FGFR2, FLT3, HER3, EGFR, mTOR, CDK4, HER2, RET, HADH, ZFP3, DDR2, AURKA, VEGFA, CDK6, JAK2, BRAF, and SRC; (c) based on the determining, generating a subject-specific CNV profile; and (d) based on the subject-specific CNV profile, selecting a cancer therapy for the subject. In some embodiments, the determining a presence or absence of a CNV comprises use of any of the foregoing methods. In some embodiments, the determining comprises a digital PCR assay. In some embodiments, the digital PCR assay comprises use of any of the foregoing oligonucleotide probes. In some embodiments, the oligonucleotide probe comprises a nucleotide sequence of any of SEQ ID NOS: 61, 64, 67, 70, 73, 76, 79, 82, 85, 88, 91, 94, 97, 100, 103, 106, 109, 112, 115, or 118. In some embodiments, the digital PCR assay comprises use of any of the foregoing primers. In some embodiments, the primer comprises a nucleotide sequence of any of SEQ ID NOS. 59, 60, 62, 63, 65, 66, 68, 69, 71, 72, 74, 75, 77, 78, 80, 81, 83, 84, 86, 87, 89, 90, 92, 93, 95, 96, 98, 99, 101, 102, 104, 105, 107, 108, 110, 111, 113, 114, 116, or 117. In some embodiments, the method comprises determining of presence or absence of a CNV in at least 10, 12, or 18 genes. In some embodiments, the biological sample is suspected of harboring nucleic acids originating from the cancer. In some embodiments, the biological sample is a solid tissue sample. In some embodiments, the solid tissue sample is a formalin fixed, paraffin embedded sample. In some embodiments, the biological sample is a liquid biological sample. In some embodiments, the liquid biological sample is selected from the group consisting of blood, serum, plasma, urine, sweat, tears, saliva, and sputum.

The invention also provides a computer system, comprising: (a) a memory unit configured to receive data from a sample, wherein the data is generated by any of the foregoing methods; (b) computer executable instructions for analysis of the data; and (c) computer executable instructions to determine the presence, absence, or amount of a mutation in the sample based on the analysis. In some embodiments, the computer system further comprises computer executable instructions to generate a report of the presence, absence, or amount of a mutation in the sample. In some embodiments, the computer system further comprises computer executable instructions to generate a report of therapeutic options based on the presence, absence, or amount of a mutation in the sample. In some embodiments, the computer system further comprises a user interface configured to communicate or display the report to a user.

The invention also provides a kit, comprising: (a) at least one primer/probe set, wherein the primer/probe set comprises (i) a forward primer designed to hybridize to a genomic region at a first location, (ii) a reverse primer designed to hybridize to the genomic region at a second location, and (iii) an oligonucleotide probe as previously set forth, wherein the oligonucleotide probe is designed to hybridize to the genomic region at a third location located between the first and second location; and (b) instructions for use.

The invention also provides an oligonucleotide probe as set forth in any of SEQ ID NO: 4-21, 23, 24, 61, 64, 67, 70, 73, 76, 79, 82, 85, 88, 91, 94, 97, 100, 103, 106, 109, 112, 115, or 118.

The invention also provides a target-selective oligonucleotide as set forth in any of SEQ. ID. NOS: 1948-5593.

The invention also provides an oligonucleotide primer having a sequence as set forth in SEQ ID NO: 25 or 26.

The invention also provides an oligonucleotide primer having a sequence as set forth in any of SEQ ID NOS. 1-3, 22, 27-58, 59, 60, 62, 63, 65, 66, 68, 69, 71, 72, 74, 75, 77, 78, 80, 81, 83, 84, 86, 87, 89, 90, 92, 93, 95, 96, 98, 99, 101, 102, 104, 105, 107, 108, 110, 111, 113, 114, 116, or 117.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts an exemplary workflow of a method for assessing cancer in a subject.

FIG. 2 depicts an exemplary workflow of a method for sequencing a tumor cell and a normal cell in a subject. FIG. 2 discloses SEQ ID NOS 119-120, respectively, in order of appearance.

FIG. 3 depicts an exemplary workflow for a method of preparing a DNA library from a tumor sample of a subject.

FIG. 4 depicts an exemplary embodiment of a method of preparing a DNA library from a tumor sample of a subject.

FIG. 5 depicts an exemplary embodiment of a method of assessing tumor-specific mutations in cell-free DNA from a blood sample of a subject

FIG. 6 depicts an exemplary workflow for allele detection in a sample.

FIG. 7 depicts an exemplary workflow for wild-type and mutant allele detection in a sample.

FIG. 8 depicts an exemplary embodiment of a subject-specific report of tumor-specific mutations in a subject.

FIG. 9 depicts an exemplary computer system of the invention.

FIG. 10A depicts an exemplary workflow of a ligation method of the invention.

FIG. 10B depicts an exemplary method for preparing a single-stranded DNA library.

FIG. 11 depicts an exemplary embodiment of a ligation method of the invention.

FIG. 12 depicts an exemplary workflow of a method of preparing a nucleic acid library for sequencing.

FIGS. 13A and 13B depict exemplary embodiments of a method of preparing a single-adaptor nucleic acid library for sequencing.

FIGS. 14A and 14B depict exemplary embodiments of a method of ligating a second adaptor sequence to a single-adaptor ligated library member.

FIG. 15 depicts an exemplary method of cloning an insert into a plasmid vector using a high efficiency ligation method.

FIG. 16 depicts an exemplary workflow of a method for sensitive detection of amplicons.

FIG. 17 depicts an exemplary embodiment of a method for sensitive detection of amplicons.

FIG. 18 depicts an exemplary embodiment of a real-time detection method for sensitive detection of amplicons.

FIG. 19 depicts an exemplary embodiment of an exponential PCR-based detection method for sensitive detection of amplicons.

FIG. 20 depicts an exemplary embodiment of a linear PCR-based detection method for sensitive detection of amplicons.

FIG. 21 depicts an exemplary embodiment of a PCR-based detection method that utilizes exponential amplification followed by linear amplification.

FIGS. 22A-22B depict an exemplary embodiment of an allele discrimination assay.

FIG. 23 depicts another exemplary embodiment of an allele discrimination assay.

FIG. 24 depicts a method used to assess a cancer in a subject with colon cancer.

FIG. 25 and FIGS. 26A-26D depict results from a validation assay for a tumor-specific mutation in the subject with colon cancer.

FIG. 27 depicts an exemplary embodiment of a method for quantitating efficiency of a ligation method described herein.

FIG. 28 depicts ddPCR results for the 5′ end adaptor ligation and 3′ end adaptor ligation reactions, respectfully.

FIG. 29 depicts results from a ligation experiment testing adaptor length and PEG-8000 on Ligation Efficiency.

FIG. 30 depicts results from a ligation experiment testing the effect of Mn²⁺ vs. incubation temperature.

FIG. 31 depicts an exemplary embodiment of sequencing using an Illumina NGS platform.

FIGS. 32 and 33 depict exemplary embodiments of a target-selective oligonucleotide (TSO) primer. FIGS. 32 and 33 disclose SEQ ID NOS 121-124, respectively, in order of appearance.

FIGS. 34A-34D depict results from an experiment for the assessment of low Tm probe designs. FIGS. 34A-34D disclose SEQ ID NOS 6-8, 10, 12, 9, 11, 13, 15-16, 14, 17-18, 20, 19 and 21, respectively, in order of appearance.

FIGS. 35A-35B, 36A-36B, 37A-37B, and 38A-38B depict results from ddPCR assays testing various primer/probe designs for detection of BRAF alleles.

FIGS. 39-40 demonstrate detection limits of the BRAF low Tm universal probes with barcoded primers.

FIG. 41 depicts results from a numerical analysis to determine exemplary input amounts for a 20,000 partition digital PCR experiment.

FIGS. 42A and 42B and 43A-43D depict use of CNV ddPCR panel for selecting effective cancer treatment in a patient with colon cancer which has metastasized to the liver.

FIGS. 44A-44B depict results from a single assay which can detect copy number variation and mutation of a gene.

### DETAILED DESCRIPTION OF THE INVENTION

The practice of the present invention will employ, unless otherwise indicated, conventional techniques of molecular biology, microbiology and recombinant DNA techniques, which are within the skill of the art. Such techniques are explained fully in the literature. See, e.g., Sambrook, Fritsch & Maniatis, Molecular Cloning: A Laboratory Manual, Fourth Edition (2012); Oligonucleotide Synthesis (M. J. Gait, ed., 1984); Nucleic Acid Hybridization (B. D. Hames & S. J. Higgins, eds., 1984); A Practical Guide to Molecular Cloning (B. Perbal, 1984); and a series, Methods in Enzymology (Academic Press, Inc.). All patents, patent applications, and publications mentioned herein, both supra and infra, are hereby incorporated by reference.

### Definitions

As used in the specification and claims, the singular forms “a”, “an” and “the” can include plural references unless the context clearly dictates otherwise. For example, the term “a cell” can include a plurality of cells, including mixtures thereof.

The term “subject”, as used herein, generally refers to a biological entity containing expressed genetic materials. The biological entity can be a plant, animal, or microorganism, including, e.g., bacteria, viruses, fungi, and protozoa. The subject can be tissues, cells and their progeny of a biological entity obtained in vivo or cultured in vitro. The subject can be a mammal. The mammal can be a human. The human may be diagnosed or suspected of being at high risk for a disease. The disease can be cancer. The human may not be diagnosed or suspected of being at high risk for a disease.

As used herein, a “sample” or “nucleic acid sample” can refer to any substance containing or presumed to contain nucleic acid. The sample can be a biological sample obtained from a subject. The nucleic acids can be RNA, DNA, e.g., genomic DNA, mitochondrial DNA, viral DNA, synthetic DNA, or cDNA reverse transcribed from RNA. The nucleic acids in a nucleic acid sample generally serve as templates for extension of a hybridized primer. In some embodiments, the biological sample is a liquid sample. The liquid sample can be whole blood, plasma, serum, ascites, cerebrospinal fluid, sweat, urine, tears, saliva, buccal sample, cavity rinse, or organ rinse. The liquid sample can be an essentially cell-free liquid sample (e.g., plasma, serum, sweat, plasma, urine, sweat, tears, saliva, sputum). In other embodiments, the biological sample is a solid biological sample, e.g., feces or tissue biopsy, e.g., a tumor biopsy. A sample can also comprise in vitro cell culture constituents (including but not limited to conditioned medium resulting from the growth of cells in cell culture medium, recombinant cells and cell components). The sample can comprise a single cell, e.g., a cancer cell, a circulating tumor cell, a cancer stem cell, and the like.

“Nucleotides” and “nt” are used interchangeably herein to generally refer to biological molecules that can form nucleic acids. Nucleotides can have moieties that contain not only the known purine and pyrimidine bases, but also other heterocyclic bases that have been modified. Such modifications include methylated purines or pyrimidines, acylated purines or pyrimidines, alkylated riboses, or other heterocycles. In addition, the term “nucleotide” includes those moieties that contain hapten, biotin, or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the like. Modified nucleosides or nucleotides can also include peptide nucleic acid (PNA). Peptide nucleic acid generally refers to oligonucleotides in which the deoxyribose backbone has been replaced with a backbone having peptide linkages. Each subunit generally has attached a naturally occurring or non-naturally occurring base. One exemplary PNA backbone is constructed of repeating units of N-(2-aminoethyl)glycine linked through amide bonds. PNA can bind both DNA and RNA to form PNA/DNA or PNA/RNA duplexes. The resulting PNA/DNA or PNA/RNA duplexes can be bound with greater affinity than corresponding DNA/DNA or DNA/RNA duplexes as evidence by their higher melting temperatures (Tm). The neutral backbone of the PNA also can render the Tm of PNA/DNA(RNA) duplexes to be largely independent of salt concentration in a reaction mixture. Thus the PNA/DNA duplex can offer an advantage over DNA/DNA duplex interactions which are highly dependent on ionic strength. Exemplary embodiments of PNA are described in U.S. Pat. Nos. 7,223,833 and 5,539,083, which are hereby incorporated by reference.

“Nucleotides” can also include nucleotides comprising a Tm-enhancing base (e.g., a Tm-base enhancing nucleotide). Exemplary Tm-enhancing base nucleotides include, but are not limited to nucleotides with Superbases™, locked nucleic acids (LNA) or bridged nucleic acids (BNA). BNA and LNA generally refer to modified ribonucleotides wherein the ribose moiety is modified with a bridge connecting the 2′ oxygen and 4′ carbon. Generally, the bridge “locks” the ribose in the 3′-endo (North) conformation, which is often found in the A-form duplexes. The term “locked nucleic acid” (LNA) generally refers to a class of BNAs, where the ribose ring is “locked” with a methylene bridge connecting the 2′-O atom with the 4′-C atom. LNA nucleosides containing the six common nucleobases (T, C, G, A, U and mC) that appear in DNA and RNA are able to form base-pairs with their complementary nucleosides according to the standard Watson-Crick base pairing rules. Accordingly, Tm-enhancing base nucleotides such as BNA and LNA nucleotides can be mixed with DNA or RNA bases in an oligonucleotide whenever desired. The locked ribose conformation enhances base stacking and backbone pre-organization. Base stacking and backbone pre-organization can give rise to an increased thermal stability (e.g., increased Tm) and discriminative power of duplexes. LNA can discriminate single base mismatches under conditions not possible with other nucleic acids. Locked nucleic acid is disclosed for example in WO 99/14226, hereby incorporated by reference. Nucleotides can also include modified nucleotides as described in European Patent Application No. EP1995330, hereby incorporated by reference.

Other modified nucleotides can include 5-Me-dC-CE phosphoramidite, 5-Me-dC-CPG, 2-Amino-dA-CE phosphoramidite, N4-Et-dC-CE Phosphoramidite, N4-Ac-N4-Et-dC-CE Phosphoramidite, N6-Me-dA-CE Phosphoramidite, N6-Ac-N6-Me-dA-CE Phosphoramidite, Zip nucleic acids (ZNA®, described in U.S. patent application Ser. No. 12/086,599, hereby incorporated by reference), 5′-Trimethoxystilbene Cap Phosphoramidite, 5′-Pyrene Cap Phosphoramidite, 3′-Uaq Cap CPG. (Glen Research).

Yet other modified nucleotides can include nucleotides with modified nucleoside bases such as, e.g., 2-Aminopurine, 2,6-Diaminopurine, 5-Bromo-deoxyuridine, deoxyuridine, Inverted dT, inverted ddT, ddC, 5-Methyl deoxyCytidine, deoxyInosine, 5-Nitroindole, 2′-O-Methyl RNA bases, Hydroxmethyl dC, Iso-dG and Iso-dC (Eragen Biosciences, Inc), 2′ Fluoro bases having a fluorine modified ribose.

The terms “polynucleotides”, “nucleic acid”, “nucleotides” and “oligonucleotides” can be used interchangeably. They can refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “target polynucleotide,”, “target region”, or “target”, as use herein, generally refers to a polynucleotide of interest under study. In certain embodiments, a target polynucleotide contains one or more sequences that are of interest and under study. A target polynucleotide can comprise, for example, a genomic sequence. The target polynucleotide can comprise a target sequence whose presence, amount, and/or nucleotide sequence, or changes in these, are desired to be determined.

The target polynucleotide can be a region of gene associated with a disease. In some embodiments, the region is an exon. In some embodiments, the gene is a druggable target. The term “druggable target”, as used herein, generally refers to a gene or cellular pathway that is modulated by a disease therapy. The disease can be cancer. Accordingly, the gene can be a known cancer-related gene. In some embodiments, the cancer-related gene is selected from the group consisting of ABCA1, BRAF, CHD5, EP300, FLT1, ITPA, MYC, PIK3R1, SKP2, TP53, ABCA7, BRCA1, CHEK1, EPHA3, FLT3, JAK1, MYCL1, PIK3R2, SLC19A1, TP73, ABCB1, BRCA2, CHEK2, EPHA5, FLT4, JAK2, MYCN, PKHD1, SLC1A6, TPM3, ABCC2, BRIP1, CLTC, EPHA6, FN1, JAK3, MYH2, PLCB1, SLC22A2, TPMT, ABCC3, BUB1B, COL1A1, EPHA7, FOS, JUN, MYH9, PLCG1, SLCO1B3, TPO, ABCC4, Clorf144, COPS5, EPHA8, FOXO1, KBTBD11, NAV3, PLCG2, SMAD2, TPR, ABCG2, CABLES1, CREB1, EPHB1, FOXO3, KDM6A, NBN, PML, SMAD3, TRIO, ABL1, CACNA2D1, CREBBP, EPHB4, FOXP4, KDR, NCOA2, PMS2, SMAD4, TRRAP, ABL2, CAMKV, CRKL, EPHB6, GAB1, KIT, NEK11, PPARG, SMARCA4, TSC1, ACVR1B, CARD11, CRLF2, EPO, GATA1, KLF6, NF1, PPARGC1A, SMARCB1, TSC2, ACVR2A, CARM1, CSF1R, ERBB2, GLI1, KLHDC4, NF2, PPP1R3A, SMO, TTK, ADCY9, CAV1, CSMD3, ERBB3, GLI3, KRAS, NKX2-1, PPP2R1A, SOCS1, TYK2, AGAP2, CBFA2T3, CSNK1G2, ERBB4, GNA11, LMO2, NOS2, PPP2R1B, SOD2, TYMS, AKT1, CBL, CTNNA1, ERCC1, GNAQ, LRP1B, NOS3, PRKAA2, SOS1, UGT1A1, AKT2, CCND1, CTNNA2, ERCC2, GNAS, LRP2, NOTCH1, PRKCA, SOX10, UMPS, AKT3, CCND2, CTNNB1, ERCC3, GPR124, LRP6, NOTCH2, PRKCZ, SOX2, USP9X, ALK, CCND3, CYFIP1, ERCC4, GPR133, LTK, NOTCH3, PRKDC, SP1, VEGF, ANAPC5, CCNE1, CYLD, ERCC5, GRB2, MAN1B1, NPM1, PTCH1, SPRY2, VEGFA, APC, CD40LG, CYP19A1, ERCC6, GSK3B, MAP2K1, NQO1, PTCH2, SRC, VHL,APC2, CD44, CYP1B1, ERG, GSTP1, MAP2K2, NR3C1, PTEN, ST6GAL2, WRN, AR, CD79A, CYP2C19, ERN2, GUCY1A2, MAP2K4, NRAS, PTGS2, STAT1, WT1, ARAF, CD79B, CYP2C8, ESR1, HDAC1, MAP2K7, NRP2, PTPN11, STAT3, XPA, ARFRP1, CDC42, CYP2D6, ESR2, HDAC2, MAP3K1, NTRK1, PTPRB, STK11, XPC, ARID1A, CDC42BPB, CYP3A4, ETV4, HGF, MAPK1, NTRK2, PTPRD, SUFU, ZFY, ATM, CDC73, CYP3A5, EWSR1, HIF1A, MAPK3, NTRK3, RAD50, SULT1A1, ZNF521,ATP5A1, CDH1, DACH2, EXT1, HM13, MAPK8, OMA1, RAD51, SUZ12, ATR, CDH10, DCC, EZH2, HMGA1, MARK3, OR1OR2, RAF1, TAF1, AURKA, CDH2, DCLK3, FANCA, HNF1A, MCL1, PAK3, RARA, TBX22, AURKB, CDH20, DDB2, FANCD2, HOXA3, MDM2, PARP1, RB1, TCF12, BAI3, CDH5, DDR2, FANCE, HOXA9, MDM4, PAX5, REM1, TCF3, BAP1, CDK2, DGKB, FANCF, HRAS, MECOM, PCDH15, RET, TCF4, BARD1, CDK4, DGKZ, FAS, HSP90AA1, MEN1, PCDH18, RICTOR, TEK, BAX, CDK6, DIRAS3, FBXW7, IDH1, MET, PCNA, RIPK1, TEP1, BCL11A, CDK7, DLG3, FCGR3A, IDH2, MITF, PDGFA, ROR1, TERT, BCL2, CDK8, DLL1, FES, IFNG, MLH1, PDGFB, ROR2, TET2, BCL2A1, CDKN1A, DNMT1, FGFR1, IGF1R, MLL, PDGFRA, ROS1, TGFBR2, BCL2L1, CDKN1B, DNMT3A, FGFR2, IGF2R, MLL3, PDGFRB, RPS6KA2, THBS1, BCL2L2, CDKN2A, DNMT3B, FGFR3, IKBKE, MPL, PDZRN3, RPTOR, TNFAIP3, BCL3, CDKN2B, DOT1L, FGFR4, IKZF1, MRE11A, PHLPP2, RSPO2, TNKS, BCL6, CDKN2C, DPYD, FH, IL2RG, MSH2, PIK3C3, RSPO3, TNKS2, BCR, CDKN2D, E2F1, FHOD3, INHBA, MSH6, PIK3CA, RUNX1, TNNI3K, BIRC5, CDX2, EED, FIGF, INSR, MTHFR, PIK3CB, SDHB, TNR, BIRC6, CEBPA, EGF, FLG2, IRS1, MTOR, PIK3CD, SF3B1, TOP1, BLM, CERK, EGFR, FLNC, IRS2, MUTYH, PIK3CG, SHC1, and TOP2A.

The term “genomic sequence”, as used herein, generally refers to a sequence that occurs in a genome. Because RNAs are transcribed from a genome, this term encompasses sequence that exist in the nuclear genome of an organism, as well as sequences that are present in a cDNA copy of an RNA (e.g., an mRNA) transcribed from such a genome.

The terms “anneal”, “hybridize” or “bind,” can refer to two polynucleotide sequences, segments or strands, and can be used interchangeably and have the usual meaning in the art. Two complementary sequences (e.g., DNA and/or RNA) can anneal or hybridize by forming hydrogen bonds with complementary bases to produce a double-stranded polynucleotide or a double-stranded region of a polynucleotide.

As used herein, the term “complementary” generally refers to a relationship between two antiparallel nucleic acid sequences in which the sequences are related by the base-pairing rules: A pairs with T or U and C pairs with G. A first sequence or segment that is “perfectly complementary” to a second sequence or segment is complementary across its entire length and has no mismatches. A first sequence or segment is “substantially complementary” to a second sequence of segment when a polynucleotide consisting of the first sequence is sufficiently complementary to specifically hybridize to a polynucleotide consisting of the second sequence.

The term “duplex,” or “duplexed,” as used herein, can describe two complementary polynucleotides that are base-paired, e.g., hybridized together.

As used herein, the term “Tm” generally refers to the melting temperature of an oligonucleotide duplex at which half of the duplexes remain hybridized and half of the duplexes dissociate into single strands. See Sambrook and Russell (2001; Molecular Cloning: A Laboratory Manual, 3^{rd} ed., Cold Spring Harbor Press, Cold Spring Harbor N.Y., ch. 10).

As used herein, “amplification” of a nucleic acid sequence generally refers to in vitro techniques for enzymatically increasing the number of copies of a target sequence. Amplification methods include both asymmetric methods (in which the predominant product is single-stranded) and conventional methods (in which the predominant product is double-stranded). A “round” or “cycle” of amplification can refer to a PCR cycle in which a double stranded template DNA molecule is denatured into single-stranded templates, forward and reverse primers are hybridized to the single stranded templates to form primer/template duplexes, primers are extended by a DNA polymerase from the primer/template duplexes to form extension products. In subsequent rounds of amplification the extension products are denatured into single stranded templates and the cycle is repeated.

The terms “template”, “template strand”, “template DNA” and “template nucleic acid” can be used interchangeably herein to refer to a strand of DNA that is copied by an amplification cycle.

The term “denaturing,” as used herein, generally refers to the separation of a nucleic acid duplex into two single strands.

The term “extending”, as used herein, generally refers to the extension of a primer hybridized to a template nucleic acid by the addition of nucleotides using an enzyme, e.g., a polymerase.

A “primer” is generally a nucleotide sequence (e.g., an oligonucleotide), generally with a free 3′—OH group, that hybridizes with a template sequence (such as a target polynucleotide, or a primer extension product) and is capable of promoting polymerization of a polynucleotide complementary to the template. A primer can be, for example, a sequence of the template (such as a primer extension product or a fragment of the template created following RNase cleavage of a template-DNA complex) that is hybridized to a sequence in the template itself (for example, as a hairpin loop), and that is capable of promoting nucleotide polymerization. Thus, a primer can be an exogenous (e.g., added) primer or an endogenous (e.g., template fragment) primer.

The terms “determining”, “measuring”, “evaluating”, “assessing,” “assaying,” and “analyzing” can be used interchangeably herein to refer to any form of measurement, and include determining if an element is present or not. These terms can include both quantitative and/or qualitative determinations. Assessing may be relative or absolute. “Assessing the presence of” can include determining the amount of something present, as well as determining whether it is present or absent.

The term “free in solution,” as used here, can describe a molecule, such as a polynucleotide, that is not bound or tethered to a solid support.

The term “genomic fragment”, as used herein, can refer to a region of a genome, e.g., an animal or plant genome such as the genome of a human, monkey, rat, fish or insect or plant. A genomic fragment may or may not be adaptor ligated. A genomic fragment may be adaptor ligated (in which case it has an adaptor ligated to one or both ends of the fragment, to at least the 5′ end of a molecule), or non-adaptor ligated.

“Pre-amplification”, as used herein, generally refers to non-clonal amplification of nucleic acids. For example, pre-amplification of a nucleic acid library is generally performed prior to clonal amplification of the library and/or loading onto a sequencer.

The term “ligase”, as used herein, generally refers to an enzyme that is commonly used to join polynucleotides together or to join the ends of a single polynucleotide.

The term “ligation”, as used herein, generally refers to the joining of two ends of polynucleotides or the joining of ends of a single polynucleotide by the formation of a covalent bond between the ends to be joined. The covalent bond can be a phosphodiester bond.

The term “ATP-dependent ligation”, as used herein, generally refers to ligation by an ATP-dependent ligase. An exemplary mechanism of ATP-dependent ligation is described herein.

“Donor” and “acceptor” nucleic acid species generally refer to two distinct populations of nucleic acid molecules to be joined in a ligation reaction. The “donor” species generally refers to a population of nucleic acid molecules which may accept a nucleoside monophosphate(NMP) at either a 5′ or 3′ end. The “acceptor” species generally refers to a second population of nucleic acid molecules containing a 3′ or 5′ OH group which may be ligated to the “donor” species via the NMP at either the 5′ or 3′ end of the donor species.

The donor and acceptor species can be any nucleic acid species. They can be, for example, polynucleotides isolated from a biological source. The biological source can be a subject. Exemplary biological sources and subjects are described herein. They can be oligonucleotides. Methods for preparing oligonucleotides of specific sequence are known in the art, and include, for example, cloning and restriction of appropriate sequences and direct chemical synthesis. Chemical synthesis methods may include, for example, the phosphotriester method described by Narang et al., 1979, Methods in Enzymology 68:90, the phosphodiester method disclosed by Brown et al., 1979, Methods in Enzymology 68:109, the diethylphosphoramidate method disclosed in Beaucage et al., 1981, Tetrahedron Letters 22:1859, and the solid support method disclosed in U.S. Pat. No. 4,458,066. They can be RNA or DNA. The DNA can be partially or fully denatured DNA. The DNA can be single stranded (ss)DNA. Partially denatured can be “frayed” at ends such that a “frayed” end can comprise 1, 2, 3, 4, 5, or more than 5 non-annealed nucleotides.

The donor and/or acceptor nucleic acid species can be of any size, ranging from, e.g., 1-50 nt, 10-100 nt, 50-200 nt, 100-400 nt, 200-600 nt, 500-1000 nt, 800-2000 nt, or greater than 2000 nt. In some embodiments, the donor and/or acceptor nucleic acid species is over 120 nt long.

The donor or acceptor nucleic acid species can include, e.g., genomic nucleic acids, adaptor sequences, and/or barcode sequences. The donor or acceptor nucleic acid species can include oligonucleotides. The donor or acceptor nucleic acid species can comprise a detectable label or affinity tag.

The detectable label can be any molecule that enables detection of a molecule to be detected. Non-limiting examples of detectable labels include, e.g., chelators, photoactive agents, radioactive moieties (e.g., alpha, beta and gamma emitters), fluorescent agents, luminescent agents, paramagnetic ions, or enzymes that produce a detectable signal in the presence of certain reagents (e.g., horseradish peroxidase, alkaline phosphatase, glucose oxidase).

Exemplary fluorescent compounds include, e.g., fluorescein isothiocyanate, rhodamine, phycoerytherin, phycocyanin, allophycocyanin, o-phthaldehyde, fluorescamine, and commercially available fluorophores such as Alexa Fluor 350, Alexa Fluor 488, Alexa Fluor 532, Alexa Fluor 546, Alexa Fluor 568, Alexa Fluor 594, Alexa Fluor 647, DyLight dyes such as DyLight 488, DyLight 594, DyLight 647, and BODIPY dyes such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine and Texas Red. Such compounds are commercially available (see, e.g., Molecular Probes, Inc.).

The affinity tag can be selected to have affinity to a capture moiety. The affinity tag can comprise, by way of non-limiting example only, biotin, desthiobiotin, histidine, polyhistidine, my c, hemagglutinin (HA), FLAG, a fluorescence tag, a tandem affinity purification (TAP) tag, a FLAG tag, a glutathione S transferase (GST) tag, or derivatives thereof. The capture moiety can comprise, e.g., avidin streptavidin, Neutravidin™, nickel, or glutathione or other molecule capable of binding the affinity tag.

In some embodiments, the acceptor species and the donor species can be the same species. For example, in some embodiments a user may desire to circularize a linear nucleic acid or to form concatemers of a single nucleic acid species.

The term “reaction mixture” as used herein generally refers to a mixture of components necessary to effect a desired reaction. The mixture may further comprise a buffer (e.g., a Tris buffer). The reaction mixture may further comprise a monovalent salt. The reaction mixture may further comprise a cation, e.g., Mg²⁺ and/or Mn²⁺. The concentration of each component is well known in the art and can be further optimized by an ordinary skilled artisan. In some embodiments, the reaction mixture also comprises additives including, but not limited to, non-specific background/blocking nucleic acids (e.g., salmon sperm DNA), non-specific background/blocking proteins (e.g., bovine serum albumin, non-fat dry milk) biopreservatives (e.g. sodium azide), PCR enhancers (e.g. Betaine, Trehalose, etc.), and inhibitors (e.g. RNAse inhibitors). In some embodiments, a nucleic acid sample is admixed with the reaction mixture.

A “primer binding site” can refer to a site to which a primer hybridizes in an oligonucleotide or a complementary strand thereof.

The term “separating”, as used herein, can refer to physical separation of two elements (e.g., by size, affinity, degradation of one element etc.).

The term “sequencing”, as used herein, can refer to a method by which the identity of at least 10 consecutive nucleotides (e.g., the identity of at least 20, at least 50, at least 100, at least 200, or at least 500 or more consecutive nucleotides) of a polynucleotide are obtained.

The term “adaptor-ligated”, as used herein, can refer to a nucleic acid that has been ligated to an adaptor. The adaptor can be ligated to a 5′ end or a 3′ end of a nucleic acid molecule, or can be added to an internal region of a nucleic acid molecule.

The term “bridge PCR” can refer to a solid-phase polymerase chain reaction in which the primers that are extended in the reaction are tethered to a substrate by their 5′ ends. During amplification, the amplicons form a bridge between the tethered primers. Bridge PCR (which may also be referred to as “cluster PCR”) is used in Illumina's Solexa platform. Bridge PCR and Illumina's Solexa platform are generally described in a variety of publications, e.g., Gudmundsson et al (Nat. Genet. 2009 41:1122-6), Out et al (Hum. Mutat. 2009 30:1703-12) and Turner (Nat. Methods 2009 6:315-6), U.S. Pat. No. 7,115,400, and publication application publication nos. US20080160580 and US20080286795.

The term “barcode sequence” as used herein, generally refers to a unique sequence of nucleotides that can encode information about an assay. A barcode sequence can encode information relating to the identity of an interrogated allele, identity of a target polynucleotide or genomic locus, identity of a sample, a subject, or any combination thereof. A barcode sequence can be a portion of a primer, a reporter probe, or both. A barcode sequence may be at the 5′-end or 3′-end of an oligonucleotide, or may be located in any region of the oligonucleotide. A barcode sequence may or may not be part of a template sequence. Barcode sequences may vary widely in size and composition; the following references provide guidance for selecting sets of barcode sequences appropriate for particular embodiments: Brenner, U.S. Pat. No. 5,635,400; Brenner et al, Proc. Natl. Acad. Sci., 97: 1665-1670 (2000); Shoemaker et al, Nature Genetics, 14: 450-456 (1996); Morris et al, European patent publication 0799897A1; Wallace, U.S. Pat. No. 5,981,179. A barcode sequence may have a length of about 4 to 36 nucleotides, about 6 to 30 nucleotides, or about 8 to 20 nucleotides.

The term “mutation”, as used herein, generally refers to a change of the nucleotide sequence of a genome. Mutations can involve large sections of DNA (e.g., copy number variation). Mutations can involve whole chromosomes (e.g., aneuploidy). Mutations can involve small sections of DNA. Examples of mutations involving small sections of DNA include, e.g., point mutations or single nucleotide polymorphisms, multiple nucleotide polymorphisms, insertions (e.g., insertion of one or more nucleotides at a locus), multiple nucleotide changes, deletions (e.g., deletion of one or more nucleotides at a locus), and inversions (e.g., reversal of a sequence of one or more nucleotides).

The term “locus”, as used herein, can refer to a location of a gene, nucleotide, or sequence on a chromosome. An “allele” of a locus, as used herein, can refer to an alternative form of a nucleotide or sequence at the locus. A “wild-type allele” generally refers to an allele that has the highest frequency in a population of subjects. A “wild-type” allele generally is not associated with a disease. A “mutant allele” generally refers to an allele that has a lower frequency that a “wild-type allele” and may be associated with a disease. A “mutant allele” may not have to be associated with a disease. The term “interrogated allele” generally refers to the allele that an assay is designed to detect.

The term “single nucleotide polymorphism”, or “SNP”, as used herein, generally refers to a type of genomic sequence variation resulting from a single nucleotide substitution within a sequence. “SNP alleles” or “alleles of a SNP” generally refer to alternative forms of the SNP at particular locus. The term “interrogated SNP allele” generally refers to the SNP allele that an assay is designed to detect.

The term “copy number variation” or “CNV” refers to differences in the copy number of genetic information. In many aspects it refers to differences in the per genome copy number of a genomic region. For example, in a diploid organism the expected copy number for autosomal genomic regions is 2 copies per genome. Such genomic regions should be present at 2 copies per cell. For a recent review see Zhang et al. Annu. Rev. Genomics Hum. Genet. 2009. 10:451-81. CNV is a source of genetic diversity in humans and can be associated with complex disorders and disease, for example, by altering gene dosage, gene disruption, or gene fusion. They can also represent benign polymorphic variants. CNVs can be large, for example, larger than 1 Mb, but many are smaller, for example between 100 bases and 1 Mb. More than 38,000 CNVs greater than 100 bases (and less than 3 Mb) have been reported in humans. Along with SNPs these CNVs account for a significant amount of phenotypic variation between individuals. In addition to having deleterious impacts, e.g. causing disease, they may also result in advantageous variation.

In certain cases, an oligonucleotide used in the method described herein may be designed using a reference genomic region, i.e., a genomic region of known nucleotide sequence, e.g., a chromosomal region whose sequence is deposited at NCBI's Genbank database or other database, for example.

The term “genotyping”, as used herein, generally refers to a process of determining differences in the genetic make-up (genotype) of an individual by examining the individual's DNA sequence using biological assays and comparing it to another individual's sequence or a reference sequence.

A “plurality” generally contains at least 2 members. In certain cases, a plurality may have at least 10, at least 100, at least 100, at least 10,000, at least 100,000, at least 1000000, at least 10000000, at least 100000000, or at least 1000000000 or more members.

The term “separating”, as used herein, generally refers to physical separation of two elements (e.g., by cleavage, hydrolysis, or degradation of one of the two elements).

The terms “label” and “detectable moiety” can be used interchangeably herein to refer to any atom or molecule which can be used to provide a detectable signal, and which can be attached to a nucleic acid or protein. Labels may provide signals detectable by fluorescence, radioactivity, colorimetry, gravimetry, X-ray diffraction or absorption, magnetism, enzymatic activity, and the like.

### Overview

Aspects of the invention relate to methods and kits that improve the monitoring and treatment of a subject suffering from a disease. The disease can be a cancer, e.g., a tumor, a leukemia such as acute leukemia, acute t-cell leukemia, acute lymphocytic leukemia, acute myelocytic leukemia, myeloblastic leukemia, promyelocytic leukemia, myelomonocytic leukemia, monocytic leukemia, erythroleukemia, chronic leukemia, chronic myelocytic (granulocytic) leukemia, or chronic lymphocytic leukemia, polycythemia vera, lymphomas such as Hodgkin's lymphoma, follicular lymphoma or non-Hodgkin's lymphoma, multiple myeloma, Waldenström's macroglobulinemia, heavy chain disease, solid tumors, sarcomas, carcinomas such as, e.g., fibrosarcoma, myxosarcoma, liposarcoma, chondrosarcoma, osteogenic sarcoma, lymphangiosarcoma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic, carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, craniopharyngioma, ependymoma, pinealoma, hemangioblastoma, acoustic neuroma, oligodendroglioma, meningioma, melanoma, neuroblastoma, retinoblastoma, endometrial cancer, non small cell lung cancer,

The subject can be suspected or known to harbor a solid tumor, or can be a subject who previously harbored a solid tumor.

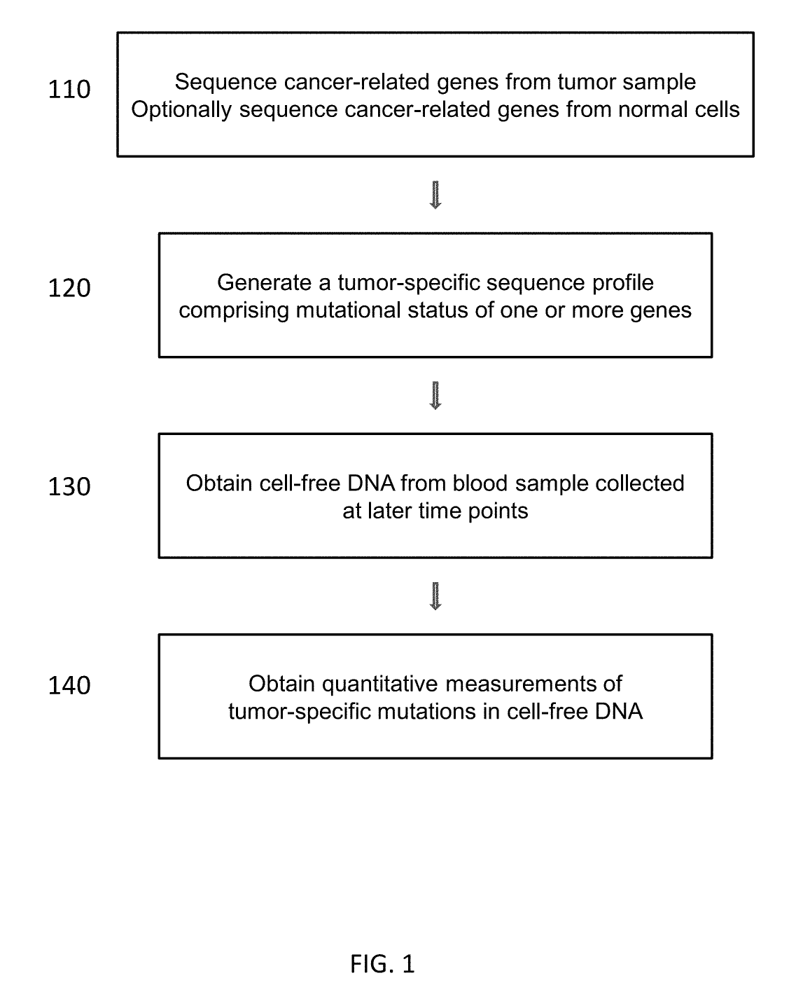

FIG. 1 depicts an exemplary workflow of a method for assessing cancer. In step 110, the method comprises sequencing cancer-related genes from a tumor sample isolated from said subject and optionally sequencing a set of cancer-related genes from normal cells isolated from said subject. The tumor sample can be a solid tumor sample. The normal cells can be blood cells isolated from a blood sample from the subject. In step 120, sequence data from the tumor can be used to determine a tumor-specific sequence profile. In some embodiments, sequence data from the tumor is compared to sequence data from normal cells to generate the tumor-specific sequence profile. In some embodiments, the tumor-specific sequence profile comprises mutational status of one or more genes in the set. The method can further comprise generating a report describing the tumor-specific sequence profile. In some embodiments, the method further comprises choosing a subset of 2-4 genes known to harbor tumor-specific mutations for further monitoring. In some embodiments, the method comprises choosing a subset of no more than 4 genes known to harbor tumor-specific mutations. In step 130, cell-free DNA is obtained from a blood sample collected from the subject prior to treatment (e.g., tumor removal or therapeutic intervention) as well as prior to treatment (tumor removal or therapeutic intervention) as well as at a later time point. In step 140, the cell-free DNA from the blood sample is assayed for the 2-4 genes in the subset to obtain quantitative measurement of the tumor-specific mutations.

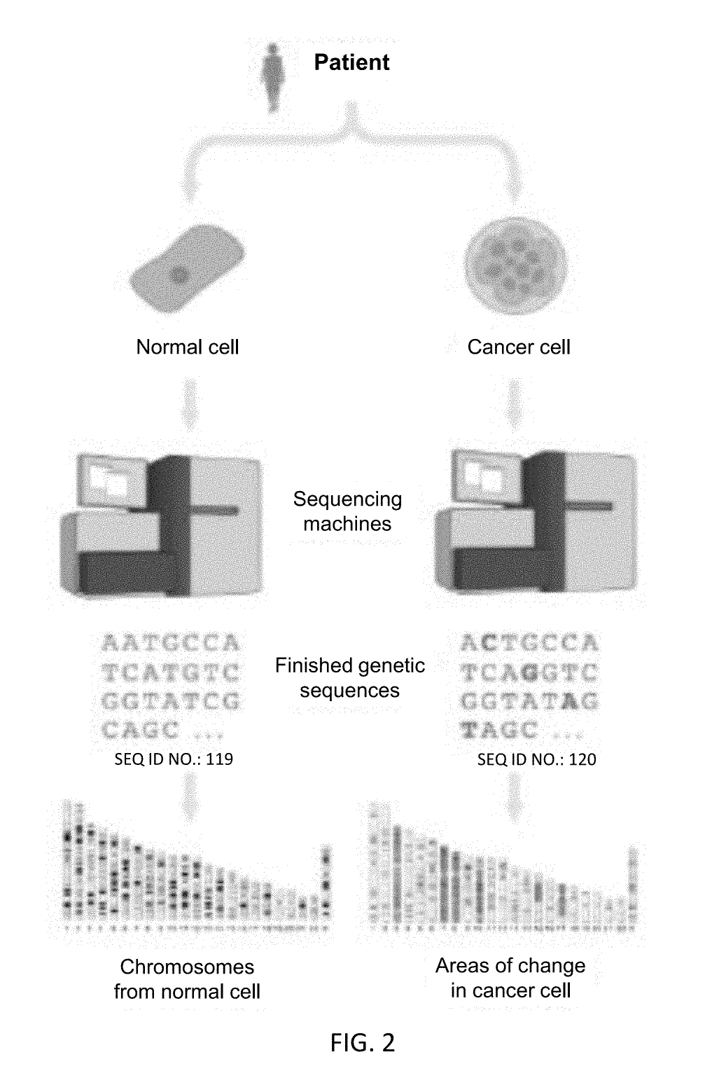

FIG. 2 is a depiction of an exemplary workflow of a method as described in FIG. 1, from steps 110-120, for sequencing a tumor cell and a normal cell in a subject.

The tumor sample can be processed prior to sequencing by fixation in a formalin solution, followed by embedding in paraffin (e.g., is a FFPE sample). In some embodiments, the tumor sample is frozen prior to sequencing. In some embodiments, the tumor sample is neither fixed nor frozen. The unfixed, unfrozen tumor sample can be stored in a storage solution configured for the preservation of nucleic acid at room temperature. The storage solution can be a commercially available storage solution. Exemplary storage solutions include, but are not limited to, DNA storage solutions from Biomatrica (see, e.g., WO/2012/018638, WO/2009/038853, US20080176209), hereby incorporated by reference.

Further embodiments of the sequencing methods and assays for determining mutational status in the blood are described herein.

### Next-Generation Sequencing

In some embodiments, the tumor sample and normal cells from the subject are sequenced. In some embodiments, nucleic acid is isolated from the tumor sample and normal cells using any methods known in the art. The nucleic acid is DNA. The DNA from the tumor sample and normal cells can be used to prepare a subject-specific tumor DNA library and/or normal DNA library. DNA libraries can be used for sequencing by a sequencing platform. The sequencing platform can be a next-generation sequencing (NGS) platform. In some embodiments, the method further comprises sequencing the nucleic acid libraries using NGS technology. NGS technology can involve sequencing of clonally amplified DNA templates or single DNA molecules in a massively parallel fashion (e.g. as described in Volkerding et al. Clin Chem 55:641-658 [2009]; Metzker M Nature Rev 11:31-46 [2010]). In addition to high-throughput sequence information, NGS provides digital quantitative information, in that each sequence read is a countable “sequence tag” representing an individual clonal DNA template or a single DNA molecule.

### Next Generation Sequencing Platforms

The next-generation sequencing platform can be a commercially available platform. Commercially available platforms include, e.g., platforms for sequencing-by-synthesis, ion semiconductor sequencing, pyrosequencing, reversible dye terminator sequencing, sequencing by ligation, single-molecule sequencing, sequencing by hybridization, and nanopore sequencing. Platforms for sequencing by synthesis are available from, e.g., Illumina, 454 Life Sciences, Helicos Biosciences, and Qiagen. Illumina platforms can include, e.g., Illumina's Solexa platform, Illumina's Genome Analyzer, and are described in Gudmundsson et al (Nat. Genet. 2009 41:1122-6), Out et al (Hum. Mutat. 2009 30:1703-12) and Turner (Nat. Methods 2009 6:315-6), U.S. Patent Application Pub nos. US20080160580 and US20080286795, U.S. Pat. Nos. 6,306,597, 7,115,400, and 7,232,656. 454 Life Science platforms include, e.g., the GS Flex and GS Junior, and are described in U.S. Pat. No. 7,323,305. Platforms from Helicos Biosciences include the True Single Molecule Sequencing platform. Platforms for ion seminconductor sequencing include, e.g., the Ion Torrent Personal Genome Machine (PGM) and are described in U.S. Pat. No. 7,948,015. Platforms for pryosequencing include the GS Flex 454 system and are described in U.S. Pat. Nos. 7,211,390; 7,244,559; 7,264,929. Platforms and methods for sequencing by ligation include, e.g., the SOLiD sequencing platform and are described in U.S. Pat. No. 5,750,341. Platforms for single-molecule sequencing include the SMRT system from Pacific Bioscience and the Helicos True Single Molecule Sequencing platform.

While the automated Sanger method is considered as a ‘first generation’ technology, Sanger sequencing including the automated Sanger sequencing, can also be employed by the method of the invention. Additional sequencing methods that comprise the use of developing nucleic acid imaging technologies e.g. atomic force microscopy (AFM) or transmission electron microscopy (TEM), are also encompassed by the method of the invention. Exemplary sequencing technologies are described below.

The DNA sequencing technology can utilize the Ion Torrent sequencing platform, which pairs semiconductor technology with a sequencing chemistry to directly translate chemically encoded information (A, C, G, T) into digital information (0, 1) on a semiconductor chip. Without wishing to be bound by theory, when a nucleotide is incorporated into a strand of DNA by a polymerase, a hydrogen ion is released as a byproduct. The Ion Torrent platform detects the release of the hydrogen atom as a change in pH. A detected change in pH can be used to indicate nucleotide incorporation. The Ion Torrent platform comprises a high-density array of micro-machined wells to perform this biochemical process in a massively parallel way. Each well holds a different library member, which may be clonally amplified. Beneath the wells is an ion-sensitive layer and beneath that an ion sensor. The platform sequentially floods the array with one nucleotide after another. When a nucleotide, for example a C, is added to a DNA template and is then incorporated into a strand of DNA, a hydrogen ion will be released. The charge from that ion will change the pH of the solution, which can be identified by Ion Torrent's ion sensor. If the nucleotide is not incorporated, no voltage change will be recorded and no base will be called. If there are two identical bases on the DNA strand, the voltage will be double, and the chip will record two identical bases called. Direct identification allows recordation of nucleotide incorporation in seconds. Library preparation for the Ion Torrent platform generally involves ligation of two distinct adaptors at both ends of a DNA fragment.

The DNA sequencing technology utilizes an Illumina sequencing platform, which generally employs cluster amplification of library members onto a flow cell and a sequencing-by-synthesis approach. Cluster-amplified library members are subjected to repeated cycles of polymerase-directed single base extension. Single-base extension can involve incorporation of reversible-terminator dNTPs, each dNTP labeled with a different removable fluorophore. The reversible-terminator dNTPs are generally 3′ modified to prevent further extension by the polymerase. After incorporation, the incorporated nucleotide can be identified by fluorescence imaging. Following fluorescence imaging, the fluorophore can be removed and the 3′ modification can be removed resulting in a 3′ hydroxyl group, thereby allowing another cycle of single base extension. Library preparation for the Illumina platform generally involves ligation of two distinct adaptors at both ends of a DNA fragment.

The DNA sequencing technology that is used in one or more methods of the invention can be the Helicos True Single Molecule Sequencing (tSMS), which can employ sequencing-by-synthesis technology. In the tSMS technique, a polyA adaptor can be ligated to the 3′ end of DNA fragments. The adapted fragments can be hybridized to poly-T oligonucleotides immobilized on the tSMS flow cell. The library members can be immobilized onto the flow cell at a density of about 100 million templates/cm2. The flow cell can be then loaded into an instrument, e.g., HeliScope™ sequencer, and a laser can illuminate the surface of the flow cell, revealing the position of each template. A CCD camera can map the position of the templates on the flow cell surface. The library members can be subjected to repeated cycles of polymerase-directed single base extension. The sequencing reaction begins by introducing a DNA polymerase and a fluorescently labeled nucleotide. The polymerase can incorporate the labeled nucleotides to the primer in a template directed manner. The polymerase and unincorporated nucleotides can be removed. The templates that have directed incorporation of the fluorescently labeled nucleotide can be discerned by imaging the flow cell surface. After imaging, a cleavage step can remove the fluorescent label, and the process can be repeated with other fluorescently labeled nucleotides until a desired read length is achieved. Sequence information can be collected with each nucleotide addition step.

The DNA sequencing technology can utilize a 454 sequencing platform (Roche) (e.g. as described in Margulies, M. et al. Nature 437:376-380 [2005]). 454 sequencing generally involves two steps. In a first step, DNA can be sheared into fragments. The fragments can be blunt-ended. Oligonucleotide adaptors can be ligated to the ends of the fragments. The adaptors generally serve as primers for amplification and sequencing of the fragments. At least one adaptor can comprise a capture reagent, e.g., a biotin. The fragments can be attached to DNA capture beads, e.g., streptavidin-coated beads. The fragments attached to the beads can be PCR amplified within droplets of an oil-water emulsion, resulting in multiple copies of clonally amplified DNA fragments on each bead. In a second step, the beads can be captured in wells, which can be pico-liter sized. Pyrosequencing can be performed on each DNA fragment in parallel. Pyrosequencing generally detects release of pyrophosphate (PPi) upon nucleotide incorporation. PPi can be converted to ATP by ATP sulfurylase in the presence of adenosine 5′ phosphosulfate. Luciferase can use ATP to convert luciferin to oxyluciferin, thereby generating a light signal that is detected. A detected light signal can be used to identify the incorporated nucleotide.

The DNA sequencing technology can utilize a SOLiD™ technology (Applied Biosystems). The SOLiD platform generally utilizes a sequencing-by-ligation approach. Library preparation for use with a SOLiD platform generally comprises ligation of adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations can be prepared in microreactors containing beads, primers, template, and PCR components. Following PCR, the templates can be denatured. Beads can be enriched for beads with extended templates. Templates on the selected beads can be subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide can be removed and the process can then be repeated.

The DNA sequencing technology can utilize a single molecule, real-time (SMRT™) sequencing platform (Pacific Biosciences). In SMRT sequencing, the continuous incorporation of dye-labeled nucleotides can be imaged during DNA synthesis. Single DNA polymerase molecules can be attached to the bottom surface of individual zero-mode wavelength identifiers (ZMW identifiers) that obtain sequence information while phospolinked nucleotides are being incorporated into the growing primer strand. A ZMW generally refers to a confinement structure which enables observation of incorporation of a single nucleotide by DNA polymerase against a background of fluorescent nucleotides that rapidly diffuse in an out of the ZMW on a microsecond scale. By contrast, incorporation of a nucleotide generally occurs on a milliseconds timescale. During this time, the fluorescent label can be excited to produce a fluorescent signal, which is detected. Detection of the fluorescent signal can be used to generate sequence information. The fluorophore can then be removed, and the process repeated. Library preparation for the SMRT platform generally involves ligation of hairpin adaptors to the ends of DNA fragments.

The DNA sequencing technology can utilize nanopore sequencing (e.g. as described in Soni G V and Meller A. Clin Chem 53: 1996-2001 [2007]). Nanopore sequencing DNA analysis techniques are being industrially developed by a number of companies, including Oxford Nanopore Technologies (Oxford, United Kingdom). Nanopore sequencing is a single-molecule sequencing technology whereby a single molecule of DNA is sequenced directly as it passes through a nanopore. A nanopore can be a small hole, of the order of 1 nanometer in diameter. Immersion of a nanopore in a conducting fluid and application of a potential (voltage) across can result in a slight electrical current due to conduction of ions through the nanopore. The amount of current which flows is sensitive to the size and shape of the nanopore and to occlusion by, e.g., a DNA molecule. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore represents a reading of the DNA sequence.

The DNA sequencing technology can utilize a chemical-sensitive field effect transistor (chemFET) array (e.g., as described in U.S. Patent Application Publication No. 20090026082). In one example of the technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be discerned by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

The DNA sequencing technology can utilize transmission electron microscopy (TEM). The method, termed Individual Molecule Placement Rapid Nano Transfer (IMPRNT), generally comprises single atom resolution transmission electron microscope imaging of high-molecular weight (150 kb or greater) DNA selectively labeled with heavy atom markers and arranging these molecules on ultra-thin films in ultra-dense (3 nm strand-to-strand) parallel arrays with consistent base-to-base spacing. The electron microscope is used to image the molecules on the films to determine the position of the heavy atom markers and to extract base sequence information from the DNA. The method is further described in PCT patent publication WO 2009/046445. The method allows for sequencing complete human genomes in less than ten minutes.

The method can utilize sequencing by hybridization (SBH). SBH generally comprises contacting a plurality of polynucleotide sequences with a plurality of polynucleotide probes, wherein each of the plurality of polynucleotide probes can be optionally tethered to a substrate. The substrate might be flat surface comprising an array of known nucleotide sequences. The pattern of hybridization to the array can be used to determine the polynucleotide sequences present in the sample. In other embodiments, each probe is tethered to a bead, e.g., a magnetic bead or the like. Hybridization to the beads can be identified and used to identify the plurality of polynucleotide sequences within the sample.

The length of the sequence read can vary depending on the particular sequencing technology utilized. NGS platforms can provide sequence reads that vary in size from tens to hundreds, or thousands of base pairs. In some embodiments of the method described herein, the sequence reads are about 20 bases long, about 25 bases long, about 30 bases long, about 35 bases long, about 40 bases long, about 45 bases long, about 50 bases long, about 55 bases long, about 60 bases long, about 65 bases long, about 70 bases long, about 75 bases long, about 80 bases long, about 85 bases long, about 90 bases long, about 95 bases long, about 100 bases long, about 110 bases long, about 120 bases long, about 130, about 140 bases long, about 150 bases long, about 200 bases long, about 250 bases long, about 300 bases long, about 350 bases long, about 400 bases long, about 450 bases long, about 500 bases long, about 600 bases long, about 700 bases long, about 800 bases long, about 900 bases long, about 1000 bases long, or more than 1000 bases long.

Partial sequencing of DNA fragments present in the sample can be performed, and sequence tags comprising reads that map to a known reference genome can be counted. Only sequence reads that uniquely align to the reference genome can be counted as sequence tags. In one embodiment, the reference genome is the human reference genome NCBI36/hg18 sequence, which is available on the world wide web at genome.ucsc.edu/cgi-bin/hgGateway?org=Human&db=hgl 8&hgsid=166260105). Other sources of public sequence information include GenBank, dbEST, dbSTS, EMBL (the European Molecular Biology Laboratory), and the DDBJ (the DNA Databank of Japan). The reference genome can also comprise the human reference genome NCBI36/hgl 8 sequence and an artificial target sequences genome, which includes polymorphic target sequences. In yet another embodiment, the reference genome is an artificial target sequence genome comprising polymorphic target sequences.

Mapping of the sequence tags can be achieved by comparing the sequence of the tag with the sequence of the reference genome to determine the chromosomal origin of the sequenced nucleic acid (e.g. cell free DNA) molecule, and specific genetic sequence information is not needed. A number of computer algorithms are available for aligning sequences, including without limitation BLAST (Altschul et al., 1990), BLITZ (MPsrch) (Sturrock & Collins, 1993), FASTA (Person & Lipman, 1988), BOWTIE (Langmead et al, Genome Biology 10:R25.1-R25.10 [2009]), or ELAND (Illumina, Inc., San Diego, Calif., USA). In one embodiment, one end of the clonally expanded copies of the DNA molecule is sequenced and processed by bioinformatic alignment analysis for the Illumina Genome Analyzer, which uses the Efficient Large-Scale Alignment of Nucleotide Databases (ELAND) software. Additional software includes SAMtools (SAMtools, Bioinformatics, 2009, 25(16):2078-9), and the Burroughs-Wheeler block sorting compression procedure which involves block sorting or preprocessing to make compression more efficient.

The sequencing platforms described herein generally comprise a solid support immobilized thereon surface-bound oligonucleotides which allow for the capture and immobilization of sequencing library members to the solid support. Surface bound oligonucleotides generally comprise sequences complementary to the adaptor sequences of the sequencing library.

Nucleic acid samples can be used to prepare nucleic acid libraries for sequencing. Preparation of nucleic acid libraries can comprise any method known in the art or as described herein. As used herein, the terms “library” or “sequencing library” are used interchangeably herein and can refer to a plurality of nucleic acid fragments obtained from a biological sample. Generally, the fragments are modified with an adaptor sequence which affects coupling (e.g., capture and/or immobilization) of the fragments to a sequencing platform. An adaptor sequence can comprise a defined oligonucleotide sequence that affects coupling of a library member to a sequencing platform. By way of example only, the adaptor can comprise a sequence that is at least 25% complementary or identical to an oligonucleotide sequence immobilized onto a solid support (e.g., a sequencing flow cell or bead). An adaptor sequence can comprise a defined oligonucleotide sequence that is at least 70% complementary or identical to a sequencing primer. The sequencing primer can enable nucleotide incorporation by a polymerase, wherein incorporation of the nucleotide is monitored to provide sequencing information. The sequencing primer can be about 15-25 bases. In some embodiments, the sequencing primer is conjugated to the 3′ end of the adaptor. In some embodiments, an adaptor comprises a sequence that is at least 25% complementary or identical to an oligonucleotide sequence immobilized onto a solid support and a sequence that is at least 70% complementary or identical to a sequencing primer. Coupling can also be achieved through serially stitching adaptors together. The number of adaptors that can be stitched can be 1, 2, 3, 4 or more. The stitched adaptors can be at least 35 bases, 70 bases, 105 bases, 140 bases or more.

The adaptor can comprise a barcode sequence. At least 50%, 60%, 70%, 80%, 90%, or 100% of sequencing library members in a library can comprise the same adaptor sequence. At least 50%, 60%, 70%, 80%, 90%, or 100% of the ssDNA library members can comprise an adaptor sequence at a first end but not at a second end. In some embodiments, the first end is a 5′ end. In some embodiments, the first end is at 3′ end. The adaptor sequence can be chosen by a user according to the sequencing platform used for sequencing. By way of example only, an Illumina sequencing by synthesis platform comprises a solid support with a first and second population of surface-bound oligonucleotides immobilized thereon. Such oligonucleotides comprise a sequence for hybridizing to a first and second Illumina-specific adaptor oligonucleotide and priming an extension reaction. Accordingly, a DNA library member can comprise a first Illumina-specific adaptor that is partially or wholly complementary to a first population of surface bound oligonucleotides of an Illumina system. By way of other example only, the SOLiD system, and Ion Torrent, GS FLEX system comprises a solid support in the form of a bead with a single population of surface bound oligonucleotides immobilized thereon. Accordingly, in some embodiments the ssDNA library member comprises an adaptor sequence that is complementary to a surface-bound oligonucleotide of a SOLiD system, Ion Torrent system, or GS Flex system.

Accordingly, in one aspect, the invention provides improved methods of preparing a nucleic acid library. The nucleic acid library can be a DNA library. The method can comprise ligation of adaptor sequences to DNA fragments. The method can improve efficiency of adaptor ligation by at least 10-fold. In some embodiments, the nucleic acid library is a ssDNA library. In some embodiments, the nucleic acid library is a partial ssDNA library.

ssDNA Fragment/ssDNA Library Preparation

In some embodiments, the ssDNA fragment is a member of a ssDNA library. The single-stranded nucleic acid library is prepared from a sample of double-stranded nucleic acid using any means known in the art or described herein.

The starting sample can be a biological sample obtained from a subject. Exemplary subjects and biological samples are described herein. In particular embodiments, the sample is a solid biological sample, e.g., a tumor sample. In some embodiments, the solid biological sample is processed prior to the probe-based assay. Processing can comprise fixation in a formalin solution, followed by embedding in paraffin (e.g., is a FFPE sample). Processing can alternatively comprise freezing of the sample prior to conducting the probe-based assay. In some embodiments, the sample is neither fixed nor frozen. The unfixed, unfrozen sample can be, by way of example only, stored in a storage solution configured for the preservation of nucleic acid. Exemplary storage solutions are described herein. In some embodiments, non-nucleic acid materials can be removed from the starting material using enzymatic treatments (for example, with a protease). The sample can optionally be subjected to homogenization, sonication, French press, dounce, freeze/thaw, which can be followed by centrifugation. The centrifugation may separate nucleic acid-containing fractions from non-nucleic acid-containing fractions. In some embodiments, the sample is a liquid biological sample. Exemplary liquid biological samples are described herein. In some embodiments, the liquid biological sample is a blood sample (e.g., whole blood, plasma, or serum). In some embodiments, a whole blood sample is subjected to acellular components (e.g., plasma, serum) and cellular components by use of a Ficoll reagentm described in detail Fuss et al, Curr Protoc Immunol (2009) Chapter 7:Unit7.1, which is incorporated herein by reference.

Nucleic acid can be isolated from the biological sample using any means known in the art. For example, nucleic acid can be extracted from the biological sample using liquid extraction (e.g, Trizol, DNAzol) techniques. Nucleic acid can also be extracted using commercially available kits (e.g., Qiagen DNeasy kit, QIAamp kit, Qiagen Midi kit, QIAprep spin kit).

Nucleic acid can be concentrated by known methods, including, by way of example only, centrifugation. Nucleic acid can be bound to a selective membrane (e.g., silica) for the purposes of purification. Nucleic acid can also be enriched for fragments of a desired length, e.g., fragments which are less than 1000, 500, 400, 300, 200 or 100 base pairs in length. Such an enrichment based on size can be performed using, e.g., PEG-induced precipitation, an electrophoretic gel or chromatography material (Huber et al. (1993) Nucleic Acids Res. 21:1061-6), gel filtration chromatography, TSK gel (Kato et al. (1984) J. Biochem, 95:83-86), which publications are hereby incorporated by reference.

Polynucleotides extracted from a biological sample can be selectively precipitated or concentrated using any methods known in the art.

The nucleic acid sample can be enriched for target polynucleotides. Target enrichment can be by any means known in the art. For example, the nucleic acid sample may be enriched by amplifying target sequences using target-specific primers. The target amplification can occur in a digital PCR format, using any methods or systems known in the art. The nucleic acid sample may be enriched by capture of target sequences onto an array immobilized thereon target-selective oligonucleotides. The nucleic acid sample may be enriched by hybridizing to target-selective oligonucleotides free in solution or on a solid support. The oligonucleotides may comprise a capture moiety which enables capture by a capture reagent. Exemplary capture moieties and capture reagents are described herein. In some embodiments, the nucleic acid sample is not enriched for target polynucleotides, e.g., represents a whole genome.

Accordingly, in some aspects the invention provides a method of preparing a single-stranded nucleic acid library. The single-stranded nucleic acid library can be a single-stranded DNA library (ssDNA library) or an RNA library. A method of preparing an ssDNA library can comprise denaturing a double stranded DNA fragment into ssDNA fragments, ligating a primer docking sequence onto one end of the ssDNA fragment, hybridizing a primer to the primer docking sequence. The primer can comprise at least a portion of an adaptor sequence that couples to a next-generation sequencing platform. The method can further comprise extension of the hybridized primer to create a duplex, wherein the duplex comprises the original ssDNA fragment and an extended primer strand. The extended primer strand can be separated from the original ssDNA fragment. The extended primer strand can be collected, wherein the extended primer strand is a member of the ssDNA library. A method of preparing an RNA library can comprise ligating a primer docking sequence onto one end of the RNA fragment, hybridizing a primer to the primer docking sequence. The primer can comprise at least a portion of an adaptor sequence that couples to a next-generation sequencing platform. The method can further comprise extension of the hybridized primer to create a duplex, wherein the duplex comprises the original RNA fragment and an extended primer strand. The extended primer strand can be separated from the original RNA fragment. The extended primer strand can be collected, wherein the extended primer strand is a member of the RNA library.

dsDNA can be fragmented by any means known in the art or as described herein. dsDNA can be fragmented, for example, by mechanical shearing, by nebulization, or by sonication.

In some embodiments, cDNA is generated from RNA using random primed reverse transcription (RNaseH+) to generate randomly sized cDNA.

The nucleic acid fragments (e.g., dsDNA fragments, RNA, or randomly sized cDNA) can be less than 1000 bp, less than 800 bp, less than 700 bp, less than 600 bp, less than 500 bp, less than 400 bp, less than 300 bp, less than 200 bp, or less than 100 bp. The DNA fragments can be about 40-100 bp, about 50-125 bp, about 100-200 bp, about 150-400 bp, about 300-500 bp, about 100-500, about 400-700 bp, about 500-800 bp, about 700-900 bp, about 800-1000 bp, or about 100-1000 bp.

The ends of dsDNA fragments can be polished (e.g., blunt-ended). The ends of DNA fragments can be polished by treatment with a polymerase. Polishing can involve removal of 3′ overhangs, fill-in of 5′ overhangs, or a combination thereof. The polymerase can be a proof-reading polymerase (e.g., comprising 3′ to 5′ exonuclease activity). The proofreading polymerase can be, e.g., a T4 DNA polymerase, Pol 1 Klenow fragment, or Pfu polymerase. Polishing can comprise removal of damaged nucleotides (e.g. abasic sites), using any means known in the art.

Ligation of an adaptor to a 3′ end of a nucleic acid fragment can comprise formation of a bond between a 3′ OH group of the fragment and a 5′ phosphate of the adaptor. Therefore, removal of 5′ phosphates from nucleic acid fragments can minimize aberrant ligation of two library members. Accordingly, in some embodiments, 5′ phosphates are removed from nucleic acid fragments. In some embodiments, 5′ phosphates are removed from at least 50%, 55%, 60%, 65%, 70%, 75%, 80%, 85%, 90%, 95%, or greater than 95% of nucleic acid fragments in a sample. In some embodiments, substantially all phosphate groups are removed from nucleic acid fragments. In some embodiments, substantially all phosphates are removed from at least 50%, 55%, 60%, 65%, 70%, 75%, 80%, 85%, 90%, 95%, or greater than 95% of nucleic acid fragments in a sample. Removal of phosphate groups from a nucleic acid sample can be by any means known in the art. Removal of phosphate groups can comprise treating the sample with heat-labile phosphatase. In some embodiments, phosphate groups are not removed from the nucleic acid sample. In some embodiments ligation of an adaptor to the 5′ end of the nucleic acid fragment is performed.

### Denaturation

ssDNA can be prepared from dsDNA fragments prepared by any means in the art or as described herein, by denaturation into single strands. Denaturation of dsDNA can be by any means known in the art, including heat denaturation, incubation in basic pH, denaturation by urea or formaldehyde.

Heat denaturation can be achieved by heating a dsDNA sample to about 60 deg C or above, about 65 deg C or above, about 70 deg C or above, about 75 deg C or above, about 80 deg C or above, about 85 deg C or above, about 90 deg C or above, about 95 deg C or above, or about 98 deg C or above. The dsDNA sample can be heated by any means known in the art, including, e.g., incubation in a water bath, a temperature controlled heat block, a thermal cycler. In some embodiments the sample is heated for 0.5, 1, 2, 3, 4, 5, 6, 7, 8, 9, 10, or more than 10 minutes.

Denaturation by incubation in basic pH can be achieved by, for example, incubation of a dsDNA sample in a solution comprising sodium hydroxide (NaOH) or potassium hydroxide (KOH). The solution can comprise about 1 mM NAOH, 2 mM NAOH, 5 mM NAOH, 10 mM NAOH, 20 mM NAOH, 40 mM NAOH, 60 mM NAOH, 80 mM NAOH, 100 mM NAOH, 0.2M NaOH, about 0.3M NaOH, about 0.4M NaOH, about 0.5M NaOH, about 0.6M NaOH, about 0.7M NaOH, about 0.8M NaOH, about 0.9M NaOH, about 1.0M NaOH, or greater than 1.0M NaOH. The solution can comprise about 1 mM KOH, 2 mM KOH, 5 mM KOH, 10 mM KOH, 20 mM KOH, 40 mM KOH, 60 mM KOH, 80 mM KOH, 100 mM KOH, 0.2M KOH, 0.5M KOH, 1M KOH, or greater than 1M KOH. In some embodiments, the dsDNA sample is incubated in NaOH or KOH for 0.5, 1, 2, 3, 4, 5, 6, 7, 8, 9, 10, 15, 20, 30, 40, 50, 60, or more than 60 minutes. The dsDNA can be incubated in Na-acetate following NaOH or KOH incubation. The incubation in Na-acetate can neutralize the NaOH or KOH.

Compounds like urea and formamide contain functional groups that can form H-bonds with the electronegative centers of the nucleotide bases. At high concentrations (e.g., 8M urea or 70% formamide) of the denaturant, the competition for H-bonds favors interactions between the denaturant and the N-bases rather than between complementary bases, thereby separating the two strands.

Ligation of primer-docking oligonucleotide.

A primer-docking oligonucleotide (pdo) can be ligated onto one end of a nucleic acid fragment (e.g., ssDNA, RNA). The pdo can be ligated onto a 5′ end or a 3′ end. In some embodiments, the pdo is ligated onto a 3′ end of the nucleic acid fragment.

The pdo generally comprises a sequence that acts as a template for annealing a primer. The sequence of the pdo can comprise a sequence that is at least 70% complementary to a portion or all of an adaptor sequence for coupling to an NGS platform (NGS adaptor). The pdo can comprise a sequence complementary or identical to 5, 6, 7, 8, 9, 10, 11, 12, 13, 14, 15, 20, or more than 20 contiguous nucleotides of an NGS adaptor. In some embodiments, the pdo does not comprise a sequence complementary to a portion or all of an NGS adaptor.

The pdo can be adenylated at a 5′ end. The pdo can be conjugated to a capture moiety that is capable of forming a complex with a capture reagent. The capture moiety can be conjugated to the adaptor oligonucleotide by any means known in the art. Capture moiety/capture reagent pairs are known in the art. In some embodiments the capture reagent is avidin, streptavidin, or neutravidin and the capture moiety is biotin. In another embodiment the capture moiety/capture reagent pair is digoxigenin/wheat germ agglutinin.

Ligation of the pdo to the nucleic acid fragment can be effected by an ATP-dependent ligase. In some embodiments, the ATP-dependent ligase is an RNA ligase. The RNA ligase can be an ATP dependent ligase. The RNA ligase can be an Rnl 1 or Rnl 2 family ligase. Generally, Rnl 1 family ligases can repair single-stranded breaks in tRNA. Exemplary Rnl 1 family ligases include, e.g., T4 RNA ligase, thermostable RNA ligase 1 from Thermus scitoductus bacteriophage TS2126 (CircLigase), or CircLigase II. These ligases generally catalyze the ATP-dependent formation of a phosphodiester bond between a nucleotide 3-OH nucleophile and a 5′ phosphate group. Generally, Rnl 2 family ligases can seal nicks in duplex RNAs. Exemplary Rnl 2 family ligases include, e.g., T4 RNA ligase 2. The RNA ligase can be an Archaeal RNA ligase, e.g., an archaeal RNA ligase from the thermophilic archaeon Methanobacterium thermoautotrophicum (MthRn1).

The ligation of the pdo's to the single-stranded nucleic acid fragment can comprise preparing a reaction mixture comprising an nucleic acid fragment, a pdo, and ligase. In some embodiments the reaction mixture is heated to effect ligation of the adaptor oligonucleotides to the ss DNA fragments. In some embodiments the reaction mixture is heated to about 50 deg C, about 55 deg C, about 60 deg C, about 65 deg C, about 70 deg C, or above 70 deg C. In some embodiments the reaction mixture is heated to about 60-70 deg C. The reaction mixture can be heated for a sufficient time to effect ligation of the pdo to the nucleic acid fragment. In some embodiments, the reaction mixture is heated for about 5 min, about 10 min, about 15 min, about 20 min, about 25 min, about 30 min, about 35 min, about 40 min, about 45 min, about 50 min, about 55 min, about 60 min, about 70 min, about 80 min, about 90 min, about 120 min, about 150 min, about 180 min, about 210 min, about 240 min, or more than 240 min.

In some embodiments the pdo's are present in the reaction mixture in a concentration that is greater than the concentration of nucleic acid fragments in the mixture. In some embodiments, the pdo's are present at a concentration that is at least 10%, 20%, 30%, 40%, 60%, 60%, 70%, 80%, 90%, 100% or more than 100% greater than the concentration of nucleic acid fragments in the mixture. The pdo's can be present at concentration that is at least 10-fold, 100-fold, 1000-fold, or 10000-fold greater than the concentration of nucleic acid fragments in the mixture. The pdo's can be present at a final concentration of 0.1 uM, 0.5 uM, 1 uM, 10 uMor greater. In some embodiments the ligase is present in the reaction mixture at a saturating amount.

The reaction mixture can additionally comprise a high molecular weight inert molecule, e.g., PEG of MW 4000, 6000, or 8000. The inert molecule can be present in an amount that is about 0.5%, 1%, 2%, 3%, 4%, 5%, 7.5%, 10%, 12.5%, 15%, 17.5%, 20%, 25%, 30%, 35%, 40%, 45%, 50%, or greater than 50% weight/volume. In some embodiments, the inert molecular is present in an amount that is about 0.5-2%, about 1-5%, about 2-15%, about 10-20%, about 15-30%, about 20-50%, or more than 50% weight/volume.

After sufficient time has occurred to effect ligation of adaptors to the ss nucleic acid molecules, unreacted adaptors can be removed by any means known in the art, e.g., filtration by molecular weight cutoff, size exclusion chromatography, use of a spin column, selective precipitation with polyethylene glycol (PEG), selective precipitation with PEG onto a silica matrix, alcohol precipitation, sodium acetate precipitation, PEG and salt precipitation, or high stringency washing.

In some embodiments, the method further comprises capturing the ligated nucleic acid fragment. Capturing of the ligated nucleic acid fragment can occur prior to extension or subsequent to extension. The ligated nucleic acid fragment can be captured onto a solid support. Capturing can involve the formation of a complex comprising a capture moiety conjugated to a pdo and a capture reagent. In some embodiments, the capture reagent is immobilized onto a solid support. In some embodiments the solid support comprises an excess of capture reagent as compared to the amount of ligated nucleic acid comprising the capture moiety. In some embodiments the solid support comprises 5-fold, 10-fold, or 100-fold more available binding sites that the total number of ligated nucleic acid fragments comprising the capture moiety.

### Extension

In some embodiments, a primer is hybridized to the ligated nucleic acid fragment via the pdo. The primer can comprise a portion or entirety of an NGS adaptor sequence. Exemplary NGS adaptor sequences are described herein. In some embodiments, the primer is extended to create a duplex comprising the original nucleic acid fragment and the extended primer, wherein the extended primer comprises a reverse complement of the original nucleic acid fragment and an NGS adaptor sequence at one end. In some embodiments the NGS adaptor is at the 5′ end. Exemplary NGS adaptor sequences are described herein. In some embodiments, the NGS adaptor sequence comprises a sequence that is at least 70% identical to a surface-bound oligonucleotide of an NGS platform. In some embodiments, the NGS adaptor sequence comprises a sequence that is at least 70% complementary to a surface-bound oligonucleotide of an NGS platform. In some embodiments, the NGS adaptor sequence comprises a sequence that is at least 70% identical to a sequencing primer for use by an NGS platform. In some embodiments, the NGS adaptor sequence comprises a sequence that is at least 70% complementary to a sequencing primer for use by an NGS platform. Extension can be effected by a proofreading mesophilic or thermophilic DNA polymerase. Preferably, the polymerase is a thermophilic polymerase with 5′→3′ exonucleolytic/endonucleolytic (DNA polymerases I, II, III) or 3′→5′ exonucleolytic (family A or B DNA polymerases, DNA polymerase I, T4 DNA polymerase) activity. In some instances, the polymerase can have no exonuclease activity (Taq) In some cases, the polymerase effects linear amplification of the immobilized ligated fragment, creating a plurality of copies of the reverse complement of the immobilized ligated fragment. In other cases only one copy of the reverse complement is created. In some embodiments, the extended primer molecules are separated from the original nucleic acid template (e.g., by denaturation as described herein). The extended primer molecules are free in solution while the original nucleic acid template molecules remain immobilized to the solid support. The extended primer molecules can be easily harvested, resulting in a nucleic acid library preparation in which most of the library members comprise an NGS adaptor. At least 50%, 60%, 70%, 80%, 90%, more than 90%, or substantially all of the library members can comprise an NGS adaptor.

An exemplary workflow for preparing a single-stranded nucleic acid library (e.g., ssDNA library) is outlined below.

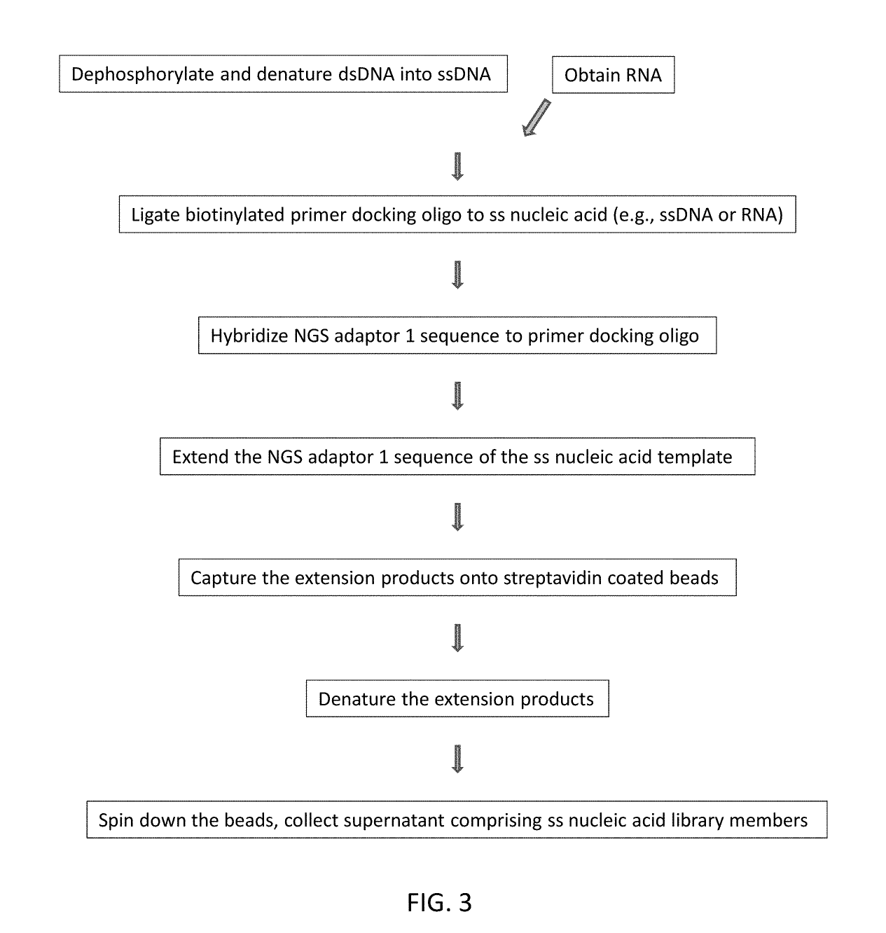

FIG. 3 depicts an exemplary embodiment of the method for preparing an nucleic acid library from nucleic acids (e.g., DNA or RNA) isolated from a biological sample (e.g., a blood, plasma, urine, stool, mucosal sample). The nucleic acids obtained can be fragmented by enzymatic or mechanical means to 100-1000, but preferably 100-500 bp fragments. The nucleic acids can be fragmented in situ. Nucleic acids can be fragmented from formalin-fixed paraffin-embedded (FFPE) tissues or circulating DNA. Nucleic acids can be isolated from FFPE and circulating by kits (Qiagen, Covaris). In some embodiments, the nucleic acids are DNA. In some embodiments, the DNA is cDNA generated from RNA isolated from a biological sample from the same samples using random primed reverse transcription (RNaseH+) to generate randomly sized cDNA. In some embodiments, then nucleic acids are RNA. Fragmented DNA can be treated with a base excision repair enzyme (Endo VIII, formamidopyrimidine DNA glycosylase (FPG)) to excise damaged bases that can interfere with polymerization. DNA can then be treated with a proof-reading polymerase (e.g. T4 DNA polymerase) to polish ends and replace damaged nucleotides (e.g. abasic sites). In some embodiments, DNA is not treated with a proof-reading polymerase to polish ends and replace damaged nucleotides.

In step 1, the nucleic acids (e.g., DNA or RNA) can be treated with heat-labile phosphatase to remove all phosphate groups from the nucleic acids. The reaction mixture can be heated to 80 deg C for 10 min to inactivate the phosphatase and polymerase and denature double stranded DNA to single strands.

In step 2, a chemically or enzymatically phosphorylated pdo containing a 3′-end affinity tag (e.g. biotin) 12 to 50 bases in length can be ligated to the fragmented single-strand nucleic acids at a final concentration of 0.5 uM or greater with saturating amount of ATP-dependent RNA ligase (T4 RNA ligase, but preferably thermophillic such as CircLigase, CircLigase II) in the presence of 10-20% (w/v) polyethylene glycol of average molecular weight 4000, 6000, or 8000. The reaction can be incubated for 1 hr @ 60-70C The pdo can comprise the following: (i) all, part or none of the sequence corresponding to a surface-bound oligonucleotide for Illumina flow cell cluster generation (ii) a 3′-end affinity group that is incapable of participating in the ligation reaction that is linked to the oligonucleotide at a sufficient distance (10 atoms or greater) to minimize steric hindrance of the interaction between the affinity ligand and the bound receptor.

The pdo can be adenlyated by any means known in the art. If an adenlyated adaptor is used, in some embodiments the ATP-dependent RNA ligase is not CircLigase or CircLigase II. The reaction can be purified by size to remove unreacted adaptor. This can be achieved through the use of a microfiltration unit with a molecular size cutoff of 10K or 3K (e.g. microcon YM-10 or YM3, or nanosep omega). Alternatively, adaptor removal can be achieved through passage through a size exclusion desalting column (agarose, polyacrylamide) with a size exclusion cutoff of 10K or less, through the use of a spin column, through selective precipitation with PEG, alcohol or salt, high stringency washing, or denaturing gel electrophoresis.

In step 6 an oligonucleotide primer either fully complementary to the adaptor or partially complementary to the adaptor at its 3′-end, but fully possessing the sequence corresponding to the Illumina flow-cell oligonucleotides, can then be used to create a reverse complement of the bound library using a proofreading mesophilic DNA polymerase. Preferably, a thermophilic polymerase with 5′→3′ exonucleolytic/endonucleolytic (Family A DNA polymerase, e.g., DNA polymerase I) or 3′→5′ exonucleolytic (family B DNA polymerases, Vent, Phusion, Pfu and their variants) activity is used to permit linear amplification of the library.

In step 7 the recovered material can then be bound to an affinity resin or support capable of binding to the 3′-end affinity tag in batch mode. The recovered material can be put into a pre-rinsed support in a 0.2 ml tube containing at least 10-fold excess and preferably 100-fold more available binding sites that the total number of tagged adaptor molecules.

In step 8 the supernatant consisting of copies of the bound library can be harvested and quantified.

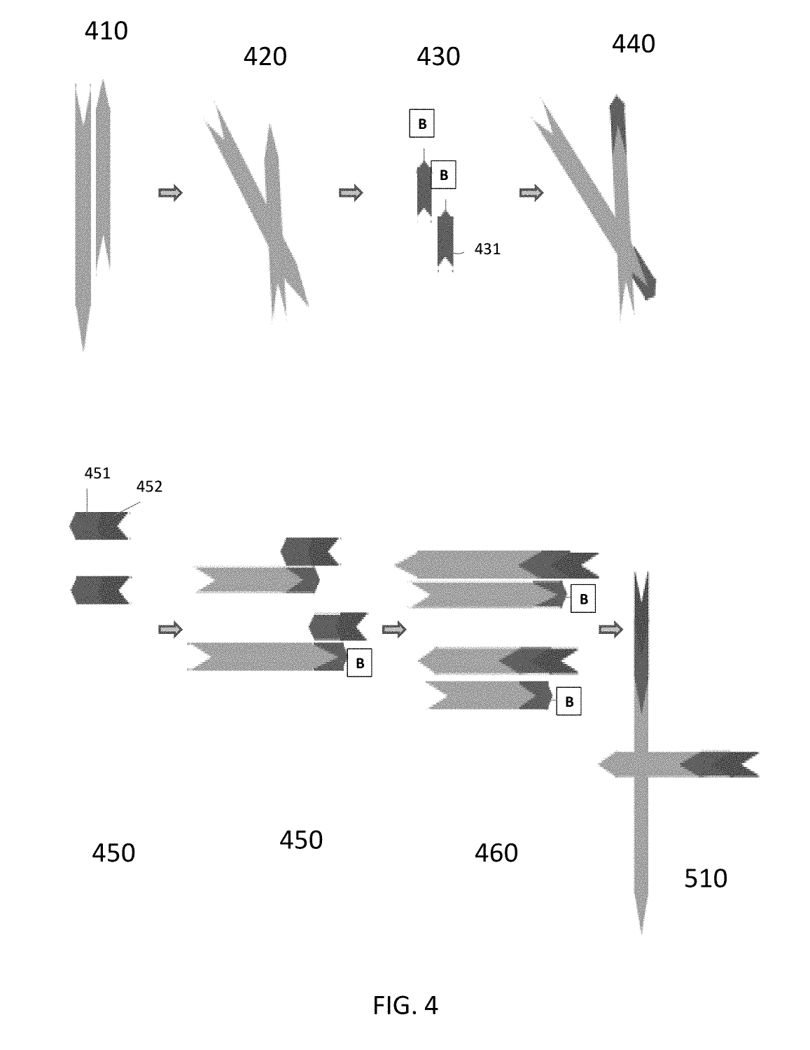

FIG. 4 is a depiction of an exemplary workflow as described in FIG. 3 for preparing an ssDNA library. In step 410 dsDNA is fragmented. In step 420 dsDNA fragments are dephosphorylated and heat-denatured into single strands. In step 430 biotinylated pdo's comprising a primer-docking sequence 431 are contacted with the nucleic acid fragments. In step 440 the pdo's are ligated to the 3′ ends of the ssDNA fragments to create library member precursors. In step 450 primers comprising sequence complementary to the pdo 451 and adaptor sequence 452 are hybridized in step 560 to the ssDNA via the pdos. In step 460 the hybridized primers are extended along the template ssDNA fragments to create duplexes. The duplexes are immobilized onto a solid support (e.g., streptavidin coated beads). Heat denaturation releases the final library members into solution while retaining the original ssDNA fragment on the bead.

Alternative Embodiments of ssDNA Library Preparation.

In another aspect, the invention provides a method of preparing a ssDNA library, comprising denaturing dsDNA fragments into ssDNA, and ligating adaptor sequences to both ends of the ssDNA molecules. Methods of fragmenting dsDNA is described herein. Methods of denaturing dsDNA fragments are described herein.

The method can comprise ligating a first adaptor that comprises a sequence that is at least 70% complementary or identical to a first surface-bound oligonucleotide. The first surface-bound oligonucleotide can be an NGS platform-specific surface bound oligonucleotide. The first adaptor can comprise a sequence complementary or identical to 5, 6, 7, 8, 9, 10, 11, 12, 13, 14, 15, 20, or more than 20 contiguous nucleotides of the surface-bound oligonucleotide. The first adaptor can further comprise a sequence that is at least 70% complementary to a first sequencing primer. In some embodiments the first adaptor is ligated to a 3′ end of an ssDNA fragment using a method described herein or any method known in the art. In some embodiments, the ssDNA fragment lacks 5′ phosphate groups. In particular embodiments, the first adaptor is ligated to the 3′ end of the ssDNA fragment by an ATP-dependent ligase. In other embodiments, the first adaptor comprises a 3′ terminal blocking group. Generally, the 3′ terminal blocking group will prevent the formation of a covalent bond between the 3′ terminal base and another nucleotide. In some embodiments, the 3′ terminal blocking group is dideoxy-dNTP or biotin. The first adaptor can be 5′ adenylated. In some embodiments, the first adaptor is ligated to a 3′ end of an ssDNA fragment by an RNA ligase as described herein. The RNA ligase can be truncated or mutated RNA ligase 2 from T4 or Mth. The method can further comprises ligating a second adaptor sequence to a 5′ end of the ssDNA fragment. The second adaptor sequence can be distinct from the first adaptor sequence. The second adaptor sequence can comprise a sequence that is at least 70% complementary to a second surface-bound oligonucleotide. The second surface-bound oligonucleotide can be an NGS platform-specific surface bound oligonucleotide. The second adaptor can comprise a sequence complementary or identical to 5, 6, 7, 8, 9, 10, 11, 12, 13, 14, 15, 20, or more than 20 contiguous nucleotides of the surface-bound oligonucleotide. The second adaptor can further comprise a sequence that is at least 70% complementary to a second sequencing primer. In some embodiments the second adaptor is ligated to the ssDNA fragment using RNA ligase, e.g., CircLigase as described herein. In some embodiments, the first and second adaptor are both at least 70% complementary to the first and second surface-bound oligonucleotides. In other embodiments, the first and second adaptor are both at least 70% identical to the first and second surface-bound oligonucleotides.

The ssDNA library produced using methods described herein can be used for whole genome sequencing or targeted sequencing. In some embodiments, the ssDNA library produced using methods described herein are enriched for target polynucleotides of interest prior to sequencing.

### Target Enrichment

In another aspect, the invention provides a method for preparing a target-enriched nucleic acid library. The method can involve hybridizing a target-selective oligonucleotide (TSO) to a single stranded DNA (ssDNA) fragment to create a hybridization product, and amplifying the hybridization product in a single round of amplification to create an extension strand.

The method of target enrichment can be as described in US. Patent Application Pub. No. 20120157322, hereby incorporated by reference.

The hybridizing and amplifying can occur in a reaction mixture. The term “reaction mixture” as used herein generally refers to a mixture of components necessary to amplify at least one amplicon from nucleic acid template molecules. The mixture may comprise nucleotides (dNTPs), a polymerase and a target-selective oligonucleotide. In some embodiments, the mixture comprises a plurality of target-selective oligonucleotides. The mixture may further comprise a Tris buffer, a monovalent salt, and Mg2+. The concentration of each component is well known in the art and can be further optimized by an ordinary skilled artisan. The reaction mixture can also comprise additives including, but not limited to, non-specific background/blocking nucleic acids (e.g., salmon sperm DNA), biopreservatives (e.g. sodium azide), PCR enhancers (e.g. Betaine, Trehalose, etc.), and inhibitors (e.g. RNAse inhibitors). In some embodiments, a nucleic acid sample (e.g., a sample comprising an ssDNA fragment) is admixed with the reaction mixture. Accordingly, in some embodiments the reaction mixture further comprises a nucleic acid sample.

The ssDNA fragment can be a member of an ssDNA library. The ssDNA library can be prepared using a method as described herein. The ssDNA fragment can comprise a first single-stranded adaptor sequence located at a first end but not at a second end. In some embodiments, the first end is a 5′ end. In some embodiments, the TSO comprises a second single-stranded adaptor sequence located at a first end but not a second end. The first end can be a 5′ end. In some embodiments, the first adaptor sequence comprises a sequence that is at least 70% identical to a first surface-bound oligonucleotide. In some embodiments, the first adaptor sequence comprises a sequence that is at least 70% identical to a sequencing primer. In some embodiments the first adaptor further comprises a barcode sequence. In some embodiments, the second adaptor comprises a sequence that is at least 70% identical to a second surface-bound oligonucleotide. In some embodiments, the second adaptor comprises a sequence that is at least 70% identical to a sequencing primer

The target-selective oligonucleotide (tso) can be designed to at least partially hybridize to a target polynucleotide of interest. In some embodiments, the tso is designed to selectively hybridize to the target polynucleotide. The tso can be at least about 70%, 75%, 80%, 85%, 90%, 95%, or more than 95% complementary to a sequence in the target polynucleotide. In some embodiments, the tso is 100% complementary to a sequence in the target polynucleotide. The hybridization can result in a tso/target duplex with a Tm. The Tm of the tso/target duplex can be between 0-100 deg C, between 20-90 deg C, between 40-80 deg C, between 50-70 deg C, or between 55-65 deg C. The tso generally is sufficiently long to prime the synthesis of extension products in the presence of a polymerase. The exact length and composition of a tso can depend on many factors, including temperature of the annealing reaction, source and composition of the primer, and ratio of primer:probe concentration. The tso can be, for example, 8-50, 10-40, or 12-24 nucleotides in length.

### Amplification

The method can comprise amplification of the target in the reaction mixture. The amplification can be primed by a tso in a tso/target duplex. In some embodiments amplification is carried out utilizing a nucleic acid polymerase. The nucleic acid polymerase can be a DNA polymerase. In particular embodiments, the DNA polymerase is a thermostable DNA polymerase. The polymerase can be a member of A or B family DNA proofreading polymerases (Vent, Pfu, Phusion, and their variants), a DNA polymerase holoenzyme (DNA pol III holoenzyme), a Taq polymerase, or a combination thereof.

Amplification can be carried out as an automated process wherein the reaction mixture comprising template DNA is cycled through a denaturing step, a primer annealing step, and a synthesis step, whereby cleavage and displacement occurs simultaneously with primer-dependent template extension. The automated process may be carried out using a PCR thermal cycler. Commercially available thermal cycler systems include systems from Bio-Rad Laboratories, Life technologies, Perkin-Elmer, among others. In some embodiments, one cycle of amplification is performed.

Amplification of the tso/target duplex can result in an extension product comprising the original ssDNA fragment comprising the target sequence, and an extended strand comprising the second adaptor sequence, the tso, a reverse complement of the target sequence, and a reverse complement of the first adaptor sequence. If the first adaptor sequence of the original ssDNA fragment was 70% or more identical to a first surface-bound oligonucleotide, then the extended strand would comprise a first adaptor sequence that is 70% or more complementary to the first surface-bound oligonucleotide, and thereby would be hybridizable to the first surface-bound oligonucleotide. The extended strands, can comprise the target-enriched library.

The extension products in the reaction mixture can be denatured. The denatured extension products can be contacted with a surface immobilized thereon at least a first surface-bound oligonucleotide. In some embodiments, the extended strand is captured by the first surface-bound oligonucleotide, which can anneal to the first adaptor sequence on the extended strand.

The first surface-bound oligonucleotide can prime the extension of the captured extended strand. In some embodiments, extension of the captured extended strand results in a captured extension product. The captured extension product comprises the first surface bound oligonucleotide, the target sequence, and a second adaptor sequence that is at least 70% or more complementary to a second surface-bound oligonucleotide.

In some embodiments, the captured extension product hybridizes to the second surface-bound oligonucleotide, forming a bridge. In some embodiments, the bridge is amplified by bridge PCR. Bridge PCR methods can be carried out using methods known to the art.

### Kits for Library Preparation and Target Enrichment

Also provided are kits for practicing a method of library preparation as described herein or target-enrichment as described herein.

In one aspect, the invention provides kits for preparing a ssDNA library. In one embodiment, the kit comprises a pdo as described herein. In some embodiments, the kit comprises instructions, e.g., instructions for ligating a pdo to an ssDNA fragment. The kit can further comprise a ligase. The ligase can be an Rnl 1 or Rnl 2 family ligase, as described herein. The kit can further comprise a primer which can hybridize to the pdo. Primers hybridizable to the pdo are described herein. In some embodiments, the kit provides a solid support, e.g., a bead immobilized thereon a capture reagent. In some embodiments, the kit provides a polymerase for conducting an extension reaction. In some embodiments, the kit provides dNTPs for conducting an extension reaction.

In another embodiment, the kit comprises a first adaptor oligonucleotide that comprises sequence that is at least 70% complementary to a first support-bound oligonucleotide coupled to a sequencing platform, a second adaptor oligonucleotide that comprises a sequence that is distinct from the first adaptor, an RNA ligase, and instructions for use, e.g., instructions for practicing a method of the invention. In some embodiments, the first adaptor comprises a 3′terminal blocking group that prevents the formation of a covalent bond between the 3′ terminal base and another nucleotide. 3′ terminal blocking groups are described herein. In some embodiments, the first is 5′ adenylated. In some embodiments, the first adaptor comprises a sequence that is at least 70% complementary to a sequencing primer. In some embodiments, the second adaptor comprises a sequence that is at least 70% complementary to a sequencing primer. In some embodiments, the second adaptor comprises a sequence that is at least 70% complementary to a second support-bound oligonucleotide coupled to a sequencing platform.

The invention provides kits for preparing a target-enriched DNA library. In some embodiments, the kit comprises a pdo, a ligase, a primer which can hybridize to the pdo, a solid support comprising a capture reagent, a polymerase, dNTPs, or any combination thereof. In some embodiments the kit further comprises a tso. The tso can be immobilized on a solid support coupled for sequencing on an NGS platform, as described in US Patent Application Pub No. 20120157322, hereby incorporated by reference.

In some embodiments, kits of the invention include a packaging material. As used herein, the term “packaging material” can refer to a physical structure housing the components of the kit. The packaging material can maintain sterility of the kit components, and can be made of material commonly used for such purposes (e.g., paper, corrugated fiber, glass, plastic, foil, ampules, etc.). Kits can also include a buffering agent, a preservative, or a protein/nucleic acid stabilizing agent.

### Sequencing

In some embodiments the target-enriched libraries are sequenced using any methods known in the art or as described herein. Sequencing can reveal the presence of mutations in one or more cancer-related genes in the set. In some embodiments a subset of 2, 3, 4 genes harboring the mutations are selected for further monitoring by assessment of cell-free DNA in a fluid sample isolated from the subject at later time points. In some embodiments a subset of no more than 4 genes harboring the mutations are selected for further monitoring by assessment of cell-free DNA in a fluid sample isolated from the subject at later time points.

### Assessment of Cell-Free DNA Over Time

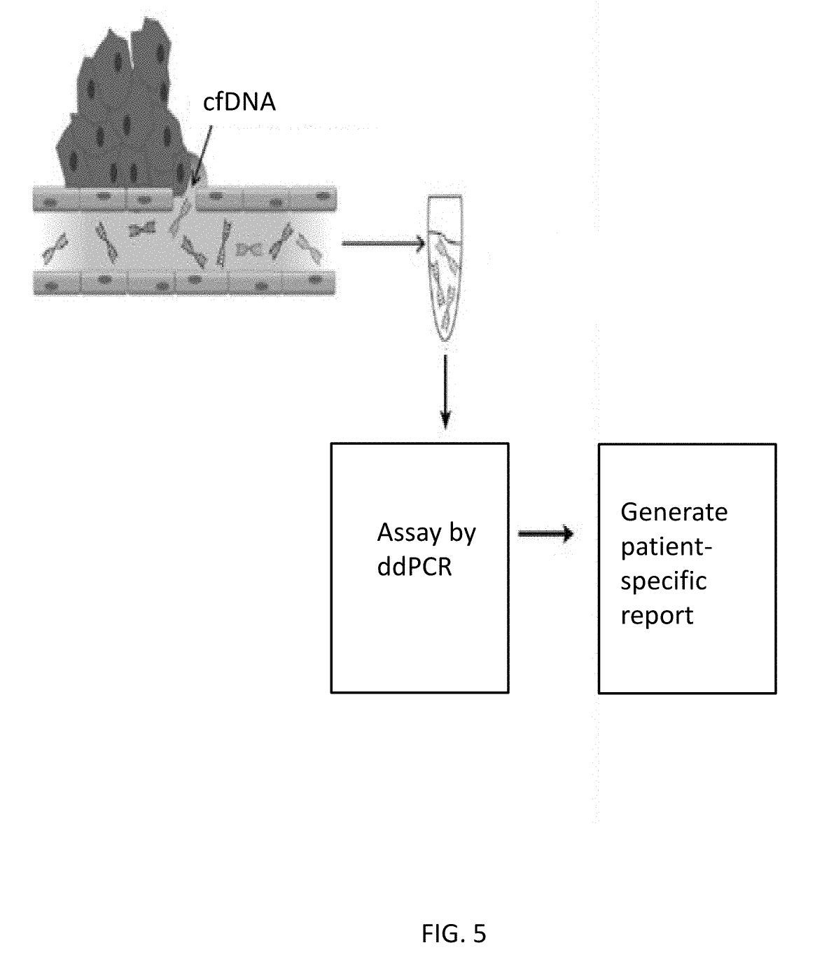

In some embodiments, assessment of cell free-DNA comprises detection and/or measurement of alleles of the subset of genes, as shown in FIG. 5. FIG. 5 depicts tumor DNA 601 entering the bloodstream of a subject. Detection of the alleles can be by any means known in the art or as described herein. The detection can be by methods as described in U.S. Pat. No. 5,538,848 (e.g., using a Taqman assay) or as described herein.

Accordingly, the present invention provides methods and kits for the sensitive detection of a mutation in a target polynucleotide. In some aspects, the methods and kits of the invention can be used for the discrimination of alleles in a target polynucleotide. For example, the invention provides methods and kits for the detection of mutant alleles in a background of high wild-type allelic ratio. For another example, the invention provides methods and kits for the detection of multiple alleles. In some embodiments, detection of an allele is enabled by release or activation of a detectable signal if the interrogated allele is present.

### Methods for Allele Detection

In some aspects, one or more methods of allele detection as described herein relate to the ability of an oligonucleotide primer to bind to a target polynucleotide region suspected of harboring the mutation. The oligonucleotide primer can partially overlay a locus of the suspected mutation. In some embodiments the oligonucleotide primer completely overlays the mutation. Accordingly, in some embodiments the mutation is small enough to be encompassed by an oligonucleotide primer. The mutation can be a single nucleotide polymorphism (SNP). The mutation can also comprise multiple nucleotide polymorphisms (e.g, double mutation or triple mutation). The mutation can be an insertion of one or more nucleotides. The mutation can be an insertion of 1, 2, 3, 4, 5, 6, 7, 8, 9, 10, 11, 12, 13,14, 15, 16, 17, 18, 19, 20, 50, 100, 500, 1000, 10000, 100000, 1000000 nucleotides. The mutation can be an insertion of 1-5, 2-10, 5-15, or 10-20 nucleotides. In some embodiments, the mutation is a deletion of one or more nucleotides. The mutation can be a deletion of 1, 2, 3, 4, 5, 6, 7, 8, 9, 10, 11, 12, 13, 14, 15, 16, 17, 18, 19, 20 nucleotides. The mutation can be a deletion of 1-5, 2-10, 5-15, or 10-20 nucleotides. The mutation can be an inversion of two or more nucleotides. In some embodiments, 2, 3, 4, 5, or more nucleotides are inverted. In some embodiments, the mutation is a copy number variation (e.g., a copy number variation of a SNP or wild-type allele).

In one aspect, the invention provides a method of detecting a mutation in a target polynucleotide region, comprising the steps of: (a) contacting a nucleic acid sample with a reaction mixture for allele detection, wherein the reaction mixture for allele detection comprises an oligonucleotide primer capable of hybridizing to the target polynucleotide region, wherein the oligonucleotide primer comprises a probe binding region and a template binding region that at least partially overlays a locus suspected of harboring the mutation and is capable of allele-specific extension by a polymerase; (b) extending the oligonucleotide primer to form an extension product; and (c) detecting the extension product, whereby the detecting the extension product indicates the presence of the mutation.

Primers for Allele Detection

The oligonucleotide primer (e.g., a forward primer) can be designed to at least partially hybridize to a target polynucleotide suspected of harboring a mutation. In some embodiments, the template binding region of the forward primer is designed to selectively hybridize to the target polynucleotide. The hybridization can result in a forward primer/template duplex with a Tm. The Tm of the primer/template duplex can be between 0-100 deg C, between 20-90 deg C, between 40-80 deg C, between 50-70 deg C, or between 55-65 deg C. The template binding region of the forward primer can be 8-50, 10-40, or 12-24 nucleotides in length. The template binding region of the forward primer can be designed to at least partially overlay a particular locus suspected of harboring a mutation. The template binding region of the forward primer can, for example, overlay about at least 0.5%, 1%, 2%, 3%, 4%, 5%, 6%, 7%, 8%, 9%, 10%, 20%, 20%, 40%, 50%, 60%, 70%, 80%, 90%, or 100% of the locus suspected of harboring the mutation. The template binding region of the forward primer can overlay at least about 0.5-2%, 1-10%, 5-20%, 10-50%, 30-70%, 50-80%, 60-90%, or 80-100% of the locus suspected of harboring the mutation. The template binding region can be located at a 3′ region of the forward primer. In some embodiments, the region of the template binding region that overlays the locus is a 3′ terminal region. In some embodiments, the 3′ terminal region that overlays the mutation locus comprises 1, 2, 3, 4, 5, or more than 5 bases of the 3′-end of the template binding region. In some embodiments, the 3′ terminal base of the forward primer overlays the locus. In some embodiments, the 3′ terminal region of the forward primer is complementary to the interrogated allele. The 3′ terminal base of the forward primer may not complementary to the interrogated allele. In some embodiments, one or more mismatches is introduced into the 3′-region adjacent to the 3′-terminal base (e.g., n−1, n−2, n−3, etc.). These mismatches can be nucleotides or modified nucleotides that increase or decrease the impact of this mismatch on primer extension.

The template binding region can at least partially overlay with a locus that is suspected of having a copy number variation. In some embodiments, the template binding region of the forward primer can overlay at least about 0.5-2%, 1-10%, 5-20%, 10-50%, 30-70%, 50-80%, 60-90%, or 80-100% of the locus suspected of having a copy number variation.

The 3′ terminal region of the forward primer can comprise nucleotides linked by phosphorothioate linkages. In some embodiments, at least 2, 3, 4, 5, or more nucleotides at the 3′ terminal region of the forward primer are linked by phosphorothioate linkages.

A forward primer can further comprise a probe-binding region. Generally, the probe-binding region of the forward primer enables use of a reporter probe that is template independent. The probe-binding region can comprise a unique sequence or barcode that does not hybridize to the template nucleic acid. The probe-binding region can, for example, be designed to avoid significant sequence similarity or complementarity to known genomic sequences of an organism of interest. Such unique sequences can be randomly generated, e.g., by a computer readable medium, and selected by BLASTing against known nucleotide databases such as, e.g., EMBL, GenBank, or DDBJ. The barcode sequence can also be designed to avoid secondary structure. Tools for probe design are known in the art, and include, e.g., mFold, Primer Express. The probe-binding region can be 5-50, 6-40, or 7-30 nucleotides in length. The probe-binding region can be 1-20, 3-15, or 6-8 nucleotides away from the template binding region of the forward primer. The probe-binding region can be located 5′ of the template binding region.

In some embodiments, the method further comprises contacting the nucleic acid sample with a reverse primer. The reverse primer can be an oligonucleotide primer that corresponds to a region of template nucleic acid that is downstream of the forward primer. In some embodiments, the reverse primer is downstream of the interrogated allele. The reverse primer can bind to a reverse complement strand of the target polynucleotide. A forward/reverse primer pair can span a target region suspected of harboring a mutation. In some embodiments, the target region is 14-1000, 20-800, 40-600, 50-500, 70-300, 90-200, or 100-150 nucleotides long.

Primers or other oligonucleotides used in the present invention may further comprise a barcode sequence. Barcode sequences are described herein. In some embodiments, a barcode sequence encodes information relating to the identity of an interrogated allele, identity of a target polynucleotide or genomic locus, identity of a sample, a subject, or any combination thereof. A barcode sequence can be a portion of a primer, a reporter probe, or both. A barcode sequence may be at the 5′-end or 3′-end of an oligonucleotide, or may be located in any region of the oligonucleotide. A barcode sequence generally is not part of a template sequence. Barcode sequences may vary widely in size and composition; the following references provide guidance for selecting sets of barcode sequences appropriate for particular embodiments: Brenner, U.S. Pat. No. 5,635,400; Brenner et al, Proc. Natl. Acad. Sci., 97: 1665-1670 (2000); Shoemaker et al, Nature Genetics, 14: 450-456 (1996); Morris et al, European patent publication 0799897A1; Wallace, U.S. Pat. No. 5,981,179. A barcode sequence may have a length of about 4 to 36 nucleotides, about 6 to 30 nucleotides, or about 8 to 20 nucleotides.

Primers used in the present invention are generally sufficiently long to prime the synthesis of extension products in the presence of the agent for polymerization. The exact length and composition of a primer can depend on many factors, including temperature of the annealing reaction, source and composition of the primer, and ratio of primer:probe concentration. The primer length can be, for example, about 5-100, 10-50, or 20-30 nucleotides, although a primer may contain more or fewer nucleotides.

Reporter Probes

In some embodiments, the reaction mixture further comprises a reporter probe. Generally, the reporter probe of the present invention is designed to produce a detectable signal indicating the presence of the interrogated allele.

The reporter probe can comprise a detectable moiety and a quencher moiety. The detectable moiety can be a dye. The dye can be a fluorescent dye, e.g., a fluorophore. The fluorescent dye can be a derivatized dye for attachment to the terminal 3′ carbon or terminal 5′ carbon of the probe via a linking moiety. The dye can be derivatized for attachment to the terminal 5′ carbon of the probe via a linking moiety. Quenching can involve a transfer of energy between the fluorophore and the quencher. The emission spectrum of the fluorophore and the absorption spectrum of the quencher can overlap. When the probe is intact, the fluorescent signal from the detectable moiety can be substantially suppressed by the quencher. Cleavage of the reporter probe, e.g., by hydrolysis, can separate the detectable moiety from the quencher moiety. The separation can enable the fluorescent moiety to produce a detectable fluorescent signal.

The reporter probes may be designed according to Livak et al., “Oligonucleotides with fluorescent dyes at opposite ends provide a quenched probe system useful for detecting PCR product and nucleic acid hybridization,” PCR Methods Appl. 1995 4: 357-362.

Reporter-quencher moiety pairs for particular probes can be selected according to, e.g., Pesce et at, editors, Fluorescence Spectroscopy (Marcel Dekker, New York, 1971); White et at, Fluorescence Analysis: A Practical Approach (Marcel Dekker, New York, 1970. Exemplary fluorescent and chromogenic molecules that may be used in reporter-quencher pairs, are described in, e.g. Berlman, Handbook of Fluorescence Sprectra of Aromatic Molecules, 2nd Edition (Academic Press, New York, 1971); Griffiths, Colour and Constitution of Organic Molecules (Academic Press, New York, 1976); Bishop, editor, Indicators (Pergamon Press, Oxford, 1972); Haugland, Handbook of Fluorescent Probes and Research Chemicals (Molecular Probes, Eugene, 1992); Pringsheim, Fluorescence and Phosphorescence (Interscience Publishers, New York, 1949).

A wide variety of reactive fluorescent reporter dyes can be used so long as they are quenched by a quencher dye of the invention. The fluorophore can be an aromatic or heteroaromatic compound. The fluorophore can be, for example, a pyrene, anthracene, naphthalene, acridine, stilbene, benzoxaazole, indole, benzindole, oxazole, thiazole, benzothiazole, canine, carbocyanine, salicylate, anthranilate, xanthenes dye, coumarin. Exemplary xanthene dyes include, e.g., fluorescein and rhodamine dyes. Exemplary fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent reporters also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl) aminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno[2,3, 4-ij: 5,6, 7-i′j′]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3, 6,7, 12,13, 16,17-octahydro-inner salt (TR or Texas Red); or BODIPY™ dyes. Exemplary fluorescent and quencher moieties are described in, e.g., WO/2005/049849, hereby incorporated by reference.

As is known in the art, suitable quenchers are selected according to the fluorescer. Exemplary reporters and quenchers are further described in Anderson et al, U.S. Pat. No. 7,601,821, hereby incorporated by reference.

Quenchers are also available from various commercial sources. Exemplary commercially available quenchers include, e.g., Black Hole Quenchers® from Biosearch Technologies and Iowa Black® or ZEN quenchers from Integrated DNA Technologies, Inc.

In some embodiments, the reporter probe comprises two quencher moieties. Exemplary probes comprising two quencher moieties include the Zen probes from Integrated DNA Technologies. Such probes comprise an internal quencher moiety that is located about 9 bases away from the detectable moiety, and generally reduce background signal associated with traditional reporter/quencher probes.

Detectable moieties and quencher moieties can be derivatized for covalent attachment to oligonucleotides via common reactive groups or linking moieties. Methods for derivatization of detectable and quencher moieties are described in, e.g., Ullman et al, U.S. Pat. No. 3,996,345; Khanna et al, U.S. Pat. No. 4,351,760; Eckstein, editor, Oligonucleotides and Analogues: A Practical Approach (IRL Press, Oxford, 1991); Zuckerman et al, Nucleic Acids Research, 15: 5305-5321 (1987) (3′ thiol group on oligonucleotide); Sharma et al, Nucleic Acids Research, 19:3019 (1991) (3′ sulfhydryl); Giusti et al, PCR Methods and Applications, 2:223-227 (1993) and Fung et al, U.S. Pat. No. 4,757,141 (5′ phosphoamino group via Aminolink™ II available from Applied Biosystems, Foster City, Calif.); Stabinsky, U.S. Pat. No. 4,739,044 (3′ aminoalkylphosphoryl group); Agrawal et al, Tetrahedron Letters, 31:1543-1546 (1990) (attachment via phosphoramidate linkages); Sproat et al, Nucleic Acids Research, 15:4837 (1987)(5′ mercapto group); Nelson et al, Nucleic Acids Research, 17:7187-7194 (1989) (3′ amino group), all of which are hereby incorporated by reference).

In some embodiments, commercially available linking moieties can be attached to an oligonucleotide during synthesis, e.g. linking moieties available through Clontech Laboratories (Palo Alto, Calif.). By way of example only, rhodamine and fluorescein dyes can be derivatized with a phosphoramidite moiety for attachment to a 5′ hydroxyl of an oligonucleotide (see, e.g., Woo et al, U.S. Pat. No. 5,231,191; and Hobbs, Jr. U.S. Pat. No. 4,997,928, all of which are hereby incorporated by reference).

In some embodiments, the detectable moiety produces a non-fluorescent signal. For example, any probe for which hydrolysis of the probe results in a detectable separation of a signal moiety from the detection probe-amplicon complex may be used. For example, release of the signal moiety may be detected electronically (e.g., as an electrode surface charge perturbation when a signal moiety is released from the detection probe/amplicon complex), by quantum dot sensing, by luminescence, or chemically (e.g., by a change in pH in a solution as a signal moiety is released into solution). Likewise, any probe that binds to a probe-binding region and for which a change in signal can be detected upon separation of a detectable moiety from a quencher moiety may be used. For example, molecular beacon probes, MGB probes, or other probes are contemplated for use in the invention. Molecular beacon probes are described in, e.g., U.S. Pat. Nos. 5,925,517 and 6,103,406, hereby incorporated by reference. MGB probes are described in, e.g., U.S. Pat. No. 7,381,818, hereby incorporated by reference.

The reporter probe can be designed to selectively hybridize to a probe-binding region of a primer as described herein. Accordingly, in some embodiments the reporter probe comprises a sequence that is complementary to at least a portion of the probe-binding region. The reporter probe can be 5-50, 6-40, or 7-30 nucleotides in length. The hybridization can result in a probe/primer duplex with a Tm. The Tm of the probe/primer duplex can be higher than the Tm of the primer/template duplex. The Tm of the probe/primer duplex can be 1, 2, 3, 4, 5, 6, 7, 8 9, 10, or more than 10 deg C than the Tm of the primer/template duplex.

In some embodiments, the reporter probe selectively hybridizes to a sequence in the probe-binding region that is at least 2, 3, 4, 5, 6, 7, 8, 9, 10, 15, or 20 nucleotides apart from the template binding region of the primer.

The reporter probe can be present at a concentration that is higher than the concentration of the forward primer. The reporter probe can for example be present in a concentration that is, e.g., 1-10 fold or 1-5 fold higher than the concentration of the forward primer. The reporter probe can be present in a concentration that results in at least 50%, at least 60%, at least 70%, at least 80%, at least 90%, at least 95%, at least 96%, at least 97%, at least 98%, at least 99%, or about 100% of the forward primers occupied by the probe.

The primers and probes of the invention may be prepared by any suitable method. Methods for preparing oligonucleotides of specific sequence are known in the art, and include, for example, cloning and restriction of appropriate sequences and direct chemical synthesis. Chemical synthesis methods may include, for example, the phosphotriester method described by Narang et al., 1979, Methods in Enzymology 68:90, the phosphodiester method disclosed by Brown et al., 1979, Methods in Enzymology 68:109, the diethylphosphoramidate method disclosed in Beaucage et al., 1981, Tetrahedron Letters 22:1859, and the solid support method disclosed in U.S. Pat. No. 4,458,066, all of which publications are hereby incorporated by reference.

In some embodiments, a forward primer comprising a template binding region and a probe-binding region can be prepared using two different oligonucleotides corresponding to the template binding region and probe binding region, respectively. The two oligonucleotides can be ligated enzymatically. Ligation can be by an RNA ligase. The RNA ligase can be an ATP dependent ligase. The RNA ligase can be an Rnl 1 family ligase. Generally, Rnl 1 family ligases can repair single-stranded breaks in tRNA. Exemplary Rnl 1 family ligases include, e.g., T4 RNA ligase, thermostable RNA ligase 1 from Thermus scitoductus bacteriophage TS2126 (CircLigase), or CircLigase II. Generally, Rnl 2 family ligases can seal nicks in duplex RNAs. Exemplary Rnl 2 family ligases include, e.g., T4 RNA ligase 2. The RNA ligase can be an Archaeal RNA ligase, e.g., an archaeal RNA ligase from the thermophilic archaeon Methanobacterium thermoautotrophicum (MthRn1). Ligation can also be effected by use of a splint oligonucleotide that spans the two oligonucleotides corresponding to the template binding and probe binding regions, respectively. In some embodiments, ligation using a splint oligonucleotide can comprise use of a T4 DNA ligase. Alternatively, ligation can be mediated by an ATP-independent ligase. Exemplary ATP-independent ligases include, e.g., RNA 3′-Phosphate Cyclase (RtcA), RNA ligase RtcB, or manufactured variants thereof. In some embodiments, ligation is performed indirectly through a two-step process, in which a template binding region is adenylated (e.g., adenylated chemically during synthesis or enzymatically using a ligase), and the adenylated template binding sequence is conjugated to the probe binding region.

Ligation can also be performed with “click chemistry.” Click chemistry is a concept that involves linking smaller subunits with simple chemistry. Smaller subunits can refer to small building blocks of larger molecules such as DNA bases, RNA nucleotides, linear or circularized DNA or RNA oligonucleotides. (3+2) cycloadditions between azide and alkyne groups which results in the formation of 1,2,3-triazole rings (e.g., copper-catalysed alkyne-azide coupling reaction) are generally considered typical click chemistry reactions. Other chemical ligation methods include the use of cyanogen bromide, phosphorothioate-iodoacetyl, and native ligation techniques where a C-terminal a-thioester is reacted in a chemoselective manner with an unprotected peptide containing an N-terminal Cys residue)

Primers and/or reporter probes can also be obtained from commercial sources such as Operon Technologies, Amersham Pharmacia Biotech, Sigma, IDT Technologies, and Life Technologies. The primers can have an identical melting temperature. The lengths of the primers can be extended or shortened at the 5′ end or the 3′ end to produce primers with desired melting temperatures. Also, the annealing position of each primer pair can be designed such that the sequence and, length of the primer pairs yield the desired melting temperature. The simplest equation for determining the melting temperature of primers smaller than 25 base pairs is the Wallace Rule (Td=2(A+T)+4(G+C)). Computer programs can also be used to design primers, including but not limited to Array Designer Software (Arrayit Inc.), Oligonucleotide Probe Sequence Design Software for Genetic Analysis (Olympus Optical Co.), NetPrimer, and DNAsis from Hitachi Software Engineering. The Tm (melting or annealing temperature) of each primer can be calculated using software programs such as Oligo Design, available from Invitrogen Corp.

The annealing temperature of the primers can be recalculated and increased after any cycle of amplification, including but not limited to cycle 1, 2, 3, 4, 5, cycles 6-10, cycles 10-15, cycles 15-20, cycles 20-25, cycles 25-30, cycles 30-35, or cycles 35-40. After the initial cycles of amplification, part of the primers may be incorporated into the products from each loci of interest, thus the TM can be recalculated based on the part of the primer incorporated into the product.

Reaction Mixture for Allele Detection

The term “reaction mixture for allele detection” as used herein generally refers to a mixture of components necessary to amplify at least one amplicon from nucleic acid template molecules. The mixture for allele detection may comprise nucleotides (dNTPs), a polymerase and primers. The mixture for allele detection may further comprise a Tris buffer, a monovalent salt, and Mg2+. The concentration of each component is well known in the art and can be further optimized by an ordinary skilled artisan. In some embodiments, the reaction mixture for allele detection also comprises additives including, but not limited to, non-specific background/blocking nucleic acids (e.g., salmon sperm DNA), biopreservatives (e.g. sodium azide), PCR enhancers (e.g. Betaine, Trehalose, etc.), and inhibitors (e.g. RNAse inhibitors). In some embodiments, a nucleic acid sample is admixed with the reaction mixture for allele detection. Accordingly, in some embodiments the reaction mixture for allele detection further comprises a nucleic acid sample.

Amplification

The method can comprise amplification of template nucleic acid in the reaction mixture for allele detection. In some embodiments amplification is carried out utilizing a nucleic acid polymerase. The nucleic acid polymerase can be a DNA polymerase. The DNA polymerase can be a thermostable DNA polymerase.

Some aspects of the allele detection methods described herein relate to the ability of a DNA polymerase to separate a detectable moiety and quencher moiety in a reporter probe. Exemplary reporter probes are described herein. Separation of the detectable and quencher moiety can occur by cleavage of the reporter probe by the DNA polymerase. Cleavage of the reporter probe can occur by a 5′ exonuclease activity of the DNA polymerase. Accordingly, in some embodiments, the DNA polymerase comprises 5′→3′ exonuclease activity. As used herein, “5′→3′ nuclease activity” or “5′ to 3′ nuclease activity” can refer to an activity of a template-specific nucleic acid polymerase whereby nucleotides are removed from the 5′ end of an oligonucleotide in a sequential manner. DNA polymerases with 5′→3′ exonuclease activity are known in the art and include, e.g., DNA polymerase isolated from Thermus aquaticus (Taq DNA polymerase).

Some aspects of the allele detection methods described herein further relate to the discriminative ability of a primer to be extended by a nucleic acid polymerase (e.g., a DNA polymerase) in an amplification step, depending on the presence or absence of a mismatch between the terminal 3′ base of the primer and its hybridized template polynucleotide. In cases wherein there is no mismatch between the terminal 3′ base of the primer and template nucleotide, extension of the primer by DNA polymerase can efficiently occur during an amplification reaction. In cases wherein there is a mismatch between the terminal 3′ base of the primer and template nucleotide (e.g., the bases are not complementary), extension of the primer by DNA polymerase does not occur. In some embodiments extension of the mismatched primer does not occur if the DNA polymerase lacks 3′→5′ exonuclease activity. 3′→5′ exonuclease activity, as used herein, generally refers to an activity of a DNA polymerase whereby the polymerase recognizes a mismatched basepair and moves backward by one base to excise the incorrect nucleotide. Accordingly, the DNA polymerase can lack 3′→5′ exonuclease activity. Exemplary DNA polymerases lacking 3′→5′ exonuclease activity include, but are not limited to BST DNA polymerase I, BST DNA polymerase I (large fragment), Taq polymerase, Streptococcus pneumoniae DNA polymerase I, Klenow Fragment (3′→5′ exo-), PyroPhage® 3173 DNA Polymerase, Exonuclease Minus (Exo-) (available from Lucigen), T4 DNA Polymerase, Exonuclease Minus (Lucigen). In some embodiments, the DNA polymerase is a recombinant DNA polymerase that has been engineered to lack exonuclease activity.

In other embodiments, extension of the mismatched primer by DNA polymerase does not occur wherein the DNA polymerase has 3′→5′ exonuclease activity. In particular embodiments, extension of the mismatched primer by DNA polymerase having 3′→5′ exonuclease activity does not occur if the 3′ terminal region of the mismatch primer comprises nucleotides linked by phosphorothioate linkages. Exemplary primers comprising nucleotides linked by phosphorothioate linkages are described herein.

In some embodiments, the PCR process is carried out as an automated process wherein the reaction mixture comprising template DNA is cycled through a denaturing step, a reporter probe and primer annealing step, and a synthesis step, whereby cleavage and displacement occurs simultaneously with primer-dependent template extension. The automated process may be carried out using a PCR thermal cycler. Commercially available thermal cycler systems include systems from Bio-Rad Laboratories, Life technologies, Perkin-Elmer, among others.

Repeated cycles of denaturation, primer/probe annealing, primer extension, and reporter probe cleavage can result in the exponential accumulation of detectable signal. Sufficient cycles are run to achieve detection of the detectable signal, which can be several orders of magnitude greater than background signal.

The present invention is compatible, however, with other amplification systems, such as the transcription amplification system, in which one of the PCR primers encodes a promoter that is used to make RNA copies of the target sequence. In similar fashion, the present invention can be used in a self-sustained sequence replication (3SR) system, in which a variety of enzymes are used to make RNA transcripts that are then used to make DNA copies, all at a single temperature. By incorporating a polymerase with 5′ exonuclease activity into a ligase chain reaction (LCR) system, together with appropriate primer/probe sets, one can also employ the present invention to detect LCR products.

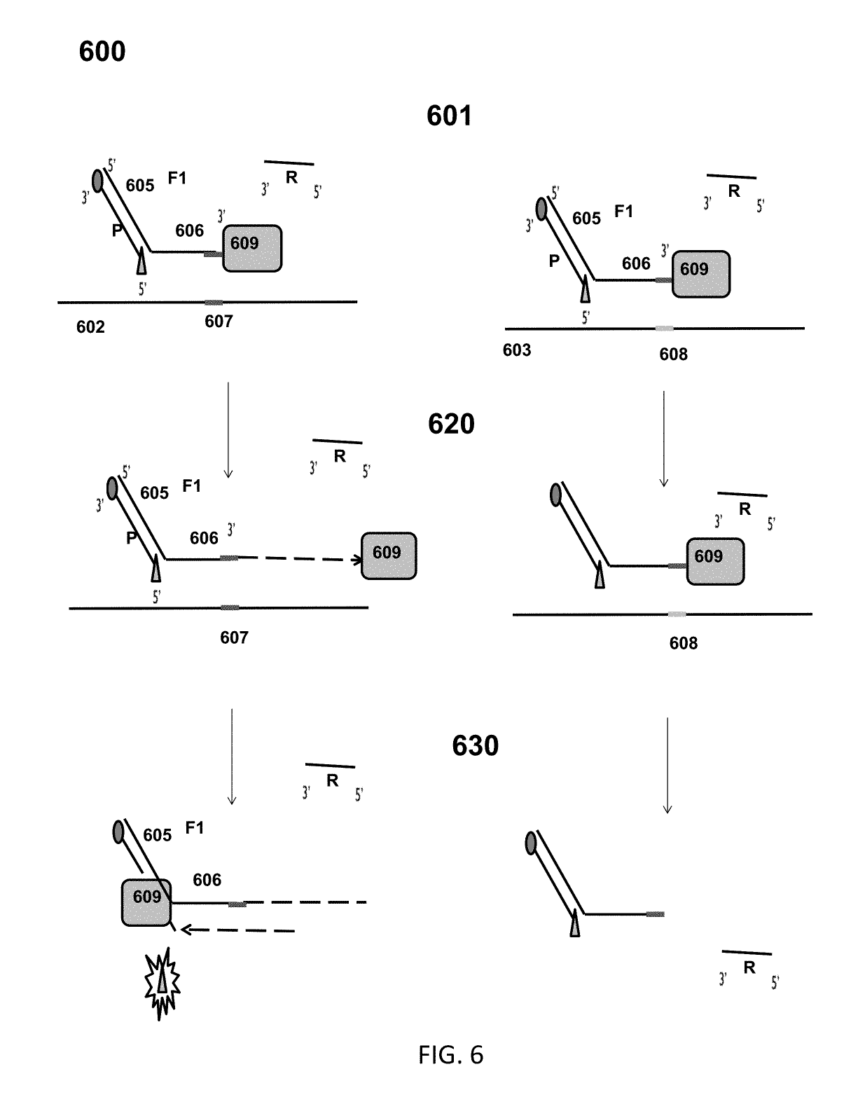

FIG. 6 depicts an exemplary embodiment of a method of the present invention. In a first step 601, a DNA sample comprising template DNA molecules 602 and 603 are contacted with a reaction mixture comprising dNTPs (not shown), a thermostable DNA polymerase 609 comprising 5′→3′ exonuclease activity and not comprising 3′→5′ exonuclease activity, a forward primer F1 comprising a probe-binding region 605 and a template binding region 606, and a reverse primer R. The 3′ terminal base of the forward primer F1 is complementary to a mutant allele 607 which resides on template molecule 602. By contrast, template molecule 603 has a wild-type allele 608 which is mismatched to the 3′ terminal base of forward primer F1. Also comprised in the reaction mixture is a reporter probe P which comprises a 5′ fluorescent moiety (triangle) and a 3′ quencher moiety (circle). In a first round of amplification (step 620), an annealing step is carried out wherein reporter probe P hybridizes to probe-binding region 605, resulting in a primer/reporter duplex P/F1. Additionally, F1 hybridizes to template molecules 602 and 603, resulting in complexes P/F1/102 and P/F1/103. During a synthesis step, DNA polymerase 609 promotes efficient extension of the P/F1/102 complex due to complementarity of the 3′terminal base of F1 with mutant allele 607. The extension of F1 from template molecule 602 results in a chimeric extension product comprising the extended primer F1 and the hybridized reporter probe P. The extended primer F1 further comprises a primer binding site for reverse primer R. By contrast, extension of P/F1/103 does not occur because of a mismatch between wild-type allele 608 and the 3′ terminal base of F1. Accordingly, no chimeric extension product comprising the extended primer F1 and hybridized reporter probe P is produced from a template molecule containing the wild-type allele. In a second (and any subsequent round) of amplification (step 630), reverse primer R hybridizes to the chimeric extension product. DNA polymerase 609 promotes extension of reverse primer R, and the 5′→3′ exonuclease activity of polymerase 609 separates the fluorescent moiety from the quencher moiety, e.g., by hydrolysis, resulting in a detectable signal.

In some embodiments, a reaction mixture can comprise multiple primers and probes for multiplex detection. By way of example only, a reaction mixture can comprise a common reverse primer and two or more forward primers, wherein each of the forward primers hybridizes to the same region in the template polynucleotide but differs from the other forward primers in the 5′ probe-binding region, wherein each forward primer comprises a unique probe-binding region, and wherein the template binding region of each of the forward primers differs from the other forward primers in the 3′ terminal base, which is complementary to either a wild-type allele or to one or another mutant alleles. Accordingly, the reaction mixture can also comprise two or more different reporter probes, each probe having a sequence corresponding to one of the two or more unique probe-binding regions on the two or more forward primers and comprising a distinct detectable moiety that is detectably distinct from any other detectable moiety in the reaction mixture.

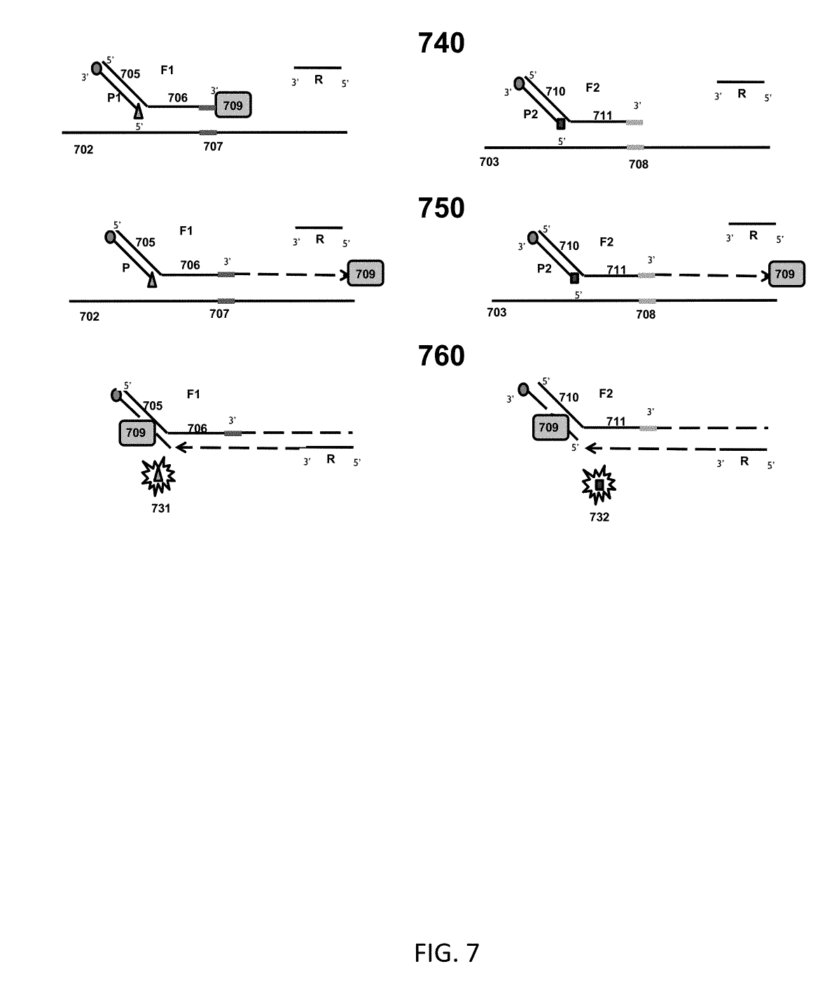

An exemplary embodiment of a multiplex assay detecting multiple alleles at a single locus is depicted in FIG. 7. In a first step 740, a DNA sample comprising template DNA molecules 702 and 703 are contacted with a reaction mixture comprising dNTPs (not shown), a thermostable DNA polymerase 709 comprising 5′→3′ exonuclease activity and not 3′→5′ exonuclease activity, a forward primer F1 comprising a probe-binding region 705 and a template binding region 706, a forward primer F2 comprising a probe-binding region 710 and a template binding region 711. The template binding regions 706 and 711 are identical except for the 3′ terminal base, which in F1 is complementary to a mutant allele 707 which resides on template molecule 702 and in F2 is complementary to a wild-type allele 708 which resides on template molecule 703. Accordingly, there is a mismatch between the 3′ terminal base of 706 and wild-type allele 708, and a mismatch between the 3′ terminal base of 711 and mutant allele 707. Also comprised in the reaction mixture is reporter probe P1 which comprises a 5′ fluorescent moiety (triangle) and a 3′ quencher moiety (circle) and reporter probe P2 which comprises a spectrally distinct 5′ fluorescent moiety (square) and a 3′ quencher moiety (circle). The reporter probe P1 hybridizes to probe-binding region 705, resulting in a P1/F1 duplex, and reporter probe P2 hybridizes to probe-binding region 710, resulting in a P2/F2 duplex. In a first round of amplification (step 750), F1 and F2 hybridize to template molecules 702 and 703, which can result in P1/F1/702, P1/F1/703, P2/F2/702, and P2/F2/703 complexes. DNA polymerase 709 can promote efficient extension of P1/F1/702 and P2/F2/703, which can result in chimeric extension products comprising the extended primer F1 and the hybridized reporter probe P1 (F1-P1) and/or extended primer F2 and the hybridized reporter probe P2 (F2-P2), respectively. The extended primers F1-P1 and F2-P2 may each further comprise a primer binding site for reverse primer R. By contrast, no extension of P1/F1/703 or P2/F2/702 occurs due to the presence of a mismatch between the 3′ terminal base of the forward primers and the template DNA. Accordingly, no chimeric extension product comprising the extended primer F1 and hybridized reporter probe P2 or comprising extended primer F2 and hybridized reporter P1 is produced. In a second (and any subsequent round) of amplification (step 760), reverse primer R can hybridize to the chimeric extension products F1-P1 and F2-P2. DNA polymerase 709 can promote extension of reverse primer R, and the 5′→3′ exonuclease activity of polymerase 709 separates the fluorescent moiety from the quencher moiety of each probe P1 and P2, resulting in spectrally distinct signals 731 and 732.

By way of other example only, a reaction mixture can comprise a plurality of primer/probe sets, wherein each set comprises a plurality of forward primers for the detection of multiple alleles at a particular locus, each forward primer harboring a unique probe-binding sequence and a template binding region, the 3′ terminal base of the template binding region corresponding to an allele of the locus, a common reverse primer, and detectably distinct reporter probes specific for each forward primer in the set. Such a reaction mixture can be used for the multiplex detection of multiple alleles at a plurality of loci. Accordingly, in some embodiments the invention provides a method of detecting up to 2, 3, 4, 5, 6, 7, 8, 9, 10, 20, 30, 40, 50, 60, 70, 80, 90, 100 alleles in a single multiplex assay.

In some embodiments, a reaction mixture comprises a plurality of primer/probe sets, wherein each set comprises a forward primer harboring a unique probe-binding sequence and a template binding region, a reverse primer that binds to a region downstream of said forward primer, and a detectably distinct reporter probe specific for the forward primer. Such a reaction mixture can be used for the multiplex detection of multiple loci. Multiplex detection of multiple loci can be used to assay copy number variation. For example, a first locus can be a region suspected of having a copy number variation and second locus can be a region that is predicted to not have a copy number variation. Comparison of detectable signal corresponding to the first and second loci can be used to measure copy number variation.

The detectable signal can be monitored in real-time during each amplification cycle. As used herein, “real-time PCR” can refer to PCR methods wherein an amount of detectable signal is monitored with each cycle of PCR. In some embodiments, a cycle threshold (Ct) wherein a detectable signal reaches a detectable level is determined. Generally, the lower the Ct value, the greater the concentration of the interrogated allele. Generally, data is collected during the exponential growth (log) phase of PCR, wherein the quantity of the PCR product is directly proportional to the amount of template nucleic acid. Systems for real-time PCR are known in the art and include, e.g., the ABI 7700 and 7900HT Sequence Detection Systems (Applied Biosystems, Foster City, Calif.). The increase in signal during the exponential phase of PCR can provide a quantitative measurement of the amount of templates containing the mutant allele.

In other embodiments, the detectable signal is monitored after amplification cycles have terminated (e.g., endpoint detection).

### Partitioning/Digital PCR

The method also can comprise partitioning the reaction mixture and nucleic acid sample into discrete volumes prior to amplification. Discrete volumes can contain template nucleic acid molecules from a starting nucleic acid sample. The starting nucleic acid sample can be diluted such that discrete volumes contain on average less than five, four, three, two, or one nucleic acid molecule. Partitions can contain no nucleic acid molecule. Partitions with no nucleic acids enable the use of Poisson statistics to determine original input DNA concentration. In some embodiments, discrete volumes can comprise a reaction mixture. Reaction mixtures are described herein. The method can comprise partitioning a nucleic acid sample into one set of discrete volumes, partitioning a reaction mixture into a second set of discrete volumes, and merging single discrete volumes from the first set with single discrete volumes from the second set to produce merged discrete volumes comprising a template nucleic acid molecule and a reaction mixture. In other embodiments, the method comprises admixing a nucleic acid sample with a reaction mixture to produce an admixture, and partitioning the admixture into discrete volumes. Discrete volumes can be independently assayed for the detection of one or more alleles.

Specific methods for partitioning are not critical to the practice of the invention. For example, partitioning can be carried out by manual pipetting. In a particular example, reaction mixture and nucleic acid sample can be distributed to individual tubes or well by manual pipetting. In another example, robotic methods can be used for the partitioning step. Microfluidic methods can also be used for the partitioning step.

A discrete volume can be, e.g., a tube, a well, a perforated hole, a reaction chamber, or a droplet, such as a droplet of an aqueous phase dispersed in an immiscible liquid, such as described in U.S. Pat. No. 7,041,481. Discrete volumes can be arranged into arrays of discrete volumes. Exemplary arrays include the Open array digital PCR system by Life Technologies (described in http://tools.invitrogen.com/content/sfs/manuals/cms_088717.pdf) and array systems by Fluidigm (www.fluidigm.com).

Partitioning a sample into small reaction volumes can confer many advantages. For example, the partitioning may enable the use of reduced amounts of reagents, thereby lowering the material cost of the analysis. By way of other example, partitioning can also improve sensitivity of detection. Without wishing to be bound by theory, partitioning of the reaction mixture and template DNA into discrete reaction volumes can give rare molecules greater proportional access to reaction reagents, thereby enhancing detection of rare molecules. For example, partitioning can enable the detection of a rare allele in a background of high wild-type allelic ratio. Accordingly, in some embodiments a reaction volume can be less than 1 ml, less than 500 microliters (ul), less than 100 ul, less than 10 ul, less than 1 ul, less than 0.5 ul, less than 0.1 ul, less than 50 nl, less than 10 nl, less than 1 nl, less than 0.1 nl, less than 0.01 nl, less than 0.001 nl, less than 0.0001 nl, less than 0.00001 nl, or less than 0.000001 nl. In some embodiments, a reaction volume can be 1-100 picoliters (pl), 50-500 pl, 0.1-10 nanoliters (nl), 1-100 nl, 50-500 nl, 0.1-10 microliters (ul), 5-100 ul, 100-1000 ul, or more than 1000 ul. In some embodiments, the reaction volumes are droplets. Without wishing to be bound by theory, the use of small droplets can enable the processing of large numbers of reactions in parallel. Accordingly, in some cases, the droplets have an average diameter of about, 0.000000000000001, 0.0000000000001, 0.00000000001, 0.000000001, 0.0000001, 0.000001, 0.00001, 0.0001, 0.001, 0.01, 0.05, 0.1, 1, 5, 10, 20, 30, 40, 50, 60, 70, 80, 100, 120, 130, 140, 150, 160, 180, 200, 300, 400, or 500 microns.

In some embodiments, the method comprises detection and/or measurement of an allele by digital PCR. The term “digital PCR”, as used herein, generally refers to a PCR amplification which is carried out on a nominally single, selected template molecule, wherein a number of individual single molecules are each isolated into discrete reaction volumes. In some embodiments, a large number of reaction volumes are used to produce higher statistical significance. Generally, PCR amplification in a reaction volume containing at least a single template (such as, e.g., a well, chamber, bead, emulsion, etc.) can have either a negative result, e.g., no detectable signal if no starting molecule is present, or a positive result, e.g., a detectable signal, if the targeted starting molecule is present. By analyzing a number of reaction areas indicating a positive result, insight into the number of starting molecules can be obtained. Such an analysis can be used for measurement of an amount of wild-type or mutant alleles in a sample, or be used for a measurement of copy number variation of a locus in a sample.

In particular embodiments, the method comprises droplet digital PCR methods. “Droplet digital PCR” generally refers to digital PCR wherein the reaction volumes are droplets. The droplets provided herein can prevent mixing between reaction volumes.

The droplets described herein can include emulsion compositions. The term “emulsion”, as used herein, generally refers to a mixture of immiscible liquids (such as oil and an aqueous solution, e.g., water). In some embodiments, the emulsion comprise aqueous droplets within a continuous oil phase. In other embodiments, the emulsion comprises oil droplets within a continuous aqueous phase. The mixtures or emulsions described herein may be stable or unstable. In preferred embodiments, the emulsions are relatively stable.

In some embodiments the emulsions exhibit minimal coalescence. “Coalescence” refers to a process in which droplets combine to form progressively larger droplets. In some cases, less than 0.00001%, 0.00005%, 0.00010%, 0.00050%, 0.001%, 0.005%, 0.01%, 0.05%, 0.1%, 0.5%, 1%, 2%, 2.5%, 3%, 3.5%, 4%, 4.5%, 5%, 6%, 7%, 8%, 9%, or 10% of droplets exhibit coalescence. The emulsions may also exhibit limited flocculation, a process by which the dispersed phase comes out of suspension in flakes. In some cases, less than 0.00001%, 0.00005%, 0.00010%, 0.00050%, 0.001%, 0.005%, 0.01%, 0.05%, 0.1%, 0.5%, 1%, 2%, 2.5%, 3%, 3.5%, 4%, 4.5%, 5%, 6%, 7%, 8%, 9%, or 10% of droplets exhibit flocculation.

The droplets can either be monodisperse (e.g., of substantially similar size and dimensions) or polydisperse (e.g., of substantially variable size and dimensions. In some embodiments, the droplets are monodisperse droplets. In some cases, the droplets are generated such that the size of the droplets does not vary by more than plus or minus 5% of the average size of the droplets. In some cases, the droplets are generated such that the size of the droplets does not vary by more than plus or minus 2% of the average size of the droplets. In some cases, a droplet generator will generate a population of droplets from a single sample, wherein none of the droplets vary in size by more than plus or minus 0.1%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% of the average size of the total population of droplets.

In some embodiments, the present invention provides systems, devices, and methods for droplet generation. In some embodiments, microfluidic systems are configured to generate monodisperse droplets (see, e.g., Kiss et al. Anal Chem. 2008 December 1; 80(23): 8975-8981). In some embodiments, the present invention provides micro fluidics systems for manipulating and/or partitioning samples.

In some embodiments, a microfluidics system comprises one or more of channels, valves, pumps, etc. (U.S. Pat. No. 7,842,248, herein incorporated by reference in its entirety). In some embodiments, a microfluidics system is a continuous-flow microfluidics system (see, e.g., Kopp et al., Science, vol. 280, pp. 1046-1048,1998, hereby incorporated by reference). In some embodiments, microarchitecture of the present invention includes, but is not limited to microchannels, microfluidic plates, fixed microchannels, networks of microchannels, internal pumps; external pumps, valves, centrifugal force elements, etc. In some embodiments, the microarchitecture of the present invention (e.g. droplet microactuator, microfluidics platform, and/or continuous-flow microfluidics) is complemented or supplemented with droplet manipulation techniques, including, but not limited to electrical (e.g., electrostatic actuation, dielectrophoresis), magnetic, thermal (e.g., thermal Marangoni effects, thermocapillary), mechanical (e.g., surface acoustic waves, micropumping, peristaltic), optical (e.g., opto-electrowetting, optical tweezers), and chemical means (e.g., chemical gradients). In some embodiments, a droplet microactuator is supplemented with a microfluidics platform (e.g. continuous flow components) and such combination approaches involving discrete droplet operations and microfluidics elements are within the scope of the invention.

In some embodiments, methods of the invention utilize a droplet microactuator. In some embodiments, a droplet microactuator is capable of effecting droplet manipulation and/or operations, such as, e.g., dispensing, splitting, transporting, merging, mixing, agitating. In some embodiments the invention employs droplet operation structures and techniques described in, e.g., U.S. Pat. Nos. 6,911,132, 6,773,566, and 6,565,727; U.S. patent application Ser. No. 11/343,284, and U.S. Patent Publication No. 20060254933, all of which are hereby incorporated by reference.

Droplet digital PCR techniques enable a high density of discrete PCR amplification reactions in a single volume. In some embodiments, greater than 100,000, 500,000, 1,000,000, 1,500,000, 2,000,000, 2,500,000, 5,000,000, or 10,000,000 separate reactions may occur per ul.

### Detection

Fluorescence detection can be achieved using a variety of detector devices equipped with a module to generate excitation light that can be absorbed by a fluorescer, as well as a module to detect light emitted by the fluorescer. In some cases, samples (such as droplets) may be detected in bulk. For example, samples may be allocated in plastic tubes that are placed in a detector that measures bulk fluorescence from plastic tubes. The samples can be distributed in a monolayer. Monolayer distributed samples can be detected by scanning users high resolution scanners (e.g., microarray scanners, GenePix 4000B Microarray Scanner (Molecular Devices), SureScan Microarray Scanner (Agilent)). If the sample is distributed in multiple layers, the sample can be detected with confocal imaging (e.g., confocal microscopy, spinning-disk confocal microscopy, confocal laser scanning microscopy). In some cases, one or more samples (such as droplets) may be partitioned into one or more wells of a plate, such as a 96-well or 384-well plate, and fluorescence of individual wells may be detected using a fluorescence plate reader.

In some embodiments amplification of the droplets, e.g., in a thermal cycle results in the generation of one or more detectable signals in a number of droplets. During the amplification reaction, a droplet comprising a template DNA molecule containing an interrogated allele can exhibit an increase in fluorescence relative to droplets that do not contain an interrogated allele. Droplets can be processed individually and fluorescence data collected from the droplets. For example, data relating to fluorescent signals from spectrally distinct fluorophores may be collected from each droplet.

A number of commercial instruments are available for analysis of fluorescently labeled materials. For instance, the ABI Gene Analyzer can be used to analyze attomole quantities of DNA tagged with fluorophores such as ROX (6-carboxy-X-rhodamine), rhodamine-NHS, TAMRA (5/6-carboxytetramethyl rhodamine NHS), and FAM (5′-carboxyfluorescein NHS). These compounds are attached to the probe by an amide bond through a 5′-alkylamine on the probe. Attachment can also occur through phosphoramidite precursors (e.g., 2-methoxy-3-trifluoroacetyl-1,3,2-oxazaphosphacydopentane or N-(3-(N,N-diisopropyiaminomethoxyphosphinyloxy)propyl)-2,2,2-trifluoroacetamide) which is a method to conjugate amino-derivatized polymers, especially oligonucleotides. Other useful fluorophores include CNHS (7-amino-4-methyl-coumarin-3-acetic acid, succinimidyl ester), which can also be attached through an amide bond.

Following digital PCR, the number of positive samples having a particular allele and the number of positive samples having any other allele (e.g., a wild-type allele) can be counted. In some cases, quantitative determinations are made by measuring the fluorescence intensity of individual partitions, while in other cases, measurements are made by counting the number of partitions containing detectable signal. In some embodiments, control samples can be included to provide background measurements that can be subtracted from all the measurements to account for background fluorescence. In other embodiments, 1, 2, 3, 4, 5, 6, 7, 8, 9, 10, or more than 10 different colors can be used to detect and measure different alleles, such as by using fluorophores of different colors on different PCR primers matched to probes recognizing different sequences.

In another embodiment of the invention, detection of a hydrolyzed reporter probe can be accomplished using, for example, luminescence (e.g., using Yttrium or Berrilium conjugates of EDTA), time-resolved fluorescence spectroscopy, a technique in which fluorescence is monitored as a function of time after excitation, or fluorescence polarization, a technique to differentiate between large and small molecules based on molecular tumbling. Large molecules (e.g., intact labeled probe) tumble in solution much more slowly than small molecules. Upon linkage of a fluorescent moiety to the molecule of interest (e.g., the 5′ end of a labeled probe), this fluorescent moiety can be measured (and differentiated) based on molecular tumbling, thus differentiating between intact and digested probe. Detection may be measured directly during PCR or may be performed post PCR.

### Kits for Allele Detection

Also provided in the invention are kits for the detection of one or more alleles of a locus. Kits may include one or more oligonucleotide primers as described herein, wherein each of the primers is capable of selectively detecting an individual allele of a locus. Kits may also include one or more reporter probes, as described herein. Kits can include, for example, one or more primer/probe sets. Exemplary primer/probe sets are described herein. Kits may further comprise instructions for use of the one or more primer/probe sets, e.g., instructions for practicing a method of the invention. In some embodiments, the kit includes a packaging material. As used herein, the term “packaging material” can refer to a physical structure housing the components of the kit. The packaging material can maintain sterility of the kit components, and can be made of material commonly used for such purposes (e.g., paper, corrugated fiber, glass, plastic, foil, ampules, etc.). Kits can also include a buffering agent, a preservative, or a protein/nucleic acid stabilizing agent. Kits can also include other components of a reaction mixture as described herein. For example, kits may include one or more aliquots of thermostable DNA polymerase as described herein, and/or one or more aliquots of dNTPs. Kits can also include control samples of known amounts of template DNA molecules harboring the individual alleles of a locus. In some embodiments, the kit includes a negative control sample, e.g., a sample that does not contain DNA molecules harboring the individual alleles of a locus. In some embodiments, the kit includes a positive control sample, e.g., a sample containing known amounts of one or more of the individual alleles of a locus.

### Systems for Allele Detection

Also provided in the invention are systems for the detection of one or more alleles in a sample. The system can provide a reaction mixture as described herein. In some embodiments the reaction mixture is admixed with a DNA sample and comprises template DNA. In some embodiments, the system further provides a droplet generator, which partitions the template DNA molecules, probes, primers, and other reaction mixture components into multiple droplets within a water-in-oil emulsion. Examples of some droplet generators useful in the present disclosure are provided in International Application No. PCT/US2009/005317. The system can further provide a thermocycler, which reacts the droplets via, e.g., PCR, to allow amplification and generation of one or more detectable signals. During the amplification reaction, a droplet comprising a template DNA molecule containing an interrogated allele exhibits an increase in fluorescence relative to droplets that do not contain an interrogated allele. In some embodiments, the system further provides a droplet reader, which processes the droplets individually and collects fluorescence data from the droplets. The droplet reader may, for example, detect fluorescent signals from spectrally distinct fluorophores. In some cases, the droplet reader further comprises handling capabilities for droplet samples, with individual droplets entering the detector, undergoing detection, and then exiting the detector. For example, a flow cytometry device can be adapted for use in detecting fluorescence from droplet samples. In some cases, a microfluidic device equipped with pumps to control droplet movement is used to detect fluorescence from droplets in single file. In some cases, droplets are arrayed on a two-dimensional surface and a detector moves relative to the surface, detecting fluorescence at each position containing a single droplet. Exemplary droplet readers useful in the present disclosure are provided in International Application No. PCT/US2009/005317.

Other exemplary systems for use with the method of the invention is described, for example, PCT Patent Application Pubs. WO 2007/091228 (U.S. Ser. No. 12/092,261); WO 2007/091230 (U.S. Ser. No. 12/093,132); and WO 2008/038259. Systems useful in practicing the invention include, e.g., systems from Stokes Bio (www.stokebio.ie), Fluidigm (www.fluidigm.com), Bio-Rad Laboratories, (www.bio-rad.com) RainDance Technologies (www.raindancetechnologies.com), Microfluidic Systems (www.microfluidicsystems.com); Nanostream (www.nanostream.com); and Caliper Life Sciences (www.caliperls.com). Other exemplary systems suitable for use with the methods of the invention are described, for example, in Zhang et al. Nucleic Acids Res., 35(13):4223-4237 (2007), Wang et al., J. Micromech. Microeng., 15:1369-1377 (2005); Jia et al., 38:2143-2149 (2005); Kim et al., Biochem. Eng. J., 29:91-97; Chen et al., Anal. Chem., 77:658-666; Chen et al., Analyst, 130:931-940 (2005); Munchow et al., Expert Rev. Mol. Diagn., 5:613-620 (2005); and Charbert et al., Anal. Chem., 78:7722-7728 (2006); and Dorfman et al., Anal. Chem, 77:3700-3704 (2005).

In some embodiments, the system further comprises a computer which stores and processes data. A computer-executable logic may be employed to perform such functions as subtraction of background fluorescence, assignment of target and/or reference sequences, and quantification of the data. For example, the number of droplets containing fluorescence corresponding to the presence of a particular allele (e.g., a mutant allele) in the sample may be counted and compared to the number of droplets containing fluorescence corresponding to the presence of another allele at the locus (such as, e.g., a wild-type allele).

### Subject-Specific Report

In some embodiments, methods for assessing cancer as described herein further comprise generating a subject-specific report on the tumor profile. The tumor profile can comprise a mutational status of one or more genes in the set of genes sequenced. The method can further comprise generation a subject-specific report on mutational status of the subset of genes over time. The subject-specific report can comprise information on dynamics of the tumor over time, based on a change in the level of cell-free DNA harboring the mutations in the subset of genes over time. An increase over time of cell-free DNA harboring the mutations can indicate an increase in tumor or cancer burden. A decrease over time of cell-free DNA harboring the mutations can indicate a decrease in tumor or cancer burden.

In some embodiments, the report provides a stratification and/or annotation of treatment options for the subject, based on the subject's tumor-specific profile. The stratification and/or the annotation can be based on clinical information for the subject. The stratification can include ranking drug treatment options with a higher likelihood of efficacy higher than drug treatment options with a lower likelihood of efficacy or for which no information exists with regard to treating subjects with the determined status of the one or more molecular markers. The stratification can include indicating on the report one or more drug treatment options for which scientific information suggests the one or more drug treatment options will be efficacious in a subject, based on the status of one or more tumor-specific mutations from the subject. The stratification can include indicating on a report one or more drug treatment options for which some scientific information suggests the one or more drug treatment options will be efficacious in the subject, and some scientific information suggests the one or more drug treatment options will not be efficacious in the subject, based on the status of one or more tumor-specific mutations in the sample from the subject. The stratification can include indicating on a report one or more drug treatment options for which scientific information indicates the one or more drug treatment options will not be efficacious for the subject, based on the status of one or more tumor-specific mutations in the sample from the subject. The stratification can include color coding the listed drug treatment options on the report based on the rank of the predicted efficacy of the drug treatment options.

The annotation can include annotation a report for a condition in the NCCN Clinical Practice Guidelines in Oncology™ or the American Society of Clinical Oncology (ASCO) clinical practice guidelines. The annotation can include listing one or more FDA-approved drugs for off-label use, one or more drugs listed in a Centers for Medicare and Medicaid Services (CMS) anti-cancer treatment compendia, and/or one or more experimental drugs found in scientific literature, in the report. The annotation can include connecting a listed drug treatment option to a reference containing scientific information regarding the drug treatment option. The scientific information can be from a peer-reviewed article from a medical journal. The annotation can include using information provided by Ingenuity® Systems. The annotation can include providing a link to information on a clinical trial for a drug treatment option in the report. The annotation can include presenting information in a pop-up box or fly-over box near provided drug treatment options in an electronic based report. The annotation can include adding information to a report selected from the group consisting of one or more drug treatment options, scientific information concerning one or more drug treatment options, one or more links to scientific information regarding one or more drug treatment options, one or more links to citations for scientific information regarding one or more drug treatment options, and clinical trial information regarding one or more drug treatment options. An exemplary embodiment of a subject-specific report is depicted in FIG. 8.

### Computer Systems

In another aspect, the invention provides computer systems for the monitoring of a cancer, generating a subject report, and/or communicating the report to a caregiver. In some embodiments, the invention provides computer systems for determining prognosis or determining efficacy of a therapy for a cancer in a subject in need thereof. The computer system can provide a report communicating said prognosis or therapy efficacy for said cancer. In some embodiments, the computer system executes instructions contained in a computer-readable medium. In some embodiments, the processor is associated with one or more controllers, calculation units, and/or other units of a computer system, or implanted in firmware. In some embodiments, one or more steps of the method are implemented in hardware. In some embodiments, one or more steps of the method are implemented in software. Software routines may be stored in any computer readable memory unit such as flash memory, RAM, ROM, magnetic disk, laser disk, or other storage medium as described herein or known in the art. Software may be communicated to a computing device by any known communication method including, for example, over a communication channel such as a telephone line, the internet, a wireless connection, or by a transportable medium, such as a computer readable disk, flash drive, etc. The one or more steps of the methods described herein may be implemented as various operations, tools, blocks, modules and techniques which, in turn, may be implemented in firmware, hardware, software, or any combination of firmware, hardware, and software. When implemented in hardware, some or all of the blocks, operations, techniques, etc. may be implemented in, for example, an application specific integrated circuit (ASIC), custom integrated circuit (IC), field programmable logic array (FPGA), or programmable logic array (PLA).

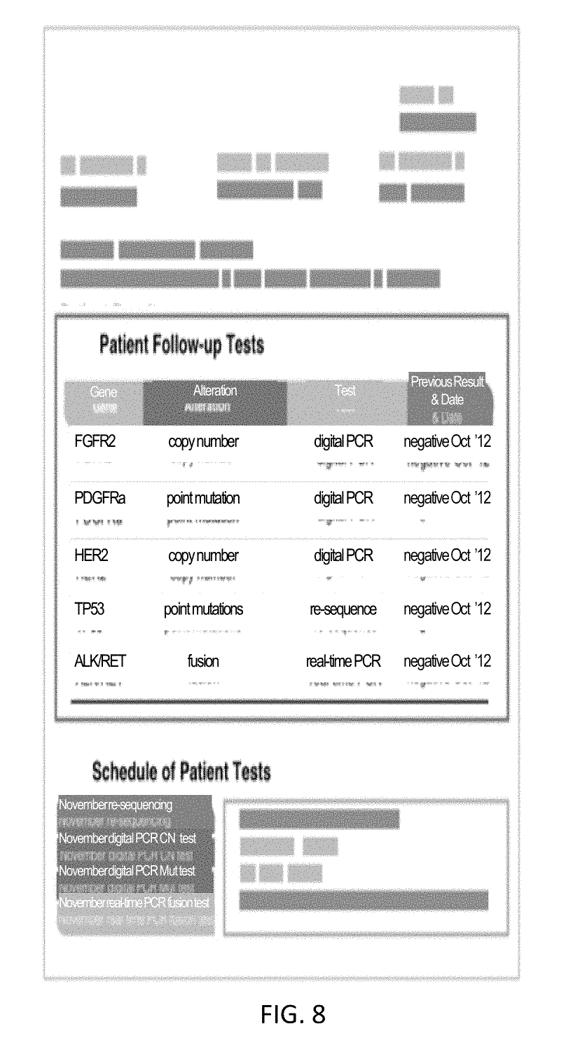

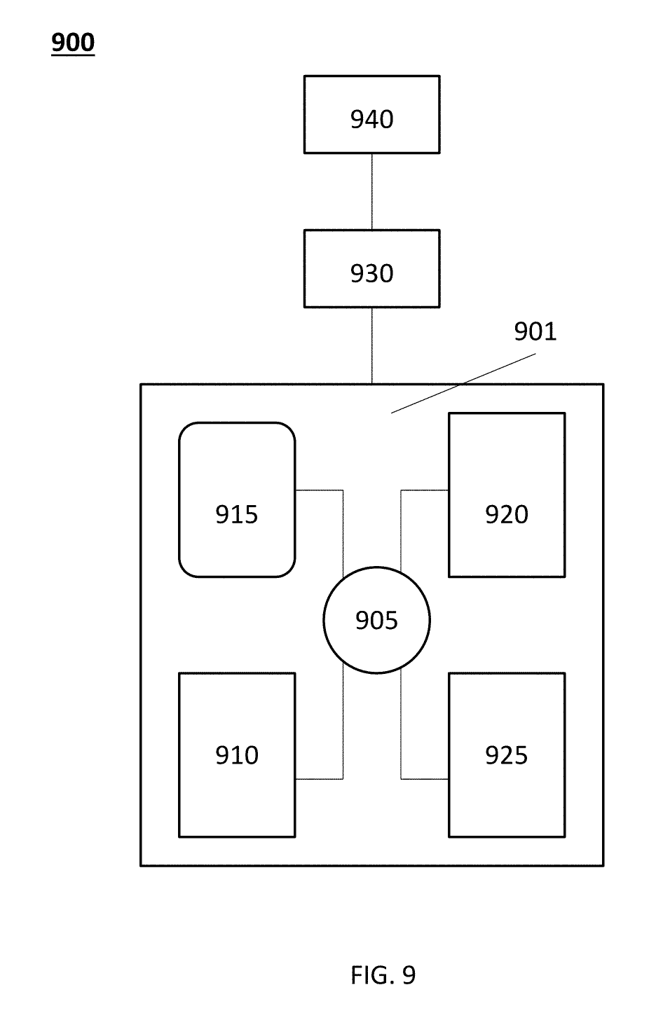

FIG. 9 depicts a computer system 900 adapted to enable a user to detect, analyze, and process patient data. The system 900 includes a central computer server 901 that is programmed to implement exemplary methods described herein. The server 901 includes a central processing unit (CPU, also “processor”) 905 which can be a single core processor, a multi core processor, or plurality of processors for parallel processing. The server 901 also includes memory 910 (e.g. random access memory, read-only memory, flash memory); electronic storage unit 915 (e.g. hard disk); communications interface 920 (e.g. network adaptor) for communicating with one or more other systems; and peripheral devices 925 which may include cache, other memory, data storage, and/or electronic display adaptors. The memory 910, storage unit 915, interface 920, and peripheral devices 925 are in communication with the processor 905 through a communications bus (solid lines), such as a motherboard. The storage unit 915 can be a data storage unit for storing data. The server 901 is operatively coupled to a computer network (“network”) 930 with the aid of the communications interface 920. The network 930 can be the Internet, an intranet and/or an extranet, an intranet and/or extranet that is in communication with the Internet, a telecommunication or data network. The network 930 in some cases, with the aid of the server 901, can implement a peer-to-peer network, which may enable devices coupled to the server 901 to behave as a client or a server.

The storage unit 915 can store files, such as subject reports, and/or communications with the caregiver, sequencing data, data about individuals, or any aspect of data associated with the invention.

The server can communicate with one or more remote computer systems through the network 930. The one or more remote computer systems may be, for example, personal computers, laptops, tablets, telephones, Smart phones, or personal digital assistants.

In some situations the system 900 includes a single server 901. In other situations, the system includes multiple servers in communication with one another through an intranet, extranet and/or the Internet.

The server 901 can be adapted to store sequencing information, or patient information, such as, for example, polymorphisms, mutations, patient history and demographic data and/or other information of potential relevance. Such information can be stored on the storage unit 915 or the server 901 and such data can be transmitted through a network.

Methods as described herein can be implemented by way of machine (or computer processor) executable code (or software) stored on an electronic storage location of the server 901, such as, for example, on the memory 910, or electronic storage unit 915. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910. Alternatively, the code can be executed on a second computer system 940.

Aspects of the systems and methods provided herein, such as the server 901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless likes, optical links, or the like, also may be considered as media bearing the software. As used herein, unless restricted to non-transitory, tangible “storage” media, terms such as computer or machine “readable medium” can refer to any medium that participates in providing instructions to a processor for execution.

Hence, a machine readable medium, such as computer-executable code, may take many forms, including but not limited to, tangible storage medium, a carrier wave medium, or physical transmission medium. Non-volatile storage media can include, for example, optical or magnetic disks, such as any of the storage devices in any computer(s) or the like, such may be used to implement the system. Tangible transmission media can include: coaxial cables, copper wires, and fiber optics (including the wires that comprise a bus within a computer system). Carrier-wave transmission media may take the form of electric or electromagnetic signals, or acoustic or light waves such as those generated during radio frequency (RF) and infrared (IR) data communications. Common forms of computer-readable media therefore include, for example: a floppy disk, a flexible disk, hard disk, magnetic tape, any other magnetic medium, a CD-ROM, DVD, DVD-ROM, any other optical medium, punch cards, paper tame, any other physical storage medium with patterns of holes, a RAM, a ROM, a PROM and EPROM, a FLASH-EPROM, any other memory chip or cartridge, a carrier wave transporting data or instructions, cables, or links transporting such carrier wave, or any other medium from which a computer may read programming code and/or data. Many of these forms of computer readable media may be involved in carrying one or more sequences of one or more instructions to a processor for execution.

The results of monitoring of a cancer, generating a subject report, and/or communicating the report to a caregiver can be presented to a user with the aid of a user interface, such as a graphical user interface.

A computer system may be used for one or more steps, including, e.g., sample collection, sample processing, sequencing, allele detection, receiving patient history or medical records, receiving and storing measurement data regarding a detected level of tumor-specific mutations in a subject or sample obtained from a subject, analyzing said measurement data determine a diagnosis, prognosis, or therapeutic efficacy, generating a report, and reporting results to a receiver.

A client-server and/or relational database architecture can be used in the invention. In general, a client-server architecture is a network architecture in which each computer or process on the network is either a client or a server. Server computers can be powerful computers dedicated to managing disk drives (file servers), printers (print servers), or network traffic (network servers). Client computers can include PCs (personal computers) or workstations on which users run applications, as well as example output devices as disclosed herein. Client computers can rely on server computers for resources, such as files, devices, and even processing power. The server computer handles all of the database functionality. The client computer can have software that handles front-end data management and receive data input from users.

After performing a calculation, a processor can provide the output, such as from a calculation, back to, for example, the input device or storage unit, to another storage unit of the same or different computer system, or to an output device. Output from the processor can be displayed by a data display, e.g., a display screen (for example, a monitor or a screen on a digital device), a print-out, a data signal (for example, a packet), a graphical user interface (for example, a webpage), an alarm (for example, a flashing light or a sound), or a combination of any of the above. In an embodiment, an output is transmitted over a network (for example, a wireless network) to an output device. The output device can be used by a user to receive the output from the data-processing computer system. After an output has been received by a user, the user can determine a course of action, or can carry out a course of action, such as a medical treatment when the user is medical personnel. In some embodiments, an output device is the same device as the input device. Example output devices include, but are not limited to, a telephone, a wireless telephone, a mobile phone, a PDA, a flash memory drive, a light source, a sound generator, a fax machine, a computer, a computer monitor, a printer, an iPod, and a webpage. The user station may be in communication with a printer or a display monitor to output the information processed by the server. Such displays, output devices, and user stations can be used to provide an alert to the subject or to a caregiver thereof.

Data relating to the present disclosure can be transmitted over a network or connections for reception and/or review by a receiver. The receiver can be but is not limited to the subject to whom the report pertains; or to a caregiver thereof, e.g., a health care provider, manager, other healthcare professional, or other caretaker; a person or entity that performed and/or ordered the genotyping analysis; a genetic counselor. The receiver can also be a local or remote system for storing such reports (e.g. servers or other systems of a “cloud computing” architecture). In one embodiment, a computer-readable medium includes a medium suitable for transmission of a result of an analysis of a biological sample.

An exemplary embodiment of a subject-specific report is depicted in FIG. 8. The computer system can comprise a user accessible module which enables the ability for clinicians to request a service be performed. Clinicians can enter patient demographic and medical history information into the computer system. The computer system can process the entered information and create a barcode label that can be applied to the sample being analyzed. The barcoded-sample be sent for analysis to a third party analyzer. The barcoded information would be inaccessible to the third party analyzer to maintain accountability with The Health Insurance Portability and Accountability Act (HIPAA) compliancy. Information that can be anonymized can be accessible to the third party analyzer. The barcode can be used to track the progression of the sample through the analysis workflow resulting in the generation of an encrypted final report. The encrypted final report can be decrypted and made accessible to the clinician who originally entered the sample information.

### Ligation Method:

In some aspects, the invention provides methods and kits for performing highly efficient ligation reactions. In some embodiments, the methods comprise ligation of donor nucleic acids to acceptor nucleic acids. In some embodiments, the methods improve ligation efficiency by over 2-fold, 5-fold, 10-fold, 50-fold, 100-fold, 500-fold, 1000-fold, or more than 1000-fold as compared to current methods. The methods described herein can, for example, increase ligation efficiency to over 10%, 20%, 30%, 40%, 50%, 60%, 70%, 80%, 90%, 95%, 97%, 98%, 99%, 99.5%, or 99.9% efficiency. In some embodiments, the methods described herein can increase the specificity of a ligation reaction, resulting in, for example, over 30%, over 40%, over 50%, over 60%, over 70%, 80%, over 85%, over 90%, over 95%, over 97%, over 98%, over 99%, over 99.5%, over 99.9%, or substantially all of ligation products resulting from a desired donor-acceptor ligation, as compared to undesired ligation products, e.g., unwanted donor-donor or acceptor-acceptor concatamers. The methods described herein can result in ligation of over 50%, over 60%, over 70%, over 80%, over 85%, over 90%, over 95%, over 97%, over 98%, over 99%, over 99.5%, over 99.9%, or substantially all of the plurality of the donor or acceptor nucleic acid molecules, respectively, to the acceptor or donor nucleic acid molecules. A nucleic acid molecule (donor or acceptor) in the ligation reaction can be over 120 nucleotides in length. Such highly efficient ligation methods can be used to improve a wide range of applications, some of which are described herein by example.

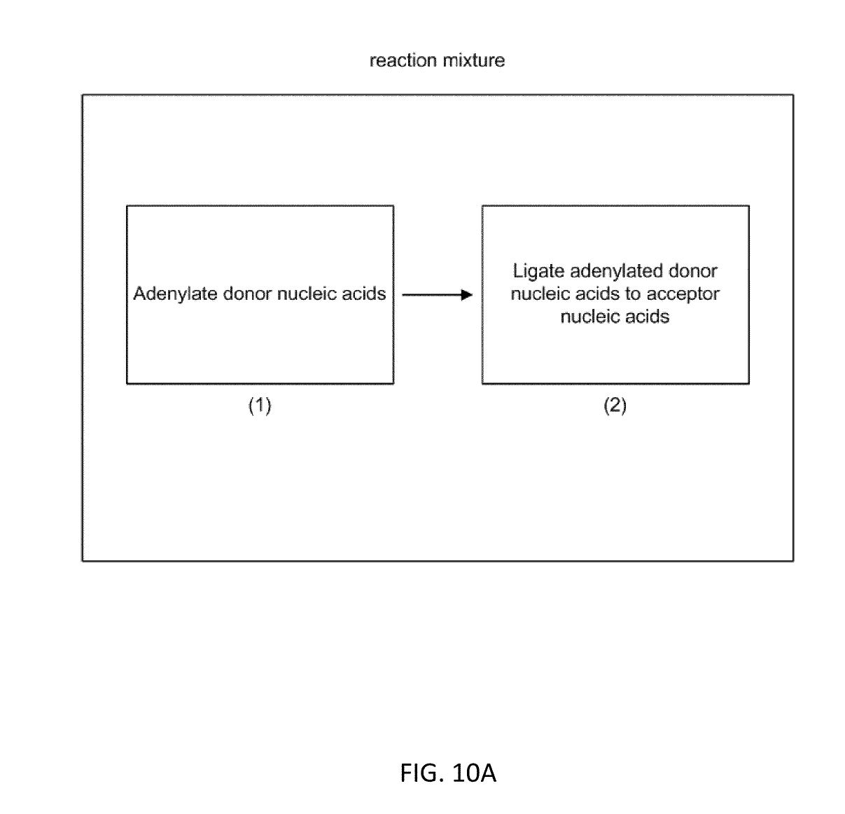

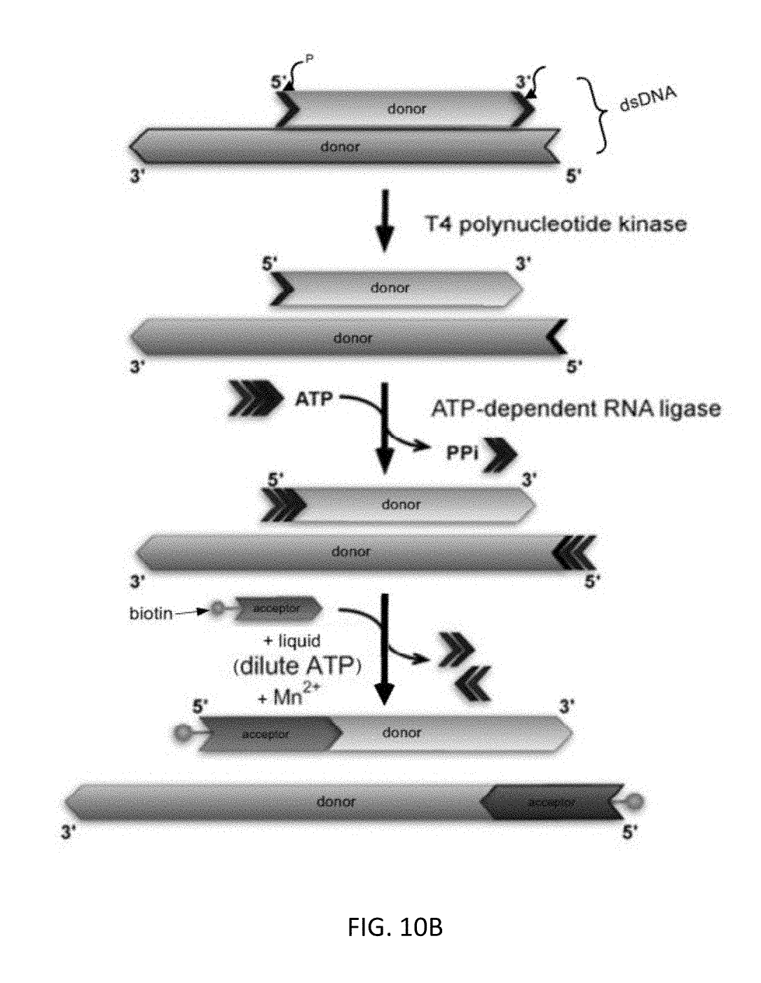

FIG. 10A depicts an exemplary embodiment of a method of the invention. In a first step (1), the method comprises transferring a nucleotide monophosphate (NMP) to an amount of donor nucleic acid molecules in a reaction mixture for a time sufficient to effect an accumulation of NMP-carrying donor nucleic acid molecules. In some embodiments, N=A. In some embodiments, N=G. A donor nucleic acid molecule can comprise a 5′ or 3′ phosphate group. In some embodiments, N=A, and a donor nucleic acid molecule comprises a 5′ phosphate group. In some embodiments, N=G, and a donor nucleic acid molecule comprises a 3′ phosphate group. In some embodiments, the reaction results in transfer of NMP to at least 10%, 20%, 30%, 40%, 50%, 60%, 70%, 80%, or 90% of the donor nucleic acid molecules present in the reaction mixture. In a second step (2), the method further comprises effecting formation of a covalent bond between an acceptor nucleic molecule and the NMP-carrying donor nucleic acid molecule (e.g., ligating an acceptor nucleic acid molecule to the NMP-carrying donor nucleic acid molecule. In some embodiments, the adenylation and ligation steps are carried out serially in a single reaction mixture. In some embodiments, the adenylated donor nucleic acid molecules are not separated from the reaction mixture prior to the second step (e.g., ligation step). In some embodiments, the first and second steps are carried out serially in the reaction mixture. In some embodiments, the ligation step is carried out after completion of the adenylation step. In some embodiments, over 10%, over 20%, over 30%, over 40%, over 50%, over 60%, over 70%, over 80%, over 90%, over 95%, over 97%, over 98%, over 99%, over 99.5%, over 99.9%, or substantially all of the donor nucleic acid molecules are carrying an NMP molecule upon commencement of the ligation step.

In some embodiments the donor and/or acceptor nucleic acid molecules are fully or partially denatured. Full or partial denaturation can be achieved by any means known in the art, including, e.g., heat denaturation, incubation in basic pH, denaturation in formamide, and/or urea denaturation. Heat denaturation can be achieved by heating a nucleic acid sample to about 60 deg C or above, about 65 deg C or above, about 70 deg C or above, about 75 deg C or above, about 80 deg C or above, about 85 deg C or above, about 90 deg C or above, about 95 deg C or above, or about 100 deg C or above. The nucleic acid sample can be heated by any means known in the art, including, e.g., incubation in a water bath, a temperature controlled heat block, or a thermal cycler.

Denaturation by incubation in basic pH can comprise incubation of the nucleic acid sample in any solution (e.g., a buffer) of pH 8 or greater, 9 or greater, 10 or greater, 11 or greater, 12 or greater. Denaturation by incubation in basic pH can be achieved by, for example, incubation of a nucleic acid sample in a solution comprising sodium hydroxide (NaOH), potassium hydroxide (KOH), sodium bicarbonate, sodium phosphate, Tris. The solution can comprise about 1 mM NAOH, 2 mM NAOH, 5 mM NAOH, 10 mM NAOH, 20 mM NAOH, 40 mM NAOH, 60 mM NAOH, 80 mM NAOH, 100 mM NAOH, 0.2M NaOH, about 0.3M NaOH, about 0.4M NaOH, about 0.5M NaOH, about 0.6M NaOH, about 0.7M NaOH, about 0.8M NaOH, about 0.9M NaOH, about 1.0M NaOH, or greater than 1.0M NaOH. The solution can comprise about 1 mM KOH, 2 mM KOH, 5 mM KOH, 10 mM KOH, 20 mM KOH, 40 mM KOH, 60 mM KOH, 80 mM KOH, 100 mM KOH, 0.2M KOH, 0.5M KOH, 1M KOH, or greater than 1M KOH. In some embodiments, the nucleic acid sample is incubated in NaOH or KOH for about 0.5, 1, 1.5, 2, 2.5, 3, 3.5, 4, 4.5, 5, 6, 7, 8, 9, 10, 12, 14, 16, 18, 20, 25, or 30 minutes. In some embodiments, the nucleic acid sample is incubated in Na-acetate following NaOH or KOH incubation.

Compounds like urea and formamide contain functional groups that can form hydrogen bonds with the electronegative centers of the nucleotide bases. At high concentrations (e.g., 8M urea or 70% formamide) of the denaturant, the competition for hydrogen bonds favors interactions between the denaturant and the N-bases rather than between complementary bases, thereby separating the two strands.

Without wishing to be bound by theory, in a typical ligation method, the intermediate steps of (1) transferring a NMP to the ligase and (2) transferring the NMP to the donor nucleic acid molecule, generally co-occur with the ligation step (3), and are reversible at neutral pH. The co-occurrence of all three steps and the reversibility of steps (1) and (2) can lead to poor ligation efficiency and poor specificity of the ligation products due to several factors, such as, e.g., the possibility of transferring NMP (e.g., adenylation, guanylylation) to both donor and acceptor species, removal of NMP from the ligase and/or donor (or acceptor) species (e.g., de-adenylation or de-guanylylation) of ligase and/or de-adenylation or de-guanylylation of the donor (or acceptor) species before ligation can occur. However, by performing the step of transferring NMP to the donor nucleic acid monoeclule and the step of ligation serially, it is possible to increase ligation efficiency by effecting an accumulation of NMP-carrying donor nucleic acid molecules prior to ligation to an acceptor species.

In some embodiments, reversibility of intermediate steps 1 & 2 is exploited to control the outcome of the reaction. In some embodiments, reversibility is controlled by modulating the relative concentrations of each component of the reaction mixture (e.g., ligase, nucleoside triphosphate (NTP), donor, and acceptor) to promote, e.g., adenylation over de-adenylation. By way of example only, if donor and acceptor nucleic acid species are present in adenylation reaction and comprise phosphorylated 5′ termini, the adenylation step becomes non-specific for donor and acceptor species, which can lead to non-specific formation of unwanted ligation products. However, if only the donor species is present for the adenylation step then adenylation can be made specific for the donor species. In such cases, the amount of ATP and ligase also affect the predominance of adenylation vs. de-adenylation. For example, self-ligation of the donor species can predominate at low concentrations of ligase, where high concentrations of ATP (e.g., less than the amount of donor nucleic acid molecules), can lead to unwanted concatenation of donor species. Limiting the amount of ATP can control the extent of concatenation observed. Accordingly, in some embodiments, the NMP transfer steps occur in a reaction mixture comprising an amount of donor nucleic acid molecules and an amount of a ligase that is at least equimolar to or in excess of the amount of donor nucleic acid molecules. Donor nucleic acid molecules in the reaction mixture prior to the ligating step can be present in an amount of 0.1-10, 5-30, 10-50, 20-100, 50-200, 100-500, 200-1000 ng/μl. Donor nucleic acid molecules in the reaction mixture prior to the ligating step can be present in an amount to provide about 0.01 pmol, 0.05 pmol, 0.1 pmol, 0.15 pmol, 0.2 pmol, 0.25 pmol, 0.5 pmol, 0.55 pmol, 0.6 pmol, 0.65 pmol, 0.7 pmol, 0.75 pmol, 0.8 pmol, 0.85 pmol, 0.9 pmol, 0.95 pmol, 1 pmol, 1.1 pmol, 1.2 pmol, 1.3 pmol, 1.4 pmol, 1.5 pmol, 1.6 pmol, 1.7 pmol, 1.8 pmol, 1.9 pmol, 2 pmol, 5 pmol, 10 pmol, 15 pmol, 20 pmol, 25 pmol, 30 pmol, 35 pmol, 40 pmol, 45 pmol, 50 pmol, 55 pmol, 60 pmol, 65 pmol, 70 pmol, 75 pmol, 80 pmol, 85 pmol, 90 pmol, 95 pmol, 100 pmol, 110 pmol, 120 pmo1,130 pmol, 140 pmol, 150 pmol, 160 pmol, 170 pmol, 180 pmol, 190 pmol, 200 pmol, 300 pmol, 400 pmol, 500 pmol, 600 pmol, 700 pmol, 800 pmol, 900 pmol, 1000 pmol (1 nmol), 2 nmol, 5 nmol, 10 nmol, or more than 10 nmol of 5′ termini. In some embodiments, the amount of ligase is at least 1×, 1.25×, 1.5×, 2×, 3×, 4×, 5×, 7.5×, 10×, 15×, 20×, or over 20× the amount of donor nucleic acid molecules. In some embodiments, the amount of ligase is 1-5×, 2-10×, 5-20× or over 20× the amount of donor nucleic acid molecules. In some embodiments, the amount of ligase in the reaction mixture is about 0.01, 0.05, 0.1, 0.5 1, 1.5, 2, 4, 6, 8, 10, or more than 10 μM. In some embodiments, the adenylation steps occur in a reaction mixture comprising an amount of donor nucleic acid molecules and an amount of ligase that is at least 0.25-fold higher, 0.5-fold higher, 1-fold higher, 1.5-fold higher, 2-fold higher, 3-fold higher, 4-fold higher, 5-fold higher, 6-fold higher, 7-fold higher, 8-fold higher, 9-fold higher, 10-fold higher, 15-fold higher, 20-fold higher, or more than 20-fold higher than the amount of donor nucleic acid molecules.

The ligase can be an ATP-dependent ligase. The ATP-dependent ligase can be an RNA ligase. The RNA ligase can be, e.g., an Archaeal RNA ligase, e.g., an archaeal RNA ligase from the thermophilic archaeon Methanobacterium thermoautotrophicum (MthRn1). The RNA ligase can be an Rnl 1 family ligase. Generally, Rnl 1 family ligases can repair single-stranded breaks in tRNA. Exemplary Rnl 1 family ligases include, e.g., T4 RNA ligase, thermostable RNA ligase 1 from Thermus scitoductus bacteriophage TS2126 (CircLigase), or CircLigase II). Such ligases can be described in WIPO Patent Application Publication No. WO2010094040, hereby incorporated by reference. The RNA ligase can be an Rnl 2 family ligase. Generally, Rnl 2 family ligases can seal nicks in duplex RNAs. Exemplary Rnl 2 family ligases include, e.g., T4 RNA ligase 2. In some embodiments, the ATP-dependent ligase is an ATP-dependent DNA ligase. The ATP-dependent DNA ligase can be a T4 DNA ligase. These ligases generally catalyze the ATP-dependent formation of a phosphodiester bond between a nucleotide 3′-OH nucleophile and a phosphate of a 5′ AMP·P group.

In some embodiments, the ligase is a GTP-dependent ligase. The GTP-dependent ligase can be an RNA ligase. The GTP-dependent RNA ligase can be RtcB RNA ligase. The RtcB ligase can catalyze a GTP=dependent formation of a phosphodiester bond between a phosphate of a 3′ GMP·P group and a nucleotide 5′-OH nucleophile.

In some embodiments, the reaction mixture comprises an amount of NTP sufficient to promote transfer of NMP to donor nucleic acid molecules over removal of NMP from the donor nucleic acid molecules (e.g., promotes adenylation or guanylylation over de-adenylation or de-guanylylation). In some embodiments, the amount of NTP is sufficient to inhibit formation of a covalent bond between adenylated donor nucleic acid molecules. In some embodiments, the adenylation steps occur in a reaction mixture comprising an amount of donor nucleic acid molecules, an amount of NTP-dependent ligase, and an amount of NTP that is at least 2-fold, 3-fold, 4-fold, 5-fold, 6-fold, 7-fold, 8-fold, 9-fold, or 10-fold higher than a Michaelis constant (Km) of the NTP-dependent ligase. In some embodiments, the adenylation steps occur in a reaction mixture comprising an amount of donor nucleic acid molecules an amount of NTP-Michaelis constant (Km)dependent ligase that is at least equimolar to or in excess of the amount of donor nucleic acid molecules, and an amount of NTP that is at least 2-fold, 3-fold, 4-fold, 5-fold, 6-fold, 7-fold, 8-fold, 9-fold, or 10-fold higher than the Michaelis constant (Km) of the NTP-dependent ligase. In particular embodiments, about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1000 μM of NTP is present in the reaction mixture. Such amounts of NTP may inhibit the ligation step.

The reaction mixture in which adenylation occurs can further comprise a cation. The cation can be Mg²⁺, or can be Mn²⁺. In some embodiments, the cation is Mg²⁺. The Mg²⁺ can be present in the reaction mixture at a final concentration of 0.1 mM-1 mM, 1 mM-10 mM, 5-20 mM, 10-50 mM, 30-100 mM, or more than 100 mM. The Mg^{t} can be present in the reaction mixture at a final concentration of about 10 mM. In some embodiments, the cation is present in an amount sufficient to catalyze adenylation of the ligase and subsequent adenylation of the donor nucleic acid molecules.

In some embodiments the reaction mixture further comprises a high molecular weight inert molecule, e.g., PEG of MW 4000, 6000, or 8000. In some embodiments, the inert molecule is present in an amount that is about 0.5%, 1%, 2%, 3%, 4%, 5%, 7.5%, 10%, 12.5%, 13%, 13.5%, 14%, 14.5%, 15%, 15.5%, 16%, 16,5%, 17%, 17.5%, 18%, 18.5%, 19%, 19.5%, 20%, 25%, 30%, 35%, 40%, 45%, 50%, or greater than 50% weight/volume. In some embodiments, the inert molecule is present in an amount that is about 0.5-2%, about 1-5%, about 2-15%, about 10-20%, about 15-30%, about 20-50%, or more than 50% weight/volume.

The NMP transfer steps described herein can effect an accumulation of NMP-carrying donor nucleic acid molecules. The accumulation of NMP-carrying donor nucleic acid molecules can result in at least 10%, at least 20%, at least 30%, at least 40%, at least 50%, at least 60%, at least 70%, at least 80%, at least 90%, at least 95%, at least 97%, at least 98%, at least 99%, at least 99.5%, at least 99.9%, or substantially all of the plurality of the donor nucleic acid molecules present in the reaction mixture carrying an NMP.

During the NMP transfer steps, unwanted ligation products resulting from, e.g., donor/donor circularization or concatenation can be minimized or prevented by any means. Unwanted ligation can be minimized or prevented, for example, by carrying out the adenylation reaction in the presence of an amount of NTP sufficient to inhibit formation of a covalent bond (e.g., ligation) between adenylated donor nucleic acid molecules. Exemplary amounts of NTP which may inhibit ligation are described herein. Unwanted ligation can also be prevented by modification of the 3′terminal group of the donor nucleic acid molecules. 3′ terminal groups of the donor nucleic acid molecules can be modified with a 3′ terminal blocking group by any means known in the art. Generally, the 3′ terminal blocking group will prevent the formation of a covalent bond between the 3′ terminal base and another nucleotide. In some embodiments, the 3′ terminal blocking group is dideoxy-dNTP, biotin, 3′ amino moiety, a “reversed” nucleoside base. In some embodiments, the ligase is a T4 RNA ligase and a donor nucleic acid molecule comprises a modified 3′ terminal group. In other embodiments, the ligase is a T4 RNA ligase and donor nucleic acid molecules comprise unmodified 3′ terminal groups. In yet other embodiments, the ligase is not a T4 RNA ligase and donor nucleic acid molecules comprise unmodified 3′ terminal groups.

In some embodiments, adenylation occurs in the reaction mixture for a time sufficient to effect accumulation of adenylated donor nucleic acid molecules. In some embodiments, the reaction mixture is incubated for about 1 minutes, about 2 minutes, about 3 minutes, about 4 minutes, 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 70 minutes, about 80 minutes, about 90 minutes, about 120 minutes, about 150 minutes, about 180 minutes, about 210 minutes, about 240 minutes, or more than 240 minutes. In some embodiments, the reaction mixture is incubated for 2-10 minutes, 5-20 minutes, 10-30 minutes, 20-60 minutes, 30-90 minutes, 60-150 minutes, 120-240 minutes, or more than 240 minutes.

In some embodiments the reaction mixture is incubated at a desired temperature to facilitate adenylation of donor nucleic acid molecules. In some embodiments the reaction mixture is heated to about 50 deg C, about 51 deg C, about 52 deg C, about 53 deg C, about 54 deg C, about 55 deg C, about 56 deg C, about 57 deg C, about 58 deg C, about 59 deg C, about 60 deg C, about 61 deg C, about 62 deg C, about 63 deg C, about 64 deg C, about 65 deg C, about 66 deg C, about 67 deg C, about 68 deg C, about 69 deg C, about 70 deg C, or above 70 deg C. In some embodiments the reaction mixture is heated to about 60-70 deg C. In other embodiments adenylation can occur at room temperature (e.g., 20-25 deg C) or can occur at about 35-40 deg C (e.g., 37 deg C). In some embodiments the reaction mixture is incubated at 0-4 deg C, 4-15 deg C, or 10-20 deg C. In some embodiments the reaction mixture is incubated for about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 70 minutes, about 80 minutes, about 90 minutes, about 120 minutes, about 150 minutes, about 180 minutes, about 210 minutes, about 240 minutes, or more than 240 minutes. In some embodiments, the reaction mixture is incubated for 2-10 minutes, 5-20 minutes, 10-30 minutes, 20-60 minutes, 30-90 minutes, 60-150 minutes, 120-240 minutes, or more than 240 minutes. In particular embodiments the reaction mixture is heated to 65 deg C for about 60 minutes.

After accumulation of adenylated donor nucleic acid molecules, ligation of an acceptor nucleic acid molecule to an adenylated donor nucleic acid molecule can be effected without separating (e.g., purifying) the adenylated donor nucleic acid molecules from the reaction mixture. In some embodiments ligation is effected by further adding to the reaction mixture liquid in an amount sufficient to dilute NTP. In some embodiments NTP is diluted 2-fold, 3-fold, 4-fold, 5-fold, 6-fold, 7-fold, 8-fold, 9-fold, 10-fold, 12-fold, 15-fold, 20-fold, 50-fold, 100-fold, or more than 100-fold. The liquid can comprise water, buffer, monovalent ion, cation, a high molecular weight inert molecule, or any combination thereof. For example, further amounts of buffer, monovalent ion, cation, high molecular weight inert molecule, or any combination thereof, can be added to the reaction mixture in order to preserve the original concentration of these reaction mixture components upon dilution of NTP. The dilution of NTP can release NTP-mediated inhibition of the ligase, thereby allowing the ligation step to proceed. In some embodiments ligation is effected by further adding to the reaction mixture a cation. The cation can be Mg²⁺, or can be Mn²⁺. In some embodiments the cation is Mn²⁺. In some embodiments the cation facilitates the ligation step. In some embodiments Mn²⁺ is present in the reaction mixture at a final concentration of 0 mM-2 mM, 1 mM-2.5 mM, 2.5 mM-5 mM, 5 mM-7.5 mM, or greater than 7.5 mM. In some embodiments Mn²⁺ is present in the reaction mixture at a final concentration of 2.5 mM, 3 mM, 3.5 mM, 4 mM, 4.5 mM, 5 mM, 5.5 mM, 6 mM, 6.5 mM, 7 mM, 7.5 mM, or more than 7.5 mM. In some embodiments the method further comprises adding to the reaction mixture an amount of acceptor nucleic acid molecules. In some embodiments the acceptor nucleic acid molecules are added in an amount that is excess as compared to the amount of donor nucleic acid molecules. For example, the acceptor nucleic acid molecules can be added in an amount that is 1.5×-10×, 2×-50×, 5×-100×, 50×-500×, or more than 500× the amount of donor nucleic acid molecules in the reaction mixture. In other embodiments the acceptor nucleic acid molecules are added in an amount such that the amount of donor nucleic acid molecules are in excess as compared to the amount of acceptor nucleic acid molecules. For example, the donor nucleic acid molecules can be present in an amount that is 1.5×-10×, 2×-50×, 5×-100×, 50×-500×, or more than 500× the amount of acceptor nucleic acid molecules in the reaction mixture. In some embodiments, additional amounts of ligase can be added to the reaction mixture. In some embodiments, no additional ligase is added to the reaction mixture.

In some embodiments, the reaction mixture is incubated for a time sufficient to effect ligation of the NMP-carrying donor nucleic acid molecules to the acceptor nucleic acid molecules. In some embodiments, the reaction mixture is incubated for about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 70 minutes, about 80 minutes, about 90 minutes, about 120 minutes, about 150 minutes, about 180 minutes, about 210 minutes, about 240 minutes, or more than 240 minutes. In some embodiments, the reaction mixture is incubated for 2-10 minutes, 5-20 minutes, 10-30 minutes, 20-60 minutes, 30-90 minutes, 60-150 minutes, 120-240 minutes, or more than 240 minutes.

In some embodiments the reaction mixture is incubated at a desired temperature to facilitate ligation. In some embodiments the reaction mixture is heated to about 50 deg C, about 51 deg C, about 52 deg C, about 53 deg C, about 54 deg C, about 55 deg C, about 56 deg C, about 57 deg C, about 58 deg C, about 59 deg C, about 60 deg C, about 61 deg C, about 62 deg C, about 63 deg C, about 64 deg C, about 65 deg C, about 66 deg C, about 67 deg C, about 68 deg C, about 69 deg C, about 70 deg C, or above 70 deg C. In some embodiments the reaction mixture is heated to about 60-70 deg C. In other embodiments ligation can occur at cold temperatures (e.g., about 0-4 deg C, about 4 deg C, about 4-15 deg C, about 12 deg C, or about 10-20 deg C), at room temperature (e.g., 20-25 deg C) or can occur at about 35-40 deg C (e.g., 37 deg C). In some embodiments the reaction mixture is incubated at the desired temperature for about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 70 minutes, about 80 minutes, about 90 minutes, about 120 minutes, about 150 minutes, about 180 minutes, about 210 minutes, about 240 minutes, or more than 240 minutes. In some embodiments, the reaction mixture is incubated at the desired temperature for 2-10 minutes, 5-20 minutes, 10-30 minutes, 20-60 minutes, 30-90 minutes, 60-150 minutes, 120-240 minutes, or more than 240 minutes. In particular embodiments the reaction mixture is heated to 65 deg C for about 60 minutes.

Following incubation, the method can further comprise inactivating the ligase by any means known in the art. Inactivation of the ligase can be effected by heat-inactivation. For example, the reaction mixture can be heated to 65, 70, 75, 80, 85, 90, 95, or more than 95 deg C for 1, 2, 3, 4, 5, 6, 7, 8, 9, 10, or more than 10 minutes. In particular embodiments, the reaction mixture is heated to 80 deg C for 10 minutes, followed by 95 deg C for 3 minutes. Inactivation of the ligase can also be effected by, e.g., incubation with EDTA, incubation with formamide, incubation with urea, or incubation with protease.

Following inactivation of the ligase, the desired ligation products can be purified or separated from the reaction mixture by any means known in the art. For example, proteins of the reaction mixture can be removed, for example, by treating the reaction mixture with a protease. Protease treatment can involve incubating the reaction mixture with a protease for about 1, 2, 3, 4, 5, 10, 15, 20, 25, 30, 35, 40, 45, 50, 55, 60 minutes, or over 60 minutes at 20-25 deg C, 35-40 deg C (e.g., 37 deg C), or more than 40 deg C. The protease can then be inactivated, e.g., by incubating for 10-20 minutes at 75 deg C. The desired reaction products can be further purified, for example, by precipitation, by column purification, by centrifugation, or any other method known in the art.

An exemplary embodiment of a method for high-efficiency ligation is depicted in FIG. 10A-10B. In a first step (optional), double-stranded DNA fragments (e.g., donor) are partially denatured and treated with T4 polynucleotide kinase. The T4 polynucleotide kinase catalyzes the addition of phosphate groups to the 5′ termini of donor nucleic acid molecules and removal of phosphate groups from the 3′ termini of donor nucleic acid molecules. The donor may or may not be purified at this point. In a next step, the donor molecules are added to a reaction mixture comprising excess ATP-dependent RNA ligase, excess ATP, and Mg²⁺. The ligase catalyzes transfer of an adenylyl monophosphate to the 5′ phosphate of the donor molecules, releasing PPi. The reaction mixture is incubated under conditions sufficient to effect an accumulation of adenylated donor nucleic acid molecules. In a next step following adenylation, liquid is added to the reaction mixture to dilute ATP at least 10-fold. The liquid may comprise further components, including but not limited to water, monovalent salts, Mg²⁺, PEG. Also added to the reaction mixture are nucleic acid molecules to be ligated to the donor molecules (e.g., acceptor) and Mn²⁺. The acceptor nucleic acids may or may not comprise a detectable tag (e.g., biotin). The detectable tag may be used for detecting and/or affinity binding. Both the dilution of ATP and addition of Mn²⁺ drive the ligation reaction to completion, resulting in ligation products comprising acceptor-donor molecules.

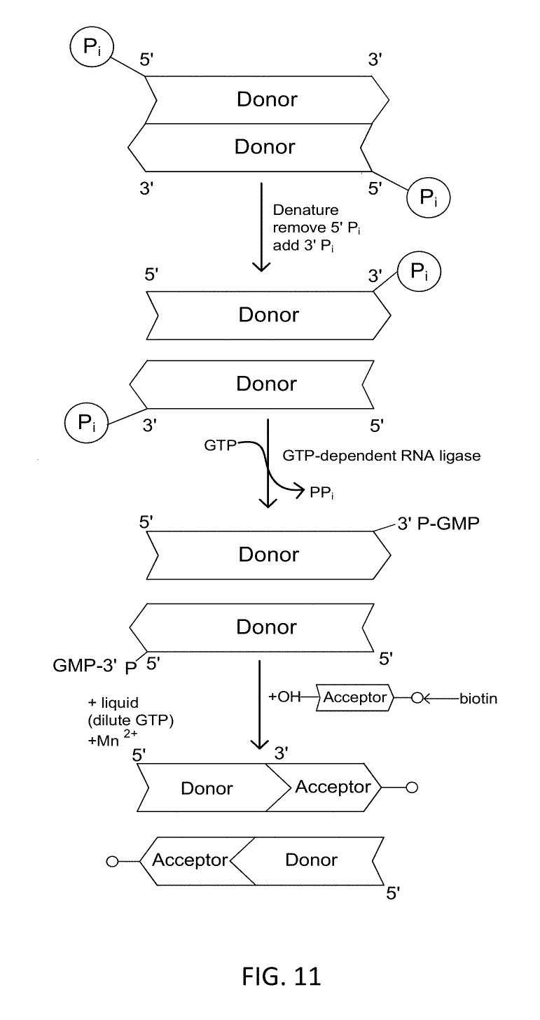

Another exemplary embodiment of a method for high-efficiency ligation is depicted in FIG. 11. In a first step (optional), double-stranded DNA fragments (e.g., donor) are partially denatured and treated with an enzyme that catalyzes the addition of phosphate groups to the 3′ adenylation of at least 10%, 20%, 30%, 40%, 50%, 60%, 70%, 80%, or 90% termini of donor nucleic acid molecules and removal of phosphate groups from the 5′ termini of donor nucleic acid molecules. The donor may or may not be purified at this point. In a next step, the donor molecules are added to a reaction mixture comprising excess GTP-dependent RNA ligase (e.g., RtcB), excess GTP, and Mn²⁺. The ligase catalyzes transfer of an guanylyl monophosphate to the 3′ phosphate of the donor molecules, releasing PPi. The reaction mixture is incubated under conditions sufficient to effect an accumulation of guanylylated donor nucleic acid molecules. In a next step following adenylation, liquid is added to the reaction mixture to dilute GTP at least 10-fold. The liquid may comprise further components, including but not limited to water, monovalent salts, Mn²⁺, PEG. Also added to the reaction mixture are nucleic acid molecules to be ligated to the donor molecules (e.g., acceptor) and Mn²⁺. The acceptor nucleic acids may or may not comprise a detectable tag (e.g., biotin). The detectable tag may be used for detecting and/or affinity binding. Both the dilution of GTP and addition of Mn²⁺ drive the ligation reaction to completion, resulting in ligation products comprising acceptor-donor molecules.

### Exemplary Applications

The high-efficiency ligation methods are useful for a wide range of applications. For example, the high efficiency ligation methods are useful for any applications in which tagging of nucleic acids with a detectable tag or an affinity tag is desired. For other example, the high efficiency ligation methods are useful for any applications in which linking of one nucleic acid species to another nucleic acid species is desired. The high efficiency ligation methods are also useful for the preparation of nucleic acid libraries for analysis, e.g., for analysis by sequencing, by array hybridization assays, including comparative genome hybridization (CGH) assays. Such high efficiency preparation methods confer many advantages to downstream analysis, for example, by allowing for the direct analysis of a starting sample of nucleic acids without significant loss of starting material, by allowing for direct analysis of nucleic acids without requiring pre-amplification, by allowing for analysis of nucleic acids without introducing labeling or amplification bias which can be associated with pre-amplification, and lowering potential bioinformatic load. Such high efficiency ligation methods and kits may also be useful for, e.g., molecular cloning purposes, or for barcoding applications.

### Sequencing Applications/High Efficiency Library Preparation

The high efficiency ligation methods and kits as described herein can be applied to the preparation of nucleic acid libraries for sequencing. Such preparation methods enable digital sequencing of the nucleic acids without significant loss of starting material, particularly for sequencing utilizing emulsion based sequencing platforms. Such preparation methods can also enable detection of DNA methylation without the use of bisulfite treatment. An exemplary method of DNA methylation detection is described in Flusberg et. al., Nature Methods 2010 June: 7(6):461-465, which is hereby incorporated by reference. Accordingly, further aspects of the invention relate to methods, kits, and systems for high-efficiency nucleic acid library preparation. The nucleic acid library can be used for sequencing by a sequencing platform. The sequencing platform can be a next-generation sequencing (NGS) platform. In some embodiments, the method further comprises sequencing the nucleic acid library using NGS technology. Exemplary NGS technologies and sequencing platforms are described herein.

In one aspect, the invention provides methods of preparing a nucleic acid library from a plurality of template nucleic acids isolated from a biological source. The plurality of template nucleic acids can comprise genomic material. The genomic material can comprise genomic DNA (gDNA), RNA, or cDNA reverse-transcribed from RNA. The nucleic acid library can be a DNA library, an RNA library, a single-stranded DNA library, or a double-stranded DNA library. In some embodiments, the method comprises ligation of adaptor sequences to template nucleic acids. In some embodiments, the method improves efficiency of adaptor ligation by over 10-fold, 50-fold, 100-fold, 500-fold, 1000-fold, or more than 1000-fold. The methods described herein can, for example, increase adaptor ligation efficiency to over 10%, 20%, 30%, 40%, 50%, 60%, 70%, 80%, 90%, 95%, 97%, 98%, 99%, 99.5%, or 99.9% efficiency. In some embodiments, the methods results in correct ligation of adaptors to over 80%, over 85%, over 90%, over 95%, over 97%, over 98%, over 99%, over 99.5%, over 99.9%, or substantially all of the plurality of template nucleic acids. Such highly efficient ligation methods as described herein can enable the preparation of nucleic acid libraries that accurately represent substantially all of the desired nucleic acids (e.g., gDNA, RNA, or cDNA) isolated from the biological source. Furthermore, the methods described herein can obviate the necessity of library pre-amplification, and avoid the introduction of pre-amplification bias and sequencing errors resulting from pre-amplification. Such methods can pave the way for digital sequencing capabilities, e.g., the capability to provide a digital readout of sequence reads for each individual template nucleic acid isolated from a biological source, and can improve the sensitivity for detection of rare mutations (e.g., rare single nucleotide polymorphisms (SNPs) or rare copy number variants). Accordingly, in some aspects the invention provides a method of sequencing a plurality of nucleic acids isolated from a biological source, comprising ligating sequencing adaptors to at least 10%, 20%, 30%, 40%, 50%, 60%, 70%, 80%, 90%, 95%, 99%, or substantially all of the plurality of nucleic acids, thereby creating a nucleic acid library, and sequencing the nucleic acid library without pre-amplification of the library.

In some embodiments, the method comprises ligating an adaptor sequence to a first end of at least 10%, 20%, 30%, 40%, 50%, 60%, 70%, 80%, or 90% of a plurality of template nucleic acids, thereby creating a nucleic acid library. An adaptor sequence can comprise a defined oligonucleotide sequence that effects coupling of a library member to a sequencing platform. By way of example only, the adaptor can comprise a sequence that is at least 70% complementary or identical to an oligonucleotide sequence immobilized onto a solid support (e.g., a sequencing flow cell or bead). An adaptor sequence can comprise a defined oligonucleotide sequence that is at least 70% complementary or identical to a sequencing primer. The sequencing primer can enable nucleotide incorporation by a polymerase, wherein incorporation of the nucleotide is monitored to provide sequencing information. In some embodiments, an adaptor comprises a sequence that is at least 70% complementary or identical to an oligonucleotide sequence immobilized onto a solid support and a sequence that is at least 70% complementary or identical to a sequencing primer. In some embodiments, the adaptor can comprise a barcode sequence. In some embodiments, at least 10%, 20%, 30%, 40%, 50%, 60%, 70%, 80%, 90%, or 100% of sequencing library members in a library comprise the same adaptor sequence. In some embodiments, at least 10%, 20%, 30%, 40%, 50%, 60%, 70%, 80%, 90%, or 100% of sequencing library members comprise an adaptor sequence at a first end but not at a second end. In some embodiments, the first end is a 5′ end. In some embodiments, the first end is at 3′ end. In some embodiments, at least 10%, 20%, 30%, 40%, 50%, 60%, 70%, 80%, 90%, or 100% of sequencing library members comprise an adaptor sequence at a first and at a second end. The adaptor sequence at the first end may be distinct from the adaptor sequence at the second end. The adaptor sequence can be chosen by a user according to the sequencing platform used for sequencing. In some embodiments, the method of ligating an adaptor to a first end of a nucleic acid comprises a high efficiency ligation method as described herein.

In some embodiments, following ligation of a first adaptor at a first end of a template nucleic acid, ligation of a second adaptor at a second end of the template nucleic acid is performed using any of the methods as described herein. By way of example only, an Illumina sequencing by synthesis platform comprises a solid support with a first and second population of surface-bound oligonucleotides immobilized thereon. Such oligonucleotides comprise a sequence for hybridizing to a first and second Illumina-specific adaptor oligonucleotide and priming an extension reaction. Accordingly, in some embodiments the library member comprises a first Illumina-specific adaptor that is partially or wholly complementary to a first population of surface bound oligonucleotides of an Illumina system. The library member may further comprise a second Illumina-specific adaptor that is partially or wholly complementary to a second population of surface bound oligonucleotides of an Illumina system. By way of other example only, the SOLiD system, and Ion Torrent, GS FLEX system comprises a solid support in the form of a bead with surface bound oligonucleotides immobilized thereon. Accordingly, in some embodiments the nucleic acid library member comprises an adaptor sequence that is complementary to a surface-bound oligonucleotide of a SOLiD system, Ion Torrent system, or GS Flex system.

The plurality of template nucleic acids can comprise a template nucleic acid that is over 120 nt long. The plurality of template nucleic acids can have an average length of >120 nt. The plurality of template nucleic acids can have an average length of 50-100, 75-125, 120-150, 130-170, 150-250, 200-500, 300-700, 500-1000, 800-2000, 1500-5000, 4000-10000, or over 10000 nt. The plurality of template nucleic acids can comprise genomic DNA. The plurality of template nucleic acids can comprise single-stranded (ss) nucleic acid fragments, such as, e.g., ssDNA. In some embodiments, the method can result in ligation of an adaptor sequence to a first end of at least 95%, 96%, 97%, 98%, 99%, 99.5%, or greater than 99.5% of the plurality of template nucleic acids.

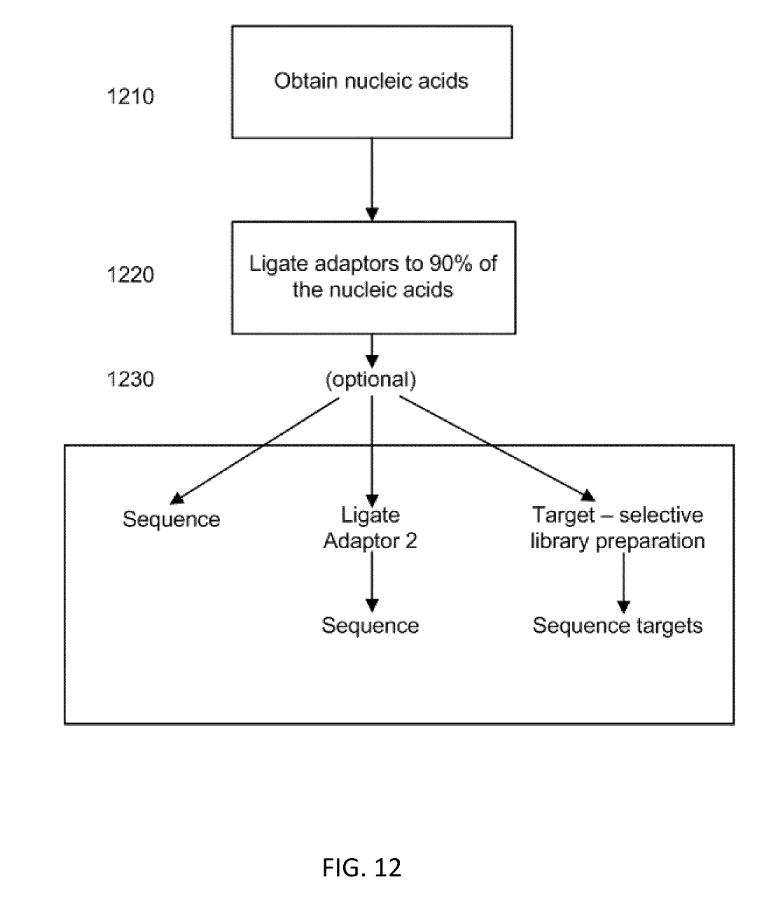

FIG. 12 depicts an exemplary workflow for preparing a nucleic acid library. In a first step 1210, nucleic acids are obtained from a biological source. The biological source can be a subject. Exemplary biological sources and subjects are described herein. In a second step 1220, adaptors are ligated to 90% of the obtained nucleic acids using any of the methods described herein. In a third step 1230 (optional), the library may be sequenced, or may be adaptor-ligated to a second adaptor using any of the methods as described herein, or undergo target-selective library preparation. Target-selective library preparation may be by any means known in the art. Exemplary target-selective library preparation methods are described in, e.g., U.S. Pat. Nos. 6,063,604; 6,090,591; 8,349,563; US Patent Application Pub. Nos. 2009010508, 20110244455 2012003657, 20120157322, 20130045872, and PCT Publication No. WO2012103154, all of which are hereby incorporated by reference. In some embodiments, the library is subjected to a method for preparing a target-enriched nucleic acid library as described herein.

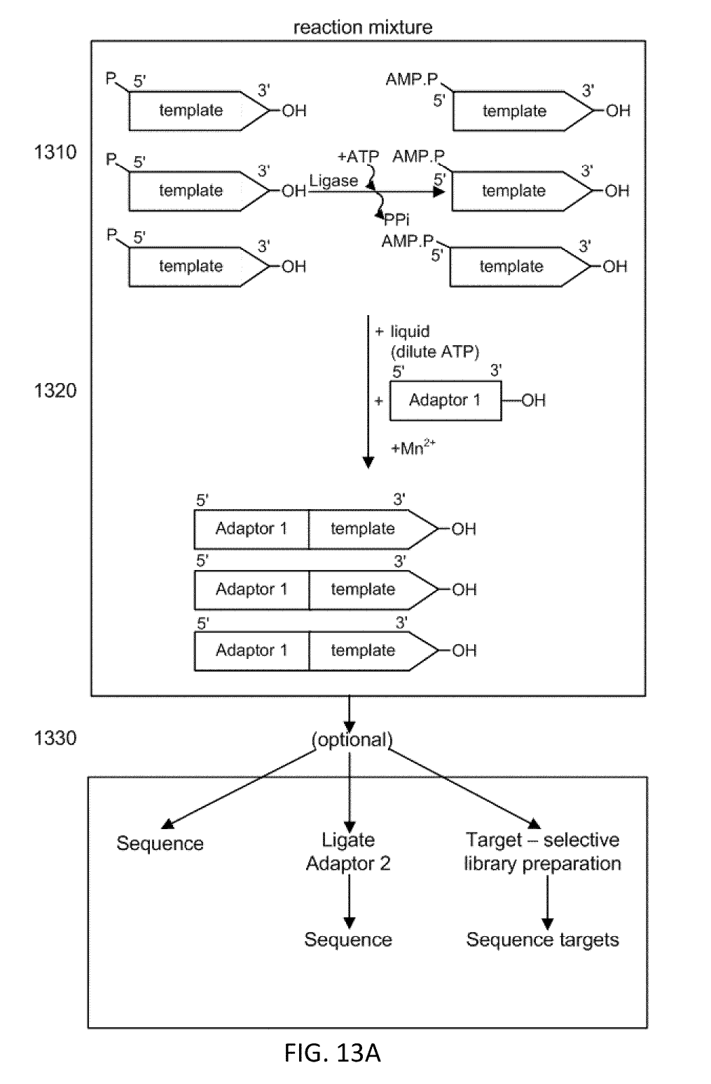

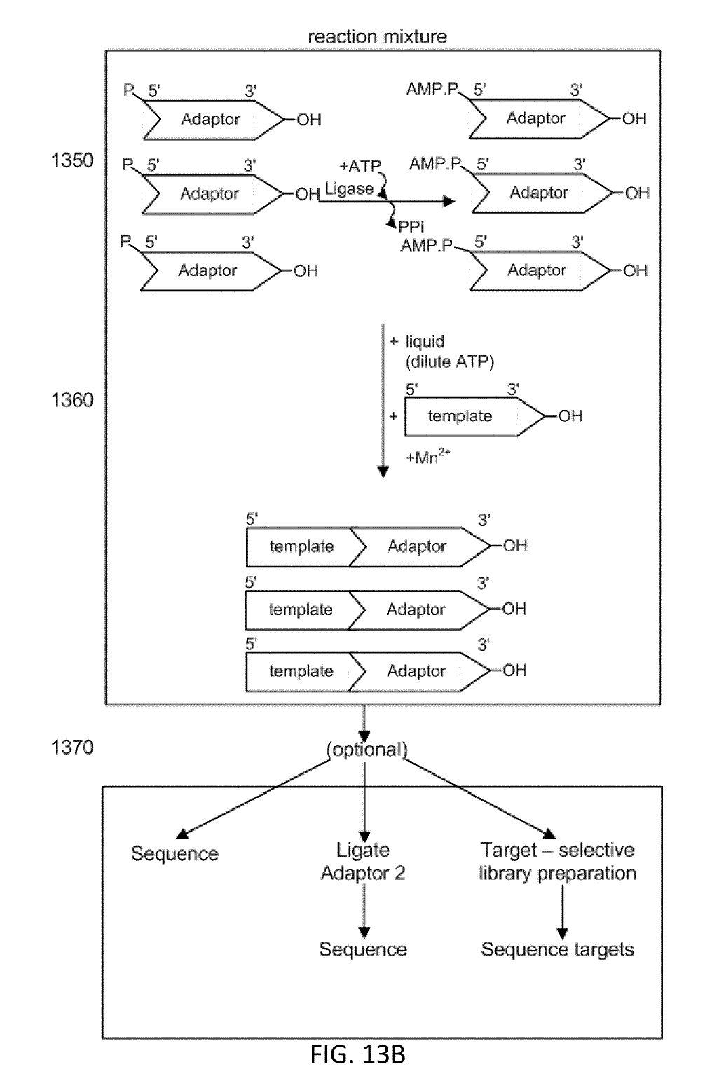

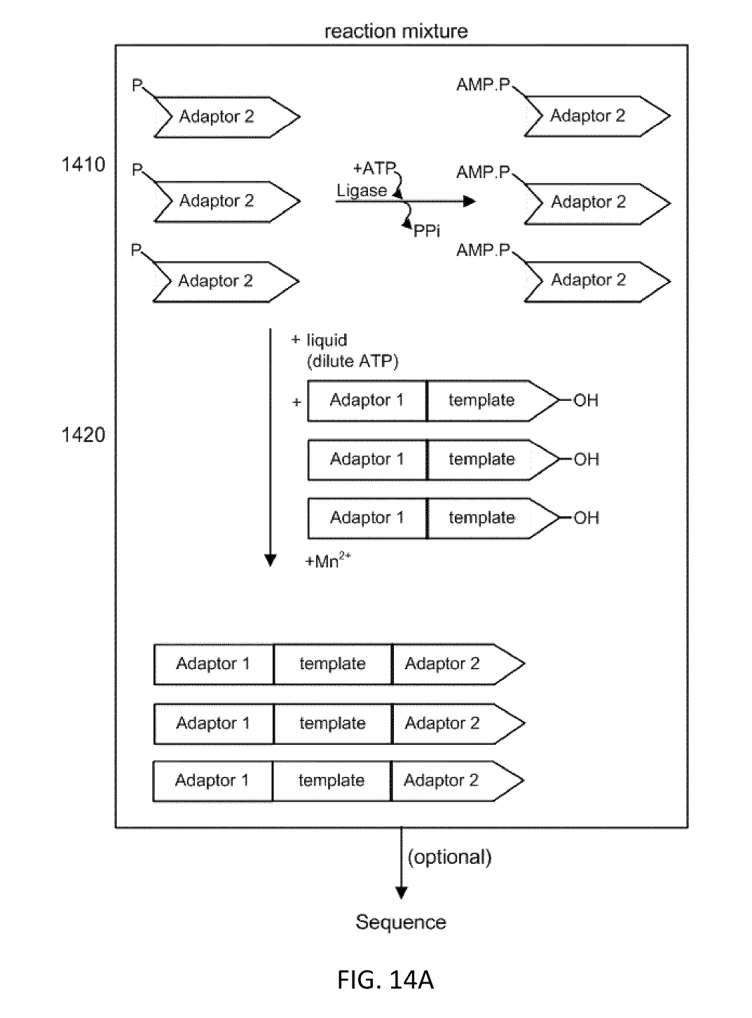

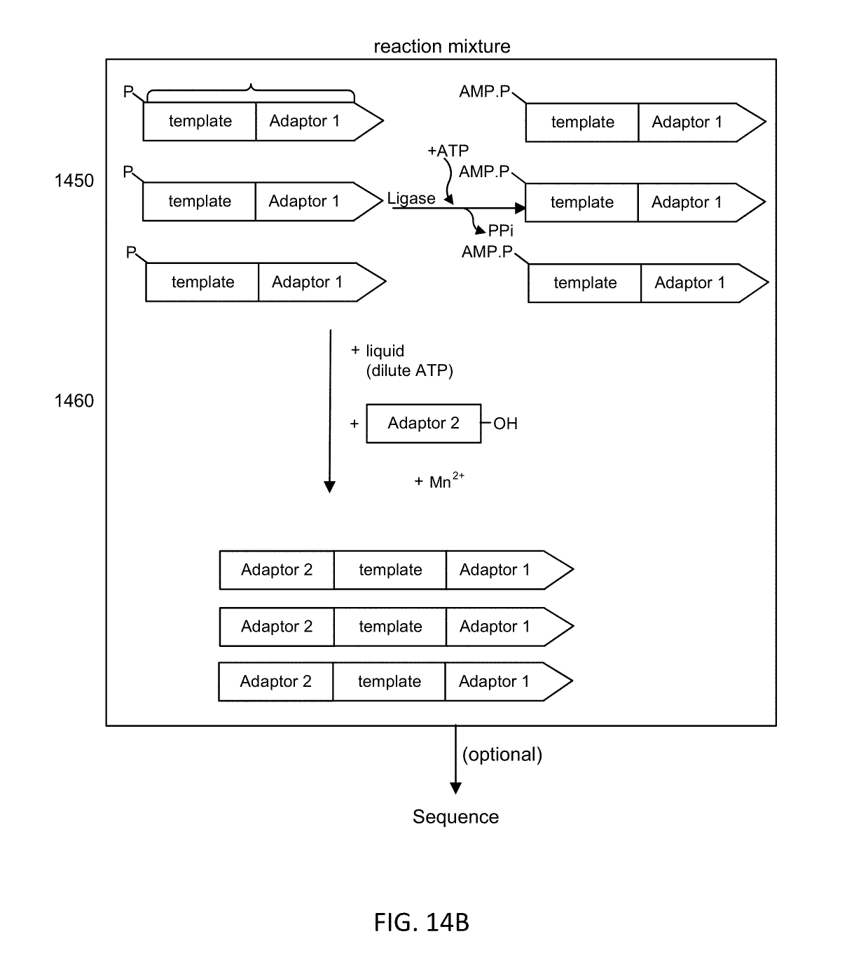

FIG. 13A depicts an exemplary embodiment of a method for preparing a nucleic acid library, comprising ligating a first adaptor to a 5′ end of nucleic acid fragments. In a first step 1310 a plurality of template nucleic acid fragments (e.g., DNA fragments) comprising a 5′ phosphate is incubated in a reaction mixture containing an excess amount of ligase and excess ATP. The template DNA fragments may be fully or partially denatured. The ligase catalyzes transfer of AMP to the 5′ phosphate of the template nucleic acid fragments (e.g., adenylates the template DNA fragments), releasing PPi in the process. The reaction is incubated under conditions sufficient to result in adenylation of at least 10%, 20%, 30%, 40%, 50%, 60%, 70%, 80%, or 90% of the template nucleic acid fragments. In a next step 1320, liquid is added to the reaction mixture in an amount sufficient to dilute ATP at least 10-fold. The liquid may comprise components such as, e.g., water, monovalent salts, Mg²⁺, PEG. Also added to the reaction mixture are the adaptor oligonucleotides to be ligated to the donor molecules (e.g., Adaptor 1) and Mn²⁺. The adaptor oligonucleotides may or may not comprise a detectable tag. The detectable tag may be used for detecting and/or affinity binding. The adaptor oligonucleotides may comprise 3′ OH groups. Both the dilution of ATP and addition of Mn²⁺ may drive the ligation reaction to completion, resulting in ligation products comprising, in the 5′→3′ direction, Adaptor1-template nucleic acid. The ligation products may then be collected and optionally further processed in step 1330 by sequencing, by ligation of a second adaptor sequence to a 3′ end (as described in, e.g., FIG. 14A), followed by sequencing, or by target-selective library preparation as described herein. In some embodiments, the library is subjected to a method for preparing a target-enriched nucleic acid library as described herein.

FIG. 13B depicts another exemplary embodiment of a method for preparing a nucleic acid library, comprising ligating a first adaptor to a 3′ end of nucleic acid fragments. In a first step 1350 a plurality of oligonucleotide adaptors (e.g., Adaptor) comprising a 5′ phosphate is incubated in a reaction mixture containing an excess amount of ligase and excess ATP. The Adaptor oligonucleotides may be fully or partially denatured. The Adaptor oligonucleotides may or may not comprise a detectable tag. The detectable tag may be used for detecting and/or affinity binding. The ligase catalyzes transfer of AMP to the 5′ phosphate of the Adaptor 1 oligonucleotides (e.g., adenylates Adaptor 1), releasing PPi in the process. The reaction is incubated under conditions sufficient to result in adenylation of at least 90% of Adaptor. In a next step 1360, liquid is added to the reaction mixture in an amount sufficient to dilute ATP at least 10-fold. The liquid may comprise components such as, e.g., water, monovalent salts, Mg²⁺, PEG. Also added to the reaction mixture are the sample of template nucleic acids (e.g., template) and Mn²⁺. The template nucleic acids may comprise 3′ OH groups. Both the dilution of ATP and addition of Mn²⁺ drive the ligation reaction to completion, resulting in ligation products comprising, in the 5′→3′ direction, template DNA-Adaptor. The ligation products may then be collected and optionally further processed by sequencing, by ligation of a second adaptor sequence to a 3′ end followed by sequencing, or by target-selective library preparation as described herein. Both the dilution of ATP and addition of Mn²⁺ may drive the ligation reaction to completion, resulting in ligation products comprising, in the 5′→3′ direction, Template nucleic acid-Adaptor. The ligation products may then be collected and optionally further processed in step 1370 by sequencing, by ligation of a second adaptor sequence to a 5′ end as described in FIG. 14B, followed by sequencing, or by target-selective library preparation as described herein. In some embodiments, the library is subjected to a method for preparing a target-enriched nucleic acid library as described herein.

FIG. 14A depicts an exemplary embodiment of a method for ligating a second adaptor sequence to Adaptor1-template nucleic acid molecules prepared as described in FIG. 13A. In a first step 1410, a plurality of oligonucleotides comprising a second adaptor sequence (“Adaptor 2”) comprising a 5′ phosphate is incubated in a reaction mixture containing an excess amount of ligase and excess ATP. The oligonucleotides may be fully or partially denatured. The ligase catalyzes transfer of AMP to the 5′ phosphate of the oligonucleotides (e.g., adenylates the Adaptor 2 oligonucleotides), releasing PPi in the process. The reaction is incubated under conditions sufficient to result in adenylation of at least 10%, 20%, 30%, 40%, 50%, 60%, 70%, 80%, or 90% of the Adaptor 2 oligonucleotides. In a next step 1420, liquid is added to the reaction mixture in an amount sufficient to dilute ATP at least 10-fold. The liquid may comprise components such as, e.g., water, monovalent salts, Mg²⁺, PEG. Also added to the reaction mixture are the Adaptor1-template nucleic acid molecules (e.g., as described in FIG. 4A) and Mn²⁺. The Adaptor1-template nucleic acid molecules may comprise 3′ OH groups. Both the dilution of ATP and addition of Mn²⁺ drive the ligation reaction to completion, resulting in ligation products comprising Adaptor1-template nucleic acid-Adaptor 2 library members. The ligation products may optionally be sequenced.

FIG. 14B depicts an exemplary embodiment of a method for ligating a second adaptor sequence to template nucleic acid-Adaptor 1 molecules prepared as described in FIG. 13B. In a first step 1450, the template-Adaptor 1 molecules comprising a 5′ phosphate is incubated in a reaction mixture containing an excess amount of ligase and excess ATP. The template-Adaptor 1 molecules may be fully or partially denatured. The ligase catalyzes transfer of AMP to the 5′ phosphate of the template-Adaptor 1 molecules (e.g., adenylates the template-Adaptor 1 molecules), releasing PPi in the process. The reaction is incubated under conditions sufficient to result in adenylation of at least 10%, 20%, 30%, 40%, 50%, 60%, 70%, 80%, or 90% of the template-Adaptor 1 molecules. In a next step 1460, liquid is added to the reaction mixture in an amount sufficient to dilute ATP at least 10-fold. The liquid may comprise components such as, e.g., water, monovalent salts, Mg²⁺, PEG. Also added to the reaction mixture are Adaptor 2 oligonucleotides comprising a second adaptor sequence and Mn²⁺. The Adaptor 2 oligonucleotides may comprise 3′ OH groups. Both the dilution of ATP and addition of Mn²⁺ drive the ligation reaction to completion, resulting in ligation products comprising Adaptor2-template-Adaptor 1 library members. The library members may also be constructed as Adaptor1-template-Adaptor 2 using the methods as described herein. The ligation products may optionally be sequenced.

### Target-Enriched Library Preparation

In another aspect, the invention provides a method for preparing a target-enriched DNA library. The method can involve hybridizing a target-selective oligonucleotide to a sequencing library member to create a hybridization product. The method can further comprise amplifying the hybridization product in a single round of amplification to create an extension strand.

The method of target enrichment can be as described in US. Patent Application Pub. No. 20120157322, hereby incorporated by reference.

The hybridizing and amplifying can occur in a reaction mixture. The mixture may comprise nucleotides (dNTPs), a polymerase and a target-selective oligonucleotide. In some embodiments, the mixture comprises a plurality of target-selective oligonucleotides. The mixture can comprise, for example, 1-10, 5-20, 10-50, 40-100, 80-200, 150-500, 300-1000, 800-2000, 1000-5000, 4000-10000, 8000-20000, or more than 20000 target-selective oligonucleotides. The mixture may further comprise a Tris buffer, a monovalent salt, and Mg²⁺. The concentration of each component can be optimized by an ordinary skilled artisan. The reaction mixture can also comprise additives including, but not limited to, non-specific background/blocking nucleic acids (e.g., salmon sperm DNA), biopreservatives (e.g. sodium azide), PCR enhancers (e.g. Betaine, Trehalose, etc.), and inhibitors (e.g. RNAse inhibitors). In some embodiments, a nucleic acid sample (e.g., a sample comprising a library member) is admixed with the reaction mixture.

The library member can be fully or partially denatured. The library member can comprise a first single-stranded adaptor sequence located at a first end but not at a second end. In some embodiments, the first end is a 5′ end. In some embodiments, the library member comprising a first adaptor sequence at a 5′ end is prepared as described in FIG. 13A. In other embodiments, the library member comprising a first adaptor sequence is prepared as described by ligating a reverse complement adaptor sequence to a 3′ end of a nucleic acid (e.g., a gDNA fragment) as described in FIG. 13B, followed by linear amplification of the resulting ligation product using a primer comprising a full adaptor sequence and hybridizable to the reverse complement. In some embodiments, the target-selective oligonucleotide comprises a second single-stranded adaptor sequence located at a first end but not a second end. The first end of the target-selective oligonucleotide can be a 5′ end. In some embodiments, the first adaptor sequence comprises a sequence that is at least 70% identical to a first surface-bound oligonucleotide. In some embodiments, the first adaptor sequence comprises a sequence that is at least 70% identical to a sequencing primer. In some embodiments the first adaptor further comprises a barcode sequence. In some embodiments, the second adaptor comprises a sequence that is at least 70% identical to a second surface-bound oligonucleotide. In some embodiments, the second adaptor comprises a sequence that is at least 70% identical to a sequencing primer.

The target-selective oligonucleotide can be designed to at least partially hybridize to a target polynucleotide of interest. In some embodiments, the target-selective oligonucleotide is designed to selectively hybridize to the target polynucleotide. The target-selective oligonucleotide can be at least about 70%, 75%, 80%, 85%, 90%, 95%, or more than 95% complementary to a sequence in the target polynucleotide. In some embodiments, the target-selective oligonucleotide is 100% complementary to a sequence in the target polynucleotide. The hybridization can result in a target-selective oligonucleotide/target duplex with a Tm. The Tm of the target-selective oligonucleotide/target duplex can be between 0-100 deg C, between 20-90 deg C, between 40-80 deg C, between 50-70 deg C, or between 55-65 deg C. The target-selective oligonucleotide can be sufficiently long to prime the synthesis of extension products in the presence of a polymerase. The exact length and composition of a target-selective oligonucleotide can depend on many factors, including temperature of the annealing reaction, source and composition of the primer, and ratio of primer:probe concentration. The target-selective oligonucleotide can be, for example, 8-50, 10-40, or 12-24 nucleotides in length.

The method can comprise extension of the target in the reaction mixture. The extension can be primed by a target-selective oligonucleotide in a target-selective oligonucleotide/target duplex. In some embodiments extension is carried out utilizing a nucleic acid polymerase. The nucleic acid polymerase can be a DNA polymerase. In particular embodiments, the DNA polymerase is a thermostable DNA polymerase. The polymerase can be a member of B family DNA proofreading polymerases (Vent, Pfu, Phusion, and their variants), a DNA polymerase holoenzyme (DNA pol III holoenzyme), a Taq polymerase, or a combination thereof.

Extension can be carried out as an automated process wherein the reaction mixture comprising template DNA is cycled through a denaturing step, an annealing step, and a synthesis step. The automated process may be carried out using a PCR thermal cycler. Commercially available thermal cycler systems include systems from Bio-Rad Laboratories, Life technologies, Perkin-Elmer, among others. In some embodiments, one cycle of amplification is performed.

Extension of the target-selective oligonucleotide/target duplex can result in a double stranded extension product comprising (1) the original ssDNA fragment comprising the target sequence, and (2) an extended strand comprising the second adaptor sequence, the target-selective oligonucleotide, a reverse complement of the target sequence, and a reverse complement of the first adaptor sequence. If the first adaptor sequence of the original ssDNA fragment was 70% or more identical to a first surface-bound oligonucleotide, then the extended strand would comprise a first adaptor sequence that is 70% or more complementary to the first surface-bound oligonucleotide, and thereby would be hybridizable to the first surface-bound oligonucleotide. The extended strands, can comprise the target-enriched library, wherein each library member comprises a first adaptor at a first end and a second adaptor at a second end.

The target-enriched library can be sequenced. The target-enriched library members in can be denatured. The denatured library members can be contacted with a surface immobilized thereon at least a first surface-bound oligonucleotide. In some embodiments, the extended strand is captured by the first surface-bound oligonucleotide, which can anneal to the first adaptor sequence on the extended strand.

The first surface-bound oligonucleotide can prime the extension of the captured extended strand. In some embodiments, extension of the captured extended strand results in a captured extension product. The captured extension product can comprise the first surface bound oligonucleotide, the target sequence, and a second adaptor sequence that is at least 70% or more complementary to a second surface-bound oligonucleotide.

In some embodiments, the captured extension product hybridizes to the second surface-bound oligonucleotide, forming a bridge. In some embodiments, the bridge is amplified by bridge PCR. Bridge PCR methods can be carried out using methods known to the art. A person skilled in the art will appreciate that the methods described herein can be adapted to any solid-phase amplification method, such as amplification on a bead.

### Array Hybridization Applications

The high efficiency ligation methods and kits described herein may also be used for the preparation of nucleic acid samples for array hybridization (e.g., nucleic acid microarray). Nucleic acid microarray techniques generally refer to techniques that rely on hybridization of nucleic acids to an array of oligonucleotide probes immobilized onto a solid or semi-solid surface. Nucleic acids (e.g., DNA) isolated from a sample are generally prepared by labeling with a detectable label. The labeled nucleic acids can then be applied to an array containing a plurality of oligonucleotides of known sequence (e.g., probes) immobilized onto addressable locations of a solid surface. The oligonucleotide probes may be hybridizable to a plurality of target regions of interest. In some embodiments, the oligonucleotide probes may be hybridizable to one or more adaptor sequences. The amount of detectable signal at a certain addressable location can indicate the amount of nucleic acids containing the target region in the sample. Exemplary microarray systems include, e.g., bead array systems (Illumina, Inc, Lynx Therapeutics, Luminex, Inc., Exiqon, Mycroarray) SNP arrays (available from, e.g., Agilent Technologies, Illumina, Inc., Affymetrix, Inc., Life Technologies, Inc., Nimblegen, Exiqon, Mycroarray), and comparative genome hybridization arrays (available from, e.g., Agilent Technologies, Illumina, Inc., Affymetrix, Inc., Life Technologies, Inc., Exiqon, Mycroarray). Bead array systems (available from, e.g., Illumina, Lynx Therapeutics, Luminex, Inc.,) generally refer to array systems comprising microsphere beads impregnated with multiple copies of oligonucleotide probes. Beads may be addressable either by deposition into microwells or by barcoding with unique combinations of fluorophores, which may be sorted and identified by any means known in the art, including, e.g., flow cytometry. Exemplary bead array systems and methods are described in U.S. Pat. Nos. 8,399,192 and 8,198,028, which are hereby incorporated by reference. SNP arrays generally refer to arrays and systems that are configured to detect SNP alleles. Exemplary SNP arrays are described in, e.g., U.S. Pat. Nos. 6,410,231; 6,858,394; US Patent Application Pub. Nos. 20090062138, and EP Patent Application No. EP1207209, all of which are hereby incorporated by reference. Comparative genome hybridization (CGH) generally refers to arrays and systems that enable high-resolution, genome-wide screening of segmental genomic copy number variations (CNVs). CGH platforms can detect aneuploidies, microdeletion/microduplication syndromes, and chromosomal rearrangements. Exemplary CGH arrays and array methods are described in, e.g., U.S. Pat. No. 6,410,243; hereby incorporated by reference.

Library preparation of nucleic acid samples (e.g., gDNA samples) for array hybridization generally involves labeling individual nucleic acid fragments with a detectable label. The labeling method traditionally involves hybridization of random primers to the nucleic acid fragments, followed by extension of the random primers by a polymerase. The extension reaction incorporates labeled nucleotides into the extension product. This method of labeling by extension by a polymerase can introduce labeling bias into the resulting library.

The high-efficiency ligation methods described herein can overcome the limitations of traditional library preparation methods for array hybridization by obviating the need for random primer hybridization and extension. Accordingly, in some aspects the invention provides methods and kits for preparing a nucleic acid library for array hybridization. In some embodiments, the method comprises ligating a labeled oligonucleat least 10%, 20%, 30%, 40%, 50%, 60%, 70%, 80%, or 90% otide to at least 10%, 20%, 30%, 40%, 50%, 60%, 70%, 80%, or 90% of nucleic acids present in a sample, utilizing any of the methods as described herein (see, e.g., FIG. 10A-10B). The labeled oligonucleotide may comprise a detectable label or capture moiety. Exemplary detectable labels and capture moieties are described herein.

### Barcoding Applications

Molecular barcoding is useful for the tracking, identification, and/or retrieval of individual nucleic acid molecules, subclasses of nucleic acid molecules, or samples of nucleic acid. Molecular barcoding generally involves tagging nucleic acid molecules with oligonucleotide sequences. The oligonucleotide sequences can be unique from sample to sample, from subclass to subclass, or from individual nucleic acid to individual nucleic acid, as desired by a user. Exemplary barcodes are described herein.

In one aspect, the high efficiency ligation method can be used to barcode a plurality of nucleic acid molecules. In some embodiments, the method comprises ligating a barcode sequence to a nucleic acid molecule using any of the methods as described herein. The methods described herein can ensure that over 80%, over 85%, over 90%, over 95%, over 97%, over 98%, over 99%, over 99.5%, over 99.9%, or substantially all of nucleic acids in a sample to be barcoded is ligated to a barcode sequence. In some embodiments, each of a plurality of nucleic acid samples are barcoded by ligation to a single barcode sequence unique to the sample. Such barcoding allows for sample origin to be identified in an assay. In other embodiments, a plurality of nucleic acids are barcoded such that each individual nucleic acid in a sample is ligated to a unique barcode sequence. Such barcoding allows for the tracking and identification of individual nucleic acids in a sample. In either method, nucleic acids in a sample can be adenylated in a reaction mixture as described herein, followed by ligation as described herein to a barcode sequence.

### Cloning Applications

Molecular cloning often involves ligation of an insert DNA sequence into a vector, e.g., a plasmid vector. Generally, insert DNA and vector are prepared by restriction digest, wherein restriction enzymes can recognize a palindromic sequence within the insert DNA or vector and digest it, producing compatible sticky ends. The digested insert and vector are then incubated together in a ligation reaction, with the goal of annealing the compatible sticky ends of the vector to insert, producing a desired product comprising the vector and insert. However, due to the palindromic sticky ends, spurious ligation products are also created during the ligation process, including, e.g., insert-insert ligations and vector/vector ligations. This reduces the efficiency and specificity of the ligation reaction. As a result, a user must often expend significant amounts of time and effort to select a large number of transformed bacterial colonies and then to screen them, for example, by restriction fragment length polymorphism (RFLP), to select for the desired ligation product.

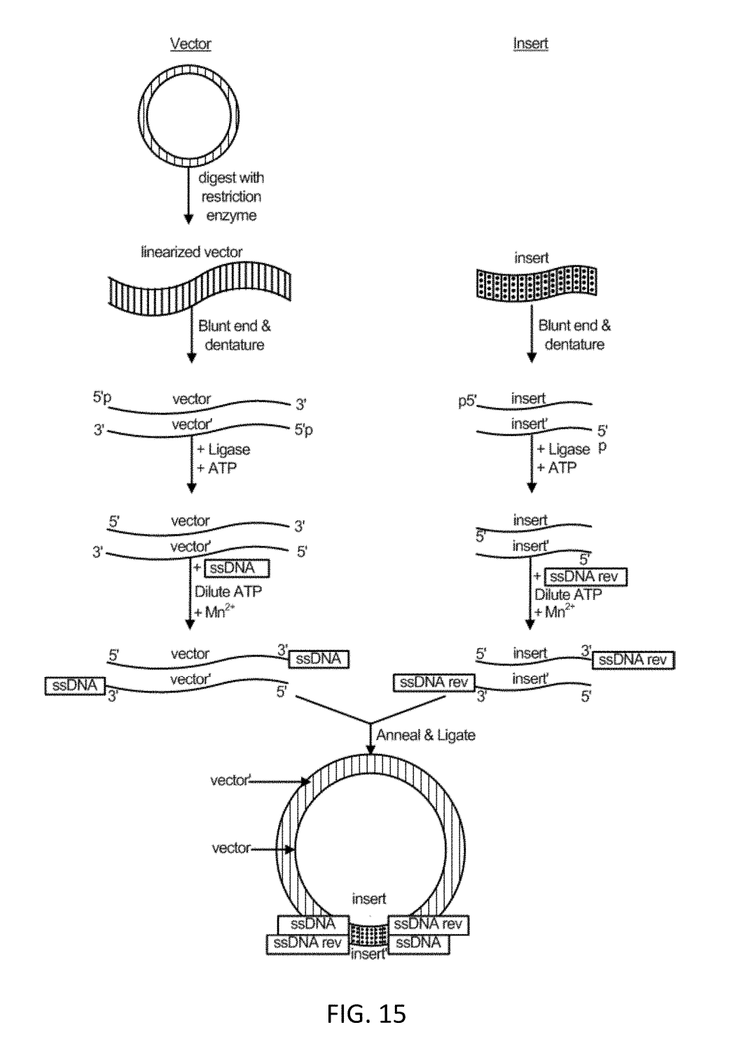

The high-efficiency ligation methods described herein can be used to improve the specificity of cloning reactions. An exemplary embodiment is depicted in FIG. 15. A vector can be linearized by any means, such as by restriction digest at a single site. The ends of the linearized vector can be blunt-ended, for example, by a DNA polymerase (e.g., T4 DNA polymerase). The 5′ terminus of a linearized vector can be phosphorylated, e.g., by T4 polynucleotide kinase. The linearized vector can be fully or partially denatured, producing at least single-stranded (e.g., frayed) ends or single-stranded linear DNA. High-efficiency ligation using any of the methods as described herein can be performed to ligate a non-palindromic short ssDNA sequence (“ssDNA”) onto the 3′ ends of the fully or partially denatured vector. An insert DNA fragment can also be blunt-ended and 5′ phosphorylated as described above. The insert DNA fragment can be fully or partially denatured. High-efficiency ligation using any of the methods as described herein is performed to insert a non-palindromic short ssDNA sequence (“ssDNArev”) onto the 3′ ends of the fully or partially denatured insert. The modified vector and insert can then be ligated using standard ligation protocols. Because ssDNA and ssDNArev are non-palindromic sequences, formation of spurious vector/vector or insert/insert products do not occur, and any ligation will be between a single vector and a single insert. Alternatively, non-palindromic short ssDNA sequences can be ligated onto 5′ ends of the vector or insert. Such specificity can obviate the need for screening colonies by RFLP techniques, and greatly enhance workflow for molecular cloning.

### Diagnostic/Therapeutic Applications

The high efficiency ligation methods and kits as described herein have general utility in a number of diagnostic/therapeutic applications. For instance, the high efficiency ligation methods of the invention are of general utility for sequence analysis of nucleic acids, which is playing an increasingly important role in the diagnosis, monitoring, and treatment of diseases. For example, the invention methods may be utilized in, e.g., the identification of subjects that have increased likelihood of developing a disease, for diagnosing a disease, for improving accuracy of disease diagnosis, for monitoring the progression of a disease, for aiding selection of a therapeutic regimen for a disease in a subject, for evaluating disease prognosis in a subject.

Is it understood that there is no limit to the diagnostic/therapeutic applications or disease types that may benefit from the invention methods. By way of example only, the application of the invention methods to a workflow for monitoring cancer is described herein.

Accordingly, the invention provides methods and kits that improve the monitoring and treatment of a subject suffering from a disease. The disease can be a cancer, e.g., a tumor, a leukemia such as acute leukemia, acute t-cell leukemia, acute lymphocytic leukemia, acute myelocytic leukemia, myeloblastic leukemia, promyelocytic leukemia, myelomonocytic leukemia, monocytic leukemia, erythroleukemia, chronic leukemia, chronic myelocytic (granulocytic) leukemia, or chronic lymphocytic leukemia, polycythemia vera, lymphomas such as Hodgkin's lymphoma, follicular lymphoma or non-Hodgkin's lymphoma, multiple myeloma, Waldenström's macroglobulinemia, heavy chain disease, solid tumors, sarcomas, carcinomas such as, e.g., fibrosarcoma, myxosarcoma, liposarcoma, chondrosarcoma, osteogenic sarcoma, lymphangiosarcoma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic, carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, craniopharyngioma, ependymoma, pinealoma, hemangioblastoma, acoustic neuroma, oligodendroglioma, meningioma, melanoma, neuroblastoma, retinoblastoma, endometrial cancer, or non small cell lung cancer.

The subject can be suspected or known to harbor a solid tumor, or can be a subject who previously harbored a solid tumor.

The method can comprise sequencing a set of cancer-related genes from a tumor sample isolated from the subject and, optionally, sequencing a set of cancer-related genes from normal cells isolated from the subject. The tumor sample can be a solid tumor sample. The normal cells can be, e.g., blood cells isolated from a blood sample from the subject.

Generally, a library of nucleic acids isolated from the subject is sequenced. Standard sequencing protocols often comprise pre-amplification of the nucleic acid library to achieve a desired read depth. However, pre-amplification can introduce amplification bias due to variable amplification efficiency of individual nucleic acid library members, which can result in over-representation of some genomic regions and under-representation of other genomic regions (e.g., regions with high or low GC content. Pre-amplification can also introduce sequencing errors due to intrinsic error rates of polymerases used for PCR. Accordingly, the invention provides, in some aspects, methods of sequencing a library of nucleic acids isolated from a biological source without pre-amplification of the library. In some embodiments the library is not pre-amplified prior to loading onto a sequencer.

Upon sequencing, sequence data from the tumor can be compared to sequence data from normal cells to generate a tumor-specific sequence profile. In some embodiments, the tumor-specific sequence profile comprises mutational status of one or more genes in the set. The mutational status may include SNP or CNV identification. The method can further comprise generating a report describing the tumor-specific sequence profile. In some embodiments, the method further comprises choosing a subset of 2-4 genes known to harbor tumor-specific mutations for further monitoring. In other embodiments, the method comprises choosing a subset of 4-15, 10-30, 20-50, 40-80, 70-125, 100-200, or more than 200 genes known to harbor tumor-specific mutations for further monitoring. In some embodiments, the method comprises selecting the entirety of the set of cancer-related genes for further monitoring. In other embodiments, the method comprises use of whole genome sequencing for the purposes of further monitoring.

### Sensitive Detection of Amplicons

The present invention provides reagents, methods and kits for the sensitive, accurate detection and/or quantification of a mutation in a target polynucleotide. For example, the present invention provides reagents, methods, and kits for probe-based PCR assays that substantially obviate the influence of a probe on efficiency of a PCR reaction. The present invention provides reagents, methods, and kits for probe-based PCR assays that substantially obviate the influence of a probe on kinetics of a PCR reaction. Such reagents, methods, and kits can improve the accuracy and sensitivity of detection as compared to conventional probe-based assays, and thus can have wide applicability in the life sciences, in genotyping approaches, and in diagnostic/therapeutic approaches.

Aspects of the invention relate to probe-based PCR assays in which a probe does not impact primer annealing or primer extension during PCR. Without wishing to be bound by theory, hybridization of a probe to a template nucleic acid during PCR can alter the kinetics of primer extension, and therefore can alter efficiency of the PCR reaction. Furthermore, binding of a probe to a template nucleic acid downstream of an annealed primer can impact extension of the primer by a polymerase, as sufficient endonuclease activity may be required to displace the annealed probe. Accordingly, described herein are probes designed to obviate probe hybridization during a PCR annealing and/or extension phase. Such probes can increase the efficiency of PCR amplification. Such probes can minimize extension bias related to probe binding during a PCR annealing and/or extension phase.

A probe for sensitive detection of amplicons as described herein can provide highly accurate and sensitive detection of a mutation. The mutation can be a single nucleotide polymorphisms (SNP), insertion, deletion, translocation, and/or copy number variation. Probes of the invention can detect a rare mutation in a heterogeneous sample. A probe for sensitive detection of amplicons can detect a rare mutation in a sample having a frequency of less than 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.01%, 0.005%, 0.001%, 0.0005%, 0.0001%, 0.00005%, 0.00001%, 0.000005%, 0.000001%, 0.0000005%, 0.0000001% of the sample. For example, a probe for sensitive detection of amplicons can detect a rare SNP in a sample having a frequency of less than 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.01%, 0.005%, 0.001%, 0.0005%, 0.0001%, 0.00005%, 0.00001%, 0.000005%, 0.000001%, 0.0000005%, 0.0000001% of the sample. For example, a probe for sensitive detection of amplicons can detect a rare insertion mutation in a sample having a frequency of less than 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.01%, 0.005%, 0.001%, 0.0005%, 0.0001%, 0.00005%, 0.00001%, 0.000005%, 0.000001%, 0.0000005%, 0.0000001% of the sample. For example, a probe for sensitive detection of amplicons can detect a rare deletion mutation in a sample having a frequency of less than 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.01%, 0.005%, 0.001%, 0.0005%, 0.0001%, 0.00005%, 0.00001%, 0.000005%, 0.000001%, 0.0000005%, 0.0000001% of the sample. For example, a probe for sensitive detection of amplicons can detect a rare inversion mutation in a sample having a frequency of less than 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.01%, 0.005%, 0.001%, 0.0005%, 0.0001%, 0.00005%, 0.00001%, 0.000005%, 0.000001%, 0.0000005%, 0.0000001% of the sample. For example, a probe for sensitive detection of amplicons can detect a rare copy number variation of a gene in a sample, the rare copy number variation comprising a fold change in copy number of as low as 1.01-fold.

Also provided herein are methods for the detection of a rare mutation in a sample having a frequency of less than 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.01%, 0.005%, 0.001%, 0.0005%, 0.0001%, 0.00005%, 0.00001%, 0.000005%, 0.000001%, 0.0000005%, 0.0000001% of the sample. For example, a method of the invention can detect a rare SNP in a sample having a frequency of less than 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.01%, 0.005%, 0.001%, 0.0005%, 0.0001%, 0.00005%, 0.00001%, 0.000005%, 0.000001%, 0.0000005%, 0.0000001% of the sample. For example, a method of the invention can detect a rare insertion mutation in a sample having a frequency of less than 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.01%, 0.005%, 0.001%, 0.0005%, 0.0001%, 0.00005%, 0.00001%, 0.000005%, 0.000001%, 0.0000005%, 0.0000001% of the sample. For example, a method of the invention can detect a rare deletion mutation in a sample having a frequency of less than 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.01%, 0.005%, 0.001%, 0.0005%, 0.0001%, 0.00005%, 0.00001%, 0.000005%, 0.000001%, 0.0000005%, 0.0000001% of the sample. For example, a method of the invention can detect a rare inversion mutation in a sample having a frequency of less than 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.01%, 0.005%, 0.001%, 0.0005%, 0.0001%, 0.00005%, 0.00001%, 0.000005%, 0.000001%, 0.0000005%, 0.0000001% of the sample. For example, a method of the invention can detect a rare copy number variation of a gene in a sample, the rare copy number variation comprising a fold change in copy number of as low as 1.01-fold.

### Probes for Sensitive Detection of Amplicons

The invention provides probes for probe-based hybridization assays. The probe-based hybridization assay can be a probe-based PCR assay, although any probe-based hybridization assay is contemplated. In some embodiments, probes are designed to have minimal to zero impact on kinetics and/or efficiency of a PCR amplification reaction. The impact of a probe on kinetics and/or efficiency of a PCR amplification reaction can relate to an ability of the probe to hybridize or not hybridize to a target polynucleotide during an annealing and/or extension phase of a PCR reaction. The impact of a probe on kinetics and/or efficiency of a PCR amplification reaction can relate to an ability of the probe to hybridize or not hybridize to a target polynucleotide during PCR thermal cycling. For example, a probe for sensitive detection of amplicons can have minimal or zero impact on kinetics and/or efficiency of a PCR amplification reaction by not appreciably hybridizing to a template nucleic acid during an annealing and/or extension phase of the PCR amplification reaction.

The ability of a probe to hybridize or not to a target polynucleotide during an annealing and/or extension phase of a PCR reaction can relate to a melting temperature (Tm) of the probe. A probe for sensitive detection of amplicons can have a melting temperature (Tm) that is not higher than the Tm of PCR primers used in a PCR probe-based assay. A probe for sensitive detection of amplicons can have a melting temperature (Tm) that is not at least 5-10° C. higher than the average Tm of PCR primers for use in a probe-based PCR assay.

Generally, a probe with a Tm that is lower than a PCR annealing temperature would be expected to exhibit reduced probe hybridization during a PCR annealing phase. A probe for sensitive detection of amplicons can have a melting temperature (Tm) that is not higher than a temperature of a PCR annealing phase. A probe for sensitive detection of amplicons can have a melting temperature (Tm) that is lower than a temperature of a PCR annealing phase. A probe with a Tm that is at least 5 degrees lower than a PCR annealing temperature can be expected to exhibit significantly reduced hybridization during a PCR annealing phase. Accordingly, the Tm of a probe for sensitive detection of amplicons can be at least 5° C. less, at least 10° C. less, at least 15° C. less, at least 20° C. less, or more than 20° C. less than a temperature of a PCR annealing phase. A probe for sensitive detection of amplicons can be a low Tm probe. The Tm of a low Tm probe can be at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more than 40° C. less than an annealing temperature of a PCR thermal cycling round. The Tm of a low Tm probe can be about 5-10° C. less, about 10-15° C. less, about 15-20° C. less, about 20-25° C. less, about 25-30° C. less than an annealing temperature of a PCR thermal cycling round. In some cases, a low Tm probe does not hybridize to a complementary template nucleic acid at an ambient temperature above 55° C., above 60° C., above 65° C., or above 70° C.

A low Tm probe can have a Tm that is below 55° C., below 54° C., below 53° C., below 52° C., below 51° C., 50° C., below 49° C., below 48° C., below 47° C., below 46° C., below 44° C., below 43° C., below 42° C., below 41° C., below 40° C., below 39° C., below 38° C., below 37° C., below 36° C., below 35° C., below 34° C., below 33° C., below 32° C., below 31° C., or below 30° C.

A low Tm probe can be designed to hybridize readily to a template nucleic acid at about room temperature. Such a probe design can ensure sufficient hybridization of the probe to its target polynucleotide so as to enable adequate detection of the probe. Generally, a probe can hybridize readily to a template nucleic acid at about room temperature if the Tm of the probe/template duplex is higher than room temperature. Accordingly, a low Tm probe can be designed to have a Tm that is 5° C. higher, 10° C. higher, 15° C. higher, or 20° C. higher, or more than 20° C. higher than room temperature (e.g., a room temperature of 25° C.). Such a Tm can ensure at least 70%, at least 75%, at least 80%, at least 85%, at least 90%, at least 95%, or about 100% of probe hybridization to template nucleic acid at room temperature. In some embodiments, a low Tm probe has a Tm that is above 25° C., above 26° C., above 27° C., above 28° C., above 29° C., above 30° C., above 31° C., above 32° C., above 33° C., above 34° C., above 35° C., above 36° C., above 37° C., above 38° C., above 39° C., above 40° C., above 41° C., above 42° C., above 43° C., above 44° C., or above 45° C.

In some embodiments, a low Tm probe has a Tm that is about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C. The low Tm probe can have a Tm that is 30-35° C., 33-40° C., 36-45° C., or 40-50° C. The low Tm probe can have a Tm that is between 30-45° C.

The probe for sensitive detection of amplicons can comprise a detectable moiety and a quencher moiety. A detectable moiety can be a chemiluminescent, radioactive, metal ion, chemical ligand, fluorescent, or colorimetric moiety, or can be an enzymatic group which, upon incubation with an appropriate substrate, provides a chemiluminescent, fluorescent, radioactive, electrical, or colorimetric signal. In some cases, the detectable moiety is a dye. The dye can be a fluorescent dye, e.g., a fluorophore. The fluorescent dye can be a derivatized dye for attachment to the terminal 3′ carbon or terminal 5′ carbon of the probe via a linking moiety. In some embodiments, the dye is derivatized for attachment to a terminal 5′ carbon of the probe via a linking moiety. The quencher can be a fluorescent dye. Alternatively, the quencher may be a non-fluorescent moiety. Quenching can involve a transfer of energy between the fluorophore and the quencher. The emission spectrum of the fluorophore and the absorption spectrum of the quencher can overlap.

The probe for sensitive detection of amplicons can be designed according to Livak et al., “Oligonucleotides with fluorescent dyes at opposite ends provide a quenched probe system useful for detecting PCR product and nucleic acid hybridization,” PCR Methods Appl. 1995 4: 357-362, which is hereby incorporated by reference.

Reporter-quencher moiety pairs for particular probes can be selected according to, e.g., Pesce et at, editors, Fluorescence Spectroscopy (Marcel Dekker, New York, 1971); White et at, Fluorescence Analysis: A Practical Approach (Marcel Dekker, New York, 1970. Exemplary fluorescent and chromogenic molecules that may be used in reporter-quencher pairs, are described in, e.g. Berlman, Handbook of Fluorescence Sprectra of Aromatic Molecules, 2nd Edition (Academic Press, New York, 1971); Griffiths, Colour and Constitution of Organic Molecules (Academic Press, New York, 1976); Bishop, editor, Indicators (Pergamon Press, Oxford, 1972); Haugland, Handbook of Fluorescent Probes and Research Chemicals (Molecular Probes, Eugene, 1992); Pringsheim, Fluorescence and Phosphorescence (Interscience Publishers, New York, 1949), which are hereby incorporated by reference.

A wide variety of reactive fluorescent reporter dyes can be used so long as they are quenched by a quencher dye of the invention. The fluorophore can be an aromatic or heteroaromatic compound. The fluorophore can be, for example, a pyrene, anthracene, naphthalene, acridine, stilbene, benzoxaazole, indole, benzindole, oxazole, thiazole, benzothiazole, canine, carbocyanine, salicylate, anthranilate, xanthenes dye, or coumarin. Exemplary xanthene dyes include, e.g., fluorescein and rhodamine dyes. Exemplary fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent reporters also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl) aminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno[2,3, 4-ij: 5,6, 7-i′j′]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3, 6,7, 12,13, 16,17-octahydro-inner salt (TR or Texas Red); or BODIPY′ dyes. Exemplary fluorescent and quencher moieties are described in, e.g., WO/2005/049849, which is hereby incorporated by reference.

As is known in the art, suitable quenchers are selected according to the fluoresce moiety. Exemplary reporters and quenchers are further described in Anderson et al, U.S. Pat. No. 7,601,821, hereby incorporated by reference.

Quenchers are also available from various commercial sources. Exemplary commercially available quenchers include, e.g., Black Hole Quenchers® from Biosearch Technologies and Iowa Black® or ZEN quenchers from Integrated DNA Technologies, Inc.

In some embodiments, The probe for sensitive detection of amplicons comprises two quencher moieties. Exemplary probes comprising two quencher moieties include the Zen probes from Integrated DNA Technologies. Such probes comprise an internal quencher moiety that is located about 9 bases away from the detectable moiety, and generally reduce background signal associated with traditional reporter/quencher probes.

Detectable moieties and quencher moieties can be derivatized for covalent attachment to oligonucleotides via common reactive groups or linking moieties. Methods for derivatization of detectable and quencher moieties are described in, e.g., Ullman et al, U.S. Pat. No. 3,996,345; Khanna et al, U.S. Pat. No. 4,351,760; Eckstein, editor, Oligonucleotides and Analogues: A Practical Approach (IRL Press, Oxford, 1991); Zuckerman et al, Nucleic Acids Research, 15: 5305-5321 (1987) (3′ thiol group on oligonucleotide); Sharma et al, Nucleic Acids Research, 19:3019 (1991) (3′ sulfhydryl); Giusti et al, PCR Methods and Applications, 2:223-227 (1993) and Fung et al, U.S. Pat. No. 4,757,141 (5′ phosphoamino group via Aminolink™ II available from Applied Biosystems, Foster City, Calif.); Stabinsky, U.S. Pat. No. 4,739,044 (3′ aminoalkylphosphoryl group); Agrawal et al, Tetrahedron Letters, 31:1543-1546 (1990) (attachment via phosphoramidate linkages); Sproat et al, Nucleic Acids Research, 15:4837 (1987)(5′ mercapto group); Nelson et al, Nucleic Acids Research, 17:7187-7194 (1989) (3′ amino group); all of which are hereby incorporated by reference.

In some embodiments, commercially available linking moieties can be attached to an oligonucleotide during synthesis, e.g. linking moieties available through Clontech Laboratories (Palo Alto, Calif.).

By way of example only, rhodamine and fluorescein dyes can be derivatized with a phosphoramidite moiety for attachment to a 5′ hydroxyl of an oligonucleotide (see, e.g., Woo et al, U.S. Pat. No. 5,231,191; and Hobbs, Jr. U.S. Pat. No. 4,997,928), hereby incorporated by reference.

In some embodiments, the detectable moiety produces a non-fluorescent signal. For example, any probe for which hybridization of the probe to a template results in a detectable separation of the detectable moiety from the quenching moiety may be used. For example, release of the detectable moiety may be detected electronically, by quantum dot sensing, by luminescence, or chemically (e.g., by a change in pH in a solution resulting from probe hybridization). Likewise, any probe that binds to a probe-binding region and for which a change in signal can be detected upon separation of a detectable moiety from a quencher moiety may be used. For example, molecular beacon probes, MGB probes, Pleiades probes, Scorpion probes, or other probes are contemplated for use in the invention.

Molecular beacon probes are described in, e.g., U.S. Pat. Nos. 5,925,517 and 6,103,406, which are hereby incorporated by reference. Molecular beacon probes generally refer to hairpin or bimolecular oligonucleotide probes. A hairpin molecular beacon probe can comprise a detectable moiety at one end of the hairpin, a quencher moiety at the other end of the hairpin, wherein the hairpin comprises a template-binding region. Without wishing to be bound by theory, hybridization of the template binding region to a template can separate the hairpin structure of the probe and separate the detectable moiety from the quencher moiety, enabling detection of the detectable moiety. A bimolecular beacon probe can comprise two oligonucleotide strands having sequences that are complementary to each other at the 5′ end and 3′ end, respectively. The complementary sequences can each be conjugated to a detectable moiety and a quencher moiety, respectively. Each of the two oligonucleotide strands can further comprise a template binding sequence that bind to different regions of a target sequence. The formation of Watson-Crick bonding between the complementary strands can result in the formation of a Y structure and bring the detectable moiety in close proximity with the quencher moiety, resulting in quenching of the detectable moiety. Hybridization of the template binding sequences to the target polynucleotide can break the duplex between the complementary sequences, thus separating the detectable moiety from the quencher moiety and resulting in dequenching of the detectable moiety,

MGB probes are described in, e.g., U.S. Pat. Nos. 7,582,739; 7,381,818; 6,492,346; 6,321,894; 6,303,312; and 6,221,589; which are hereby incorporated by reference. MGB probes refer to oligonucleotide probes comprising a minor groove binder (MGB). The term “minor groove binder”, as used herein, generally refers to a molecule capable of binding within the minor groove of double-stranded DNA, double-stranded RNA, DNA-RNA hybrids, DNA-PNA hybrids, hybrids in which one strand is a PNA/DNA chimera, and/or polymers containing purine and/or pyrimidine bases and/or their analogues which are capable of base-pairing to form duplex, triplex or higher order structures comprising a minor groove. The MGB domain of the probe can stabilize a duplex formed between the probe and its corresponding template polynucleotide. Incorporation of an MGB can enable the use of short probes, can enhance the stability of a probe/template duplex, and retain the specificity of an allele-specific probe. An MGB probe can have an MGB ligand and a quencher located at the 3′-end of the probe, and a fluorophore is attached at the 5′-end of the probe. Alternatively, an MGB probe can have an MGB ligand and quencher located at the 5′-end of the probe and a fluorophore at the 3′-end of the probe.

Pleiades probes are described in US Patent Publication Nos. 20046727356, 20077205105 and 20090111100, hereby incorporated by reference. Pleiades probes generally refers to MGB probes that comprise a detectable moiety, e.g., a fluorophore in close proximity to an MGB at a first end of the probe, and a quencher moiety at a second end of the probe. The detectable moiety can be quenched by the quencher moiety, and additionally can be further quenched by the MGB.

Probes for sensitive detection of amplicons can be designed to have a length. The length of a probe for sensitive detection of amplicons can be sufficiently long that the detectable moiety and quencher are in close enough proximity so as to quench the detectable moiety when the probe is free in solution (e.g., in an unhybridized state). By way of example only, a probe for sensitive detection of amplicons can, in its unhybridized state, exhibit less than 50%, less than 40%, less than 30%, less than 20%, less than 10%, less than 5%, less than 4%, less than 3%, less than 2%, less than 1%, less than 0.5%, less than 0.1%, less than 0.01%, less than 0.001%, or less than 0.0001% fluorescence as compared to the probe in a fully hybridized state. Without wishing to be bound by theory, hybridization of such probes can cause the probes to lose their coiled state and fully stretch out, increasing the distance between a probe's detectable moiety and quencher moiety, thereby activating the detectable moiety. Such hybridization-dependent activatable probes are described in, e.g., U.S. Pat. No. 6,030,787, U.S. Pat. No. 5,723,591 U.S. Pat. No. 7,485,442 and U.S. application Ser. No. 10/165,410), which are hereby incorporated by reference. The detectable moiety and the quencher can be spaced at least 7, 8, 9, 10, 11, 12, 13, 14, 15, 16, 17, 18, 19, 20, 21, 22, 23, 24, 25, 26, 27, 28, 29, 30, or more than 30 nucleotides apart. The detectable moiety and the quencher can be spaced about 7-10, 9-15, 12-20, 20-30, or more than 30 nucleotides apart. The overall length of the probe can be 7, 8, 9, 10, 11, 12, 13, 14, 15, 16, 17, 18, 19, 20, 21, 22, 23, 24, 25, 26, 27, 28, 29, 30, or more than 30 nucleotides. The overall length of the probe can be about 7-12, 12-20, 20-30, or more than 30 nucleotides.

In some embodiments, the probe comprises a nucleotide with a Tm enhancing base. The probe can comprise a Superbase™, a locked nucleoci acid, or bridge nucleic acid. Exemplary locked or bridge nucleic acids are described herein.

Probes can be designed to selectively hybridize to a target polynucleotide of interest. Probes can be designed to have at least 50, 55, 60, 65, 70, 75, 80, 85, 90, 95, 96, 97, 98, 99, or 100% complementarity to a target polynucleotide.

In some embodiments, a probe can be designed to have a length less than 15, 14, 13, 12, 11, or 10 nucleotides. In some embodiments, such a probe has a GC content that is more than 25%, 30%, 35%, 40%, 45%, 50%, 55%, 60%, 65%, 70%, 75%, or up to 80%. In some embodiments, a probe having a length less than 15, 14, 13, 12, 11, or 10 nucleotides comprises a GC content greater than 40%, such as, e.g., 40-80%. In some cases, a probe having a length less than 15, 14, 13, 12, 11, or 10 nucleotides and a GC content that is more than 25%, 30%, 35%, 40%, 45%, 50%, 55%, 60%, 65%, 70%, 75%, or up to 80% does not comprise a modified nucleotide such as a bridge or locked nucleotide. In other embodiments, a probe having a length less than 15, 14, 13, 12, 11, or 10 nucleotides comprises a GC content less than 40%, 35%, 30%, 25%. In particular embodiments, such a probe further comprises a modified nucleotide. In some cases, the modified nucleotide is a locked or bridge nucleotide. In some cases, such a probe comprises a peptide nucleic acid. In such cases, a probe does not necessarily comprise a modified nucleotide.

In other embodiments, a probe is designed to have a length of 15 or more, 16, or more, 17 or more, 18 or more, 19 or more, 20 or more, 21 or more, 22 or more, 23 or more, 24 or more, 25 or more, or 30 or more nucleotides. In particular embodiments, such probes have a GC content that is less than 80%. For example, such probes can have a GC content that is less than 80%, less that 75%, less than 70%, less than 65%, less than 60%, less than 55%, less than 50%, less than 45%, less than 40%, less than 35%, or less than 30%. In particular embodiments, a probe for sensitive detection of amplicons having a length of 15 or more, 16, or more, 17 or more, 18 or more, 19 or more, 20 or more, 21 or more, 22 or more, 23 or more, 24 or more, 25 or more nucleotides also has a GC content that is about 25%, 30%, 35%, 40%, 45%, 50%, 55%, 60%, 65%, or 70%.

A probe for sensitive detection of amplicons can be designed for highly sensitive allelic discrimination, e.g., can be an allele-specific probe. Such probes can be designed to partially or fully overlay a locus suspected of harboring a mutation such as, e.g., a SNP, insertion, deletion, or inversion. An allele-specific probe can be designed to be perfectly matched (e.g., perfectly complementary) to a template nucleic acid containing a specific allele at a locus, but to comprise a mismatch to any other allele of the locus. The mismatch can be a mismatch of 1, 2, 3, 4, 5, or more than 5 nucleotides. In some embodiments, an allele-specific probe can form a duplex with a perfectly template nucleic acid containing a specific allele at a locus. In some embodiments, the probe/perfectly matched template duplex has a first Tm. The allele-specific probe can also form a duplex with a mismatched template nucleic acid containing a different allele at the same locus. In some embodiments, the probe/mismatched template duplex has a second Tm. The difference between the first and second Tm (e.g., the binding penalty of the mismatch) can be at least 1% of the total binding energy of the probe to the template.

A probe can be designed for the sensitive and accurate detection of a target polynucleotide that is not suspected of harboring a mutation such as a SNP, insertion, deletion, or inversion. For example, the target polynucleotide may be suspected of having a copy number variation. In such cases, a probe is not necessarily designed to have a mismatch to the target polynucleotide. In some cases, the probe is designed to be perfectly matched to the target polynucleotide.

Probes can be designed to not hybridize to its target template nucleic acid during PCR. PCR generally involves repeated rounds of thermal cycling. Probes can be designed to not hybridize during the repeated rounds of thermal cycling. A user may set thermal cycling parameters to comprise repeated cycles, the repeated cycles comprising a denaturation step, an annealing step, and an extension step. In some embodiments the repeated cycles do not include any temperature step below 50° C. Following the repeated cycles, a user may also include a final extension step. In some embodiments, the final extension step is not below 50° C. In particular embodiments, the final extension step is about 65-75 degrees ° C. Following the repeated cycles, a user may include a final extension step and/or a cooling step wherein the reaction temperature is reduced to below 45° C., below 40° C., below 35° C., below 30° C., or at or below 25° C. In some embodiments, the invention probe hybridizes to its target template nucleic acid during the cooling step. In such cases, a user may perform endpoint detection of target amplicons. In some embodiments, the cooling step may comprise a controlled cooling step wherein a reaction temperature cools at a constant rate. The constant rate may be 0.01° C./second, 0.02° C./second, 0.03° C./second, 0.04° C./second, 0.05° C./second, 0.06° C./second, 0.07° C./second, 0.08° C./second, 0.09° C./second, 0.10° C./second, 0.2° C./second, 0.3° C./second, 0.4° C./second, 0.5° C./second, 0.6° C./second, 0.7° C./second, 0.8° C./second, 0.9° C./second, or 1° C./second. In such cases, a user may note a temperature at which fluorescence is detected. In some cases, the temperature at which fluorescence is detected may provide information to a user as to a mutational status of a target nucleic acid.

Alternatively, a user may include a cooling step during repeated cycling. For example, a repeated cycle may include a denaturation, annealing, extension, and a cooling step. In some embodiments, the cooling step of the repeated cycles comprises reducing the reaction temperature to below 45° C., below 40° C., below 35° C., below 30° C., or at or below 25° C. In some embodiments, the invention probe hybridizes to its target template nucleic acid during the cooling step. In such cases, a user may perform real-time detection of target amplicons.

### Reaction Mixture for Sensitive Detection of Amplicons

In another aspect, the invention provides a reaction mixture for sensitive detection of amplicons. The reaction mixture for sensitive detection of amplicons can comprise components for carrying out a PCR reaction. The reaction mixture for sensitive detection of amplicons can comprise components necessary to amplify at least one amplicon from nucleic acid template molecules. The reaction mixture for sensitive detection of amplicons may comprise nucleotides (dNTPs), a polymerase, one or more primers, and an invention probe. The reaction mixture for sensitive detection of amplicons may further comprise a Tris buffer, a monovalent salt, and one or more cation. The one or more cations can be Mg²⁺ and/or Mn²⁺. In some embodiments, the reaction mixture for sensitive detection of amplicons comprises Mg²⁺ and Mn²⁺. The concentration of each component can be optimized by an ordinary skilled artisan. In some embodiments, the reaction mixture for sensitive detection of amplicons also comprises additives including, but not limited to, non-specific background/blocking nucleic acids (e.g., salmon sperm DNA), biopreservatives (e.g. sodium azide), PCR enhancers (e.g. Betaine, Trehalose, etc.), and inhibitors (e.g. RNAse inhibitors). In some embodiments, a nucleic acid sample is admixed with the reaction mixture for sensitive detection of amplicons. Accordingly, in some embodiments the reaction mixture for sensitive detection of amplicons further comprises a nucleic acid sample.

Primers used in the present invention can comprise a template binding region that is designed to hybridize to a target polynucleotide of interest. Primers used in the present invention are generally sufficiently long to prime the synthesis of extension products in the presence of the agent for polymerization. The exact length and composition of a primer can depend on many factors, including temperature of the annealing reaction, source and composition of the primer, and ratio of primer:probe concentration. The primer length can be, for example, about 5-100, 10-50, 15-30, or 18-22 nucleotides, although a primer may contain more or fewer nucleotides.

Primers used in the present invention can also comprise a probe-binding region. Exemplary probe-binding regions are described herein.

Primers used in the present invention can further comprise a barcode sequence. The term “barcode sequence” as used herein, generally refers to a unique sequence of nucleotides that can encode information about an assay. In some embodiments, a barcode sequence encodes information relating to the identity of an interrogated allele, identity of a target polynucleotide or genomic locus, identity of a sample, a subject, or any combination thereof. In some embodiments, a barcode sequence does not hybridize to the template nucleic acid. A barcode sequence can, for example, be designed to avoid significant sequence similarity or complementarity to known genomic sequences of an organism of interest. Such unique sequences can be randomly generated, e.g., by a computer readable medium, and selected by BLASTing against known nucleotide databases such as, e.g., EMBL, GenBank, or DDBJ. The barcode sequence can also be designed to avoid secondary structure. A barcode sequence may be at a 3′-end or more preferably at a 5′ end of a primer. Barcode sequences may vary widely in size and composition; the following references provide guidance for selecting sets of barcode sequences appropriate for particular embodiments: Brenner, U.S. Pat. No. 5,635,400; Brenner et al, Proc. Natl. Acad. Sci., 97: 1665-1670 (2000); Shoemaker et al, Nature Genetics, 14: 450-456 (1996); Morris et al, European patent publication 0799897A1; Wallace, U.S. Pat. No. 5,981,179, all of which are hereby incorporated by reference. In particular embodiments, a barcode sequence may have a length of about 4 to 36 nucleotides, about 6 to 30 nucleotides, or about 8 to 20 nucleotides. The barcode sequence can have any length. In some embodiments, primers can comprise a probe-binding region as described herein.

Primers and/or probes may be prepared by any suitable method. Methods for preparing oligonucleotides of specific sequence are known in the art, and include, for example, cloning and restriction of appropriate sequences and direct chemical synthesis. Chemical synthesis methods may include, for example, the phosphotriester method described by Narang et al., 1979, Methods in Enzymology 68:90, the phosphodiester method disclosed by Brown et al., 1979, Methods in Enzymology 68:109, the diethylphosphoramidate method disclosed in Beaucage et al., 1981, Tetrahedron Letters 22:1859, and the solid support method disclosed in U.S. Pat. No. 4,458,066. The above references are hereby incorporated by reference.

Primers and/or probes can be obtained from commercial sources such as, e.g., Operon Technologies, Amersham Pharmacia Biotech, Sigma, IDT Technologies, and Life Technologies. The primers can have an identical or similar melting temperature. The lengths of the primers can be extended or shortened at the 5′ end or the 3′ end to produce primers with desired melting temperatures. Also, the annealing position of each primer pair and/or each probe can be designed such that the sequence and, length of the primer pairs and/or probes yield the desired melting temperature.

The melting temperature of the primers and/or probes can be determined empirically, e.g., by performing a melting curve analysis. Methods of performing melting curve analysis to empirically determine Tm of a primer and/or probe are known to those of skill in the art. The melting temperature of the primers and/or probes can also be predicted. By way of example only, the simplest equation for predicting the melting temperature of primers smaller than 25 base pairs is the Wallace Rule:

(Td=2(A+T)+4(G+C)).

Another method for calculating the Tm of an oligonucleotide is the nearest-neighbor method. The nearest-neighbor method generally incorporates certain variables such as salt concentration and DNA concentration. This method can incorporate reaction mixture conditions typically found in PCR applications, such as, e.g., 50 mM monovalent salt and 0.5 11M primer. Generally, the nearest-neighbor equation for DNA and RNA-based oligonucleotides is:

Tm=(1000ΔH)/A+ΔS+Rln(C/4)−273.15+16.6 log[Na+], wherein
ΔH(Kcal/mol) is the sum of the nearest-neighbor enthalpy changes for hybrids, A is a constant containing corrections for helix initiation. ΔS is the sum of the nearest-neighbor entropy changes, R is the Gas Constant (1.99 cal K−1 mol−1), and C is the concentration of the oligonucleotide.

The ΔH and ΔS values for nearest-neighbor interactions of DNA and RNA are shown in Table 1 (below).

TABLE 1
Thermodynamic parameters for nearest-
neighbor melting point formula.

| | |
|---|---|
| DNA | RNA |

| Interaction | ΔH | ΔS | ΔH | ΔS |
|---|---|---|---|---|
| AA/TT | −9.1 | −24 | −6.6 | −18.4 |
| AT/TA | −8.6 | −23.9 | −5.7 | −15.5 |
| TA/AT | −6 | −16.9 | −8.1 | −22.6 |
| CA/GT | −5.8 | −12.9 | −10.5 | −27.8 |
| GT/CA | −6.5 | −17.3 | −10.2 | −26.2 |
| CT/GA | −7.8 | −20.8 | −7.6 | −19.2 |
| GA/CT | −5.6 | −13.5 | −13.3 | −35.5 |
| CG/GC | −11.9 | −27.8 | −8 | −19.4 |
| GC/CG | −11.1 | −26.7 | −14.2 | −34.9 |
| GG/CC | −11 | −26.6 | −12.2 | −29.7 |
| Initiation | 0 | −10.8 | 0 | −10.8 |

Another equation that is generally used for predicting the Tm of a DNA oligonucleotide which is longer than, e.g., 50 bases at a pH between, e.g., 5.0 to 9.0 is the % GC method:

Tm=81.5+16.6 log[Na+]+41(X_{G}+X_{C})−500/L−0.62F

wherein [Na+] is the molar concentration of monovalent cations (in this case Na+), X_{G} and X_{C} are the mole fractions of G and C in the oligonucleotide, L is the length of the shortest strand in the duplex, and F is the percentage of formamide in the hybridization solution.

Those of skill in the art will understand that Tm can also depend on factors other than the oligonucleotide sequence. Tm can depend on, e.g., salt concentration of a reaction mixture, buffer type used in a reaction mixture, the relative concentration of the primer or probe relative to the template concentration, and other factors. Computer programs can also be used to design primers, including but not limited to Array Designer Software (Arrayit Inc.), Oligonucleotide Probe Sequence Design Software for Genetic Analysis (Olympus Optical Co.), NetPrimer, PrimerExpress, and DNAsis from Hitachi Software Engineering. The Tm (melting or annealing temperature) of each primer can be calculated using software programs such as, e.g., Oligo Design, available from Invitrogen Corp, BioMath Calculators from Promega (http://www.promega.com/techserv/tools/biomath/calc11.htm), Tm Calculator from New England Biolabs, OligoAnalyzer from Integrated DNA Technologies, among others.

The reaction mixture for sensitive detection of amplicons can comprise reaction components for performing linear amplification. Generally, during linear amplification, only one strand of a double-stranded template nucleic acid is amplified per cycle, resulting in single-stranded extension products. To enable linear amplification, a reaction mixture can, for example, comprise only one primer per target polynucleotide.

Alternatively, the reaction mixture for sensitive detection of amplicons can be configured for exponential amplification. Generally, during exponential amplification, both strands of a double-stranded template nucleic acid are amplified per cycle, resulting in the generation of 2ⁿ copies of a target polynucleotide, wherein n is the number of cycles in a PCR reaction. To enable exponential amplification, a reaction mixture can comprise a forward and reverse primer per target polynucleotide. Typically, for exponential amplification, the forward and reverse primers are present in the reaction mixture at a ratio between 1:3-3:1 ratio, between 1:2-2:1 ratio, preferably between 2:3-3:2 ratio, more preferably between 3:4-4:3 ratio, or yet even more preferably about a 1:1 ratio.

In some cases, the reaction mixture for sensitive detection of amplicons can be configured for exponential amplification followed by linear amplification. In such cases, one primer of a forward/reverse primer set can be present in an excess concentration or amount as compared to the other primer of the forward/reverse primer set. In some embodiments, the concentration of the excess primer is at least 2×, 3×, 4×, 5×, 6×, 7×, 8×, 9×, 10× the concentration of the limiting primer. In some embodiments, the concentration of the excess primer is about 2-10×, 5-50×, 20-100×, 50-500×, 100-1000×, 500-2000×, 1000-5000×, 2000-10000×, or more than 10000× the concentration of the limiting primer. In such cases, exponential amplification will proceed until exhaustion of the limiting primer, upon which linear amplification proceeds using the excess primer remaining in the reaction mixture or discrete reaction volume. Without wishing to be bound by theory, exponential-followed-by-linear amplification ensures (1) that enough amplification products are generated as to result in a detectable signal, and (2) that the PCR reaction products are predominantly single-stranded extension products which, upon cooling the reaction temperature to below, e.g., 50° C., are available to bind to a detection probe instead of, e.g., to its reverse complement strand. Accordingly, in some embodiments, upon termination of PCR thermal cycling, single stranded extension products account for at least 5%, 10%, 20%, 30%, 40%, 50%, 55%, 60%, 65%, 70%, 75%, 80%, 85%, 90%, 95%, or more that 95% of the total amount of reaction products. In some embodiments single stranded extension products do not account for at least 50% of the total amount of reaction products. In some embodiments, upon termination of PCR thermal cycling, at least 5%, at least 10%, at least 20%, at least 30%, at least 40%, at least 50%, 55%, 60%, 65%, 70%, 75%, 80%, 85%, 90%, 95%, or more that 95% of the PCR extension products are extensions of the excess primer. In such cases, linear amplification can be performed following exponential amplification without a user adding or removing components from the reaction mixture.

The reaction mixture for sensitive detection of amplicons can comprise a polymerase. In some embodiments, the polymerase is a DNA polymerase. In particular embodiments, the DNA polymerase is a thermostable polymerase. The thermostable polymerase may originate from a thermophilic bacterium or from Archaea. Exemplary thermostable polymerases include, but are not limited to, Thermus aquaticus (Taq polymerase), Pyrococcus furiosus (Pfu polymerase), Vent® DNA Polymerase gene from Thermococcus litoralis, Deep Vent™ polymerase from Pyrococcus sp., Platinum® Pfx polymerase, Tfi polymerase from Thermus filiformis, Pwo polymerase, chimeric DNA polymerases comprising a DNA binding protein (e.g., Phusion, iProof), topoisomerase. In some embodiments, the polymerase is capable of isothermal amplification. The polymerase can be, e.g., Bst DNA polymerase, Bca DNA polymerase, E. coli DNA polymerase I, the Klenow fragment of E. coli DNA polymerase I, Taq DNA polymerase, T7 DNA polymerase (Sequenase).

In some embodiments, the DNA polymerase comprises 5′→3′ exonuclease activity. As used herein, “5′→3′ nuclease activity” or “5′ to 3′ nuclease activity” can refer to an activity of a template-specific nucleic acid polymerase whereby nucleotides are removed from the 5′ end of an oligonucleotide in a sequential manner. DNA polymerases with 5′→3′ exonuclease activity are known in the art and include, e.g., DNA polymerase isolated from Thermus aquaticus (Taq DNA polymerase). In some embodiments, the DNA polymerase lacks 3′→5′ exonuclease activity. Exemplary DNA polymerases lacking 3′→5′ exonuclease activity include, but are not limited to BST DNA polymerase I, BST DNA polymerase I (large fragment), Taq polymerase, Streptococcus pneumoniae DNA polymerase I, Klenow Fragment (3′→5′ exo-), PyroPhage® 3173 DNA Polymerase, Exonuclease Minus (Exo-) (available from Lucigen), T4 DNA Polymerase, Exonuclease Minus (Lucigen). In some embodiments, the DNA polymerase is a recombinant DNA polymerase that has been engineered to lack exonuclease activity.

In some embodiments, a reaction mixture for sensitive detection of amplicons can comprise multiple primers and probes for multiplex detection. By way of example only, a reaction mixture for sensitive detection of amplicons reaction mixture can comprise a primer/probe set. In some embodiments, a primer/probe set comprises a common forward primer and optionally a reverse primer designed to amplify a target polynucleotide suspected of harboring a mutation at a locus, and further comprises a plurality of probes, wherein each probe is specific for a specific allele of the locus. Each probe in the primer/probe set can further comprise a distinct detectable moiety that is detectably distinct from any other detectable moiety in the reaction mixture. By way of other example, a reaction mixture can comprise a plurality of primer/probe sets, wherein each primer/probe set is specific for a different target polynucleotide, e.g., a different locus.

In some embodiments, the primer/probe set comprises a common reverse primer, a first allele-specific forward primer, and at least a second allele-specific forward primer designed to amplify a target polynucleotide suspected of harboring a mutation at a locus. The forward primers can each comprise a template binding region. The template binding region may overlay a mutation. The forward primers can each further comprise a probe-binding region (e.g., barcode region). One of the forward primers can be a wild-type specific forward primer that is complementary to the wild-type allele at the site that overlays the mutation. The wild-type specific forward primer can further comprise a wild-type barcode region which does not generally hybridize to a template nucleic acid. The wild-type barcode region may contain a wild-type barcode sequence that specifically hybridizes a wild-type low Tm probe, but does not substantially hybridize a mutant low Tm probe. One of the forward primers can be a mutant-specific forward primer that is complementary to the mutant allele at the site that overlays the mutation. The mutant specific forward primer can further comprise a mutant barcode region which does not generally hybridize to a template nucleic acid. The mutant barcode region may contain a mutant barcode sequence that specifically hybridizes a mutant low Tm probe, but does not substantially hybridize to the wild-type low Tm probe. The forward primers (wild-type and mutant forward primers) may further comprise a deliberate mismatch nucleotide adjacent to or within 1-3 nucleotides from the nt that overlays the mutation. However, in some cases, the forward primers do not further comprise a deliberate mismatch nucleotide adjacent to or within 1-3 nucleotides from the nt that overlays the mutation. The primer/probe set may further comprise a wild-type low Tm probe and a mutant low Tm probe. The wild-type low Tm probe may be designed to specifically hybridize to the wild-type barcode region. The mutant low Tm probe may be designed to specifically hybridize to the mutant barcode region. The wild-type and mutant low Tm probes may comprise spectrally distinct fluorphores. The primer/probe set may further comprise a common reverse primer.

### Methods for Sensitive Detection of Amplicons

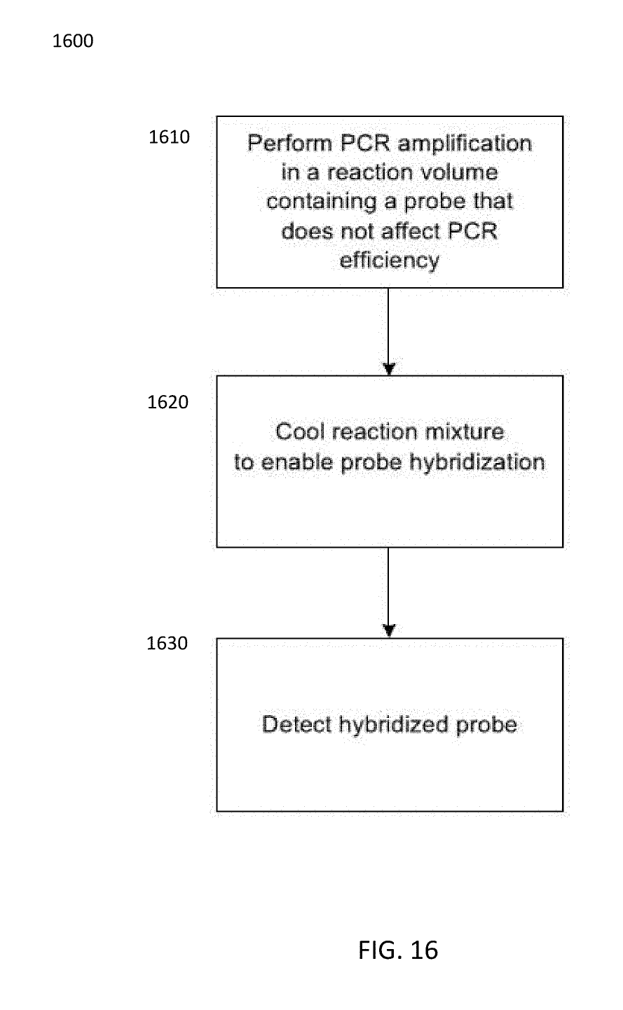

FIG. 16 depicts an exemplary workflow 1600 for a method for the sensitive detection of amplicons, comprising a first step 1610 of performing a probe-based PCR assay in a reaction mixture, wherein the probe-based PCR assay comprises thermal cycling, wherein the probe is designed to have minimal to zero impact on kinetics or efficiency of the PCR amplification reaction. In some embodiments, the probe does not hybridize to a template nucleic acid during the PCR reaction. In some embodiments, the oligonucleotide probe hybridizes to a template nucleic acid after termination of a PCR reaction. Termination of a PCR reaction can include a next step 1620 of allowing the reaction mixture to cool to a temperature that enables hybridization of the probe to a target polynucleotide. In some embodiments, probe hybridization enables detection of the hybridized probe. The method can further comprise a next step 1630 of detecting the probe.

### Amplification

In some embodiments amplification is carried out utilizing a nucleic acid polymerase. In some embodiments, the nucleic acid polymerase is a DNA polymerase. In particular embodiments, the DNA polymerase is a thermostable DNA polymerase. In other embodiments, the DNA polymerase is capable of isothermal amplification. Exemplary DNA polymerases are described herein.

In some embodiments, the reaction mixture is subjected to a PCR amplification reaction. PCR amplification can involve repeated thermal cycling. Thermal cycling can be carried out as an automated process. The automated process may be carried out using a PCR thermal cycler. Commercially available thermal cycler systems include systems from Bio-Rad Laboratories, Life Technologies, Perkin-Elmer, among others.

The thermal cycling can comprise cycling through the repeated steps of denaturation, primer annealing and primer extension. Temperatures and times for the three steps can be, e.g., 90-100° C. for 5 seconds or more for denaturation, 50-65° C. for 10-60 sec for the annealing phase, and 50-75° C. for 15-120 sec for primer extension. In some embodiments, primer annealing and primer extension are combined in a single temperature step (e.g., 60° C.). Prior to thermal cycling, a PCR reaction can include a “hot-start” initiation phase to activate a polymerase. The “hot-start” phase can comprise heating a reaction mixture to 90-100° C. Following the repeated cycles, a user may also include as part of the PCR reaction a final extension step. The final extension step can comprise a reaction temperature of 50-75° C. for, e.g., 5, 6, 7, 8, 9, 10, or more than 10 minutes.

Thermal cycling parameters can be set by a user. In some embodiments, a user sets thermal cycling parameters so as to enable endpoint detection of a low Tm probe. For example, a user can set thermal cycling parameters such that the repeated cycles do not include any temperature step below 50° C. Such parameters can minimize hybridization of the low Tm probe during the PCR reaction. Following the repeated cycles, a user may also include a final extension step. In some embodiments, the final extension step is not below 50° C. In particular embodiments, the final extension step is about 50-75° C. Following the repeated cycles, a user may include a final extension step and/or a cooling step wherein the reaction temperature is reduced to below 45° C., below 40° C., below 35° C., below 30° C., or at or below 25° C. In some embodiments, the low Tm probe hybridizes to its target template nucleic acid during the cooling step. In such cases, a user may perform endpoint detection of target amplicons. In some embodiments, the cooling step may comprise a controlled cooling step wherein a reaction temperature cools at a constant rate. The constant rate may be as described herein. In such cases, a user may note a temperature at which fluorescence is detected. In some cases, the temperature at which fluorescence is detected may provide information to a user as to a mutational status of a target nucleic acid.

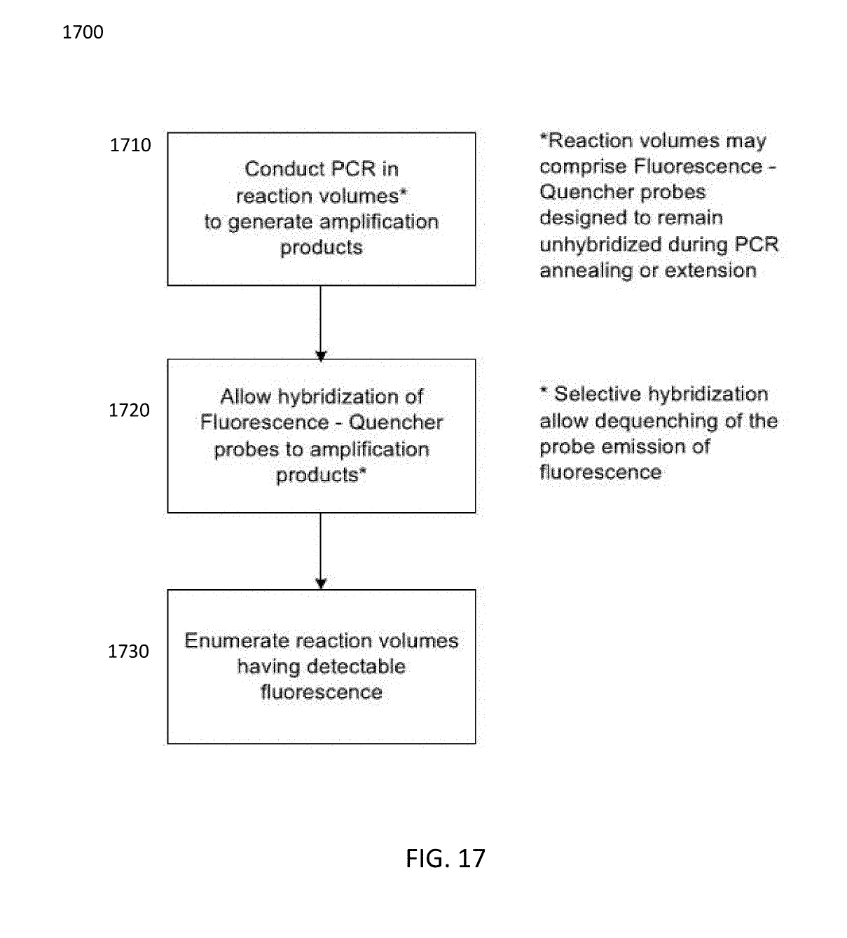

FIG. 17 depicts an exemplary workflow 1700 for an endpoint detection method of the invention, comprising a first step 1710 of conducting a PCR reaction in a plurality of reaction volumes. In some embodiments, one or more of the reaction volumes comprise a probe for sensitive detection of amplicons (e.g., a low Tm probe) comprising a fluorescent moiety and a quencher moiety. In some embodiments, the probe is configured to remain unhybridized during a PCR annealing or extension phase. In some embodiments the PCR thermal cycling phases do not comprise any temperature phase that is less than 5° C. above the Tm of the low Tm probe. In some embodiments, the PCR reaction results in the generation of amplification products. In a next step 1720, the reaction volumes are cooled to a temperature that enables hybridization of the low Tm probe to the amplification products. In some embodiments, the selective hybridization of the low Tm probe to its target polynucleotide allows dequenching of fluorescence emission from the detectable moiety of the probe. In a next step 1730, the reaction volumes having detectable fluorescence are enumerated.

Alternatively, a user may introduce a cooling step into the repeated thermal cycles. For example, a repeated cycle may include a denaturation step, annealing step, extension step, and a cooling step. In another example, a repeated cycle may include a first denaturation step, annealing step, extension step, second denaturation step, and a cooling step. In some embodiments, the cooling step of the repeated cycles comprises reducing the reaction temperature to below 45° C., below 40° C., below 35° C., below 30° C., or at or below 25° C. In some embodiments, the low Tm probe hybridizes to its target template nucleic acid during the cooling step. In such cases, a user may perform real-time PCR detection of target amplicons by detecting a level of hybridized probe during each cooling step. As used herein, “real-time PCR” refers to PCR methods wherein an amount of detectable signal is monitored with each cycle of PCR. In some embodiments, a cycle threshold (Ct) wherein a detectable signal reaches a detectable level is determined. Generally, the lower the Ct value, the greater the concentration of the interrogated allele. Systems for real-time PCR are known in the art and include, e.g., the ABI 7700 and 7900HT Sequence Detection Systems (Applied Biosystems, Foster City, Calif.). The increase in signal during the exponential phase of PCR can provide a quantitative measurement of the amount of templates containing the mutant allele.

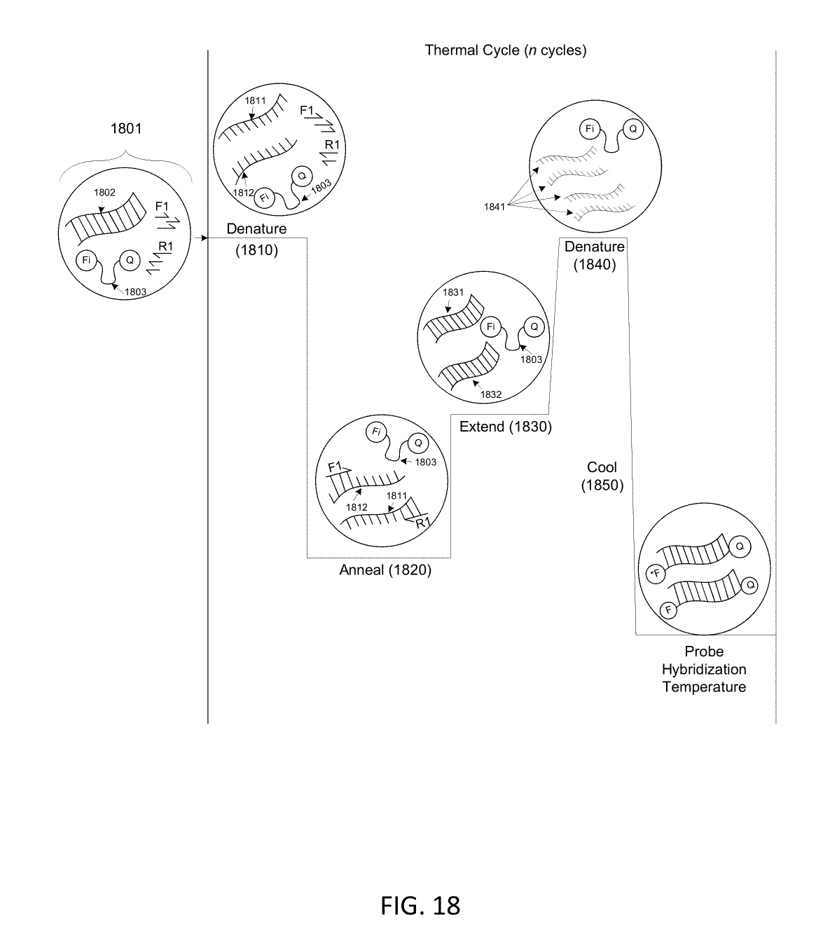

FIG. 18 depicts an exemplary method of the invention comprising real-time detection, comprising thermal cycling a reaction mixture 1801 comprising template nucleic acid 1802, forward and reverse primers F1 and R1, respectively, a probe 1803 for sensitive detection of amplicons comprising a fluorescence moiety F and quencher moiety Q, dNTPs (not shown), and any other reaction components necessary for carrying out a PCR reaction (e.g., a polymerase, not shown). In some embodiments, the fluorescent moiety of the probe when the probe is in an unhybridized state is quenched (denoted by Fi). A PCR reaction may or may not be initiated by a “hot-start” (not shown). Thermal cycling may be initiated following the “hot-start”. The repeated thermal cycles can comprise a first denaturation phase 1810 which denatures the double-stranded template nucleic acid into single-stranded template strands 1811 and 1812. The first denaturation phase can be followed by a primer annealing phase 1820 in which the forward and reverse primers F1 and R1 are allowed to hybridize to their target strands 1811 and 1812. During the annealing phase, a probe 1803 for sensitive detection of amplicons generally does not exhibit significant hybridization to its target template. The annealing phase can be followed by an extension phase 1830, wherein a polymerase extends the F1 and R1 primers, thereby creating two copies of the target polynucleotide 1831 and 1832. During this phase, a probe 1803 for sensitive detection of amplicons would generally not hybridize to a template nucleic acid. The extension phase can be followed by a second denaturation phase 1840 which denatures the double-stranded template nucleic acid into single-stranded template strands 1841. The second denaturation phase can be followed by a cooling phase e.g., cooling to below 50° C. or cooling to about room temperature. Cooling the reaction mixture can enable hybridization of the low Tm probe to a target polynucleotide. Hybridization of the probe can result in full extension of the probe and release the detectable moiety from the influence of the quencher moiety (detectable moiety depicted as *F). The detectable moiety can thus be detected during each thermal cycle.

In some embodiments, repeated cycles of denaturation, primer annealing, and primer extension result in the accumulation of amplicons comprising a target polynucleotide. The amplicons may be single or double stranded. Sufficient cycles can be run to accumulate an amount of amplicons comprising the target polynucleotide sufficient to enable hybridization of detectable levels of probe. The resulting detectable signal can be 2, 5, 10, 20, 30, 40, 50, 60, 70, 80, 90, 100, 200, 300, 400, 500, 600, 700, 800, 900, 1000, 2000, 3000, 4000, 5000, 6000, 7000,8000, 9000, or 10000-fold greater or several orders of magnitude greater than background signal.

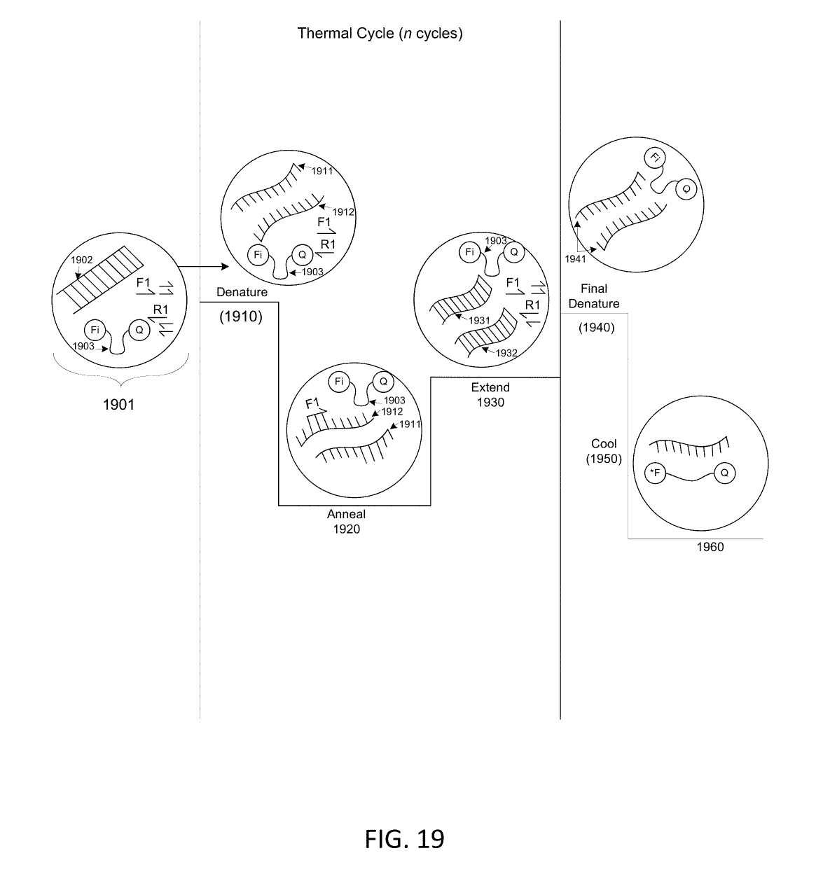

In some embodiments, the PCR amplification reaction is an exponential amplification reaction. An exemplary embodiment of a method involving exponential amplification is depicted in FIG. 19. A starting reaction mixture or volume 1901 can comprise a template nucleic acid 1902, which may be a double-stranded template nucleic acid, a probe 1903 for sensitive detection of amplicons as described herein, the probe 1903 comprising a fluorescent moiety and quencher moiety, forward and reverse primers F1 and R1 designed to amplify a target polynucleotide, dNTPs (not shown), and any other reaction components necessary for carrying out a PCR reaction (e.g., a polymerase, not shown). In some embodiments, the fluorescent moiety of the probe when the probe is in an unhybridized state is quenched (denoted by Fi). A PCR reaction may or may not be initiated by a “hot-start” (not shown). The reaction mixture may then begin thermal cycling. Each thermal cycle can comprise a denaturation phase 1910, in which a double-stranded template nucleic acid is partially or fully denatured into single strands 1911 and 1912. Generally, during this denaturation phase, neither primer hybridization nor probe hybridization occurs. After denaturation, an annealing phase 1920 can be initiated wherein the F1 and R1 primers anneal to the single strands of the target polynucleotide. During this phase, a probe 1903 for sensitive detection of amplicons would generally not hybridize to a template nucleic acid. After the annealing phase, an extension phase 1930 can be initiated wherein a polymerase extends the F1 and R1 primers, thereby creating two copies of the target polynucleotide 1931 and 1932. During this phase, a probe 1903 for sensitive detection of amplicons would generally not hybridize to a template nucleic acid. Repetition of the thermal cycles can accordingly result in the exponential amplification of the target polynucleotide. After the final repeated cycle, a final denaturation step 1940 can be initiated. The final denaturation step can fully or partially denature any double-stranded target polynucleotides into single strands 1941. Following the final denaturation step, the reaction mixture can be cooled in a cooling step 1950, e.g., cooled to below 50° C. or cooled to about room temperature. Cooling the reaction mixture can enable hybridization of the invention probe to a target polynucleotide in a final cooled phase 1960. Hybridization of the probe can result in full extension of the probe and release the detectable moiety from the influence of the quencher moiety. The detectable moiety can thus be detected.

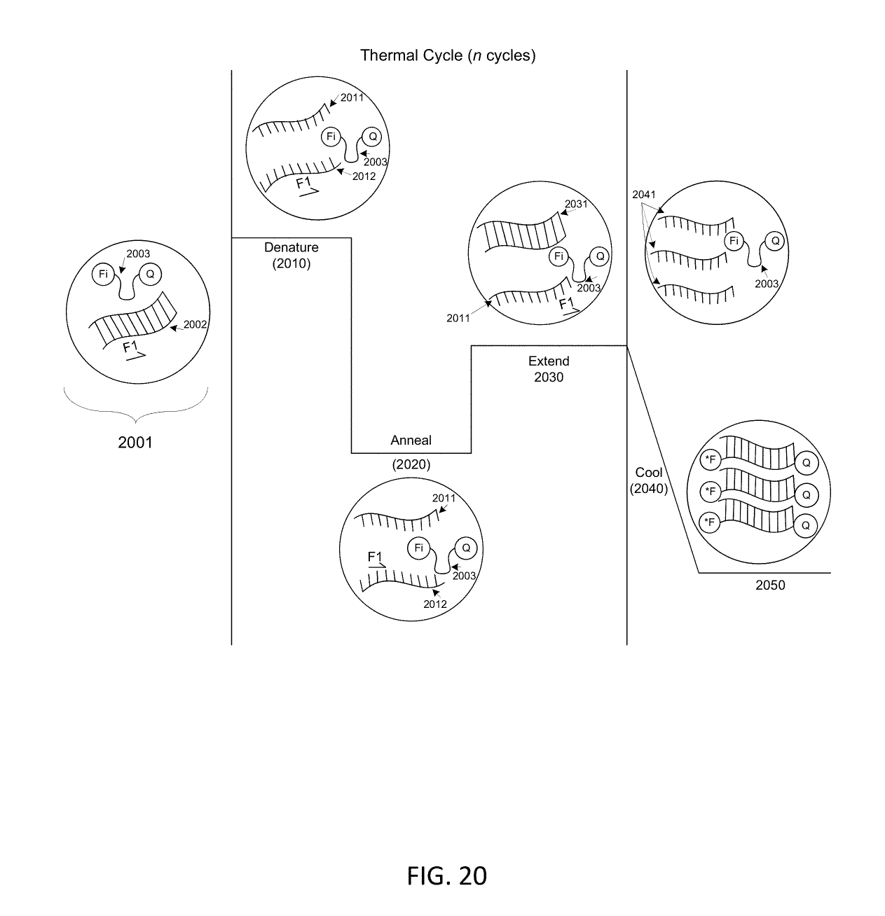

In some embodiments, the PCR amplification reaction is a linear amplification reaction. An exemplary embodiment of a method comprising linear amplification is depicted in FIG. 20. A starting reaction mixture or volume 2001 can comprise a template nucleic acid 2002, which may be a double-stranded template nucleic acid, a probe 2003 for sensitive detection of amplicons as described herein, the probe 2003 comprising a fluorescent moiety and quencher moiety, and a primer F1 designed to hybridize to a single template strand comprising a target polynucleotide in a strand-specific manner, dNTPs (not shown), and any other reaction components necessary for carrying out a PCR reaction (e.g., a polymerase, not shown). In some embodiments, the fluorescent moiety of the probe when the probe is in an unhybridized state is quenched (denoted by Fi). A PCR reaction may or may not be initiated by a “hot-start” (not shown). The reaction mixture may then begin thermal cycling. Each thermal cycle can comprise a denaturation phase 2010, in which a double-stranded template nucleic acid is partially or fully denatured into single strands 2011 and 2012. Generally, during this denaturation phase, neither primer hybridization nor probe hybridization occurs. After denaturation, an annealing phase 2020 can be initiated wherein the F1 primer anneals to a denatured strand 2012 of the target polynucleotide in a strand-specific manner. During this phase, a probe 2003 for sensitive detection of amplicons would generally not hybridize to a template nucleic acid. After the annealing phase, an extension phase 2030 can be initiated wherein a polymerase extends the F1 primer, thereby creating a copy of the target polynucleotide 2031. During this phase, a probe 2003 for sensitive detection of amplicons would generally not hybridize to a template nucleic acid. During this phase, the single strand 2011 is generally not amplified. Repetition of the thermal cycles of denaturation, annealing, and extension can accordingly result in the linear accumulation of single-stranded amplicons 2041 comprising the target polynucleotide. Upon termination of thermal cycling, which can result in the accumulation of single-stranded products 2041, the reaction mixture can be cooled in a cooling step 2040, e.g., cooled to below 50° C. or cooled to about room temperature. Cooling the reaction mixture can enable hybridization of the invention probe to a target polynucleotide in a final cooled phase 2050. Hybridization of the probe can result in full extension of the probe and release the detectable moiety from the influence of the quencher moiety. The detectable moiety can thus be detected.

In some embodiments, the PCR amplification reaction is a non-symmetric polymerase chain reaction (PCR). The non-symmetric PCR reaction can include an initial exponential amplification phase followed by a linear amplification phase. In some cases, the transition from an exponential to a linear amplification phase occurs without addition of reaction components to a reaction mixture or removal of components from the reaction mixture. In some cases, the non-symmetric PCR reaction involves subjecting a reaction mixture to repeated thermal cycles, wherein the reaction mixture comprises a polynucleotide template target, a pair of PCR primers, dNTPs, an invention probe, and a thermostable polymerase. The thermal cycles can correspond to the PCR steps of denaturation, primer annealing and primer extension, wherein, at the outset of the PCR reaction, the PCR primer pair comprises a limiting primer and an excess primer. The excess primer can be present at a concentration at least two times higher, at least three times higher, at least four times higher, at least five times higher, at least 10 times higher, at least 20 times higher, at least 30 times higher, at least 40 times higher, at least 50 times higher, at least 100 times higher, at least 200 times higher, at least 300 times higher, at least 400 times higher, at least 500 times higher, or at least 1000 times higher than the limiting primer. The excess primer can be present at a concentration that is 2-8× higher, 5-10× higher, 10-100× higher, 100-500× higher than the concentration of the limiting primer.

For example, the starting molar concentration of the limiting primer can be less than the starting molar concentration of the excess primer. The ratio of the starting concentrations of the excess primer relative to the limiting primer can be at least 2:1, 3:1, 4:1, 5:1, 10:1, 20:1, or 100:1. The ratio of excess primer to limiting primer can be 5:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, or 100:1. In some embodiments, the ratio is in the range of 20:1 to 100:1.

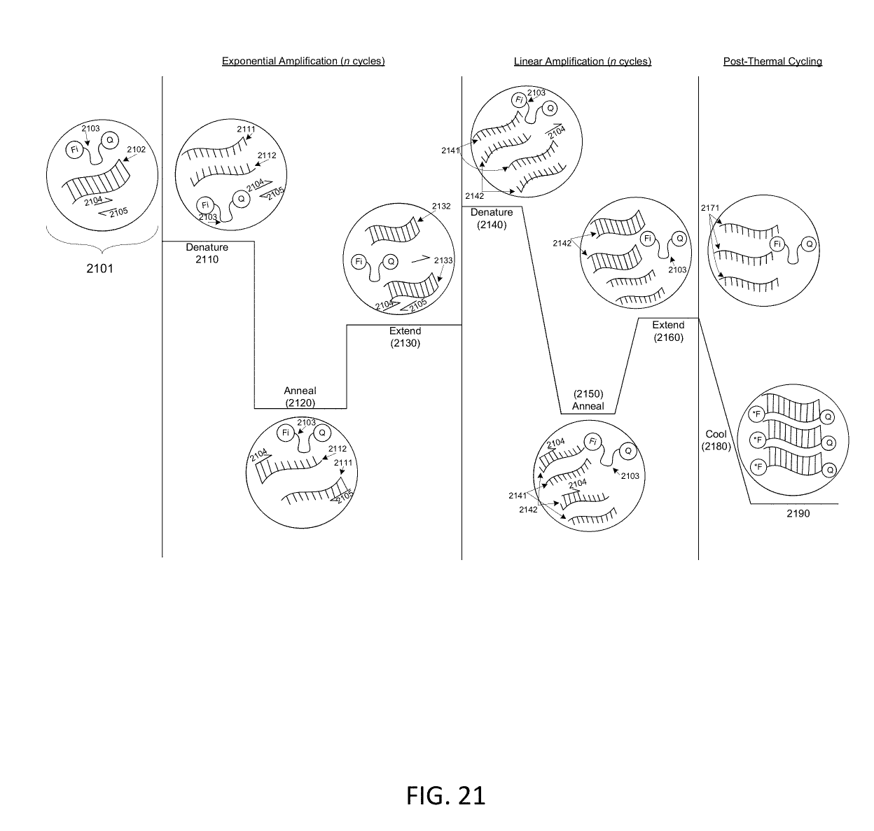

An exemplary embodiment of a method comprising exponential amplification followed by linear amplification is depicted in FIG. 21. A starting reaction mixture or volume 2101 can comprise a template nucleic acid 2102, which may be a double-stranded template nucleic acid, a probe 2103 for sensitive detection of amplicons as described herein, the invention probe comprising a fluorescent moiety and quencher moiety, an excess primer 2104, and a limiting primer 2105, designed to hybridize to opposite strands of a target polynucleotide, dNTPs (not shown), and any other reaction components necessary for carrying out a PCR reaction (e.g., a polymerase, not shown). In some embodiments, the fluorescent moiety of the probe when the probe is in an unhybridized state is quenched (denoted by Fi). A PCR reaction may or may not be initiated by a “hot-start” (not shown). The reaction mixture may then begin thermal cycling. Each thermal cycle can comprise a denaturation phase 2110, in which a double-stranded template nucleic acid is partially or fully denatured into single strands 2111 and 2112. Generally, during this denaturation phase, neither primer hybridization nor probe hybridization occurs. After denaturation, an annealing phase 2120 can be initiated wherein primers 2104 and 2105 anneal to the single strands of the target polynucleotide. During this phase, a probe 2103 for sensitive detection of amplicons would generally not hybridize to a template nucleic acid. After the annealing phase, an extension phase 2130 can be initiated wherein a polymerase extends primers 2104 and 2105, thereby creating two copies of the target polynucleotide 2131 and 2132. During this phase, a probe 2103 for sensitive detection of amplicons would generally not hybridize to a template nucleic acid. Repetition of the thermal cycles can accordingly result in the exponential amplification of the target polynucleotide until the limiting primer 2105 is exhausted, after which the thermal cycles result in linear amplification of the target polynucleotide. The thermal cycles of linear amplification can comprise the same repeated cycles of denaturation, annealing, and extension as described above. In a denaturation phase 2140, the amplified, double-stranded target polynucleotides 2131 and 2132 and denatured into single strands 2141 and 2142. Generally, during this denaturation phase, neither primer hybridization nor probe hybridization occurs. Following denaturation, an annealing phase 2150 can be initiated wherein excess primer 2104 anneals to single strands 2142. During this phase, a probe 2103 for sensitive detection of amplicons would generally not hybridize to a template nucleic acid. After the annealing phase, an extension phase 2160 can be initiated wherein a polymerase extends primer 2104, thereby creating a copy of the target polynucleotide 2161. During this phase, an invention probe would generally not hybridize to a template nucleic acid. During this phase, the single strand 2141 is generally not amplified. Repetition of the thermal cycles can accordingly result in the linear amplification of the target polynucleotide and accumulation of single-stranded products 2171. Upon termination of thermal cycling, which results in the accumulation of single-stranded products 2171, the reaction mixture can be cooled in a cooling step 2180, e.g., cooled to below 50° C. or cooled to about room temperature. Cooling the reaction mixture can enable hybridization of the probe 2103 to a target polynucleotide in a final cooled phase 2190. Hybridization of the probe can result in full extension of the probe and release the detectable moiety from the influence of the quencher moiety. The detectable moiety can thus be detected (denoted as F*).

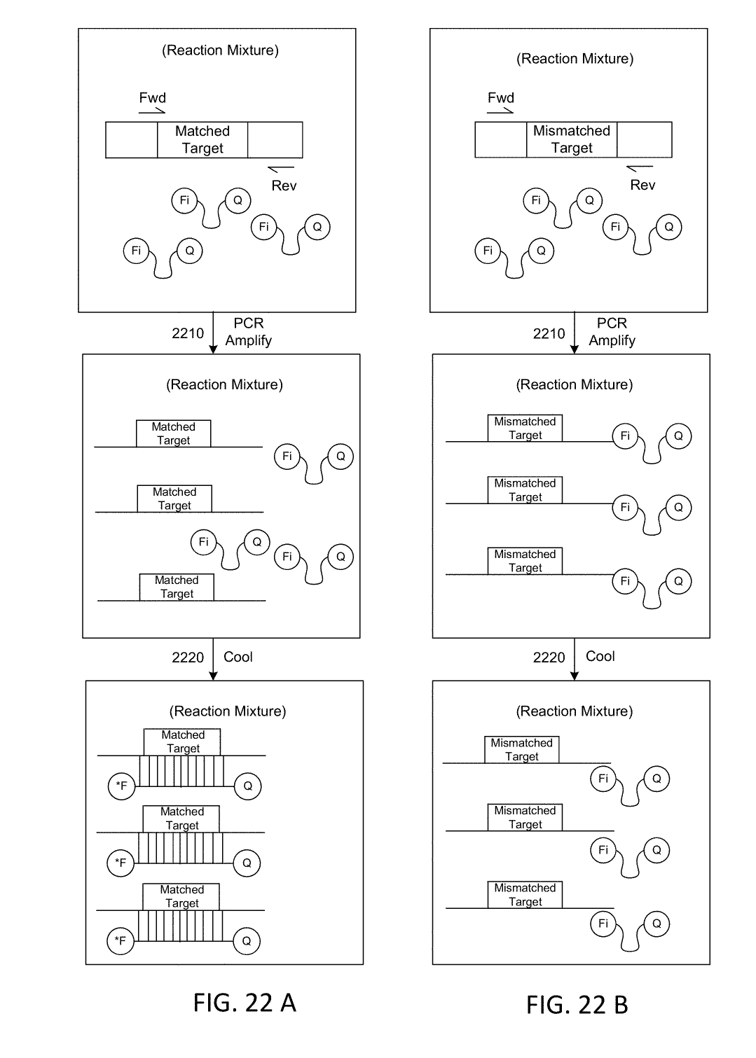

The methods described herein can be used for allelic discrimination assays. FIGS. 22A-22B depict exemplary embodiments of a method for allelic discrimination. In FIG. 22A, a reaction mixture or reaction volume can comprise a template nucleic acid, a forward primer and optionally a reverse primer designed to amplify a region comprising a locus. The locus can be suspected of harboring a mutation. The reaction mixture can further comprise a probe for sensitive detection of amplicons that, when free in solution, generally does not emit a detectable signal. The probe can be an allele-specific probe that is designed to be perfectly matched to a target harboring a particular allele of a locus. In step 2210, PCR amplification can result in the generation of a plurality of amplicons comprising the perfectly matched target. In some cases, the amplicons comprise single-stranded amplicons. In some cases, the amplicons can be double stranded amplicons. In such cases, following PCR amplification the double stranded amplicons can be denatured, e.g., by heating the reaction mixture to 90-100° C. (not shown). In some cases, PCR amplification cycling parameters are configured as to minimize hybridization of the probe to the perfectly matched template during the PCR reaction. In a next step 2220, the reaction mixture is cooled so as to allow hybridization of the probe to the perfectly matched target. In some cases, the hybridization of the probe increases the distance between the detectable moiety and the quencher, enabling detection of the detectable moiety. In FIG. 22B, the target harbors a different allele of the locus. Accordingly, the target is mismatched to the probe. In step 2210, PCR amplification can result in the generation of a plurality of amplicons comprising the mismatched target. In some cases, the amplicons comprise single-stranded amplicons. In some cases, the amplicons can be double stranded amplicons. In such cases, following PCR amplification the double stranded amplicons can be denatured, e.g., by heating the reaction mixture to 90-100° C. (not shown). In some cases, PCR amplification cycling parameters are configured as to minimize hybridization of the probe to the template during the PCR reaction. In a next step 2220, the reaction mixture is cooled so as to allow hybridization of the probe to the target. However, due to the probe/template mismatch the hybridization of the probe to the target can be reduced and/or minimized. In such cases, the probe can remain largely free in solution and therefore remain quenched. In some embodiments, a reaction mixture can comprise a plurality of probes. In particular embodiments, each probe of the plurality of probes is specific for a specific allele of a locus. In some embodiments, each probe of the plurality of probes comprises a distinct detectable moiety that is detectably distinct from other moieties of the probes.

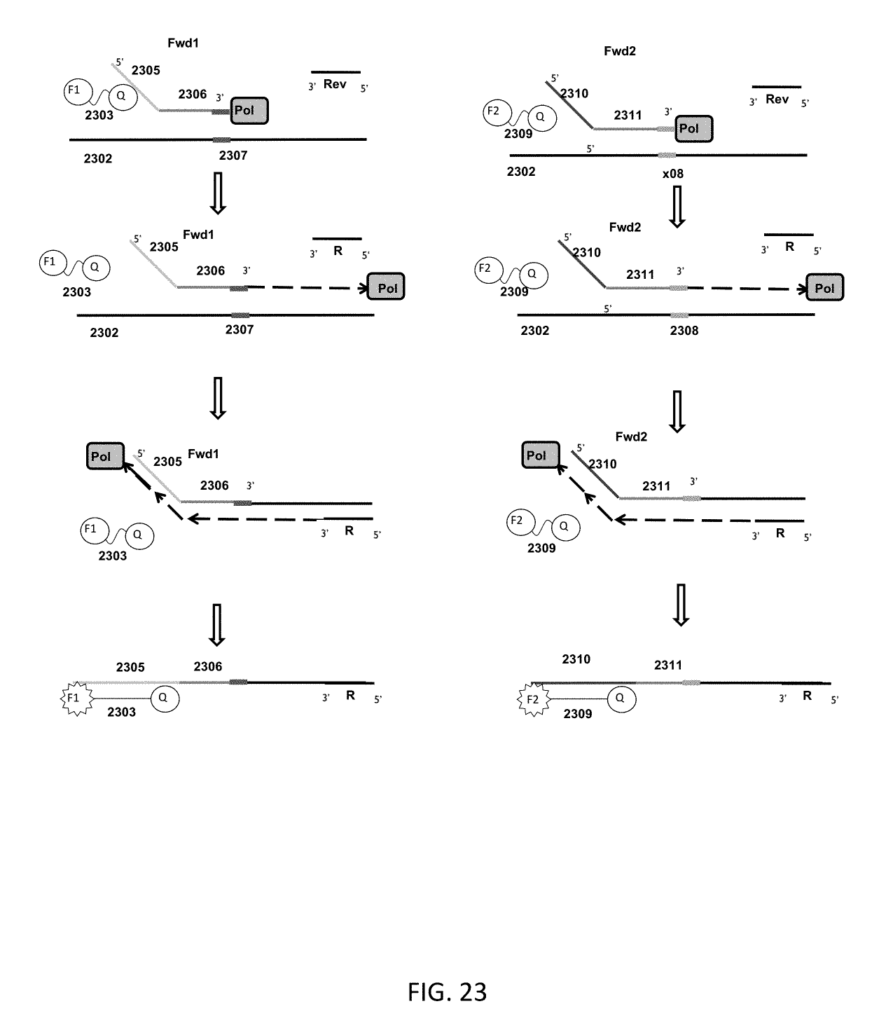

FIG. 23 depicts another exemplary embodiment of a digital PCR method for allele-detection, which utilizes low-Tm probes as described herein for sensitive detection of amplicons in combination with oligonucleotide primers as described herein which comprise (1) a template binding region and (2) a probe binding region. In FIG. 23, a reaction mixture or reaction volume can comprise a template nucleic acid 2302 which comprises either a wild-type allele 2307 or mutant allele 2308. The reaction mixture can further comprise a plurality of allele-specific forward primers. The allele-specific forward primers can include a first allele-specific forward primer Fwd1 (e.g., specific for a wild-type allele), and at least a second allele-specific forward primer Fwd2 (e.g., specific for a mutant allele), each designed to amplify a target polynucleotide 2302 suspected of harboring a mutation at a locus. Fwd1 can comprise a wild-type barcode region 2305 which does not generally hybridize to template nucleic acid 2302. The wild-type barcode region 2305 may contain a wild-type barcode sequence that specifically hybridizes a wild-type low Tm probe, but does not substantially hybridize a mutant low Tm probe. Fwd1 can further comprise a template binding region 2306 which is designed to hybridize to the target polynucleotide 2302, and which contains a nt at or near (e.g., within 1-3 nts) a 3′ end which is complementary to a wild-type allele 2307. One of the forward primers can be a mutant-specific forward primer that is complementary to the mutant allele at the site that overlays the mutation. Fwd2 can comprise a mutant barcode region 2310 which does not generally hybridize to a template nucleic acid. The mutant barcode region may contain a mutant barcode sequence that specifically hybridizes a mutant low Tm probe, but does not substantially hybridize to the wild-type low Tm probe. Fwd2 can further comprise a template binding region 2311 which is designed to hybridize to the target polynucleotide 2302, and which contains a nt at or near (e.g., within 1-3 nts) a 3′ end which is complementary to a wild-type allele 2308. The forward primers Fwd1 and Fwd2 may each further comprise a deliberate mismatch nucleotide adjacent to or within 1-3 nucleotides from the nt that overlays the mutation. However, in some cases, the forward primers do not further comprise a deliberate mismatch nucleotide adjacent to or within 1-3 nucleotides from the nt that overlays the mutation. The reaction mixture may further comprise wild-type low Tm probe 2303 and a mutant low Tm probe 2309. The wild-type low Tm probe 2303 may be designed to specifically hybridize to the reverse complement of the wild-type barcode region 2305. The mutant low Tm probe 2309 may be designed to specifically hybridize to the reverse complement of the mutant barcode region 2310. The wild-type and mutant low Tm probes 2303 and 2309 may comprise spectrally distinct fluorphores F1 and F2. The reaction mixture may further comprise a reverse primer (“Rev”). The reverse primer may be present in an excess amount as compared to the amount of forward primers, which are present in limited amounts. The reaction mixture may further comprise a stable DNA polymerase “Pol”, and dNTPs and other components for carrying out an amplification reaction. In a first step, template DNA molecules are contacted with the reaction mixture described above. Forward primers Fwd1 and Fwd2 may hybridize to template DNA containing either the wild-type allele 2307 and/or mutant allele 2308. Accordingly, there is a mismatch between the 3′ terminal base of 2306 and mutant allele 2308, and a mismatch between the 3′ terminal base of 2311 and wild-type allele 2307. In a next step, the DNA polymerase “Pol” can promote efficient extension of the Fwd1 primer annealed to template DNA containing 2307 wild-type allele, but does not promote efficient extension of the Fwd1 primer annealed to template DNA containing 2308 mutant allele (due to a greater mismatch between Fwd1 and 2308). By the same token, polymerase “Pol” can promote efficient extension of the Fwd2 primer annealed to template DNA containing the 2308 mutant allele but does not promote efficient extension of the Fwd2 primer annealed to template DNA containing the 2307 wild-type allele (due to a greater mismatch between Fwd2 and 2307). Efficient extension results in extension products comprising the reverse complement of the wild-type barcode 2305 or the reverse complement of the mutant barcode 2310. In a second (and any subsequent round) of amplification, the excess Rev primer can anneal to the extension products comprising either 2305 or 2310 and (after exhaustion of limiting primers Fwd1 and Fwd2), promote linear amplification of the extension products comprising either barcodes 2305 or 2310. During the amplification cycles, the wild-type and mutant probes low-TM probes 2303 and 2309 do not hybridize to the barcodes 2305 and/or 2310. After amplification cycles are completed, the reaction mixture can be cooled, e.g., to about 25° C., thereby allowing the probes 2303 and 2309 to hybridize to their respective barcodes 2305 and 2310. Hybridization of the probes to their respective barcode regions releases the fluorphores F1 and F2 from their quenchers (Q) and promotes fluorescence of the fluorophores.

### Applications of Sensitive Detection of Amplicons

The methods and kits of the present invention may be used for the sensitive and accurate analysis of nucleic acids isolated from a subject. Such detection and analysis can be useful for a wide range of applications, including but not limited to diagnostic and/or therapeutic purposes. By way of example only, the detection methods may be used for the detection of a mutation in a subject, for diagnosing a disease in a subject, for monitoring disease progression in a subject, for aiding in the selection of a therapeutic regimen for a disease in a subject, for determining the effectiveness of an therapy targeting a disease in a subject, or for evaluating disease prognosis in a subject. Exemplary subjects are described herein. In some embodiments, nucleic acid from a biological sample isolated from the subject is analyzed using the methods and/or kits described herein for sensitive detection of amplicons.

Exemplary biological samples are described herein. In particular embodiments, the sample is a tumor sample. In some embodiments, the tumor sample is processed prior to the probe-based assay. Processing can comprise fixation in a formalin solution, followed by embedding in paraffin (e.g., is a FFPE sample). Processing can alternatively comprise freezing of the sample prior to conducting the probe-based assay. In some embodiments, the sample is neither fixed nor frozen. The unfixed, unfrozen sample can be, by way of example only, stored in a storage solution configured for the preservation of nucleic acid.

In some embodiments, non-nucleic acid materials can be removed from the starting material using enzymatic treatments (for example, with a protease). The sample can optionally be subjected to homogenization, sonication, French press, dounce, freeze/thaw, which can be followed by centrifugation. The centrifugation may separate nucleic acid-containing fractions from non-nucleic acid-containing fractions.

Nucleic acid can be isolated from the biological sample using any means known in the art. For example, nucleic acid can be extracted from the biological sample using liquid extraction (e.g, Trizol, DNAzol) techniques. Nucleic acid can also be extracted using commercially available kits (e.g., Qiagen DNeasy kit, QIAamp kit, Qiagen Midi kit, QIAprep spin kit).

Nucleic acid can be concentrated by known methods, including, by way of example only, centrifugation. Nucleic acid can be bound to a selective membrane (e.g., silica) for the purposes of purification. Nucleic acid can also be enriched for fragments of a desired length, e.g., fragments which are less than 1000, 500, 400, 300, 200 or 100 base pairs in length. Such an enrichment based on size can be performed using, e.g., PEG precipitations, an electrophoretic gel or chromatography material (Huber et al. (1993) Nucleic Acids Res. 21:1061-6), gel filtration chromatography, TSK gel (Kato et al. (1984) J. Biochem, 95:83-86), which publications are hereby incorporated by reference.

Polynucleotides extracted from a biological sample can be selectively precipitated or concentrated using any methods known in the art.

The probes, reaction mixtures, kits, methods, and systems described herein for sensitive detection of amplicons can be utilized in the assessment of a disease in a subject. In some embodiments, the disease is a cancer. The method can comprise determining the presence, absence, or level of a mutation in any number of genes of interest. For example, the method can comprise determining the presence, absence or level of a mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, or more than 200 genes of interest. The method can comprise determining the presence, absence or level of a mutation in 1-3, 2-5, 4-10, 5-20, 10-50, 30-100, 50-150, 70-200, or more than 200 genes of interest. Genes of interest can include any cancer-related genes known in the art. Cancer-related genes are described herein. In some embodiments, the genes of interest are suspected of harboring a SNP, insertion, deletion, or translocation. In some embodiments, the genes of interest are suspected of harboring a copy number variation.

The method can involve determining the presence, absence, or level of a copy number variation in a subset of genes. The method can involve determining a copy number variation in 1, 2, 3, 4, 5, 6, 7, 8, 9, 10, 11, 12, 13, 14, 15, 16, 17, 18, 19, 20, 25, 30, 35, 40, 45, 50, or more than 50 genes, e.g., cancer-related genes, relative to a set of reference genes. In some cases, the method involves determining a copy number variation of one or more genes (e.g., 1, 2, 3, 4, 5, 6, 7, 8, 9, 10, 11, 12, 13, 14, 15, 16, 17, 18, or 19 genes). The genes can be selected from the group consisting of MET, FGFR1, FGFR2, FLT3, HER3, EGFR, mTOR, CDK4, HER2, RET, DDR2, AURKA, VEGFA, CDK6, JAK2, BRAF, and SRC. In some cases, the method involves determining a copy number variation of one or more genes (e.g., 1, 2, 3, 4, 5, 6, 7, 8, 9, 10, 11, 12, or 13 genes) selected from the group consisting of EGFR, AURKA, VEGFA, FGFR1, CDK4, EFBB2, CDK6, JAK2, MET, BRAF, ERBB3, and SRC. The reference genes can be, e.g., HADH, ZFP3, RNaseP. The method of assessing cancer can comprise conducting a probe-based assay for sensitive detection of amplicons as described herein, using a probe for sensitive detection of amplicons as described herein.

One or more methods of the invention can be used for copy number variation analysis. The methods for copy number variation can comprise two assays. The two assays can be a target assay and a reference assay. The target assay can utilize a primer/probe set that is specific for a target region that is suspected of harboring a copy number variation. The reference assay can utilize a primer/probe set that is specific for a reference region that is known or suspected to not harbor a copy number variation. The target and reference regions may be on the same or on different chromosomes. The target region can be a region in any chromosome, for example, a region in human chromosome 13, 18, 21, X, or Y. Copy number variation of the target region can be estimated by any means known in the art, for example, by a ratio between the estimated target vs. reference concentration, or by a statistical analysis of the difference in concentration of the target vs. the reference region.

In some embodiments, a method for assessing cancer comprises copy number variation analysis of 12 genes selected from the group consisting of VEGFA, EGFR, CDK6, MET, BRAF, FGFR1, JAK2, HER3, CDK4, HER2, SRC, and AURKA in a DNA sample originating from a human subject in need thereof. In some embodiments, the DNA sample is from a tumor biopsy or a tissue biopsy suspected of harboring tumor DNA. In some embodiments the DNA sample is from a liquid biological sample isolated from the subject. Exemplary liquid biological samples are described herein. In some embodiments, the DNA sample is partitioned into a plurality of reaction mixtures. The DNA sample may be partitioned such that each reaction mixture comprises 0-2 DNA template molecules. Each reaction mixture can comprise a primer/probe set for sensitive detection of amplicons as described herein. A primer/probe set can be designed to amplify a region of interest within a gene suspected of having copy number variation (e.g., a gene amplification). Each primer/probe set can comprise a forward primer, reverse primer, and probe. In particular embodiments, each primer/probe set comprises a primer in excess amounts (e.g., excess primer) compared to a reverse primer in limiting amounts (e.g., limiting primer). Each primer/probe set can comprise a low Tm probe that is designed to selectively hybridize to a region that is located between the excess and limiting primer. In some embodiments, a region suspected of having copy number variation also harbors a site of a known mutation. In some embodiments, the low Tm probe is designed to overlay the mutation site. In some embodiments, the low Tm probe is designed to correspond to the wild-type allele. In some cases the low Tm probe is designed to have a greater number of mismatches to the mutant allele than to the wild-type allele. In some embodiments, each reaction mixture also comprises a primer/probe set for a reference gene. The reference gene can be, e.g, RNaseP30, HADH, ZFP3. In some embodiments, the reference primer/probe set comprises a forward primer, reverse primer, and probe. In particular embodiments, the reference primer/probe set comprises an excess primer and a limiting primer which is designed to amplify a region of the reference gene. In some embodiments, the reference primer/probe set further comprises a low Tm probe which is designed to hybridize to a region of the reference gene that is located between the excess and limiting primer. In some embodiments, the partitioned reaction mixtures are subject to an amplification reaction. In some embodiments, the amplification reaction comprises PCR cycles, wherein the PCR cycles do not comprise a temperature step that is results in substantial annealing of the low-Tm probe. In some embodiments, a sufficient number of PCR cycles are performed to exhaust the limiting primer, thus resulting in linear amplification utilizing the excess primer. In some embodiments, following the PCR cycles, the reaction mixtures are cooled to a temperature which allows for annealing of the low Tm probes to the amplification products. In some embodiments, following annealing of the low Tm probes, the reaction mixtures are assessed and enumerated for fluorescent detection of the annealed low Tm probes. In some embodiments, a CNV call is generated based on the assessment and enumeration.

In some embodiments, one or more methods for sensitive detection of amplicons comprise partitioning the reaction mixture and nucleic acid sample into discrete volumes prior to amplification. For example, the one or more methods can comprise digital PCR. Methods, kits, and systems for partitioning/digital PCR are described herein.

### Kits for Sensitive Detection of Amplicons

Also provided in the invention are kits for the sensitive detection of amplicons. Kits may include one or more oligonucleotide primers and probes as described herein. In some embodiments, the primers and/or probes are capable of selectively detecting an individual allele of a locus. Kits can include, for example, one or more primer/probe sets. Exemplary primer/probe sets are described herein. For example, kits can include primer/probe sets for MET, FGFR1, FGFR2, FLT3, HER3, EGFR, mTOR, CDK4, HER2, RET, HADH, ZFP3, DDR2, AURKA, VEGFA, CDK6, JAK2, BRAF, SRC and RPP30. Kits may further comprise instructions for use of the one or more primer/probe sets, e.g., instructions for practicing a method of the invention. In some embodiments, the kit includes a packaging material. As used herein, the term “packaging material” refers to a physical structure housing the components of the kit. The packaging material can maintain sterility of the kit components, and can be made of material commonly used for such purposes (e.g., paper, corrugated fiber, glass, plastic, foil, ampules, etc.). Kits can also include a buffering agent, a preservative, or a protein/nucleic acid stabilizing agent. Kits can also include other components of a reaction mixture as described herein. For example, kits may include one or more aliquots of thermostable DNA polymerase as described herein, and/or one or more aliquots of dNTPs. Kits can also include control samples of known amounts of template DNA molecules harboring the individual alleles of a locus. In some embodiments, the kit includes a negative control sample, e.g., a sample that does not contain DNA molecules harboring the individual alleles of a locus. In some embodiments, the kit includes a positive control sample, e.g., a sample containing known amounts of one or more of the individual alleles of a locus.

### Systems for Sensitive Detection of Amplicons

Also provided in the invention are systems for the sensitive detection of amplicons. In some embodiments, the system provides a reaction mixture for sensitive detection of amplicons as described herein. In some embodiments the reaction mixture is admixed with a DNA sample and comprising template DNA. In some embodiments, the system further provides a droplet generator, which partitions the template DNA molecules, probes, primers, and other reaction mixture components into multiple droplets within a water-in-oil emulsion. Exemplary droplet generators are described herein.

### EXAMPLES

### Example 1

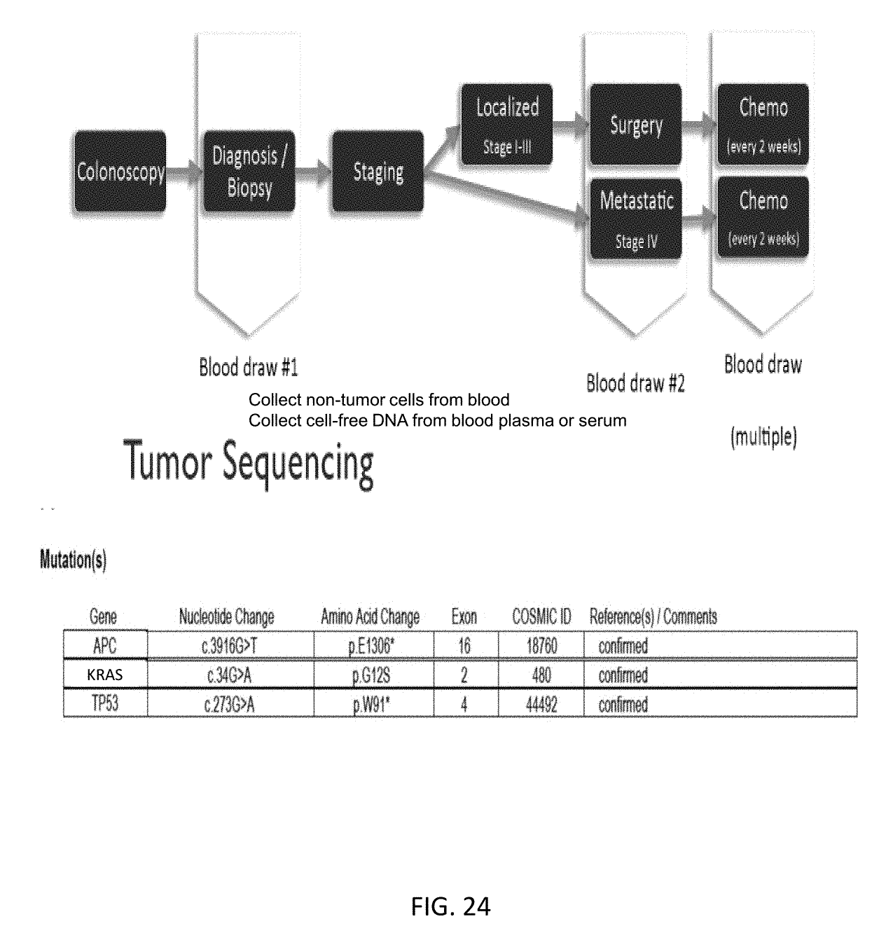

FIG. 24 depicts a method used to assess a cancer in a subject. A subject had a colonoscopy and is discovered to harbor a colon tumor. A tumor biopsy and blood draw were collected from the subject at time point 0, and are used to aid in the diagnosis of colon cancer in the subject. The tumor and normal cells from the first blood draw were sequenced. Sequencing revealed the presence of three mutations in the subject's tumor. The mutations were point mutations in the APC, KRAS, and TP53 genes. The stage of the subject's cancer was determined. The subject underwent a first treatment (surgery) to remove the tumor. Upon the first treatment, a second blood draw was performed. It was determined that the subject's tumor had metastasized. The subject was administered as second therapy (chemotherapy) to manage the cancer. Subsequent blood draws are performed to assay the mutational status of the three genes in cell-free DNA from the blood.

### Example 2: Validation Assay for a Tumor-Specific Mutation in the Subject with Colon Cancer

NCI-H1573 (CRL-5877) cell lines harboring the KRAS G12A mutation (mu) were obtained as frozen stocks from the American Type Culture Collection (ATCC). Genomic DNA (gDNA) was prepared from cell line material using a commercially available kit (DNeasy Blood & Tissue kit, QIAGEN), according to the manufacturer's suggested protocol. Estimates of DNA concentration were obtained spectrophotometrically by measuring the OD260 (NanoDrop 1000, Thermo Fisher Scientific Inc.).

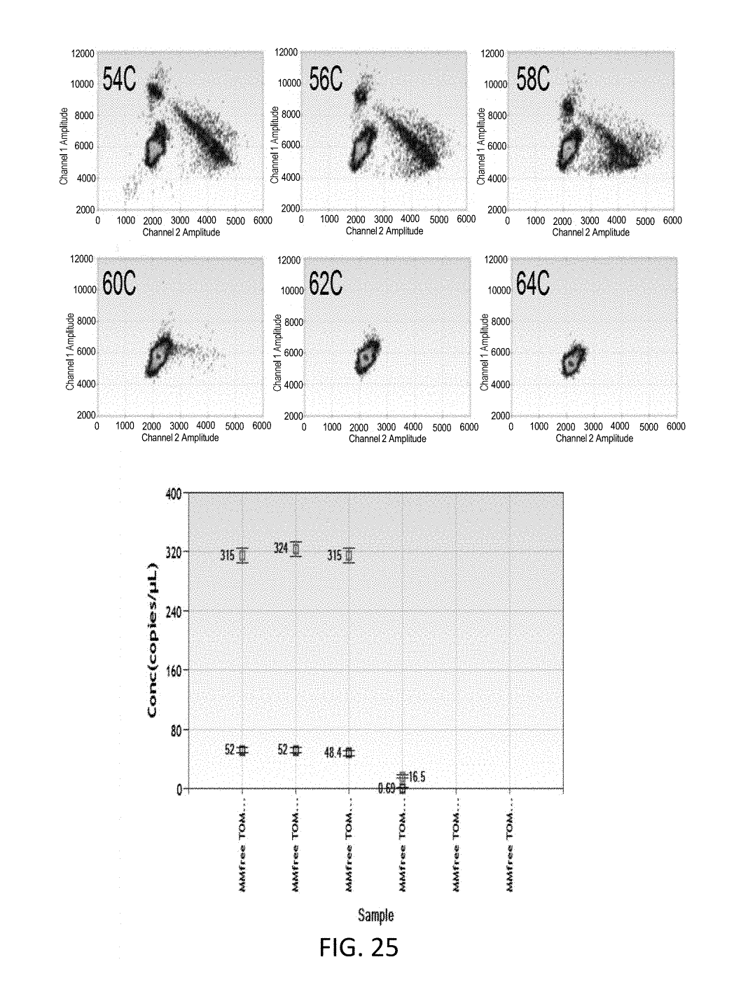

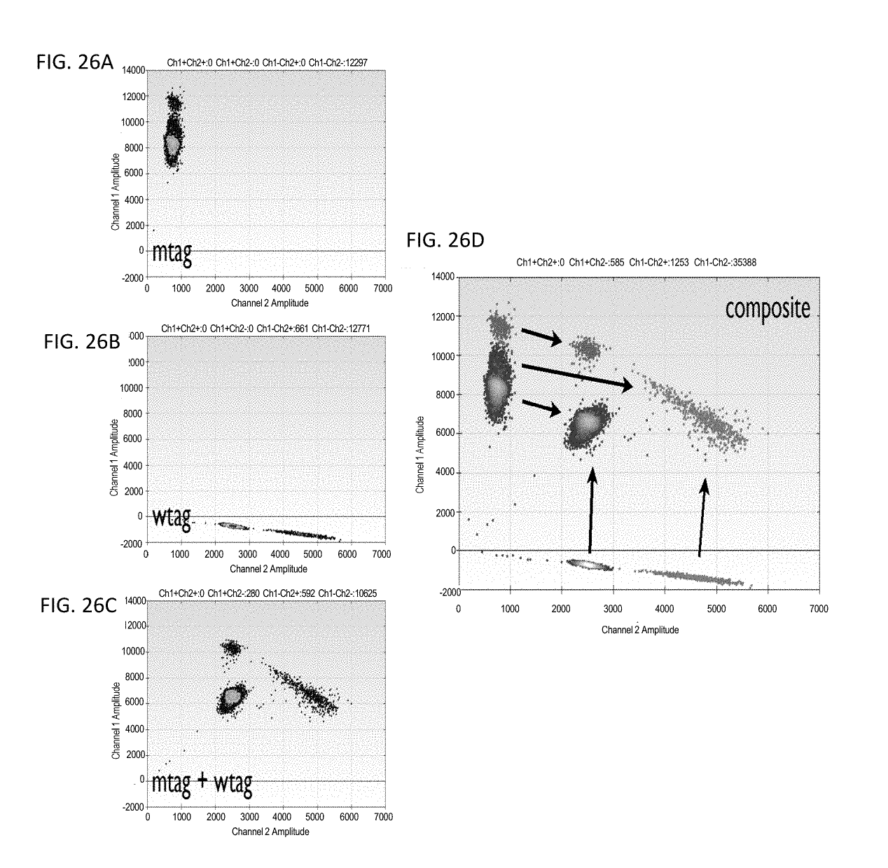

Genomic DNA from NA18507 cell lines was used as a surrogate for wild-type DNA (wt) and obtained as purified stocks (Coriell). Two microliters of a mixture containing wt (30 ng) and mu (6 ng) DNA was assembled into a 20 μl ddPCR reaction mix from 2×ddPCR supermix for probes, 0.2 uM final of each forward primer (wt: 5′-AGATTACGCGGCAATAAGGCTCGGTTGGCATTGGATACTACTTGCCTACGCCACC-3′ (SEQ ID NO: 1)); mu: 5′AATAGCTGCCTACATTGGGTTCGGTCGTAACTTAGGAACTCTTGCCTACGCCAG C-3′(SEQ ID NO: 2), 0.4 uM of reverse primer (5′-CCTGCTGAAaAATGACTGAAT-3′ (SEQ ID NO: 3)), and 1 uM each of reporter probes (wt: 5′→1-1EX-CCAACCGAG/ZEN/CCTTATTGCCG-IABkFQ-3′ (SEQ ID NO: 4); mu: 5′-FAM-ACTTACGAC/ZEN/CGAACCCAATGTAGG-1ABkFQ-3′ (SEQ ID NO: 5)). Each PCR mixture was then converted into droplets for analysis via the QX100 ddPCR system according to the manufacturer's suggestions. Annealing temperature was varied to determine the optimal conditions for segregating and quantifying the wt (HEX) and mu (FAM) droplet signals (FIG. 25). Resulting clusters were deconvoluted (FIGS. 26A-26D) by using ddPCR mixtures containing only the mu (FIG. 26A), only the wt (FIG. 26B), or both probes (FIG. 26C) to assign membership of each cluster as mu or wt.

### Example 3: DNA Sample Processing for Target-Enriched Library Preparation

100 ng (˜33000 genome equivalents) of fragmented and/or damaged DNA (e.g. from FFPE samples) was first repaired by excising oxidized and abasic sites through the use of a cocktail of repair enzymes (Endo VIII, Fpg, and UDG) in the presence of T4 polynucleotide kinase, 1 mM ATP, and 15% PEG-8000 in 1× ligase reaction buffer at a final reaction volume of 100 ul to generate DNA fragments that are terminated by a 5′-phosphate and a 3′-OH.

Repaired DNA was purified using a commercially available kit (GeneJet; Thermo Scientific). Eluted DNA (50 ul) was then concentrated via sedimentation with PEG-8000 (20% final) in the presence of LPA and Tris buffer containing 10 mM Mg²⁺. The resulting pellet was rinsed once with 0.5 ml of 70% ethanol and air-dried for 5 minutes.

### Example 4: 5′ Adaptor Ligation of DNA Fragments

Repaired DNA prepared as above was resuspended in 2 ul of nuclease-free water. Repaired DNA can then be fully or partially denatured either chemically, through brief treatment with alkali (NaOH or KOH) followed by neutralization with sodium acetate; or, preferably heat denatured with rapid cooling on ice (3 min at 95 deg C).

Repaired DNA was pre-adenylated by combining the following components in an adenylation reaction mixture as shown in Table 2:

TABLE 2
Adenylation reaction mixture (DNA sample).

| | |
|---|---|
| 10x NEB4 buffer | 0.5 μl |
| 1 mM ATP | 0.5 μl |
| Thermophilic RNA ligase | 0.5 μl |
| 50% PEG-8000 | 1.5 μl |
| DNA sample + water | 2.0 μl |

Following incubation for 1 hour at 65 deg C, the following components (Table 2) were added to the adenylation reaction mixture. To test the effect of additional ligase, 2 μl of ligase or no additional ligase was added to the ligation mix.

TABLE 3
Ligation Mix

| | | |
|---|---|---|
| 10x NEB4 buffer | 4.5 | μl |
| 100 uM adaptor | 1 | μl |
| 25 mM Manganese acetate | 5.0 | μl |
| 50% PEG-8000 | 13.5 | μl |
| Thermophilic RNA ligase | 0 or 2 | μl |

| | |
|---|---|
| water | (up to final volume of 50 μl) |

The reaction was incubated for 1 hr @ 65° C., followed by heat inactivation for 10 min @ 80° C., then by 3 min @ 95° C. 1 μl of protease was then added and the reaction incubated for 30 min @ 37° C. followed by heat inactivation for 15 min @ 75° C. The resulting ligation products were sedimented to remove unreacted adaptors and washed as described above.

### Example 5: (3′-End Adaptor Ligation)

Repaired DNA as prepared in Example 1 or 5′-adapted DNA libraries as prepared in Example 2 were resuspended in 2 μl of nuclease-free water. This can then be fully or partially denatured either chemically, through brief treatment with alkali (NaOH or KOH) followed by neutralization with sodium acetate; or, preferably heat denatured with rapid cooling on ice (3 min @ 95 C).

The 3′ adaptor DNA was pre-adenylated combining the following components in an adenylation reaction mixture as shown in Table 4:

TABLE 4
Adenylation reaction mixture (3′ adaptor)

| | |
|---|---|
| 10x NEB4 buffer | 0.5 μl |
| 50% PEG-8000 | 1.5 μl |
| 1 mM ATP | 0.5 μl |
| 100 uM adaptor | 2.0 μl |
| Thermophilic RNA ligase | 0.5 μl |

Following incubation for 1 hour at 65 deg C, the following components (Table 4), were added to the adenylation reaction mixture. Denatured DNA refers to either repaired DNA as prepared in Example 1 or 5′ adapted DNA as prepared in Example 2. To test the effect of additional ligase, 2 μl of ligase or no additional ligase was added to the ligation mix.

TABLE 5
Ligation Mix

| | | |
|---|---|---|
| Adenylation reaction mixture (3′ adaptor) | 5.0 | μl |
| 10x NEB4 buffer | 4.5 | μl |
| Denatured DNA | 2 | μl |
| 25 mM Manganese acetate | 5.0 | μl |
| 50% PEG-8000 | 13.5 | μl |
| Thermophilic RNA ligase | 0 or 2 | μl |

| | |
|---|---|
| water | up to final volume of 50 μl |

The reaction was incubated for 1 hr @ 65 deg C, followed by heat inactivation for 10 min @ 80 C, then by 3 min @ 95° C.

1 μl of protease is added and the reaction incubated for 30 min @ 37° C. followed by heat inactivation for 15 min @ 75° C.

The resulting ligation products were sedimented and washed as above to remove unreacted adaptors and resuspended in 10 μl of 1×NEB4 with 0.1% BSA.

### Example 6: Quantitation of Ligation Efficiency Via ddPCR

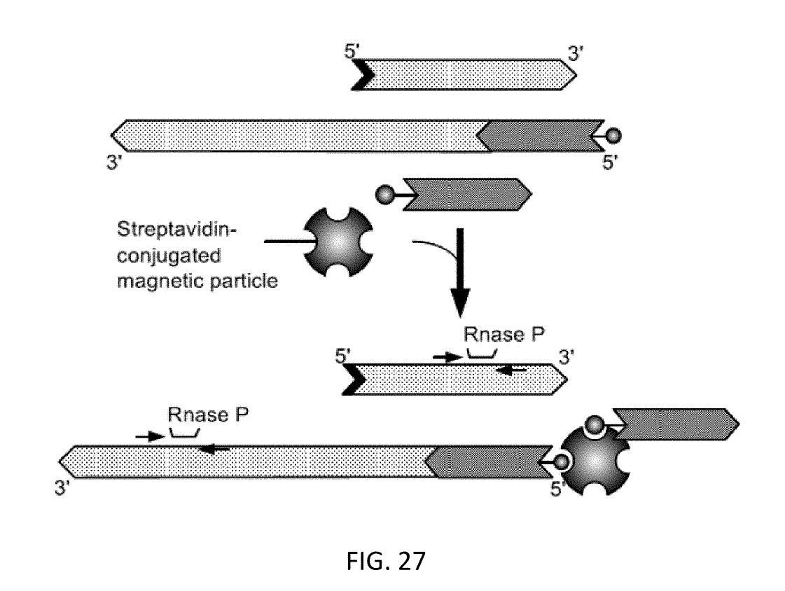

FIG. 27 depicts an exemplary embodiment of a method for quantitating efficiency of a ligation method described herein. Ligation of nucleic acid molecules (NA) to a biotinylated oligonucleotide (5′ or 3′ adaptor) was performed as described above. The ligation reaction can result in ligation products (ligated NA) comprising biotinylated oligonucleotides covalently linked to sample nucleic acids, and can possibly also result in unligated sample nucleic acids (unligated NA). Ligation products were sedimented through centrifugation for 20 min @ 22,000 g. Supernatant was removed and the pellet was resuspended in 5 ul of 0.1×TET Buffer (1 mM TrisHC1, 0.1 mM EDTA, 0.05% Tween-20, pH=8). Resuspended pellet was made up to a final volume of 50 μl in 1×NEB+0.1% BSA and 10 μl of Streptavidin-ferrofluids comprising streptavidin-conjugated magnetic particles (MagCellect, R&D Systems, Minneapolis, Minn.) pre-washed with 1×NEB4. Following incubation for 15 min at room temperature, the mixture was magnetized for 5 minutes. The supernatant containing free and therefore un-ligated sample nucleic acids was removed. The remaining bound material comprising ligation products was resuspended in 50 μl of 1×NEB4 +0.1% BSA. Five microliters of the bound and unbound fractions were interrogated via ddPCR with taqman assays designed to the RNaseP gene locus. Ligation efficiency was calculated as [bound signal]/([bound signal]+[unbound signal]).

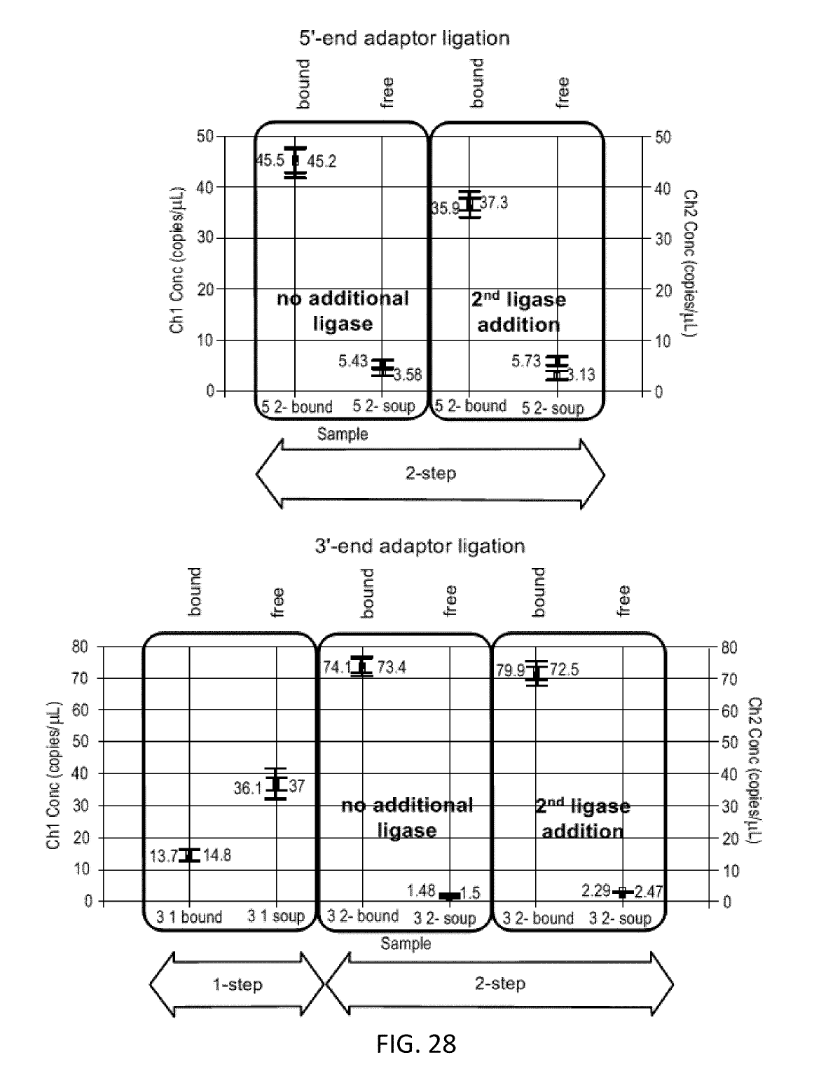

The ligation efficiencies of the 5′ and 3′ adaptor library preparations (Examples 2 and 3) were quantified as above. FIG. 28 depicts ddPCR results for the 5′ end adaptor ligation and 3′ end adaptor ligation reactions, respectfully. Results depicted in FIG. 28, top panel, indicate that 2-step 5′ end adaptor ligation reactions in which the adenylation and ligation steps were performed serially were highly efficient. Without additional ligase, the average concentration of bound signal was 45.35 copies/μl, while the average concentration of unbound signal was 4.505 copies/μl, indicating a ligation efficiency of 90.9%. With additional ligase, the average concentration of bound signal was 36.6 copies/μl, while the average concentration of unbound signal was 4.43 copies/μl, indicating a ligation efficiency of about 89%.

Results depicted in FIG. 28, bottom panel, indicate that two-step 3′ end adaptor ligation reactions in which the adenylation and ligation steps were performed serially were highly efficient. For the traditional 1-step ligation reaction in which adenylation and ligation steps co-occur in one reaction, the average concentration of bound signal was 14.25 copies/μl, while the average concentration of unbound signal was 36.55 copies/μl, indicating a ligation efficiency of 28%. By contrast, for the two-step 3′ end adaptor ligations performed without further addition of ligase, the average concentration of bound signal was 73.75 copies/μl, while the average concentration of unbound signal was 1.49 copies/μl, indicating a ligation efficiency of 98%. For the two-step 3′ end adaptor ligations performed with further addition of ligase after adenylation, the average concentration of bound signal was 71.7 copies/μl, while the average concentration of unbound signal was 2.38 copies/μl, indicating a ligation efficiency of 96.8%. From these results, the possibility of serially performing adenylation and ligation reactions in a single reaction mixture was demonstrated. Furthermore, it was determined that the two-step process in which adenylation and ligation are performed separately in a single reaction mixture greatly enhances ligation efficiency.

Another surprising result is that further addition of ligase to the reaction mixture following adenylation does not appear to enhance ligation efficiency, despite the fact that not only ATP but ligase concentration is diluted to the same degree by the further addition of reaction components (e.g., water, buffer, PEG, Mn²⁺) upon commencement of the ligation step. Without wishing to be bound by theory, it is possible that adenylated donor nucleic acid molecules remain complexed to the ligase enzyme. Upon dilution of ATP and addition of acceptor nucleic acid molecules, the complexed ligase enzyme can be released from inhibition and catalyze the ligation of an acceptor nucleic acid molecules to the adenylated nucleic acid molecule.

### Example 7: Effect of Adaptor Length and PEG-8000 on Ligation Efficiency

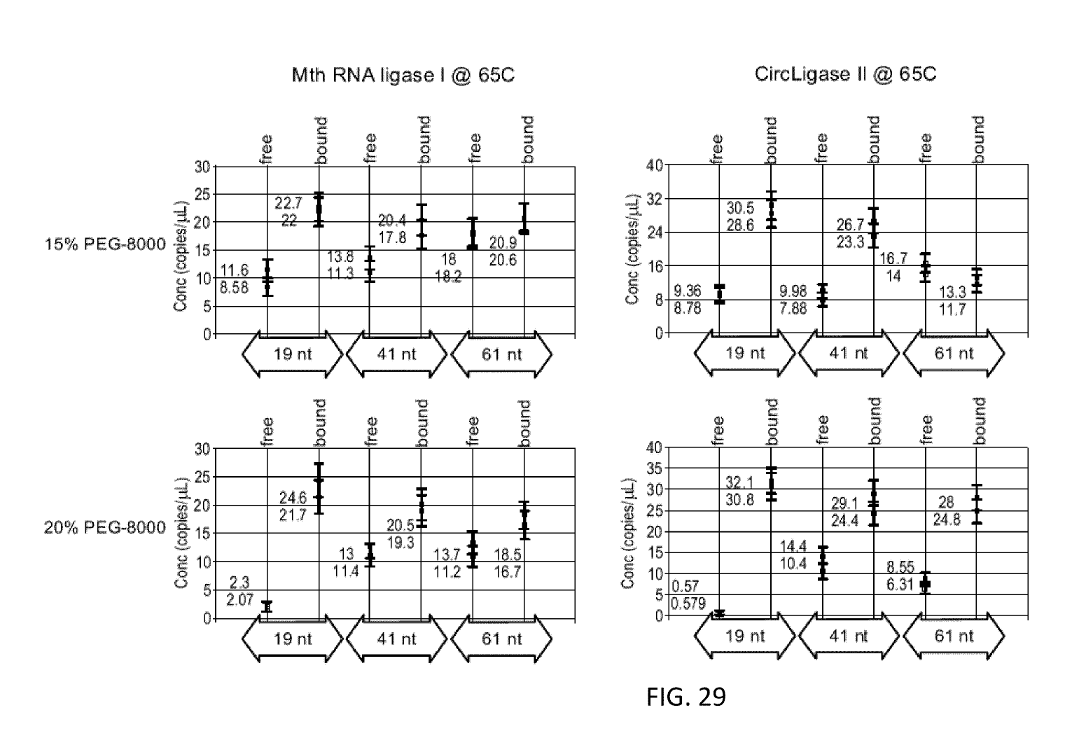

Sample DNA was prepared and adenylated as described in Example 2 in a reaction mixture comprising 15 or 20% PEG-8000. Following adenylation, adaptors of length 19 nt, 41 nt, or 61 nt were ligated to the adenylated DNA as described in Example 4. Either Mth RNA ligase or CircLigase II were used as the ATP-dependent RNA ligase. FIG. 29 depicts ddPCR results for the above ligation reaction conditions. The results indicate that adaptor length may affect ligation efficiency, and that in cases wherein CircLigase II is used as the RNA ligase, 20% PEG-8000 may be used to increase the efficiency of long (e.g., 61 nt) adaptor ligation reactions.

### Example 8: Effect of Mn²⁺ vs. Incubation Temperature in 20% PEG-8000 on Ligation Efficiency

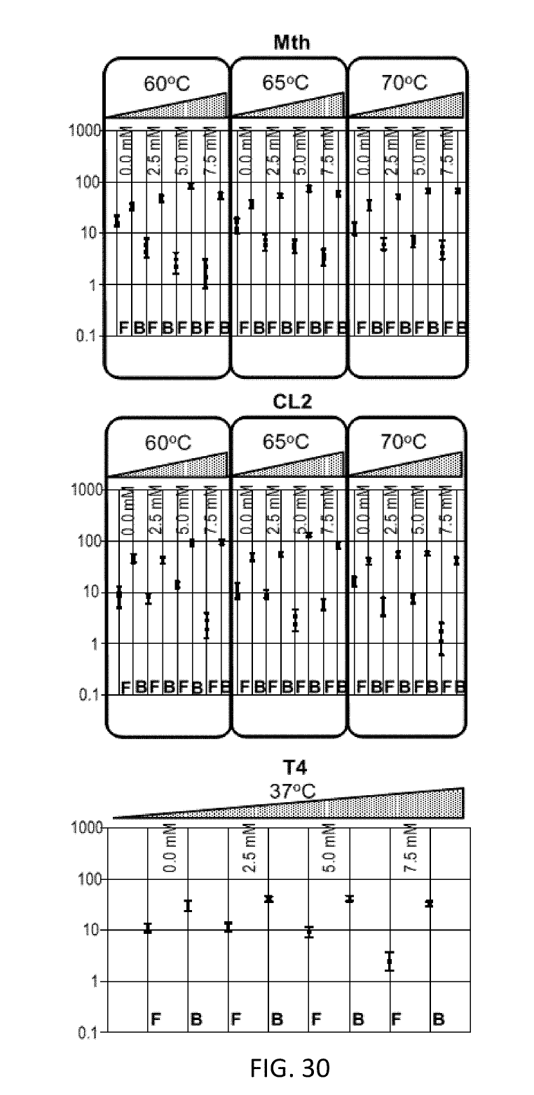

Sample DNA was prepared and adenylated using a two-step adenylation/ligation method as described in Example 4, in a reaction mixture comprising 20% PEG-8000. Either Mth RNA ligase, CircLigase II, or T4 RNA Ligase (representing commercially available ATP-dependent RNA ligases) were used. The adenylation and ligation reactions were incubated at 37, 60, 65, or 70 deg C for 1 hour each. The ligation reactions were conducted in the presence of 0, 2.5 mM, 5 mM, or 7.5 mM Mn²⁺. FIG. 30 depicts ddPCR results for the above ligation reaction conditions. The Y axis is shown in logarithmic scale. Accordingly, any differences in bound vs. free signal that is greater than the distance between the Y axis gridlines (e.g., labeled on the Y axis) indicates a ligation efficiency of 90% or greater. These results indicate that reaction conditions can be tailored to produce over 90% ligation efficiency for all commercially available ATP-dependent RNA ligases, and that Mn²⁺ appears to facilitate the ligation step.

### Example 9: (Optional) Linear Expansion of 3′-End Adapted Libraries

A 5 μl aliquot of a resuspended 3′-end library prepared according to Example 3 is assembled into the following mixture (Table 6) for linear expansion:

TABLE 6
Linear Expansion Reaction Mixture

| | |
|---|---|
| adapted DNA library | 10.0 μl |
| 5x Phusion buffer (New England Biolabs) | 20.0 μl |
| DMSO | 3.0 μl |
| 10 mM dNTP | 2.0 μl |
| 100 uM expansion primer | 0.5 μl |
| (at least partially complementary to adaptor) | |
| water | 67.0 μl |
| Phusion (2 U/μl) (New England Biolabs) | 1 μl |

The adapted library is expanded according to the following cycling parameters: 3 min at 98 deg C; 10 s at 98 deg C, 10 s at 68 deg C, 5 min at 72 deg C, 20 cycles; 5 min at 72 deg C; 4 deg C hold.

Upon completion, the entire reaction is incubated with 10 μl of Streptavidin-ferrofluids comprising streptavidin-conjugated magnetic particles (MagCellect, R&D Systems, Minneapolis, Minn.) prewashed with 1×NEB4, for 30 min at 37 deg C.

The solution is magnetized for 5 minutes, and the solution phase containing expanded library members are removed.

The solution phase is extracted with phenol:chloroform:isoamyl alcohol (25:24:1) and the aqueous layer precipitated with 1 volume of 5M NH4.acetate, and 1 volume of isopropanol.

After incubation for 20 min at −20 deg C, the solution is centrifuged for 30 min at 22,000 g at 4 deg C.

The resulting pellet is washed once with 500 μl of 70% ethanol, and air-dried for 5 minutes.

### Example 10: Oligo-Selective Finishing (Reverse OS-Seq)

DNA library members comprising a single 5′ adaptor sequence may undergo target-selective addition of a 3′ adaptor sequence. Methods for the addition of a 3′ adaptor sequence to desired target regions are described in, e.g., US Patent Application Publication No. 20120157322, hereby incorporated by reference. 5′-adapted libraries prepared according to Example 4, optionally expanded according to Example 9, are resuspended in 1×NEB4 with 0.1% BSA added to the following mix (Table 7):

TABLE 7
Annealing mixture

| | |
|---|---|
| adapted DNA library | 10.0 μl |
| 5x Phusion buffer | 16.0 μl |
| 4 uM OS-seq probeset | 5.0 μl |
| DMSO | 3.0 μl |
| water | 46.0 μl |

The above reaction mix is denatured and annealed under the following parameters: 2 min @ 95° C.; 10 s @ 95° C., −1° C./cycle, 0.1° C./s, 24 cycles; 30 min @ 72° C.

The annealed mixture is then extended by adding the following polymerase mixture (Table 8):

TABLE 8
polymerase mixture

| | |
|---|---|
| adapted DNA library | 80.0 μl |
| 5x Phusion buffer | 4.0 μl |
| 10 mM dNTPs | 2.0 μl |
| water | 13.0 μl |
| Phusion (2 U/μl) | 1.0 μl |

After incubation for 10 min @ 72° C., the reaction is brought to 37° C.

Unfinished fragments and unextended oligonucleotides can then be optionally removed by incubation with Exonuclease I or Exo-SAP IT for 30 minutes.

1 μl of protease is added and the reaction incubated for 30 min @ 37 C followed by heat inactivation for 15 min @ 75 C.

Reactions are then purified via sedimentation with 1 volume of a 2×PEGppt solution (1×NEB4, 10 ug LPA, 30% PEG-8000)

### Example 11: Oligo-Selective Finishing with Expansion (Reverse OS-Seq)

5′-adapted libraries prepared according to Example 4, optionally expanded according to Example 9, are annealed as described in Example 10.

Following incubation for 10 min @ 72° C., the products are expanded immediately according to the following cycling parameters: 10 s @ 98° C., 10 s @ 68° C., 2 min @ 72° C., 20 cycles; 5 min @ 72° C.; 4° C. hold

Extended products are then double-stranded by addition of an extension primer.

Unfinished fragments and unextended oligonucleotides are then removed by incubation with Exonuclease I or Exo-SAP IT for 30 minutes.

1 μl of protease is added and the reaction incubated for 30 min @ 37 C followed by heat inactivation for 15 min @ 75 C.

Reactions are then purified via sedimentation with 1 volume of a 2×PEGppt solution (1×NEB4, 10 ug LPA, 30% PEG-8000)

### Example 12: Library Circularization

DNA library members comprising a single adaptor sequence at a first end may undergo target-selective addition of a second adaptor sequence at a second end using a library circularization method. Exemplary library circularization methods are described in U.S. Patent Application Pub. No. 20120003657, hereby incorporated by reference. 3′-end adapted library fragments are prepared as above using a non-palindromic hexamer (e.g., as described in U.S. Patent Application Pub. No. 20120003657 as the 3′ adaptor.

A circularization adaptor (in U.S. Patent Application Pub. No. 20120003657), possessing a T7 promoter sequence and 3′-overhangs complementary to the 3′-end adaptor is annealed to the 3′-adapted library fragments at a 10-fold molar excess.

The fragments are then ligated by the addition of T4 DNA ligase, creating target region-bearing circular products. Alternatively, a polymerase can be used to create the target region-bearing circular products.

Linear products are removed through incubation with a cocktail of Exo III and Exo I.

### Example 13: Fluorescently Labeled Library

5′-end adapted library fragments are prepared as above in Example 4 using a fluorescently labeled (Cy3, Cy5, FAM, HEX etc) oligo-dT hexamers as the 5′ adaptor. The resulting ligation products can be hybridized to an array CGH system, bead-array system, etc.

### Example 14: Direct Sequencing

A 5′-adenylated oligonucleotide (chemically or enzymatically) terminated with a 3′-end blocking group “x” (dideoxy-dNTP, biotinylated, etc.) and possessing a primer site as well as a region complementary to the surface bound oligonucleotide (flow-cell or bead) is ligated to the 3′-end of the native DNA mediated by truncated or mutated RNA ligase 2 from T4 or Mth as described in Example 3:

5′-P-DNA-OH-3′+5′-Ad-adaptorB-x-3′=>5′-P-DNA-adaptorB-x-3′

This is then followed by ligation of a second ssDNA adaptor using RNA ligase or CircLigase that contains a second primer site as well as the region complementary to the other surface bound oligonucleotide (flow-cell or bead), to create a full length product that can be directly sequenced. The second ligation can be performed as described in Example 2:

5′-HO-adaptorA-OH-3′+5′-P-DNA-adaptorB-x-3′=>5′-HO-adaptorA-DNA-adaptorB-x3′

Alternatively, fragmented DNA can be dephosphorylated upon repair (as above):

5′-P-DNA-OH-3′=>5′-HO-DNA-OH-3′

Following desphosphorylation and denaturation (alkaline or heat), a phosphorylated adaptor (chemically or enzymatically) can be ligated to the fragmented DNA with CircLigase:

5′-HO-DNA-OH-3′+5′-P-adaptorB-x-3′=>5′-HO-DNA-adaptorB-x-3′

This adaptor-modified library can then be enzymatically phosphorylated with T4 polynucleotide kinase:

5′-HO-DNA-adaptorB-x-3′=>5′-P-DNA-adaptorB-x-3′

A second adaptor can then be introduced by ligation with Circligase:

5′-P-DNA-adaptorB-x-3′+5′-HO-adaptorA-OH-3′=>5′-HO-adaptorA-DNA-adaptorB-x-3′

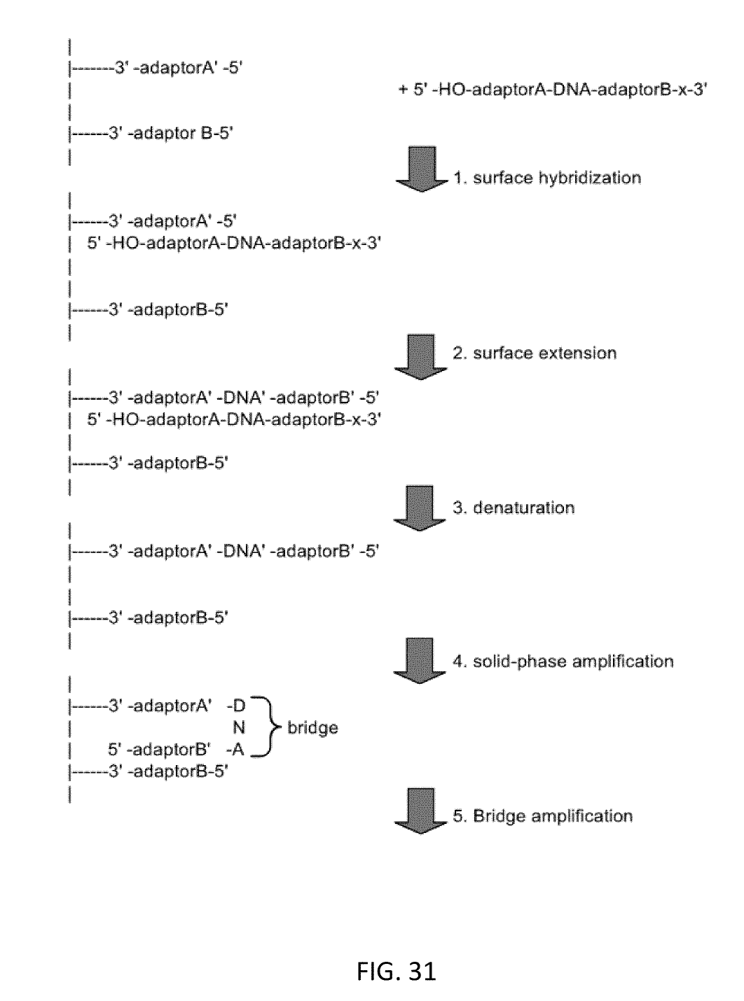

The resulting library member can then be sequenced directly as follows using either the Illumina flow-cell system or bead based systems (Ion-torrent/Roche 454). FIG. 31 depicts an exemplary embodiment of sequencing using an Illumina NGS platform.

### Example 15: Preparation of Oligonucleotide Primers for Capture and Enrichment of Target Sequences

A series of python scripts were created to generate a set of oligonucleotide primers for capture and enrichment of target sequences from a nucleic acid sample. Exon locations corresponding to genes listed in Table 9 below were curated from CCDS release 15.

TABLE 9
List of genes for exon capture

ABL1
AKT1
ALK
APC
ATM
AURKA
AURKB
AXL
BCL2
BRAF
BRCA1
BRCA2
CCND1
CDH1
CDK2
CDK4
CDK5
CDK6
CDK8
CDK9
CDK12
CDKN2A
CEBPA
CSF1R
CTNNB1
CYP2D6
DDR2
DNMT3A
DPYD
EGFR
EPCAM
ERBB2
ERBB3
ERBB4
ERCC1
ERCC2
ERCC3
ERCC5
ERCC6
EZH2
ESR1
FGFR1
FGFR2
FGFR3
FGFR4
FLT3
GNA11
GNAQ
GNAS
HNF1A
HRAS
IDH1
IDH2
JAK2
JAK3
KDR
KIT
KRAS
MAP2K1
MAP2K2
MAPK1
MET
MLH1
MPL
MRE11A
MSH2
MTOR
MSH6
MYC
MUTYH
NOTCH1
NPM1
NRAS
PARP1
PARP2
PDGFRA
PIK3CA
PMS2
PTCH1
PTCH2
PTEN
PTPN11
RB1
RET
RUNX1
SMAD4
SMARCB1
SMO
SRC
STK11
TET2
TP53
UGT1A1
VEGFA
VHL
WT1

Entries with overlapping exon locations were merged to create a single entry spanning the overlapping exons. Generated co-ordinates were then used to extract sequences from the corresponding human reference genome build (GRCh37.p13) with a 600 base pad at both the 5′ and 3′ ends. Oligonucleotide sequences for both sense and reverse complement strands flanking the exon were then identified according to the following criteria: (1) between 10 and 36 nucleotides in length; (2) possessing a 70% fractional annealing temperature between 56 and 60 C; (3) possessing a GC content between 30% and 70%; (4) possessing C or G homopolymer stretches less than 4 contiguous bases; (5) Absence of palindromic sequences of 6 or greater; (6) less than 50% self-complementarity. Upon identification of exon-flanking oligos, the largest interprobe distance less than 300 bases was calculated such that and even number of (+) and (−) oligonucleotide probes could be created If the distance between the exon-flanking oligonucleotides is greater than 300, the region between the two flanking oligos is further divided, such that the region has a minimal, even number of probes that divide the region of interest. These positions were used to create search windows to identify oligonucleotide probes according to the criteria outlined above. Capture sequences designed to tile about every 300 nt of the sense and anti-sense strands corresponding to exons of the genes in Table X (above) were identified (e.g., SEQ ID NOS 125-1947).

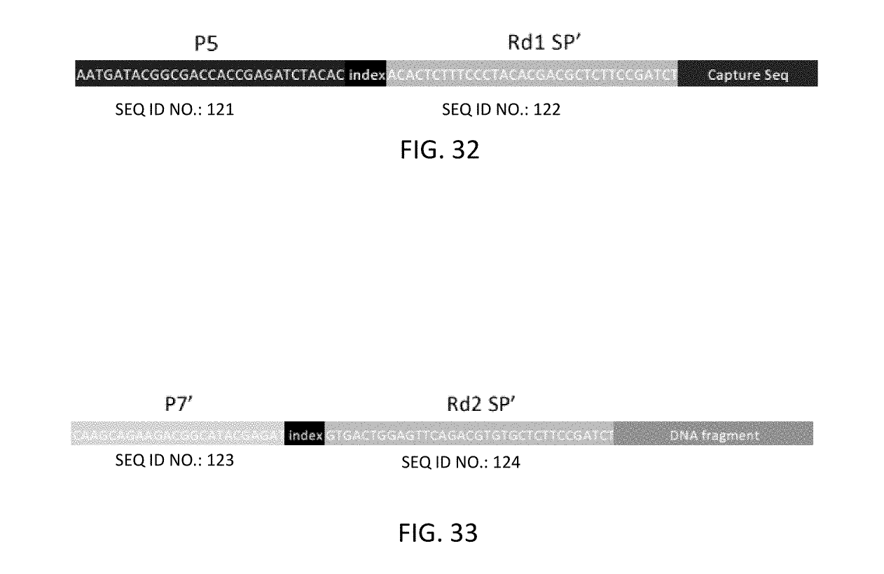

Oligonucleotide capture sequences were appended to the 3′-end of a standard barcoded Illumina P5 adaptor sequences to create the set of target-selective oligonucleotide (TSO) primers targeting sense and reverse complement strands received unique barcodes. A schematic of an exemplary TSO primer is shown in FIG. 32. Primers were individually synthesized using standard phosphoramidite chemistry, e.g., with 2 phosphorothioate linkages at the 3′-terminal and penultimate bases (Integrated DNA Technologies). TSO primers were pooled by strand. All sense strand TSOs were pooled as TSO Set 1 primers (SEQ ID NOS 1948-3770). All reverse strand TSOs were pooled as TSO Set 2 primers (SEQ ID NOS 3771-5593).

### Example 16: Multiplex Targeted Sequencing with Barcoding

The following protocol is designed to process a plurality of purified DNA samples simultaneously. These samples can be derived from formalin-fixed paraffin-embedded tissue (FPET) material, from flash frozen tissue (FFT), or from a liquid sample (e.g., whole blood or a substantially cell-free sample such as plasma or serum, urine, mucus, etc. DNA in the samples are fragmented by shearing. The average length of fragmented DNA is about 100-500 base pairs (bp) on average.

### Stage 1: DNA Repair (Approximate Time 1.5 Hrs)

Fragmented DNA samples are admixed in a reaction mixture comprising the repair enzymes formamidopyrimidine [fapy]-DNA glycosylase) (Fpg, New England Biolabs), Uracil-DNA Glycosylase (UDG, New England Biolabs), Endonuclease VIII (EndoVIII, New England Biolabs), and RNase if (New England Biolabs). The samples are then incubated at 37° C. and then heat inactivated at 75° C. according to the manufacturer's instructions. This reaction serves to remove damaged bases and to remove contaminating RNA from the sample. Upon completion of the reaction, samples are then incubated with T4 Polynucleotide kinase (PNK, New England Biolabs) in order to phosphorylate 5′ ends of the DNA fragments. Upon completion of the PNK reaction, samples are then incubated with terminal nucleotidtyl transferase (TdT) enzyme (New England Biolabs) to block 3′ hydroxyl groups of the DNA fragments with the addition of dideoxynucleotides.

Upon completion of the TdT reaction, repaired DNA fragments comprising 5′ phosphates and blocked 3′hydroxyl groups are purified using magnetic beads (SeraMAG, Thermofisher), and then quantified using, e.g., the Droplet Digital PCR PrimePCR RPP30 assay (#100-31243) or Qubit ssDNA assay kit (in conjuction with a Bioanalyzer/Experion system.

### Adaptor Ligation of Sample DNA

The purified and quantitated DNA samples are ligated to adaptor oligonucleotides comprising a sample-specific barcode. Adaptor oligonucleotides generally have sequence structure as shown in FIG. 33. 100-300 ng of repaired and 5′ phosphorylated sample DNA and adaptors are heat-denatured in separate tubes by heating to 95° C., resulting in single-stranded sample DNA and single-stranded adaptors. Sample ssDNA is then admixed with an adenylation reaction mixture comprising CircLigase II, 0.1 mM ATP, 15% PEG-8000, and other buffer components. The adenylation reaction mixture comprising the sample DNA is then incubated for at least 5 minutes at 65° C. to effect highly efficient adenylation of the sample ssDNA. Meanwhile, adaptor ssDNA is admixed with a Dilution buffer comprising 5 mM MnCl₂, 15% PEG-8000, and other buffer components. The Dilution buffer comprising adaptor ssDNA is then incubated for at least 5 minutes at 65° C. Upon completion of the adenylation reaction, adenylated sample ssDNA is diluted at least 10-fold with the Dilution buffer comprising adaptor ssDNA. This results in a final ATP concentration of 0.01 mM and addition of Mn²⁺ to the reaction, which effectively drive the ligation reaction to completion. Ligation of the single-stranded adaptors to the sample ssDNA results in creation of the ssDNA library. The adenylation and ligation reactions altogether can be completed in approximately 1.5 hours. ssDNA library members are then purified using magnetic beads (SeraMAG, ThermoFisher).

### Target Enrichment (Approximately 2 Hours)

50-150 ng of ssDNA library members are incubated in separate amplification reaction mixtures comprising 0.5 μM of either TSO Set 1 primers or with TSO Set 2 primers from Example 15. Separation of the TSO Set 1 primers and TSO Set 2 primers ensures that only linear amplification of target regions occurs. Amplification reaction mixtures also comprise a high-fidelity DNA Polymerase (Phusion Hot Start II, Thermo Scientific), dNTPs, and other reaction components necessary for conducting an amplification reaction. 40 cycles of amplification are performed using a thermocycler. Linear amplification results in capture and enrichment of selected target regions corresponding to exons of the 96 cancer genes in Table X, wherein each captured target region comprises a first adaptor comprising a sample index barcode at first end and a second adaptor comprising a strand-specific barcode at the other end. Captured targets are quantified as described herein and normalized to 1 nM (or 12×10⁶ copies/μL) for sequencing on a MiSeq sequencer (Illumina).

### Example 17: Assessment of Low Tm Probe Designs

Genomic DNA was harvested from a tumor sample known to harbor stop mutation in codon 1306 of the APC gene (c3916G>T) as determined via sequencing. Similarly, wild-type DNA (NA18507) was obtained from Coriell. Both samples were quantified with ddPCR using RPP30. To assess the performance of various probe designs targeting the APC mutation, a series of probes were designed as depicted in Table 10, below.

TABLE 1
low Tm probe designs

| | Wt | Mu |
|---|---|---|
| 5′-nuclease | HEX- | FAM- |
| | ACCCTGCAAATAGCAGAAATAA | ACCCTGCAAATAGCATAAATAAA |
| | AAGAAAAG-IBlkFq | AGAAAAG-IBlkFq |
| | (SEQ ID NO: 6) | (SEQ ID NO: 7) |
| Pleaides 1 | MGB-AP525- | MGB-FAM- |
| | TTATTTCTGCTATTTG | TTTATTTATGCTATT*T*G |
| | (SEQ ID NO: 8) | (SEQ ID NO: 9; Note: * denote |
| | | that the nt before is a superbase) |
| Pleaides 2 | MGB-AP525- | MGB-FAM- |
| | TTATTTCTGCTAT*T*T*G | TTTATTTATGCTA*TTT*GC (SEQ |
| | (SEQ ID NO: 10; Note, * | ID NO: 11; Note:* denote that |
| | denote that the nt | the nt before is a superbase) |
| | before is a superbase) | |
| Pleaides 3 | MGB-AP525- | MGB-FAM- |
| | TTATTTCTGCTAT*T*T*GC | TTTAT*T*TATGCTA*TT*T*GC |
| | (SEQ ID NO: 12) | (SEQ ID NO: 13) |
| Miniprobes | MGB-FAM-TTATT*TATGCT* | MGB-AP525-TTATTTCTGCT |
| 1 | (SEQ ID NO: 14) | (SEQ ID NO: 15) |
| Miniprobes | MGB-AP525-TTATTTCTGC | MGB-FAM-TTATT*TATGC |
| 2 | (SEQ ID NO: 16) | (SEQ ID NO: 17) |

Probes were incorporated into ddPCR reactions mixes as depicted in Table 11 below as and formed into droplets

TABLE 11
ddPCR reaction mix

| | |
|---|---|
| 2x Droplet PCR Supermix | 10.0 μl |
| Water | 3.2 μl |
| DNA | 2.0 μl |
| 10 uM sense primer (1 uM final) | 2.0 μl |
| 10 uM antisense primer (0.2 uM final) | 0.4 μl |
| 10 uM mu probe | 1.2 μl |
| 10 uM wt probe | 1.2 μl |

Thermocycling protocol was as follows:

10 min @ 95° C.; 30 s @ 95° C., 1 min @ 58C, 40 cycles; 10 min @ 98° C.; hold at 12° C.

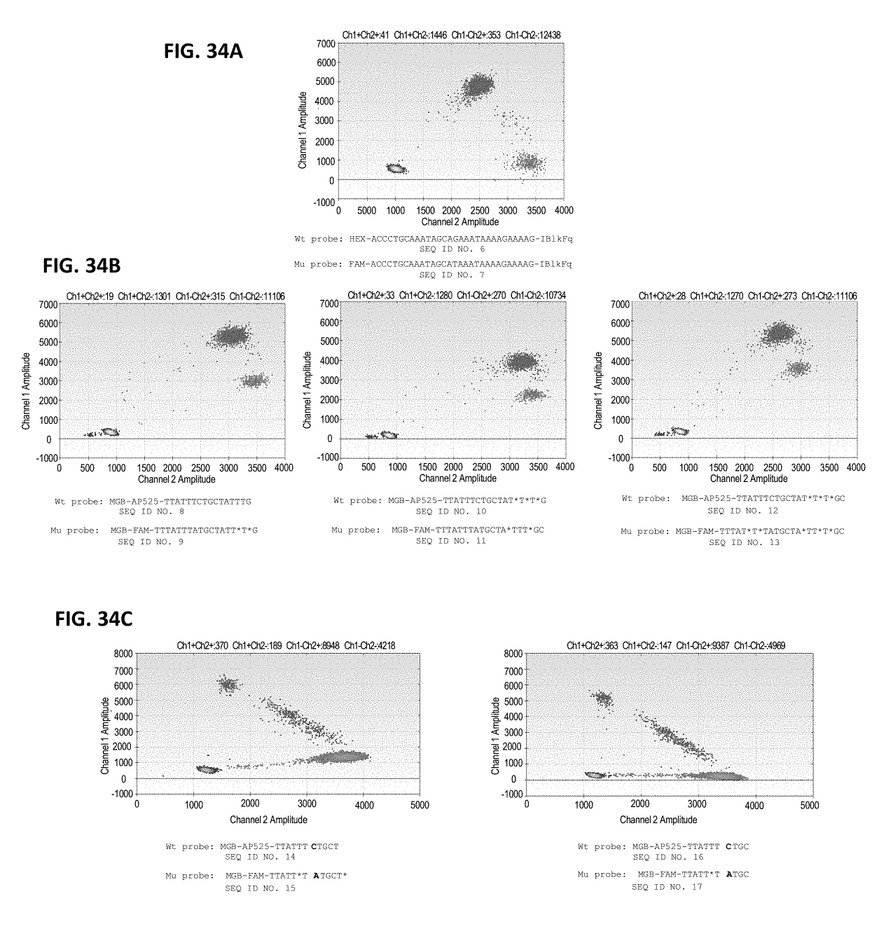

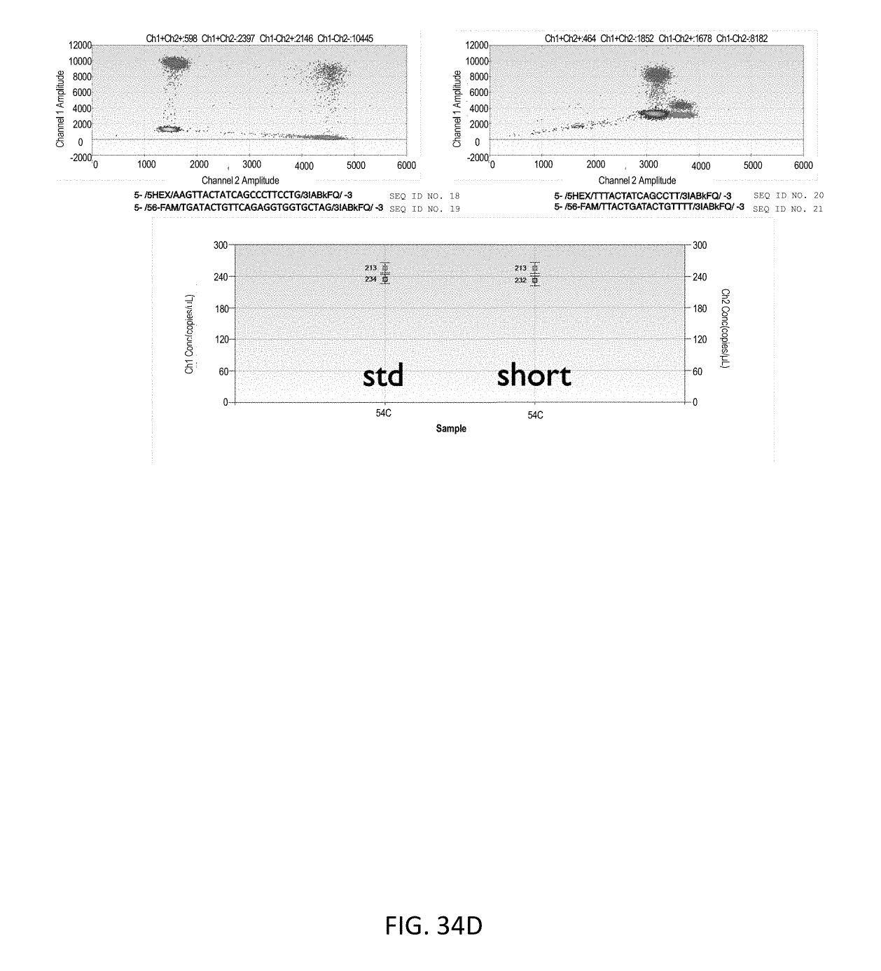

Following thermocycling, reactions were analyzed with the QX100 reader. FIG. 34A shows the use of standard 5′-nuclease probes for the APC target. FIG. 34B shows the use of 3 version of Pleiades probes for analysis, showing poorer performance relative to the standard nuclease assays. FIG. 34C shows the use of 2 versions of miniprobes, indicating a higher specificity obtained versus the Pleiades probes and the standard 5′-nuclease probes as indicated by the separation of the wild-type (green) and mutant (blue) clusters.

To determine if the use of miniprobes only required probes of sufficient length, a pair of probes to the RNaseP locus (RPP30) were designed as follows:

TABLE 12
RNaseP assay

| | Wt | Mu |
|---|---|---|
| 5′-nuclease | 5- | 5-/56- |
| | /5HEX/AAGTTACTATCAGCCC | FAM/TGATACTGTTCAGAGGTG |
| | TTCCTG/3IABkFQ/-3 | GTGCTAG/3IABkFQ/-3 |
| | (SEQ ID NO: 18) | (SEQ ID NO: 19) |
| Miniprobes | 5- | 5-/56- |
| 1 | /5HEX/TTTACTATCAGCCTT/ | FAM/TTACTGATACTGTTTT/ |
| | 3IABkFQ/-3 | 3IABkFQ/-3 |
| | (SEQ ID NO: 20) | (SEQ ID NO: 21) |

Probes were assessed as described above. As seen in FIG. 34D, while the miniprobes (right panel) exhibited higher background fluorescence, likely due to poorer quenching of the 15 mer versus the shorter 11-mer of the Pleiades-based miniprobes, separation was sufficient to discern distinct clusters, allowing reproducible concentration calls relative to the standard 5′-nuclease probes.

### Example 18: Allelic Discrimination Assay Using Low Tm Probes and Barcoded Primers

Primer/probe sets to assay the c.1799T>A (V600E) BRAF mutation were generated and tested. Each primer/probe set tested included the common anti-sense primer CATGAAGACCTCACAGTAAA (SEQ ID NO: 22), wild-type probe HEX-TAAGGCTCGGTT-BHQ (SEQ ID NO: 23), and mutant probe FAM-TTGGGTTCGGTC-BHQ (SEQ ID NO: 24). Various designs of wild-type and mutant sense primers were tested. All wild-type sense primers comprise the barcode sequence GGCAATAAGGCTCGGTTGGCATTGG (SEQ ID NO: 25) which corresponds to the wild-type probe sequence, and all mutant sense primers comprise the barcode sequence ACATTGGGTTCGGTCGTAACTTAGGAA (SEQ ID NO: 26) which corresponds to the mutant probe sequence.

Wild-type specific sense primers were designed such that the mutation site lies under the ultimate (0) or the penultimate (−1) base. Primers were therefore designed to either contain a deliberate mismatch 1-3 nts away from the mutation site or to not contain any additional mismatch.

The following BRAF wild-type sense primers were designed according to Table 13 below.

TABLE 13
BRAF wild-type sense primer designs

| Primer design | Sequence |
|---|---|
| BRAF_1799T_(-1a:-2c) | GGCAATAAGGCTCGGTTGGCATTGGCACTCCATCGAGAT |
| | TTCAC (SEQ ID NO: 27) |
| BRAF_1799T_(-1a:-2c > a) | GGCAATAAGGCTCGGTTGGCATTGGCACTCCATCGAGAT |
| | TTaAC (SEQ ID NO: 28) |
| BRAF_1799T_(-1a:-2c > g), | GGCAATAAGGCTCGGTTGGCATTGGCACTCCATCGAGAT |
| | TTgAC (SEQ ID NO: 29) |
| BRAF_1799T_(-1a:-2c > t | GGCAATAAGGCTCGGTTGGCATTGGCACTCCATCGAGAT |
| | TTtAC (SEQ ID NO: 30) |
| BRAF_1799T_(-1a:-3t) | GGCAATAAGGCTCGGTTGGCATTGGCACTCCATCGAGAT |
| | TTCAC (SEQ ID NO: 31) |
| BRAF_1799T_(-1a:-3t > a) | GGCAATAAGGCTCGGTTGGCATTGGCACTCCATCGAGAT |
| | TaCAC (SEQ ID NO: 32) |
| BRAF_1799T_(-1a:-3t > c) | GGCAATAAGGCTCGGTTGGCATTGGCACTCCATCGAGAT |
| | TcCAC (SEQ ID NO: 33) |
| BRAF_1799T_(-1a:-3t > g) | GGCAATAAGGCTCGGTTGGCATTGGCACTCCATCGAGAT |
| | TgCAC (SEQ ID NO: 34) |
| BRAF_1799T_(0a:-1c) | GGCAATAAGGCTCGGTTGGCATTGGCCACTCCATCGAGA |
| | TTTCA (SEQ ID NO: 35) |
| BRAF_1799T_(0a:-1c > a) | GGCAATAAGGCTCGGTTGGCATTGGCCACTCCATCGAGA |
| | TTTaA (SEQ ID NO: 36) |
| BRAF_1799T_(0a:-1c > g) | GGCAATAAGGCTCGGTTGGCATTGGCCACTCCATCGAGA |
| | TTTgA (SEQ ID NO: 37) |
| BRAF_1799T_(0a:-1c > t) | GGCAATAAGGCTCGGTTGGCATTGGCCACTCCATCGAGA |
| | TTTtA (SEQ ID NO: 38) |
| BRAF_1799T_(0a:-2t) | GGCAATAAGGCTCGGTTGGCATTGGCCACTCCATCGAGA |
| | TTTCA (SEQ ID NO: 39) |
| BRAF_1799T_(0a:-2t > a) | GGCAATAAGGCTCGGTTGGCATTGGCCACTCCATCGAGA |
| | TTaCA (SEQ ID NO: 40) |
| BRAF_1799T_(0a:-2t > c) | GGCAATAAGGCTCGGTTGGCATTGGCCACTCCATCGAGA |
| | TTcCA (SEQ ID NO: 41) |
| BRAF_1799T_(0a:-2t > g) | GGCAATAAGGCTCGGTTGGCATTGGCCACTCCATCGAGA |
| | TTgCA (SEQ ID NO: 42) |

The following BRAF mutant sense primers were designed according to Table 14 below.

TABLE 14
mutant BRAF sense primer designs

| Primer design | Sequence |
|---|---|
| BRAF_1799T > A_(-1a > t:- | ACATTGGGTTCGGTCGTAACTTAGGAACACTCCATCGAG |
| 2c) | ATTTCTC (SEQ ID NO: 43) |
| BRAF_1799T > A_(-1a > t:- | ACATTGGGTTCGGTCGTAACTTAGGAACACTCCATCGAG |
| 2c > a) | ATTTaTC (SEQ ID NO: 44) |
| BRAF_1799T > A_(-1a > t:- | ACATTGGGTTCGGTCGTAACTTAGGAACACTCCATCGAG |
| 2c > g) | ATTTgTC (SEQ ID NO: 45) |
| BRAF_1799T > A_(-1a > t:- | ACATTGGGTTCGGTCGTAACTTAGGAACACTCCATCGAG |
| 2c > t) | ATTTtTC (SEQ ID NO: 46) |
| BRAF_1799T > A_(-1a > t:- | ACATTGGGTTCGGTCGTAACTTAGGAACACTCCATCGAG |
| 3t) | ATTTCTC (SEQ ID NO: 47) |
| BRAF_1799T > A_(-1a > t:- | ACATTGGGTTCGGTCGTAACTTAGGAACACTCCATCGAG |
| 3t > a) | ATTaCTC (SEQ ID NO: 48) |
| BRAF_1799T > A_(-1a > t:- | ACATTGGGTTCGGTCGTAACTTAGGAACACTCCATCGAG |
| 3t > c) | ATTcCTC (SEQ ID NO: 49) |
| BRAF_1799T > A_(-1a > t:- | ACATTGGGTTCGGTCGTAACTTAGGAACACTCCATCGAG |
| 3t > g) | ATTgCTC (SEQ ID NO: 50) |
| BRAF_1799T > A_(0a > t:- | ACATTGGGTTCGGTCGTAACTTAGGAACCACTCCATCGAG |
| 1c) | ATTTCT (SEQ ID NO: 51) |
| BRAF_1799T > A_(0a > t:- | ACATTGGGTTCGGTCGTAACTTAGGAACCACTCCATCGAG |
| 1c > a) | ATTTaT (SEQ ID NO: 52) |
| BRAF_1799T > A_(0a > t:- | ACATTGGGTTCGGTCGTAACTTAGGAACCACTCCATCGAG |
| 1c > g) | ATTTgT (SEQ ID NO: 53) |
| BRAF_1799T > A_(0a > t:- | ACATTGGGTTCGGTCGTAACTTAGGAACCACTCCATCGAG |
| 1c > t) | ATTTtT (SEQ ID NO: 54) |
| BRAF_1799T > A_(0a > t:- | ACATTGGGTTCGGTCGTAACTTAGGAACCACTCCATCGAG |
| 2t) | ATTTCT (SEQ ID NO: 55) |
| BRAF_1799T > A_(0a > t:- | ACATTGGGTTCGGTCGTAACTTAGGAACCACTCCATCGAG |
| 2t > a) | ATTaCT (SEQ ID NO: 56) |
| BRAF_1799T > A_(0a > t:- | ACATTGGGTTCGGTCGTAACTTAGGAACCACTCCATCGAG |
| 2t > c) | ATTcCT (SEQ ID NO: 57) |
| BRAF_1799T > A_(0a > t:- | ACATTGGGTTCGGTCGTAACTTAGGAACCACTCCATCGAG |
| 2t > g) | ATTgCT (SEQ ID NO: 58) |

Ability to discriminate mutant from wild-type species with these primer/probe sets was assessed by digital PCR. 20× stocks of primer/probe sets were created as follows:

TABLE 15
Primer/probe set stocks

| Component | Volume (ul) |
|---|---|
| 100 uM antisense primer | 5 |
| 100 uM sense primer | 1 |
| 100 uM probe | 2 |
| TE buffer | 17 |

To prepare sample DNA, a mixture of 10% mutant (RKO-1, ATCC) in wild-type (NA18507, Coriell) genomic DNA was created (˜250 and 2500 copies/μl, respectively). Alternatively, a dilution series of mutant DNA (RKO-1) in background of a wild-type control (purified genomic DNA from whole blood) was created.

ddPCR reactions were assembled as shown in Table 16 below.

TABLE 16
ddPCR reaction

| Component | Volume (μl) |
|---|---|
| 2x droplet PCR supermix (Bio-rad) | 10 |
| 20x mutant primer/probe set | 1 |
| 20x wild-type primer/probe set | 1 |
| water | 6 |
| Sample DNA | 2 |

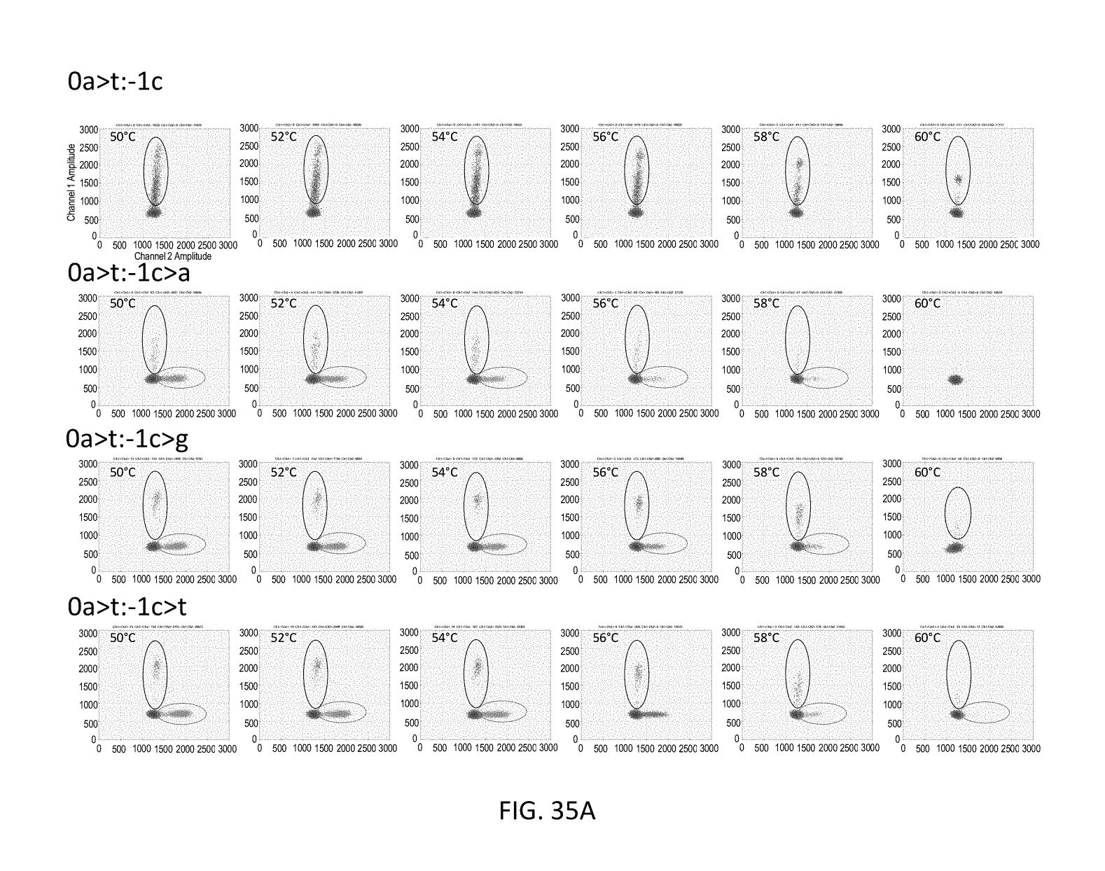

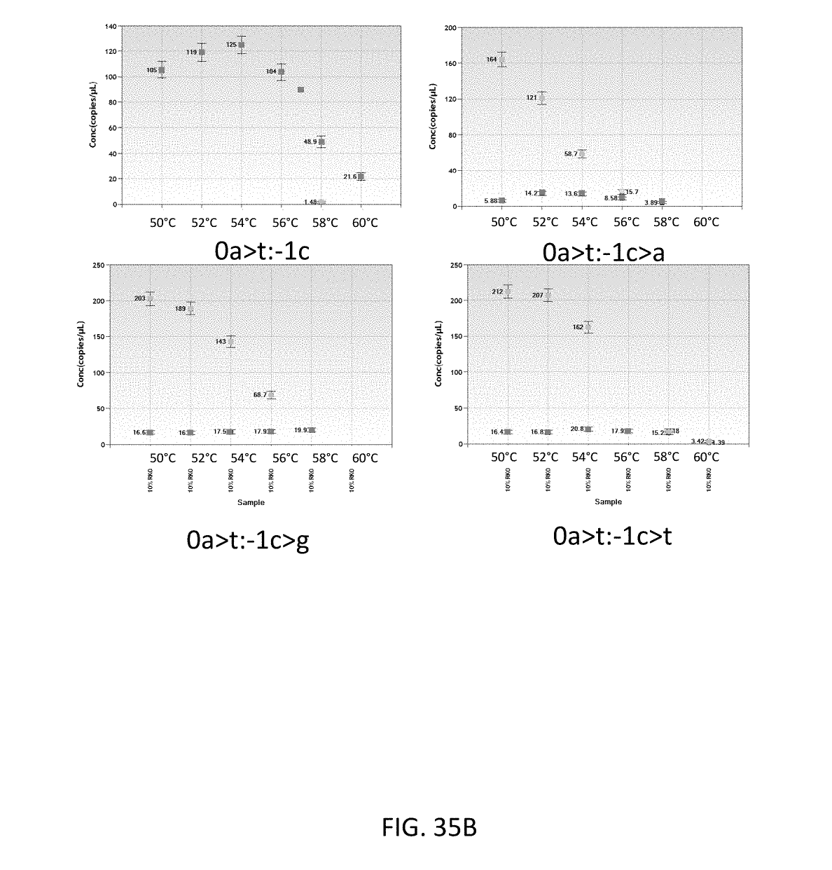

ddPCR reaction mixes were converted to droplets and cycled on a C1000 thermocycler (Bio-rad) according to the following parameters: 10 min @ 95° C.; 1 min @ 50-60° C., 45 cycles; 5 min @ 70° C.; 4° C. hold. Thermocycled reactions were then analyzed with the QX-100 ddPCR reader with Quantasoft v1.4. Results are depicted in FIGS. 35A-35B, 36A-36B, 37A-37B, 38A-38B, and 40. For FIGS. 35A, 36A, 37A and 38A, the Y axis denotes intensity of Channel 1 fluorescence (fluorescence of mutant probe, FAM), the X axis denotes intensity of Channel 2 fluorescence (fluorescence of wild-type probe, HEX). Gridlines are spaced every 500 intensity units apart, with X and Y axis maxima of 3000 intensity units. FAM fluorescence-positive droplets are circled in black ovals, HEX fluorescence-positive droplets are circled in gray ovals, and droplets that are positive for both HEX and FAM are circled in hatched ovals. For FIGS. 35B, 36B, 37B, and 38B, dark gray data points denote concentration of mutant alleles as copies/μl, and light gray data points denote concentration of wild-type alleles as copies/μl.

FIGS. 35A-35B depict results from ddPCR assays wherein the sense primers were designed to overlay the mutation site at the ultimate (0) base, and to either contain a mismatch at the base immediately adjacent to the mutation site (−1) or to not contain a further mismatch. Probes designed to overlay the mutation site at the ultimate (0) base and to have a nt mismatch adjacent to the mutation site resulted in distinguishable clusters of wild-type and mutant species, with greater separation of clusters at the lower temperatures (e.g., −50 to −58° C.).

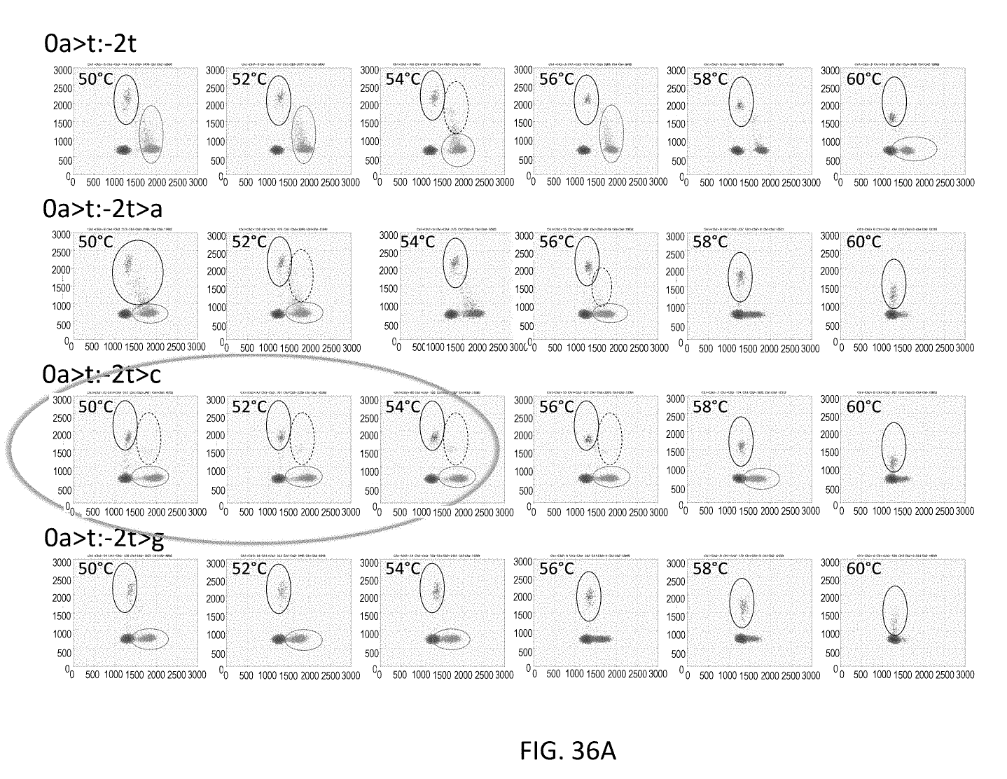

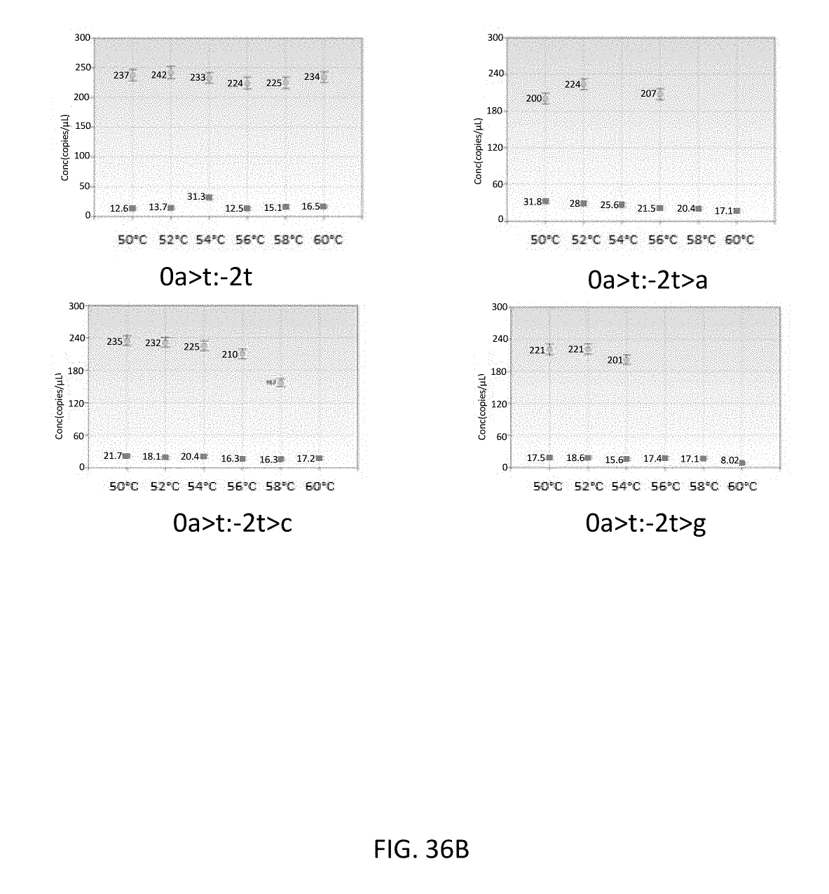

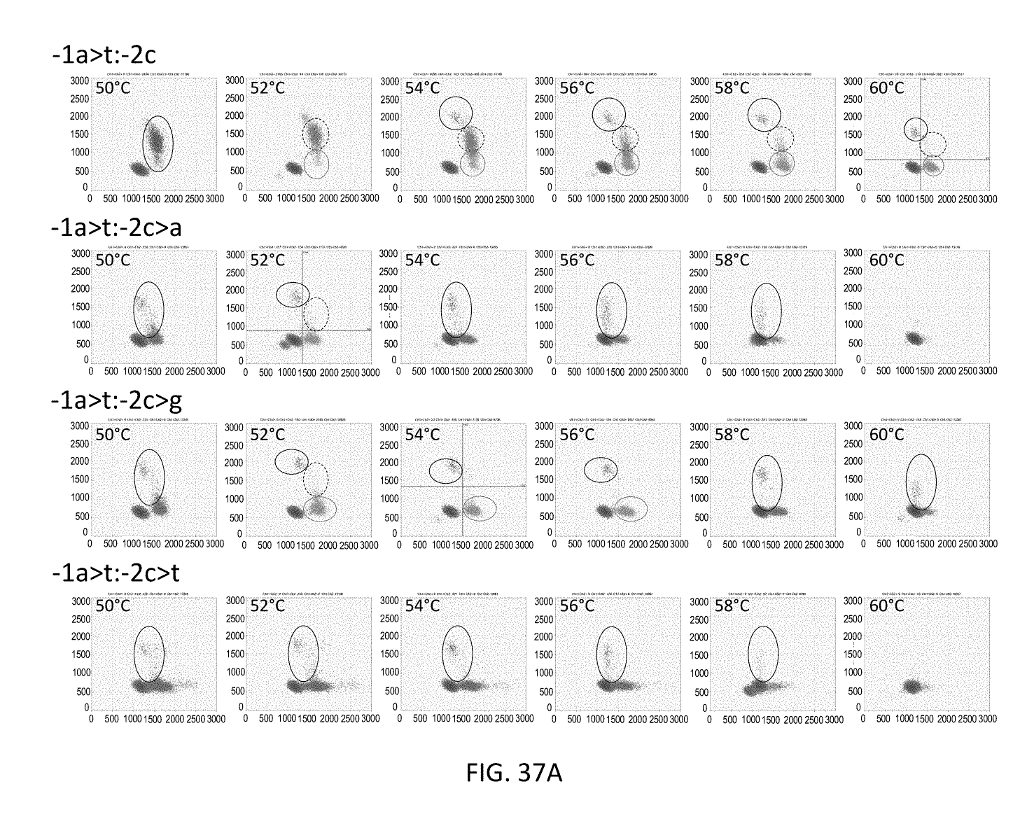

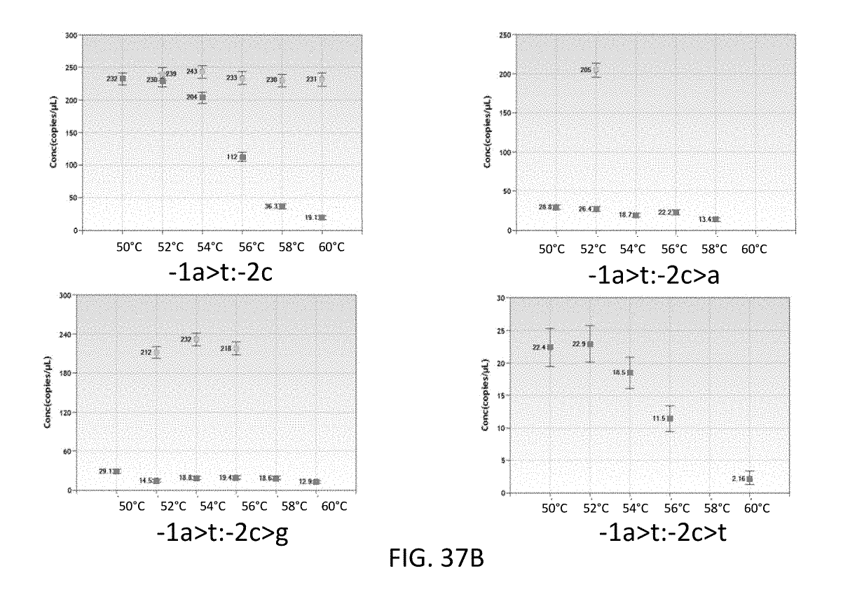

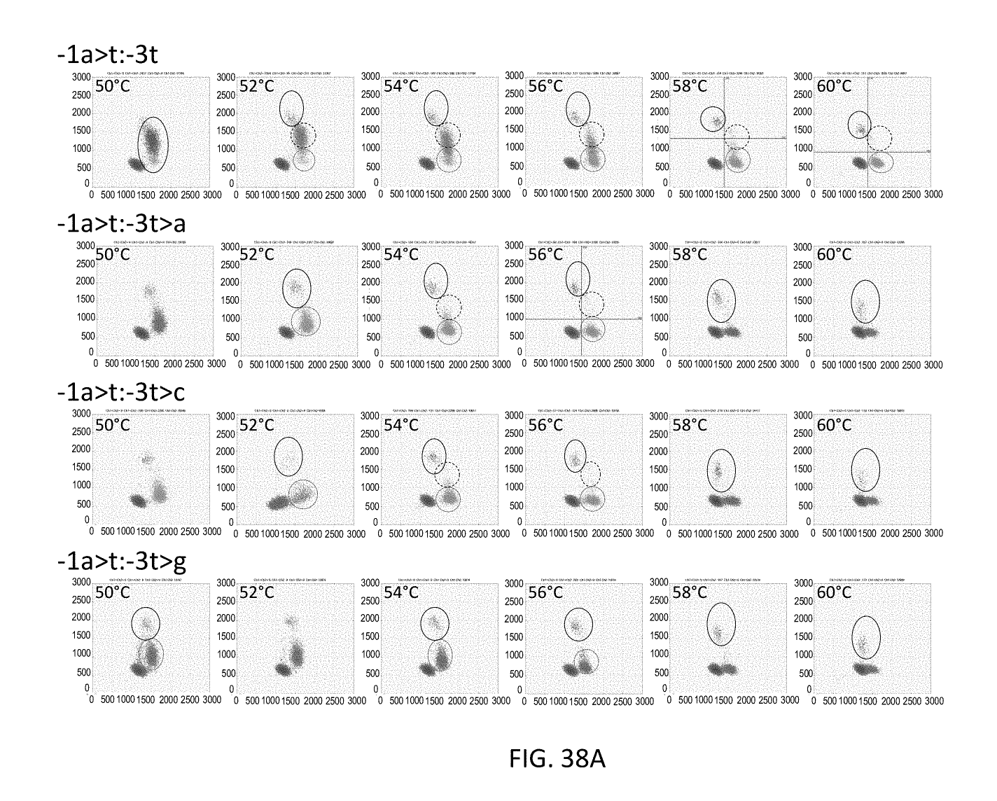

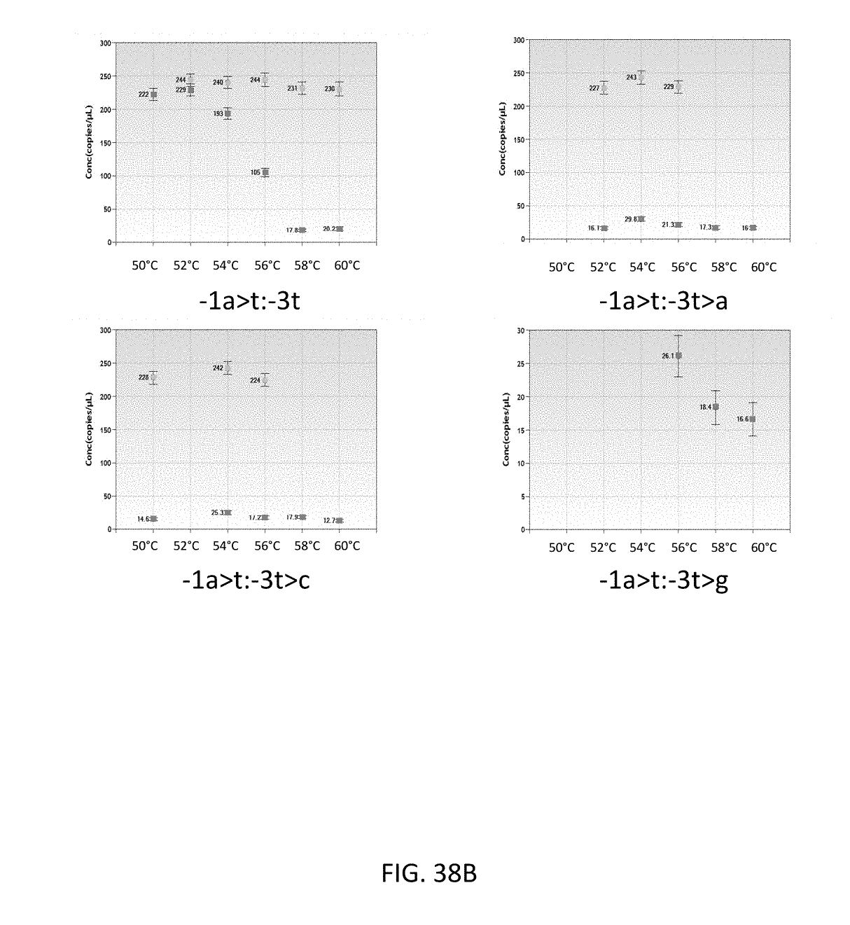

FIGS. 36A-36B depict results from ddPCR assays wherein the sense primers were designed to overlay the mutation site at the ultimate (0) base and to either to contain a mismatch 2 bases away from the mutation site (−2) or to not contain a further mismatch. FIGS. 36A-36B depict results from the assay. Sense primers which overlay the mutation at the 0 base and which contain a T to C substitution at the −2 base resulted in the most highly distinguishable clusters of wild-type and mutant species, particularly in temperature ranges from −50 to −54° C. FIGS. 37A-37B and 38A-38B demonstrate that primers designed to overlay the mutation site at the penultimate (−1) base did not perform as well as primers which overlay the mutation site at the ultimate (0) base.

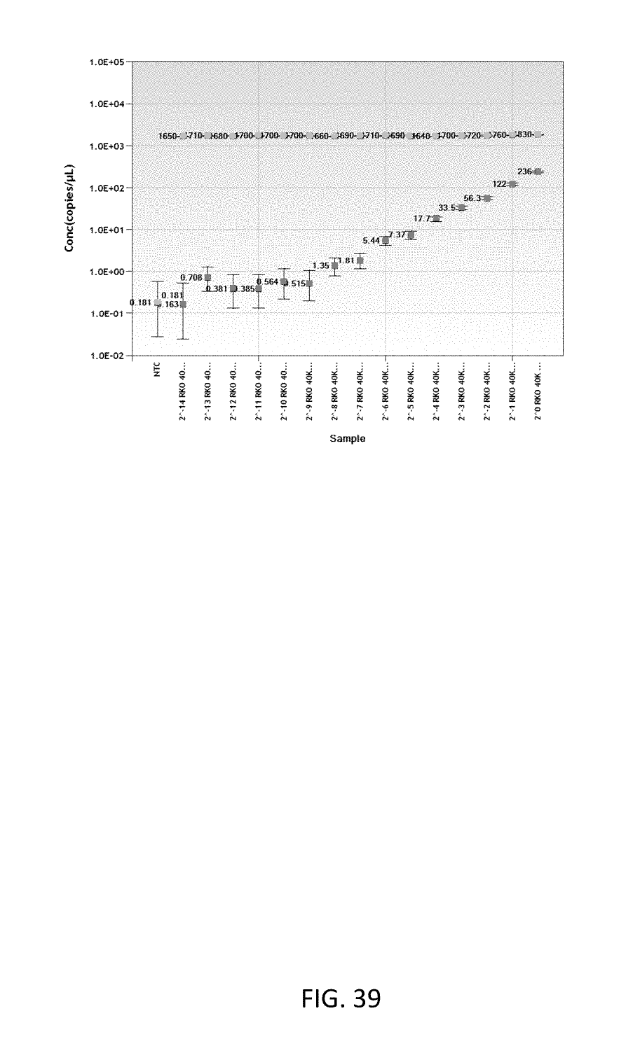

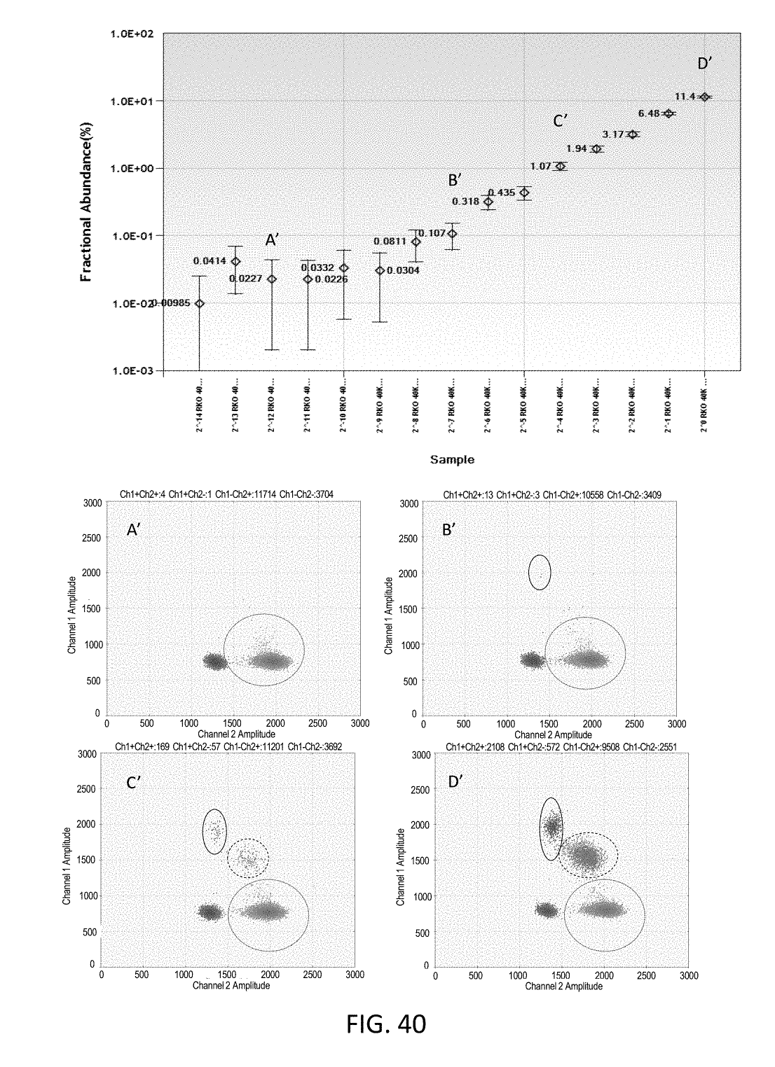

To determine the detection limits of the BRAF ddPCR assay, a dilution series of mutant DNA (RKO-1) in background of a wild-type control (purified genomic DNA from whole blood) was created with mutant DNA diluted 2-fold for every dilution. Assays consisting of a mixture of the −BRAF_1799T_(0a:−2t>c) and −BRAF_1799T>A_(0a>t:−2c) were used to interrogate a mixture of mutant BRAF genomic DNA in a background of wild-type DNA using an annealing temperature of 54° C. FIGS. 39-40 demonstrate detection limits of the BRAF low Tm universal probes with barcoded primers. FIG. 39 depicts wild-type and mutant concentration calls for each sample. Wild-type concentration calls were about 1700 copies/μl for each sample. Mutant concentration calls for each diluted sample decreased steadily, with the lowest limit of quantitation at about 1.81 copies/μl. FIG. 40 depicts fractional abundance of mutant DNA in a wild-type background, as determined by the ddPCR assay. FIG. 40 demonstrates that the ddPCR assay can detect a 0.1% fractional abundance of the BRAF mutant DNA.

### Example 19: CNV ddPCR Panel

Digital PCR probe/primer sets were designed to assay copy number variation of 19 cancer genes (MET, FGFR1, FGFR2, FLT3, HER3, EGFR, mTOR, CDK4, HER2, RET, HADH, ZFP3, DDR2, AURKA, VEGFA, CDK6, JAK2, BRAF, SRC). Of the 19 cancer genes, 9 are known to also harbor mutations within regions exhibiting cancer-related gene amplification (MET, FGFR2, EGFR, RET, DDR2, CDK6, JAK2, BRAF, SRC). For these 9 genes, probes were designed to overlay the mutation site, and to have greater complementarity to the wild-type allele than to the mutant allele. A probe/primer set was also included for the housekeeping gene RNaseP. The probes/primer sets for the CNV panel, and the genes they correspond to, are shown in Table 17.

TABLE 17
CNV Test Panel

| Gene | Chromosome | Forward Primer | Reverse Primer | |
|---|---|---|---|---|
| Name | Location | (Limiting) | (Excess) | Probe |
| MET | chr7:116423356- | AATAAATCATAA | CAGCTTTGCACCTG | GAATACT |
| | 116423525 | GGTCT*T*GCCA* | TTTTGTTGTGTAC | *ATA*G |
| | | GAGACATG | (SEQ ID NO: 60) | (SEQ ID |
| | | (SEQ ID NO: 59) | | NO: 61) |
| FGFR1 | chr8:38282028- | AATAAATCATAA | GTTCA*TGTGTAAG | ACTGGA* |
| | 38282221 | CA*CCTCGATGTG | GTGTACAGTG | TGTGC |
| | | CTTTAGC | (SEQ ID NO: 63) | (SEQ ID |
| | | (SEQ ID NO: 62) | | NO: 64) |
| FGFR2 | chr10:123279564- | GTGGTCGGAGGA | AATAAATCATAACT | TACAGTG |
| | 123279710 | GACGTAGAGT | GGATGTGGGGCTG | ATGC |
| | | (SEQ ID NO: 65) | (SEQ ID NO: 66) | (SEQ ID |
| | | | | NO: 67) |
| FLT3 | chr13:28592599- | AATAAATCATAA | GGTGA*AGATATGT | CATGATA |
| | 28592731 | GA*CAACA*TAGT | GA*CTTTGGATTG | TCTCG |
| | | *T*GGAATCAC | (SEQ ID NO: 69) | (SEQ ID |
| | | (SEQ ID NO: 68) | | NO: 70) |
| HER3 | chr12:56478768- | GAAGT*T*T*GCC | AATAAATCATAACG | ACTCCAG |
| | 56478977 | ATCTTCGTCATG | GAGCTGGCGCAGA | CCAC |
| | | (SEQ ID NO: 71) | G | (SEQ ID |
| | | | (SEQ ID NO: 72) | NO: 73) |
| EGFR | chr7:55259409- | AATAAATCATAA | GGTATTCT*T*T*CT | Probe 1: |
| | 55259571 | CA*GCATGT*CA* | CTTCCGCAC | CAAACTG |
| | | AGATCACAGAT | (SEQ ID NO: 75) | CTG |
| | | (SEQ ID NO: 74) | | TTGGGCT |
| | | | | GGC |
| | | | | (SEQ ID |
| | | | | NO: 76) |
| mTOR | chr1:11188060- | AATAAATCATAA | GCACAATGCAGCCA | TCACACA |
| | 11188185 | CTGCTGGACCAG | ACAAGATTCTG | TGTTC |
| | | GGTGTT | (SEQ ID NO: 78) | (SEQ ID |
| | | (SEQ ID NO: 77) | | NO: 79) |
| CDK4 | chr12:58142966- | AATAAATCATAA | AATAAATCATAACA | CTGAGAT |
| | 58143102 | CCAGTGCAGTCG | GCAGCTGTGCTCCC | *GGAG |
| | | GTGGTAC | GA | (SEQ ID |
| | | (SEQ ID NO: 80) | (SEQ ID NO: 81) | NO: 82) |
| HER2 | chr17:37880950- | AATAAATCATAA | AATAAATCATAAGG | TGATGGC |
| | 37881176 | CCTTGTCCCCAGG | GAGACATATGG*GG | TGG |
| | | AAGCA | AGC | (SEQ ID |
| | | (SEQ ID NO: 83) | (SEQ ID NO: 84) | NO: 85) |
| RET | chr10:43617375- | CTTTA*GGGT*CG | AATAAATCATAACG | AATGGAT |
| | 43617484 | GATTCCAGTT | T*GGT*GTAGA*TAT | GGC |
| | | (SEQ ID NO: 86) | GA*TCA | (SEQ ID |
| | | | (SEQ ID NO: 87) | NO: 88) |
| RNaseP | chr10:92632074- | AGGAAGGGCTGA | AATAAATCATAACA | GTACCCT |
| | 92632223 | *TAGTAA*CTTAG | GAAGCCGGAGCTG | TGGA |
| | | (SEQ ID NO: 89) | GA | (SEQ ID |
| | | | (SEQ ID NO: 90) | NO: 91) |
| HADH | chr4:108935580- | AATAAATCATAA | AATAAATCATAAGA | ACCAAGT |
| AP525 | 108935749 | CTC(I07)ACGATGG | TGCAGCCTCCGTTG | CTGTG |
| | | CTTCCAC | T | (SEQ ID |
| | | (SEQ ID NO: 92) | (SEQ ID NO: 93) | NO: 94) |
| ZFP3 | chr17:4994800- | AATAAATCATAA | GAGTTTGGAGCAGG | TCCAACA |
| AP525 | 4995200 | CT(I07)CCA*TGGA | ATGTGAAGAAG | TGTC |
| | | CTCTCTCGA | (SEQ ID NO: 96) | (SEQ ID |
| | | (SEQ ID NO: 95) | | NO: 97) |
| DDR2 | chr1:162745438- | AATAAATCATAA | GGA(I07)ATCT(I07)A | AGAATTA |
| AP525 | 162745640 | TGCGTACATCGCT | ATCAGT*T*T*CTTT | GGG |
| | | GGAGG | CC | (SEQ ID |
| | | (SEQ ID NO: 98) | (SEQ ID NO: 99) | NO: 100) |
| AURKA | chr20:54963161- | AATAAATCATAA | GGGTTTA*TAAATG | TAAATTG |
| AP525 | 54963260 | (I07)TGCAT*T*T*C | TGA*ATGA*GATTA | AATA*A* |
| | | A(I07)GACCTGT | CAG | (SEQ ID |
| | | (SEQ ID NO: 101) | (SEQ ID NO: 102) | NO: 103) |
| VEGFA | chr6:43745202- | GTGGTGAAGTTC | AATAAATCATAACC | GCTACTG |
| FAM | 43745408 | ATGGATGTCTATC | ACCAGGGTCTCGAT | CCATC |
| | | (SEQ ID NO: 104) | TGG | (SEQ ID |
| | | | (SEQ ID NO: 105) | NO: 106) |
| CDK6 | chr7:92403984- | CATGTCGATCAA | AATAAATCATAATC | TAAAGTT |
| AP525 | 92404134 | GACTTGACCACTT | AGTGGGCACTCCAG | CCAG |
| | | ACTT | G | (SEQ ID |
| | | (SEQ ID NO: 107) | (SEQ ID NO: 108) | NO: 109) |
| JAK2 | chr9:5073695- | CAAGCTTTCTCAC | AATAAATCATAACT | GGAGTAT |
| AP525 | 5073789 | AAGCATTTGGT | TA*CTCTCGTCTCCA | GTGTC |
| | | (SEQ ID NO: 110) | CAG | (SEQ ID |
| | | | (SEQ ID NO: 111) | NO: 112) |
| BRAF | chr7:140453074- | GACAACTGTTCA | AATAAATCATAAGG | ATTTCACT |
| AP525 | 140453195 | AACTGATGGGAC | TGATT*T*T*GGTCT | GTA |
| | | (SEQ ID NO: 113) | AGCTAC | (SEQ ID |
| | | | (SEQ ID NO: 114) | NO: 115) |
| SRC | chr20:36022571- | CGGTTACTGCTCA | AATAAATCATAAC(I07) | AACCCGA |
| FAM | 36022750 | ATGCAGAG | TGGTCTCACTTT | GAG |
| | | (SEQ ID NO: 116) | CT(I07)GCA | (SEQ ID |
| | | | (SEQ ID NO: 117) | NO: 118) |

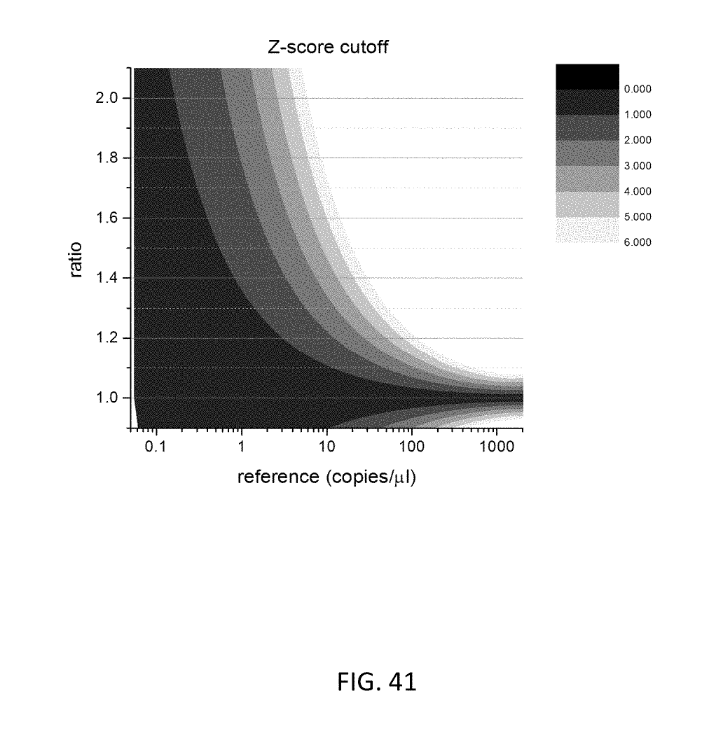

A numerical analysis was performed to determine the minimum input requirements for a 20,000 partition digital PCR experiment. This analysis examined the ability to detect a 2-fold difference in concentration between a target gene and a reference gene within a tumor population for a sample with various levels of tumor burden. Results of the numerical analysis are shown in FIG. 39. The upper and lower bounds of significance ensuring a p-value of <0.0001 (z-score≥3.891) were then determined at various input concentrations. A 2-fold difference in concentration between a target gene and a reference gene with a p-value of <0.0001 can be detected in a DNA sample originating from a tissue sample having 40% tumor burden, wherein the DNA sample comprises 20 copies/μL of RNaseP, corresponding to 0.06 ng/μL DNA (FIG. 41). Similarly, a 2-fold difference in concentration between a target gene and a reference gene with a p-value of <0.0001 can be detected in a DNA sample originating from a tissue sample having 20% tumor burden, wherein the DNA sample comprises 50 copies/μL of RNaseP, corresponding to 0.15 ng/μL DNA. Since 2.2 μL of sample is introduced per 22 μL assay volume, it is estimated that the CNV ddPCR assay can detect a gene amplification from as little as 0.6 ng/μL of purified FPET DNA material.

The CNV assay assigns a target gene i as “not amplified” if the expected values of the target gene μᵢ is the same as the expected value of the reference gene μⱼ:

H₀:μᵢ=μⱼ

If the null hypothesis is not satisfied, the target gene i is assigned as “amplified”. However, as the number of positive and negative counts follow a binomial distribution, the criteria for acceptance can be evaluated by application of a t-test to the proportion of negative droplets p_(i,neg) and p_(j,neg) from target gene i and reference gene j, respectively, to derive a standard (zi) score:

If the null hypothesis is not satisfied, the target gene i is assigned as “amplified”. However, as the number of positive and negative counts follow a binomial distribution, the criteria for acceptance can be evaluated by application of a t-test to the proportion of negative droplets P_{i,neg} and P_{j,neg} from target gene i and reference gene j, respectively, to derive a standard (zᵢ) score:

$${z}_{i} = \frac{{p}_{i , neg} - {\overline{p}}_{j , neg}}{\sqrt{{σ}_{i , neg}^{2} + {\overline{σ}}_{j , neg}^{2}}}$$

$${z}_{i} = \frac{{p}_{i , neg} - {\overline{p}}_{j , neg}}{\sqrt{{σ}_{i , neg}^{2} + {\overline{σ}}_{j , neg}^{2}}}$$

If the standard score zᵢ≥3.891, then the target gene i is “amplified” at a p<0.0001 (i.e., 99.99% CI)

For the BRAF gene, the assay is designed to a region on the BRAF gene on chromosome 7 that has an off-target homology to a region on the X chromosome. Thus, the total concentration of BRAF observed is a contribution of both targets:

C_{BRAF,tot}=C_{BRAF,chr7}+C_{BRAF,chrX}

C_{BRAF,tot}=m·C_{ref}+n·C_{ref}

C_{BRAF,tot}=(m+n)·C_{ref}

C_{BRAF,tot}=C_{BRAF,chr7}+C_{BRAF,chrX}

C_{BRAF,tot}=m·C_{ref}+n·C_{ref}

C_{BRAF,tot}=(m+n)·C_{ref}

where m represents the fold-amplification versus the reference value, and n represents the number of copies on the X-chromosome. This can be related to the expected values in Poisson space:

$$- \frac{1000}{V} ln ( {p}_{neg , BRAF} ) = - \frac{1000}{V} ( m + n ) ln ( {p}_{neg , ref} )$$

$$ln ( {p}_{neg , BRAF} ) = ( m + n ) ln ( {p}_{neg , ref} )$$

$${p}_{neg , BRAF} = {p}_{neg , ref}^{m + n}$$

For a “normal” sample, m=1. Due to the presence of a pseudogene for BRAF on the X-chromosome, n=0.5 for male, n=1 for female. Therefore, the expected “normal” value of BRAF occurs when 1+n=1.5 or 2.0

If the standard score zᵢ is ≥3.891, then the target gene i is “amplified” at a p<0.0001 (i.e., 99.99% CI)

### Example 20: Use of CNV ddPCR Panel for Selecting Effective Cancer Treatment

A patient presented with metastatic colon cancer. The colon cancer had metastasized to the patient's liver. Five different types of chemotherapy treatments had been attempted without success. A liver biopsy suspected of containing cancerous tissue was obtained from the patient and fresh frozen. DNA was extracted from the liver biopsy and quantitated. Sample DNA from the patient was then subjected to ddPCR using the primer/probe sets for VEGFA, EGFR, CDK6, MET, BRAF, FGFR1, JAK2, HER3, CDK4, HER2, SRC, and AURKA, outlined in Table 16 (above). PCR thermocycler conditions were as follows: 10 minutes at 95° C. (100% ramp rate), followed by 45 cycles of (30 seconds at 95° C., 60 seconds at 60° C.) followed by 5 minutes at 70° C., followed by 25° C. hold. Droplets were enumerated by Quantasoft. The concentration of target and reference genes were calculated using the following equation for each gene i:

$${p}_{i , neg} = \frac{{N}_{i , neg}}{{N}_{i , tot}} , {σ}_{i , neg} = \sqrt{\frac{{N}_{i , neg}}{{N}_{i , tot}} ( 1 - \frac{{N}_{i , neg}}{{N}_{i , tot}} ) / {N}_{i , tot}} \\ ∴ {p}_{i , neg , 99.99 \% CI} = {p}_{i , neg} \pm 3.891 \cdot {σ}_{i , neg}$$

$${p}_{i , neg} = \frac{{N}_{i , neg}}{{N}_{i , tot}} , {σ}_{i , neg} = \sqrt{\frac{{N}_{i , neg}}{{N}_{i , tot}} ( 1 - \frac{{N}_{i , neg}}{{N}_{i , tot}} ) / {N}_{i , tot}} \\ ∴ {p}_{i , neg , 99.99 \% CI} = {p}_{i , neg} \pm 3.891 \cdot {σ}_{i , neg}$$

where p_{i,neg} is the proportion of negative droplets, where N_{i,neg} is the number of negative events, σ_{i,neg} is the standard deviation of the proportion measurement, N_{t,tot} is the number of accepted events for each gene i as determined by QuantaSoft, and [symbol] p [symbol] _(i,neg, 99.99% CI) is the lower and upper bound of the proportion measurement. concentration of each species c was converted to concentration units (copies/μL) according to the following relationship:

$$c = - \frac{1000}{V} ln ( {p}_{neg} )$$

where V represents the volume of the partition/droplet.

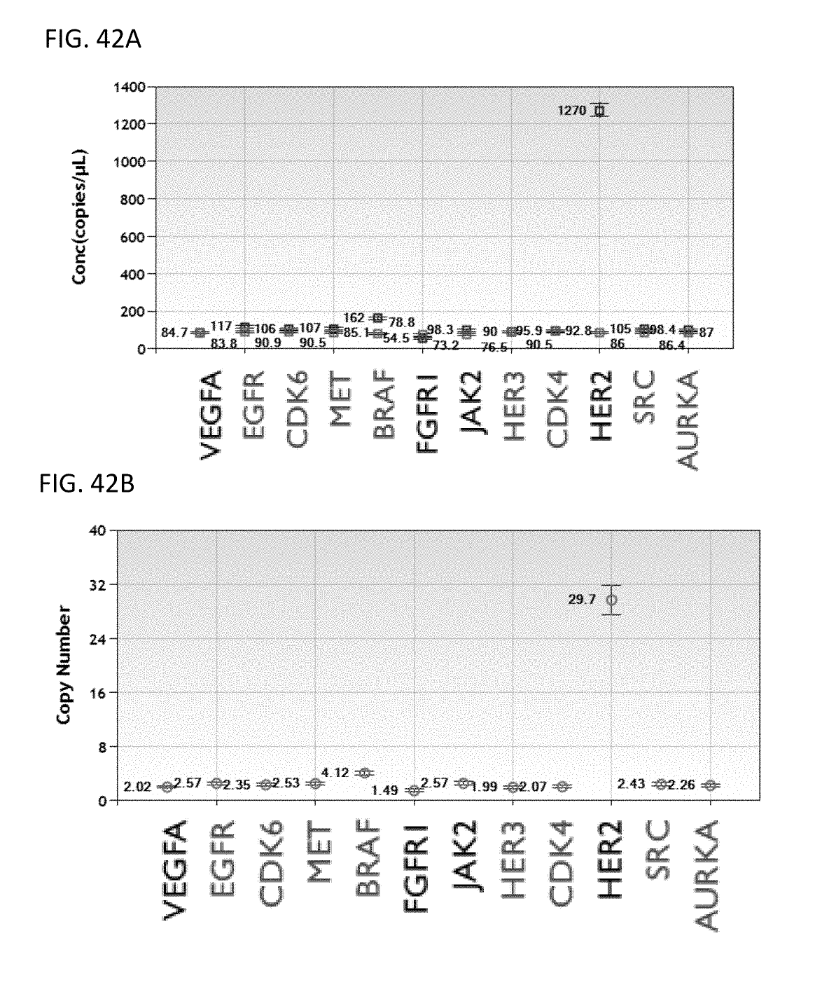

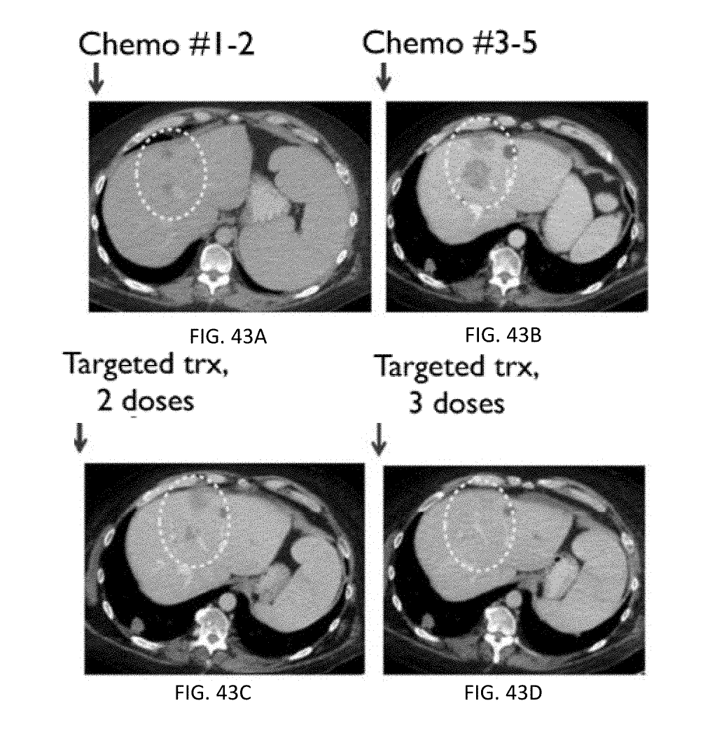

Results from the CNV ddPCR assay are shown in FIGS. 42A-42B. FIG. 42A depicts concentration of 12 of the CNV cancer genes, and FIG. 42B depicts copy number of the 12 genes in the patient sample. A dramatic amplification of the HER2 gene was revealed by the CNV ddPCR assay. The HER2 amplification was reported to the patient's doctor. Based on the results of the CNV ddPCR assay, the doctor prescribed the breast cancer drug T-DMI. FIGS. 43A-43B depict image scans of the patient's liver taken after chemotherapy treatment regimens 1 and 2 (FIG. 43A), taken after chemotherapy treatment regimens 3-5 (FIG. 43B), taken after the patient received two doses of the T-DMI (FIG. 43C), and taken after the patient received the third dose of T-DMI (FIG. 43D). FIG. 43A reveals two dark spots in the liver, indicative of cancerous tissue. FIG. 43B reveals that despite chemotherapy regimens 3-5, the cancerous growths increased dramatically in size. FIG. 43C reveals that after two doses of T-DMI, the cancerous growths had shrunk by at least ˜50%. FIG. 43D reveals that after the third dose of T-DMI, the cancerous growths were undetectable by image scan.

### Example 21: Detection of Copy Number Variation and Gene Mutation Using a Single Assay

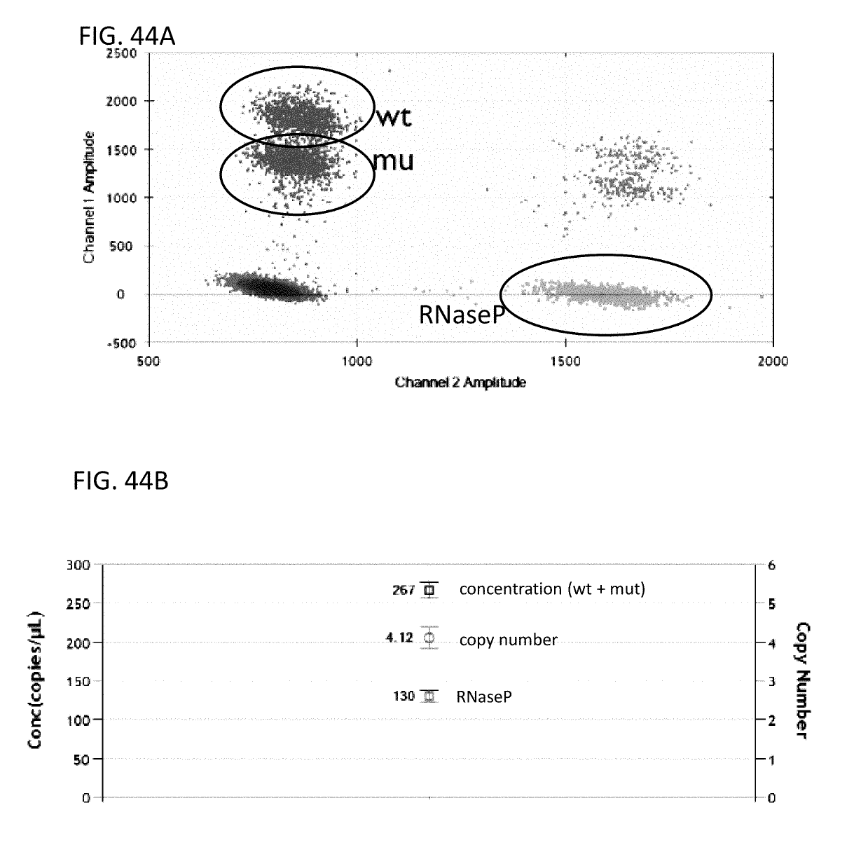

The CNV primer/probe set for EGFR as depicted in Table 1 was used to assay both copy number variation and the presence of mutant EGFR in a cancer patient sample. The EGFR probe overlays a site known to harbor a cancer-related mutation and has a sequence corresponding to the wild-type allele. ddPCR was conducted as described herein (see, e.g., Example 20). FIG. 44A depicts results of the assay. Because of a mismatch between the EGFR probe and the mutant allele, the probe had lower binding efficiency to the mutant allele, resulting in a cluster of ddPCR droplets with distinguishably lower fluorescence intensity. FIG. 44B depicts quantitation results from the assay. The high-intensity cluster of EGFR positive droplets were enumerated as wild-type, the low-intensity cluster of EGFR positive droplets were enumerated as mutant. The sample was determined to contain 267 copies/μl total EGFR (wt+mu), with an equal proportion of wt and mu EGFR. EGFR also exhibited a 2-fold gene amplification from 2 to 4.12.

What is claimed is:

1-316. (canceled)

317. A method, comprising:
a. ligating a first single-stranded adaptor to a single-stranded nucleic acid fragment, thereby generating a single-stranded nucleic acid fragment ligated to said first singled-stranded adaptor, wherein an efficiency of said ligating is at least 10%, 30%, 50%, 70%, or 90%;
b. hybridizing a target-selective oligonucleotide to said single-stranded nucleic acid fragment ligated to said first single-stranded adaptor to create a hybridization product, wherein said target-selective oligonucleotide comprises (i) a sequence that is complementary to said single-stranded nucleic acid fragment; and (ii) a second single-stranded adaptor sequence located at a first end of said target-selective oligonucleotide; and
c. extending said target-selective oligonucleotide in said hybridization product to create an extension product.

318. The method of claim 317, wherein said efficiency of said ligating is at least 30%.

319. The method of claim 317, wherein said efficiency of said ligating is at least 50%.

320. The method of claim 317, wherein said ligating comprises ligating said first single-stranded adaptor to said single-stranded nucleic acid fragment using an ATP-dependent ligase.

321. The method of claim 317, wherein said ligating comprises ligating said first single-stranded adaptor to said single-stranded nucleic acid fragment using an RNA ligase.

322. The method of claim 317, wherein said ligating comprises ligating a 3′ end of said first single-stranded adaptor to a 5′ end of said single stranded nucleic acid fragment.

323. The method of claim 317, wherein said single-stranded nucleic acid fragment comprises a 3′ terminal blocking group.

324. The method of claim 323, wherein said terminal blocking group comprises a dideoxy-NTP.

325. The method of claim 317, further comprising adding a 3′ terminal blocking group to said single-stranded nucleic acid fragment using a terminal nucleotidyl transferase.

326. The method of claim 317, wherein said single-stranded nucleic acid fragment comprises a 5′ nucleoside monophosphate.

327. The method of claim 317, further comprising phosphorylating a 5′ end of said single-stranded nucleic acid fragment.

328. The method of claim 327, wherein said phosphorylating comprises use of T4 polynucleotide kinase.

329. The method of claim 317, wherein said single-stranded nucleic acid fragment comprises genomic DNA or cDNA.

330. The method of claim 317, wherein said single-stranded nucleic acid fragment comprises RNA.

331. The method of claim 317, wherein said single-stranded nucleic acid fragment is from a biological source.

332. The method of claim 317, wherein said single-stranded nucleic acid fragment and said target-specific oligonucleotide are free-floating in a reaction mixture.

333. The method of claim 317, further comprising, before said ligating, denaturing a double-stranded nucleic acid fragment to generate said single-stranded nucleic acid fragment.

334. The method of claim 317, further comprising sequencing said extension product.

335. A method of preparing a single-stranded DNA library, comprising:
a. denaturing a double stranded DNA fragment into single stranded DNA (ssDNA) fragments;
b. ligating a first single-stranded adaptor sequence to a first end of said ssDNA fragments; and
c. ligating a second single-stranded adaptor sequence to a second end of said ssDNA fragments.

336. A kit, comprising:
a. a first adaptor oligonucleotide, wherein said first adaptor oligonucleotide comprises a sequence that is at least 70% complementary to a first support-bound oligonucleotide coupled to a sequencing platform;
b. a second adaptor oligonucleotide, wherein said second adaptor oligonucleotide comprises a sequence that is distinct from said first adaptor oligonucleotide;
c. an RNA ligase; and
d. instructions for use.
